SEQUENCE LISTING (635 sequences)

SEQ ID NO: 1 - protein, 14 aa, Artificial Sequence
  Myostatin Binding Peptide
KDKCKMWHWMCKPP

SEQ ID NO: 2 - protein, 14 aa, Artificial Sequence
  Myostatin Binding Peptide
KDLCAMWHWMCKPP

SEQ ID NO: 3 - protein, 13 aa, Artificial Sequence
  Myostatin Binding Peptide
DLCKMWKWMCKPP

SEQ ID NO: 4 - protein, 14 aa, Artificial Sequence
  Myostatin Binding Peptide
KDLCKMWHWMCKPK

SEQ ID NO: 5 - protein, 14 aa, Artificial Sequence
  Myostatin Binding Peptide
WYPCYEFHFWCYDL

SEQ ID NO: 6 - protein, 14 aa, Artificial Sequence
  Myostatin Binding Peptide
WYPCYEGHFWCYDL

SEQ ID NO: 7 - protein, 14 aa, Artificial Sequence
  Myostatin Binding Peptide
IFGCKWWDVQCYQF

SEQ ID NO: 8 - protein, 14 aa, Artificial Sequence
  Myostatin Binding Peptide
IFGCKWWDVDCYQF

SEQ ID NO: 9 - protein, 14 aa, Artificial Sequence
  Myostatin Binding Peptide
ADWCVSPNWFCMVM

SEQ ID NO: 10 - protein, 14 aa, Artificial Sequence
  Myostatin Binding Peptide
HKFCPWWALFCWDF

SEQ ID NO: 11 - protein, 14 aa, Artificial Sequence
  Myostatin Binding Peptide
KDLCKMWHWMCKPP

SEQ ID NO: 12 - protein, 14 aa, Artificial Sequence
  Myostatin Binding Peptide
IDKCAIWGWMCPPL

SEQ ID NO: 13 - protein, 14 aa, Artificial Sequence
  Myostatin Binding Peptide
WYPCGEFGMWCLNV

SEQ ID NO: 14 - protein, 11 aa, Artificial Sequence
  Myostatin Binding Peptide
WFTCLWNCDNE

SEQ ID NO: 15 - protein, 14 aa, Artificial Sequence
  Myostatin Binding Peptide
HTPCPWFAPLCVEW

SEQ ID NO: 16 - protein, 14 aa, Artificial Sequence
  Myostatin Binding Peptide
KEWCWRWKWMCKPE

SEQ ID NO: 17 - protein, 14 aa, Artificial Sequence
  Myostatin Binding Peptide
FETCPSWAYFCLDI

SEQ ID NO: 18 - protein, 14 aa, Artificial Sequence
  Myostatin Binding Peptide
AYKCEANDWGCWWL

SEQ ID NO: 19 - protein, 14 aa, Artificial Sequence
  Myostatin Binding Peptide
NSWCEDQWHRCWWL

SEQ ID NO: 20 - protein, 14 aa, Artificial Sequence
  Myostatin Binding Peptide
WSACYAGHFWCYDL

SEQ ID NO: 21 - protein, 14 aa, Artificial Sequence
  Myostatin Binding Peptide
ANWCVSPNWFCMVM

SEQ ID NO: 22 - protein, 14 aa, Artificial Sequence
  Myostatin Binding Peptide
WTECYQQEFWCWNL

SEQ ID NO: 23 - protein, 14 aa, Artificial Sequence
  Myostatin Binding Peptide
ENTCERWKWMCPPK

SEQ ID NO: 24 - protein, 14 aa, Artificial Sequence
  Myostatin Binding Peptide
WLPCHQEGFWCMNF

SEQ ID NO: 25 - protein, 14 aa, Artificial Sequence
  Myostatin Binding Peptide
STMCSQWHWMCNPF

SEQ ID NO: 26 - protein, 14 aa, Artificial Sequence
  Myostatin Binding Peptide
IFGCHWWDVDCYQF

SEQ ID NO: 27 - protein, 14 aa, Artificial Sequence
  Myostatin Binding Peptide
IYGCKWWDIQCYDI

SEQ ID NO: 28 - protein, 14 aa, Artificial Sequence
  Myostatin Binding Peptide
PDWCIDPDWWCKFW

SEQ ID NO: 29 - protein, 14 aa, Artificial Sequence
  Myostatin Binding Peptide
QGHCTRWPWMCPPY

SEQ ID NO: 30 - protein, 14 aa, Artificial Sequence
  Myostatin Binding Peptide
WQECYREGFWCLQT

SEQ ID NO: 31 - protein, 14 aa, Artificial Sequence
  Myostatin Binding Peptide
WFDCYGPGFKCWSP

SEQ ID NO: 32 - protein, 14 aa, Artificial Sequence
  Myostatin Binding Peptide
GVRCPKGHLWCLYP

SEQ ID NO: 33 - protein, 14 aa, Artificial Sequence
  Myostatin Binding Peptide
HWACGYWPWSCKWV

SEQ ID NO: 34 - protein, 14 aa, Artificial Sequence
  Myostatin Binding Peptide
GPACHSPWWWCVFG

SEQ ID NO: 35 - protein, 14 aa, Artificial Sequence
  Myostatin Binding Peptide
TTWCISPMWFCSQQ

SEQ ID NO: 36 - protein, 14 aa, Artificial Sequence
  Myostatin Binding Peptide
HKFCPPWAIFCWDF

SEQ ID NO: 37 - protein, 14 aa, Artificial Sequence
  Myostatin Binding Peptide
PDWCVSPRWYCNMW

SEQ ID NO: 38 - protein, 14 aa, Artificial Sequence
  Myostatin Binding Peptide
VWKCHWFGMDCEPT

SEQ ID NO: 39 - protein, 14 aa, Artificial Sequence
  Myostatin Binding Peptide
KKHCQIWTWMCAPK

SEQ ID NO: 40 - protein, 14 aa, Artificial Sequence
  Myostatin Binding Peptide
WFQCGSTLFWCYNL

SEQ ID NO: 41 - protein, 14 aa, Artificial Sequence
  Myostatin Binding Peptide
WSPCYDHYFYCYTI

SEQ ID NO: 42 - protein, 14 aa, Artificial Sequence
  Myostatin Binding Peptide
SWMCGFFKEVCMWV

SEQ ID NO: 43 - protein, 14 aa, Artificial Sequence
  Myostatin Binding Peptide
EMLCMIHPVFCNPH

SEQ ID NO: 44 - protein, 14 aa, Artificial Sequence
  Myostatin Binding Peptide
LKTCNLWPWMCPPL

SEQ ID NO: 45 - protein, 14 aa, Artificial Sequence
  Myostatin Binding Peptide
VVGCKWYEAWCYNK

SEQ ID NO: 46 - protein, 14 aa, Artificial Sequence
  Myostatin Binding Peptide
PIHCTQWAWMCPPT

SEQ ID NO: 47 - protein, 14 aa, Artificial Sequence
  Myostatin Binding Peptide
DSNCPWYFLSCVIF

SEQ ID NO: 48 - protein, 14 aa, Artificial Sequence
  Myostatin Binding Peptide
HIWCNLAMMKCVEM

SEQ ID NO: 49 - protein, 14 aa, Artificial Sequence
  Myostatin Binding Peptide
NLQCIYFLGKCIYF

SEQ ID NO: 50 - protein, 14 aa, Artificial Sequence
  Myostatin Binding Peptide
AWRCMWFSDVCTPG

SEQ ID NO: 51 - protein, 14 aa, Artificial Sequence
  Myostatin Binding Peptide
WFRCFLDADWCTSV

SEQ ID NO: 52 - protein, 14 aa, Artificial Sequence
  Myostatin Binding Peptide
EKICQMWSWMCAPP

SEQ ID NO: 53 - protein, 14 aa, Artificial Sequence
  Myostatin Binding Peptide
WFYCHLNKSECTEP

SEQ ID NO: 54 - protein, 14 aa, Artificial Sequence
  Myostatin Binding Peptide
FWRCAIGIDKCKRV

SEQ ID NO: 55 - protein, 14 aa, Artificial Sequence
  Myostatin Binding Peptide
NLGCKWYEVWCFTY

SEQ ID NO: 56 - protein, 14 aa, Artificial Sequence
  Myostatin Binding Peptide
IDLCNMWDGMCYPP

SEQ ID NO: 57 - protein, 14 aa, Artificial Sequence
  Myostatin Binding Peptide
EMPCNIWGWMCPPV

SEQ ID NO: 58 - protein, 18 aa, Artificial Sequence
  Myostatin Binding Peptide
WFRCVLTGIVDWSECFGL

SEQ ID NO: 59 - protein, 18 aa, Artificial Sequence
  Myostatin Binding Peptide
WFRCVLTGIVDWSECFGL

SEQ ID NO: 60 - protein, 18 aa, Artificial Sequence
  Myostatin Binding Peptide
LPWCHDQVNADWGFCMLW

SEQ ID NO: 61 - protein, 18 aa, Artificial Sequence
  Myostatin Binding Peptide
YPTCSEKFWIYGQTCVLW

SEQ ID NO: 62 - protein, 18 aa, Artificial Sequence
  Myostatin Binding Peptide
LGPCPIHHGPWPQYCVYW

SEQ ID NO: 63 - protein, 18 aa, Artificial Sequence
  Myostatin Binding Peptide
PFPCETHQISWLGHCLSF

SEQ ID NO: 64 - protein, 18 aa, Artificial Sequence
  Myostatin Binding Peptide
HWGCEDLMWSWHPLCRRP

SEQ ID NO: 65 - protein, 18 aa, Artificial Sequence
  Myostatin Binding Peptide
LPLCDADMMPTIGFCVAY

SEQ ID NO: 66 - protein, 18 aa, Artificial Sequence
  Myostatin Binding Peptide
SHWCETTFWMNYAKCVHA

SEQ ID NO: 67 - protein, 18 aa, Artificial Sequence
  Myostatin Binding Peptide
LPKCTHVPFDQGGFCLWY

SEQ ID NO: 68 - protein, 18 aa, Artificial Sequence
  Myostatin Binding Peptide
FSSCWSPVSRQDMFCVFY

SEQ ID NO: 69 - protein, 17 aa, Artificial Sequence
  Myostatin Binding Peptide
SHKCEYSGWLQPLCYRP

SEQ ID NO: 70 - protein, 18 aa, Artificial Sequence
  Myostatin Binding Peptide
PWWCQDNYVQHMLHCDSP

SEQ ID NO: 71 - protein, 18 aa, Artificial Sequence
  Myostatin Binding Peptide
WFRCMLMNSFDAFQCVSY

SEQ ID NO: 72 - protein, 18 aa, Artificial Sequence
  Myostatin Binding Peptide
PDACRDQPWYMFMGCMLG

SEQ ID NO: 73 - protein, 14 aa, Artificial Sequence
  Myostatin Binding Peptide
FLACFVEFELCFDS

SEQ ID NO: 74 - protein, 18 aa, Artificial Sequence
  Myostatin Binding Peptide
SAYCIITESDPYVLCVPL

SEQ ID NO: 75 - protein, 18 aa, Artificial Sequence
  Myostatin Binding Peptide
PSICESYSTMWLPMCQHN

SEQ ID NO: 76 - protein, 18 aa, Artificial Sequence
  Myostatin Binding Peptide
WLDCHDDSWAWTKMCRSH

SEQ ID NO: 77 - protein, 18 aa, Artificial Sequence
  Myostatin Binding Peptide
YLNCVMMNTSPFVECVFN

SEQ ID NO: 78 - protein, 18 aa, Artificial Sequence
  Myostatin Binding Peptide
YPWCDGFMIQQGITCMFY

SEQ ID NO: 79 - protein, 18 aa, Artificial Sequence
  Myostatin Binding Peptide
FDYCTWLNGFKDWKCWSR

SEQ ID NO: 80 - protein, 18 aa, Artificial Sequence
  Myostatin Binding Peptide
LPLCNLKEISHVQACVLF

SEQ ID NO: 81 - protein, 18 aa, Artificial Sequence
  Myostatin Binding Peptide
SPECAFARWLGIEQCQRD

SEQ ID NO: 82 - protein, 18 aa, Artificial Sequence
  Myostatin Binding Peptide
YPQCFNLHLLEWTECDWF

SEQ ID NO: 83 - protein, 18 aa, Artificial Sequence
  Myostatin Binding Peptide
RWRCEIYDSEFLPKCWFF

SEQ ID NO: 84 - protein, 14 aa, Artificial Sequence
  Myostatin Binding Peptide
LVGCDNVWHRCKLF

SEQ ID NO: 85 - protein, 18 aa, Artificial Sequence
  Myostatin Binding Peptide
AGWCHVWGEMFGMGCSAL

SEQ ID NO: 86 - protein, 18 aa, Artificial Sequence
  Myostatin Binding Peptide
HHECEWMARWMSLDCVGL

SEQ ID NO: 87 - protein, 18 aa, Artificial Sequence
  Myostatin Binding Peptide
FPMCGIAGMKDFDFCVWY

SEQ ID NO: 88 - protein, 18 aa, Artificial Sequence
  Myostatin Binding Peptide
RDDCTFWPEWLWKLCERP

SEQ ID NO: 89 - protein, 18 aa, Artificial Sequence
  Myostatin Binding Peptide
YNFCSYLFGVSKEACQLP

SEQ ID NO: 90 - protein, 18 aa, Artificial Sequence
  Myostatin Binding Peptide
AHWCEQGPWRYGNICMAY

SEQ ID NO: 91 - protein, 18 aa, Artificial Sequence
  Myostatin Binding Peptide
NLVCGKISAWGDEACARA

SEQ ID NO: 92 - protein, 18 aa, Artificial Sequence
  Myostatin Binding Peptide
HNVCTIMGPSMKWFCWND

SEQ ID NO: 93 - protein, 18 aa, Artificial Sequence
  Myostatin Binding Peptide
NDLCAMWGWRNTIWCQNS

SEQ ID NO: 94 - protein, 18 aa, Artificial Sequence
  Myostatin Binding Peptide
PPFCQNDNDMLQSLCKLL

SEQ ID NO: 95 - protein, 18 aa, Artificial Sequence
  Myostatin Binding Peptide
WYDCNVPNELLSGLCRLF

SEQ ID NO: 96 - protein, 18 aa, Artificial Sequence
  Myostatin Binding Peptide
YGDCDQNHWMWPFTCLSL

SEQ ID NO: 97 - protein, 18 aa, Artificial Sequence
  Myostatin Binding Peptide
GWMCHFDLHDWGATCQPD

SEQ ID NO: 98 - protein, 18 aa, Artificial Sequence
  Myostatin Binding Peptide
YFHCMFGGHEFEVHCESF

SEQ ID NO: 99 - protein, 12 aa, Artificial Sequence
  Myostatin Binding Peptide
AYWCWHGQCVRF

SEQ ID NO: 100 - protein, 19 aa, Artificial Sequence
  Myostatin Binding Peptide
SEHWTFTDWDGNEWWVRPF

SEQ ID NO: 101 - protein, 20 aa, Artificial Sequence
  Myostatin Binding Peptide
MEMLDSLFELLKDMVPISKA

SEQ ID NO: 102 - protein, 19 aa, Artificial Sequence
  Myostatin Binding Peptide
SPPEEALMEWLGWQYGKFT

SEQ ID NO: 103 - protein, 20 aa, Artificial Sequence
  Myostatin Binding Peptide
SPENLLNDLYILMTKQEWYG

SEQ ID NO: 104 - protein, 20 aa, Artificial Sequence
  Myostatin Binding Peptide
FHWEEGIPFHVVTPYSYDRM

SEQ ID NO: 105 - protein, 19 aa, Artificial Sequence
  Myostatin Binding Peptide
KRLLEQFMNDLAELVSGHS

SEQ ID NO: 106 - protein, 20 aa, Artificial Sequence
  Myostatin Binding Peptide
DTRDALFQEFYEFVRSRLVI

SEQ ID NO: 107 - protein, 20 aa, Artificial Sequence
  Myostatin Binding Peptide
RMSAAPRPLTYRDIMDQYWH

SEQ ID NO: 108 - protein, 20 aa, Artificial Sequence
  Myostatin Binding Peptide
NDKAHFFEMFMFDVHNFVES

SEQ ID NO: 109 - protein, 20 aa, Artificial Sequence
  Myostatin Binding Peptide
QTQAQKIDGLWELLQSIRNQ

SEQ ID NO: 110 - protein, 18 aa, Artificial Sequence
  Myostatin Binding Peptide
MLSEFEEFLGNLVHRQEA

SEQ ID NO: 111 - protein, 20 aa, Artificial Sequence
  Myostatin Binding Peptide
YTPKMGSEWTSFWHNRIHYL

SEQ ID NO: 112 - protein, 20 aa, Artificial Sequence
  Myostatin Binding Peptide
LNDTLLRELKMVLNSLSDMK

SEQ ID NO: 113 - protein, 20 aa, Artificial Sequence
  Myostatin Binding Peptide
FDVERDLMRWLEGFMQSAAT

SEQ ID NO: 114 - protein, 20 aa, Artificial Sequence
  Myostatin Binding Peptide
HHGWNYLRKGSAPQWFEAWV

SEQ ID NO: 115 - protein, 20 aa, Artificial Sequence
  Myostatin Binding Peptide
VESLHQLQMWLDQKLASGPH

SEQ ID NO: 116 - protein, 18 aa, Artificial Sequence
  Myostatin Binding Peptide
RATLLKDFWQLVEGYGDN

SEQ ID NO: 117 - protein, 16 aa, Artificial Sequence
  Myostatin Binding Peptide
EELLREFYRFVSAFDY

SEQ ID NO: 118 - protein, 20 aa, Artificial Sequence
  Myostatin Binding Peptide
GLLDEFSHFIAEQFYQMPGG

SEQ ID NO: 119 - protein, 20 aa, Artificial Sequence
  Myostatin Binding Peptide
YREMSMLEGLLDVLERLQHY

SEQ ID NO: 120 - protein, 20 aa, Artificial Sequence
  Myostatin Binding Peptide
HNSSQMLLSELIMLVGSMMQ

SEQ ID NO: 121 - protein, 20 aa, Artificial Sequence
  Myostatin Binding Peptide
WREHFLNSDYIRDKLIAIDG

SEQ ID NO: 122 - protein, 19 aa, Artificial Sequence
  Myostatin Binding Peptide
QFPFYVFDDLPAQLEYWIA

SEQ ID NO: 123 - protein, 20 aa, Artificial Sequence
  Myostatin Binding Peptide
EFFHWLHNHRSEVNHWLDMN

SEQ ID NO: 124 - protein, 19 aa, Artificial Sequence
  Myostatin Binding Peptide
EALFQNFFRDVLTLSEREY

SEQ ID NO: 125 - protein, 20 aa, Artificial Sequence
  Myostatin Binding Peptide
QYWEQQWMTYFRENGLHVQY

SEQ ID NO: 126 - protein, 20 aa, Artificial Sequence
  Myostatin Binding Peptide
NQRMMLEDLWRIMTPMFGRS

SEQ ID NO: 127 - protein, 20 aa, Artificial Sequence
  Myostatin Binding Peptide
FLDELKAELSRHYALDDLDE

SEQ ID NO: 128 - protein, 20 aa, Artificial Sequence
  Myostatin Binding Peptide
GKLIEGLLNELMQLETFMPD

SEQ ID NO: 129 - protein, 15 aa, Artificial Sequence
  Myostatin Binding Peptide
ILLLDEYKKDWKSWF

SEQ ID NO: 130 - protein, 50 aa, Artificial Sequence
  Myostatin Binding Peptide
QGHCTRWPWMCPPYGSGSATGGSGSTASSGSGSATGQGHCTRWPWMCPPY

SEQ ID NO: 131 - protein, 43 aa, Artificial Sequence
  Myostatin Binding Peptide
WYPCYEGHFWCYDLGSGSTASSGSGSATGWYPCYEGHFWCYDL

SEQ ID NO: 132 - protein, 50 aa, Artificial Sequence
  Myostatin Binding Peptide
HTPCPWFAPLCVEWGSGSATGGSGSTASSGSGSATGHTPCPWFAPLCVEW

SEQ ID NO: 133 - protein, 50 aa, Artificial Sequence
  Myostatin Binding Peptide
PDWCIDPDWWCKFWGSGSATGGSGSTASSGSGSATGPDWCIDPDWWCKFW

SEQ ID NO: 134 - protein, 50 aa, Artificial Sequence
  Myostatin Binding Peptide
ANWCVSPNWFCMVMGSGSATGGSGSTASSGSGSATGANWCVSPNWFCMVM

SEQ ID NO: 135 - protein, 50 aa, Artificial Sequence; identical to SEQ ID NO: 133
  Myostatin Binding Peptide

SEQ ID NO: 136 - protein, 50 aa, Artificial Sequence
  Myostatin Binding Peptide
HWACGYWPWSCKWVGSGSATGGSGSTASSGSGSATGHWACGYWPWSCKWV

SEQ ID NO: 137 - protein, 50 aa, Artificial Sequence
  Myostatin Binding Peptide
KKHCQIWTWMCAPKGSGSATGGSGSTASSGSGSATGQGHCTRWPWMCPPY

SEQ ID NO: 138 - protein, 50 aa, Artificial Sequence
  Myostatin Binding Peptide
QGHCTRWPWMCPPYGSGSATGGSGSTASSGSGSATGKKHCQIWTWMCAPK

SEQ ID NO: 139 - protein, 50 aa, Artificial Sequence; identical to SEQ ID NO: 137
  Myostatin Binding Peptide

SEQ ID NO: 140 - protein, 36 aa, Artificial Sequence
  Myostatin Binding Peptide
KKHCQIWTWMCAPKGGGGGGGGQGHCTRWPWMCPPY

SEQ ID NO: 141 - protein, 34 aa, Artificial Sequence
  Myostatin Binding Peptide
QGHCTRWPWMCPPYGGGGGGKKHCQIWTWMCAPK

SEQ ID NO: 142 - protein, 14 aa, Artificial Sequence
  Myostatin Binding Peptide
KDXCXXWHWMCKPX

SEQ ID NO: 143 - protein, 14 aa, Artificial Sequence
  Myostatin Binding Peptide
WXXCXXXGFWCLNV

SEQ ID NO: 144 - protein, 14 aa, Artificial Sequence
  Myostatin Binding Peptide
IXGCXWWDXXCYXX

SEQ ID NO: 145 - protein, 14 aa, Artificial Sequence
  Myostatin Binding Peptide
XXWCVSPXWFCXXX

SEQ ID NO: 146 - protein, 14 aa, Artificial Sequence
  Myostatin Binding Peptide
XXXCPWFAXXCVDW

SEQ ID NO: 147 - DNA, 42 bp, Artificial Sequence
  Nucleotide sequence encoding Myostatin Binding Peptide
aaagacaaatgcaaaatgtggcactggatgtgcaaaccgccg

SEQ ID NO: 148 - DNA, 42 bp, Artificial Sequence
  Nucleotide sequence encoding Myostatin Binding Peptide
aaagacctgtgcgctatgtggcactggatgtgcaaaccgccg

SEQ ID NO: 149 - DNA, 42 bp, Artificial Sequence
  Nucleotide sequence encoding Myostatin Binding Peptide
aaagacctgtgcaaaatgtggaaatggatgtgcaaaccgccg

SEQ ID NO: 150 - DNA, 42 bp, Artificial Sequence
  Nucleotide sequence encoding Myostatin Binding Peptide
aaagacctgtgcaaaatgtggcactggatgtgcaaaccgaaa

SEQ ID NO: 151 - DNA, 42 bp, Artificial Sequence
  Nucleotide sequence encoding Myostatin Binding Peptide
tggtacccgtgctacgaattccacttctggtgctacgacctg

SEQ ID NO: 152 - DNA, 42 bp, Artificial Sequence; identical to SEQ ID NO: 151
  Nucleotide sequence encoding Myostatin Binding Peptide

SEQ ID NO: 153 - DNA, 42 bp, Artificial Sequence
  Nucleotide sequence encoding Myostatin Binding Peptide
tggtacccgtgctacgaaggtcacttctggtgctacgacctg

SEQ ID NO: 154 - DNA, 42 bp, Artificial Sequence; identical to SEQ ID NO: 153
  Nucleotide sequence encoding Myostatin Binding Peptide

SEQ ID NO: 155 - DNA, 42 bp, Artificial Sequence
  Nucleotide sequence encoding Myostatin Binding Peptide
atcttcggttgcaaatggtgggacgttcagtgctaccagttc

SEQ ID NO: 156 - DNA, 42 bp, Artificial Sequence
  Nucleotide sequence encoding Myostatin Binding Peptide
atcttcggttgcaaatggtgggacgttgactgctaccagttc

SEQ ID NO: 157 - DNA, 42 bp, Artificial Sequence; identical to SEQ ID NO: 156
  Nucleotide sequence encoding Myostatin Binding Peptide

SEQ ID NO: 158 - DNA, 42 bp, Artificial Sequence
  Nucleotide sequence encoding Myostatin Binding Peptide
gctgactggtgcgtttccccgaactggttctgcatggttatg

SEQ ID NO: 159 - DNA, 42 bp, Artificial Sequence
  Nucleotide sequence encoding Myostatin Binding Peptide
cacaaattctgcccgtggtgggctctgttctgctgggacttc

SEQ ID NO: 160 - DNA, 42 bp, Artificial Sequence
  Nucleotide sequence encoding Myostatin Binding Peptide
aaagacctgtgcaaaatgtggcactggatgtgcaaaccgccg

SEQ ID NO: 161 - DNA, 42 bp, Artificial Sequence
  Nucleotide sequence encoding Myostatin Binding Peptide
atcgacaaatgcgctatctggggttggatgtgcccgccgctg

SEQ ID NO: 162 - DNA, 42 bp, Artificial Sequence
  Nucleotide sequence encoding Myostatin Binding Peptide
tggtacccgtgcggtgaattcggtatgtggtgcctgaacgtt

SEQ ID NO: 163 - DNA, 33 bp, Artificial Sequence
  Nucleotide sequence encoding Myostatin Binding Peptide
tggttcacctgcctgtggaactgcgacaacgaa

SEQ ID NO: 164 - DNA, 42 bp, Artificial Sequence
  Nucleotide sequence encoding Myostatin Binding Peptide
cacaccccgtgcccgtggttcgctccgctgtgcgttgaatgg

SEQ ID NO: 165 - DNA, 42 bp, Artificial Sequence
  Nucleotide sequence encoding Myostatin Binding Peptide
aaagaatggtgctggcgttggaaatggatgtgcaaaccggaa

SEQ ID NO: 166 - DNA, 42 bp, Artificial Sequence
  Nucleotide sequence encoding Myostatin Binding Peptide
ttcgaaacctgcccgtcctgggcttacttctgcctggacatc

SEQ ID NO: 167 - DNA, 42 bp, Artificial Sequence; identical to SEQ ID NO: 166
  Nucleotide sequence encoding Myostatin Binding Peptide

SEQ ID NO: 168 - DNA, 42 bp, Artificial Sequence
  Nucleotide sequence encoding Myostatin Binding Peptide
gcttacaaatgcgaagctaacgactggggttgctggtggctg

SEQ ID NO: 169 - DNA, 42 bp, Artificial Sequence
  Nucleotide sequence encoding Myostatin Binding Peptide
aactcctggtgcgaagaccagtggcaccgttgctggtggctg

SEQ ID NO: 170 - DNA, 42 bp, Artificial Sequence
  Nucleotide sequence encoding Myostatin Binding Peptide
tggtccgcttgctacgctggtcacttctggtgctacgacctg

SEQ ID NO: 171 - DNA, 42 bp, Artificial Sequence
  Nucleotide sequence encoding Myostatin Binding Peptide
gctaactggtgcgtttccccgaactggttctgcatggttatg

SEQ ID NO: 172 - DNA, 42 bp, Artificial Sequence
  Nucleotide sequence encoding Myostatin Binding Peptide
tggaccgaatgctaccagcaggaattctggtgctggaacctg

SEQ ID NO: 173 - DNA, 42 bp, Artificial Sequence
  Nucleotide sequence encoding Myostatin Binding Peptide
gaaaacacctgcgaacgttggaaatggatgtgcccgccgaaa

SEQ ID NO: 174 - DNA, 42 bp, Artificial Sequence
  Nucleotide sequence encoding Myostatin Binding Peptide
tggctgccgtgccaccaggaaggtttctggtgcatgaacttc

SEQ ID NO: 175 - DNA, 42 bp, Artificial Sequence
  Nucleotide sequence encoding Myostatin Binding Peptide
tccaccatgtgctcccagtggcactggatgtgcaacccgttc

SEQ ID NO: 176 - DNA, 42 bp, Artificial Sequence
  Nucleotide sequence encoding Myostatin Binding Peptide
atcttcggttgccactggtgggacgttgactgctaccagttc

SEQ ID NO: 177 - DNA, 42 bp, Artificial Sequence
  Nucleotide sequence encoding Myostatin Binding Peptide
atctacggttgcaaatggtgggacatccagtgctacgacatc

SEQ ID NO: 178 - DNA, 42 bp, Artificial Sequence
  Nucleotide sequence encoding Myostatin Binding Peptide
ccggactggtgcatcgatccggactggtggtgcaaattctgg

SEQ ID NO: 179 - DNA, 42 bp, Artificial Sequence
  Nucleotide sequence encoding Myostatin Binding Peptide
cagggtcactgcacccgttggccgtggatgtgcccgccgtac

SEQ ID NO: 180 - DNA, 42 bp, Artificial Sequence
  Nucleotide sequence encoding Myostatin Binding Peptide
tggcaggaatgctaccgtgaaggtttctggtgcctgcagacc

SEQ ID NO: 181 - DNA, 42 bp, Artificial Sequence
  Nucleotide sequence encoding Myostatin Binding Peptide
tggttcgactgctacggtccgggtttcaaatgctggtccccg

SEQ ID NO: 182 - DNA, 42 bp, Artificial Sequence
  Nucleotide sequence encoding Myostatin Binding Peptide
ggtgttcgttgcccgaaaggtcacctgtggtgcctgtacccg

SEQ ID NO: 183 - DNA, 42 bp, Artificial Sequence
  Nucleotide sequence encoding Myostatin Binding Peptide
cactgggcttgcggttactggccgtggtcctgcaaatgggtt

SEQ ID NO: 184 - DNA, 42 bp, Artificial Sequence
  Nucleotide sequence encoding Myostatin Binding Peptide
ggtccggcttgccactccccgtggtggtggtgcgttttcggt

SEQ ID NO: 185 - DNA, 42 bp, Artificial Sequence
  Nucleotide sequence encoding Myostatin Binding Peptide
accacctggtgcatctccccgatgtggttctgctcccagcag

SEQ ID NO: 186 - DNA, 42 bp, Artificial Sequence
  Nucleotide sequence encoding Myostatin Binding Peptide
cacaaattctgcccgccgtgggctatcttctgctgggacttc

SEQ ID NO: 187 - DNA, 42 bp, Artificial Sequence
  Nucleotide sequence encoding Myostatin Binding Peptide
ccggactggtgcgtttccccgcgttggtactgcaacatgtgg

SEQ ID NO: 188 - DNA, 42 bp, Artificial Sequence
  Nucleotide sequence encoding Myostatin Binding Peptide
gtttggaaatgccactggttcggtatggactgcgaaccgacc

SEQ ID NO: 189 - DNA, 42 bp, Artificial Sequence
  Nucleotide sequence encoding Myostatin Binding Peptide
aaaaaacactgccagatctggacctggatgtgcgctccgaaa

SEQ ID NO: 190 - DNA, 42 bp, Artificial Sequence
  Nucleotide sequence encoding Myostatin Binding Peptide
tggttccagtgcggttccaccctgttctggtgctacaacctg

SEQ ID NO: 191 - DNA, 42 bp, Artificial Sequence
  Nucleotide sequence encoding Myostatin Binding Peptide
tggtccccgtgctacgaccactacttctactgctacaccatc

SEQ ID NO: 192 - DNA, 42 bp, Artificial Sequence
  Nucleotide sequence encoding Myostatin Binding Peptide
tcctggatgtgcggtttcttcaaagaagtttgcatgtgggtt

SEQ ID NO: 193 - DNA, 42 bp, Artificial Sequence
  Nucleotide sequence encoding Myostatin Binding Peptide
gaaatgctgtgcatgatccacccggttttctgcaacccgcac

SEQ ID NO: 194 - DNA, 42 bp, Artificial Sequence
  Nucleotide sequence encoding Myostatin Binding Peptide
ctgaaaacctgcaacctgtggccgtggatgtgcccgccgctg

SEQ ID NO: 195 - DNA, 42 bp, Artificial Sequence
  Nucleotide sequence encoding Myostatin Binding Peptide
gttgttggttgcaaatggtacgaagcttggtgctacaacaaa

SEQ ID NO: 196 - DNA, 42 bp, Artificial Sequence
  Nucleotide sequence encoding Myostatin Binding Peptide
ccgatccactgcacccagtgggcttggatgtgcccgccgacc

SEQ ID NO: 197 - DNA, 42 bp, Artificial Sequence
  Nucleotide sequence encoding Myostatin Binding Peptide
gactccaactgcccgtggtacttcctgtcctgcgttatcttc

SEQ ID NO: 198 - DNA, 42 bp, Artificial Sequence; identical to SEQ ID NO: 197
  Nucleotide sequence encoding Myostatin Binding Peptide

SEQ ID NO: 199 - DNA, 42 bp, Artificial Sequence
  Nucleotide sequence encoding Myostatin Binding Peptide
aacctgcagtgcatctacttcctgggtaaatgcatctacttc

SEQ ID NO: 200 - DNA, 42 bp, Artificial Sequence
  Nucleotide sequence encoding Myostatin Binding Peptide
gcttggcgttgcatgtggttctccgacgtttgcaccccgggt

SEQ ID NO: 201 - DNA, 42 bp, Artificial Sequence
  Nucleotide sequence encoding Myostatin Binding Peptide
tggtttcgttgttttcttgatgctgattggtgtacttctgtt

SEQ ID NO: 202 - DNA, 42 bp, Artificial Sequence
  Nucleotide sequence encoding Myostatin Binding Peptide
gaaaaaatttgtcaaatgtggtcttggatgtgtgctccacca

SEQ ID NO: 203 - DNA, 42 bp, Artificial Sequence
  Nucleotide sequence encoding Myostatin Binding Peptide
tggttttattgtcatcttaataaatctgaatgtactgaacca

SEQ ID NO: 204 - DNA, 42 bp, Artificial Sequence
  Nucleotide sequence encoding Myostatin Binding Peptide
ttttggcgttgtgctattggtattgataaatgtaaacgtgtt

SEQ ID NO: 205 - DNA, 42 bp, Artificial Sequence; identical to SEQ ID NO: 204
  Nucleotide sequence encoding Myostatin Binding Peptide

SEQ ID NO: 206 - DNA, 42 bp, Artificial Sequence
  Nucleotide sequence encoding Myostatin Binding Peptide
attgatctttgtaatatgtgggatggtatgtgttatccacca

SEQ ID NO: 207 - DNA, 42 bp, Artificial Sequence
  Nucleotide sequence encoding Myostatin Binding Peptide
gaaatgccatgtaatatttggggttggatgtgtccaccagtt

SEQ ID NO: 208 - DNA, 54 bp, Artificial Sequence
  Nucleotide sequence encoding Myostatin Binding Peptide
tggttccgttgcgttctgaccggtatcgttgactggtccgaatgcttcggtctg

SEQ ID NO: 209 - DNA, 54 bp, Artificial Sequence
  Nucleotide sequence encoding Myostatin Binding Peptide
ggtttctcctgcaccttcggtctggacgaattctacgttgactgctccccgttc

SEQ ID NO: 210 - DNA, 54 bp, Artificial Sequence
  Nucleotide sequence encoding Myostatin Binding Peptide
ctgccgtggtgccacgaccaggttaacgctgactggggtttctgcatgctgtgg

SEQ ID NO: 211 - DNA, 54 bp, Artificial Sequence
  Nucleotide sequence encoding Myostatin Binding Peptide
tacccgacctgctccgaaaaattctggatctacggtcagacctgcgttctgtgg

SEQ ID NO: 212 - DNA, 54 bp, Artificial Sequence
  Nucleotide sequence encoding Myostatin Binding Peptide
ctgggtccgtgcccgatccaccacggtccgtggccgcagtactgcgtttactgg

SEQ ID NO: 213 - DNA, 54 bp, Artificial Sequence
  Nucleotide sequence encoding Myostatin Binding Peptide
ccgttcccgtgcgaaacccaccagatctcctggctgggtcactgcctgtccttc

SEQ ID NO: 214 - DNA, 54 bp, Artificial Sequence
  Nucleotide sequence encoding Myostatin Binding Peptide
cactggggttgcgaagacctgatgtggtcctggcacccgctgtgccgtcgtccg

SEQ ID NO: 215 - DNA, 54 bp, Artificial Sequence
  Nucleotide sequence encoding Myostatin Binding Peptide
ctgccgctgtgcgacgctgacatgatgccgaccatcggtttctgcgttgcttac

SEQ ID NO: 216 - DNA, 54 bp, Artificial Sequence
  Nucleotide sequence encoding Myostatin Binding Peptide
tcccactggtgcgaaaccaccttctggatgaactacgctaaatgcgttcacgct

SEQ ID NO: 217 - DNA, 54 bp, Artificial Sequence
  Nucleotide sequence encoding Myostatin Binding Peptide
ctgccgaaatgcacccacgttccgttcgaccagggtggtttctgcctgtggtac

SEQ ID NO: 218 - DNA, 54 bp, Artificial Sequence
  Nucleotide sequence encoding Myostatin Binding Peptide
ttctcctcctgctggtccccggtttcccgtcaggacatgttctgcgttttctac

SEQ ID NO: 219 - DNA, 51 bp, Artificial Sequence
  Nucleotide sequence encoding Myostatin Binding Peptide
tcccacaaatgcgaatactccggttggctgcagccgctgtgctaccgtccg

SEQ ID NO: 220 - DNA, 54 bp, Artificial Sequence
  Nucleotide sequence encoding Myostatin Binding Peptide
ccgtggtggtgccaggacaactacgttcagcacatgctgcactgcgactccccg

SEQ ID NO: 221 - DNA, 54 bp, Artificial Sequence
  Nucleotide sequence encoding Myostatin Binding Peptide
tggttccgttgcatgctgatgaactccttcgacgctttccagtgcgtttcctac

SEQ ID NO: 222 - DNA, 54 bp, Artificial Sequence
  Nucleotide sequence encoding Myostatin Binding Peptide
ccggacgcttgccgtgaccagccgtggtacatgttcatgggttgcatgctgggt

SEQ ID NO: 223 - DNA, 42 bp, Artificial Sequence
  Nucleotide sequence encoding Myostatin Binding Peptide
ttcctggcttgcttcgttgaattcgaactgtgcttcgactcc

SEQ ID NO: 224 - DNA, 54 bp, Artificial Sequence
  Nucleotide sequence encoding Myostatin Binding Peptide
tccgcttactgcatcatcaccgaatccgacccgtacgttctgtgcgttccgctg

SEQ ID NO: 225 - DNA, 54 bp, Artificial Sequence
  Nucleotide sequence encoding Myostatin Binding Peptide
ccgtccatctgcgaatcctactccaccatgtggctgccgatgtgccagcacaac

SEQ ID NO: 226 - DNA, 54 bp, Artificial Sequence
  Nucleotide sequence encoding Myostatin Binding Peptide
tggctggactgccacgacgactcctgggcttggaccaaaatgtgccgttcccac

SEQ ID NO: 227 - DNA, 54 bp, Artificial Sequence
  Nucleotide sequence encoding Myostatin Binding Peptide
tacctgaactgcgttatgatgaacacctccccgttcgttgaatgcgttttcaac

SEQ ID NO: 228 - DNA, 54 bp, Artificial Sequence
  Nucleotide sequence encoding Myostatin Binding Peptide
tacccgtggtgcgacggtttcatgatccagcagggtatcacctgcatgttctac

SEQ ID NO: 229 - DNA, 54 bp, Artificial Sequence
  Nucleotide sequence encoding Myostatin Binding Peptide
ttcgactactgcacctggctgaacggtttcaaagactggaaatgctggtcccgt

SEQ ID NO: 230 - DNA, 54 bp, Artificial Sequence
  Nucleotide sequence encoding Myostatin Binding Peptide
ctgccgctgtgcaacctgaaagaaatctcccacgttcaggcttgcgttctgttc

SEQ ID NO: 231 - DNA, 54 bp, Artificial Sequence
  Nucleotide sequence encoding Myostatin Binding Peptide
tccccggaatgcgctttcgctcgttggctgggtatcgaacagtgccagcgtgac

SEQ ID NO: 232 - DNA, 54 bp, Artificial Sequence
  Nucleotide sequence encoding Myostatin Binding Peptide
tacccgcagtgcttcaacctgcacctgctggaatggaccgaatgcgactggttc

SEQ ID NO: 233 - DNA, 54 bp, Artificial Sequence
  Nucleotide sequence encoding Myostatin Binding Peptide
cgttggcgttgcgaaatctacgactccgaattcctgccgaaatgctggttcttc

SEQ ID NO: 234 - DNA, 42 bp, Artificial Sequence
  Nucleotide sequence encoding Myostatin Binding Peptide
ctggttggttgcgacaacgtttggcaccgttgcaaactgttc

SEQ ID NO: 235 - DNA, 54 bp, Artificial Sequence
  Nucleotide sequence encoding Myostatin Binding Peptide
gctggttggtgccacgtttggggtgaaatgttcggtatgggttgctccgctctg

SEQ ID NO: 236 - DNA, 54 bp, Artificial Sequence
  Nucleotide sequence encoding Myostatin Binding Peptide
caccacgaatgcgaatggatggctcgttggatgtccctggactgcgttggtctg

SEQ ID NO: 237 - DNA, 54 bp, Artificial Sequence
  Nucleotide sequence encoding Myostatin Binding Peptide
ttcccgatgtgcggtatcgctggtatgaaagacttcgacttctgcgtttggtac

SEQ ID NO: 238 - DNA, 54 bp, Artificial Sequence
  Nucleotide sequence encoding Myostatin Binding Peptide
cgtgatgattgtactttttggccagaatggctttggaaactttgtgaacgtcca

SEQ ID NO: 239 - DNA, 54 bp, Artificial Sequence
  Nucleotide sequence encoding Myostatin Binding Peptide
tataatttttgttcttatctttttggtgtttctaaagaagcttgtcaacttcca

SEQ ID NO: 240 - DNA, 54 bp, Artificial Sequence
  Nucleotide sequence encoding Myostatin Binding Peptide
gctcattggtgtgaacaaggtccatggcgttatggtaatatttgtatggcttat

SEQ ID NO: 241 - DNA, 54 bp, Artificial Sequence
  Nucleotide sequence encoding Myostatin Binding Peptide
aatcttgtttgtggtaaaatttctgcttggggtgatgaagcttgtgctcgtgct

SEQ ID NO: 242 - DNA, 54 bp, Artificial Sequence
  Nucleotide sequence encoding Myostatin Binding Peptide
cataatgtttgtactattatgggtccatctatgaaatggttttgttggaatgat

SEQ ID NO: 243 - DNA, 54 bp, Artificial Sequence
  Nucleotide sequence encoding Myostatin Binding Peptide
aatgatctttgtgctatgtggggttggcgtaatactatttggtgtcaaaattct

SEQ ID NO: 244 - DNA, 54 bp, Artificial Sequence
  Nucleotide sequence encoding Myostatin Binding Peptide
ccaccattttgtcaaaatgataatgatatgcttcaatctctttgtaaacttctt

SEQ ID NO: 245 - DNA, 54 bp, Artificial Sequence
  Nucleotide sequence encoding Myostatin Binding Peptide
tggtatgattgtaatgttccaaatgaacttctttctggtctttgtcgtcttttt

SEQ ID NO: 246 - DNA, 54 bp, Artificial Sequence
  Nucleotide sequence encoding Myostatin Binding Peptide
tatggtgattgtgatcaaaatcattggatgtggccatttacttgtctttctctt

SEQ ID NO: 247 - DNA, 54 bp, Artificial Sequence
  Nucleotide sequence encoding Myostatin Binding Peptide
ggttggatgtgtcattttgatcttcatgattggggtgctacttgtcaaccagat

SEQ ID NO: 248 - DNA, 54 bp, Artificial Sequence
  Nucleotide sequence encoding Myostatin Binding Peptide
tattttcattgtatgtttggtggtcatgaatttgaagttcattgtgaatctttt

SEQ ID NO: 249 - DNA, 36 bp, Artificial Sequence
  Nucleotide sequence encoding Myostatin Binding Peptide
gcttattggtgttggcatggtcaatgtgttcgtttt

SEQ ID NO: 250 - DNA, 57 bp, Artificial Sequence
  Nucleotide sequence encoding Myostatin Binding Peptide
tccgaacactggaccttcaccgactgggacggtaacgaatggtgggttcgtccgttc

SEQ ID NO: 251 - DNA, 60 bp, Artificial Sequence
  Nucleotide sequence encoding Myostatin Binding Peptide
atggaaatgctggactccctgttcgaactgctgaaagacatggttccgatctccaaagct

SEQ ID NO: 252 - DNA, 57 bp, Artificial Sequence
  Nucleotide sequence encoding Myostatin Binding Peptide
tccccgccggaagaagctctgatggaatggctgggttggcagtacggtaaattcacc

SEQ ID NO: 253 - DNA, 60 bp, Artificial Sequence
  Nucleotide sequence encoding Myostatin Binding Peptide
tccccggaaaacctgctgaacgacctgtacatcctgatgaccaaacaggaatggtacggt

SEQ ID NO: 254 - DNA, 60 bp, Artificial Sequence
  Nucleotide sequence encoding Myostatin Binding Peptide
ttccactgggaagaaggtatcccgttccacgttgttaccccgtactcctacgaccgtatg

SEQ ID NO: 255 - DNA, 57 bp, Artificial Sequence
  Nucleotide sequence encoding Myostatin Binding Peptide
aaacgtctgctggaacagttcatgaacgacctggctgaactggtttccggtcactcc

SEQ ID NO: 256 - DNA, 60 bp, Artificial Sequence
  Nucleotide sequence encoding Myostatin Binding Peptide
gacacccgtgacgctctgttccaggaattctacgaattcgttcgttcccgtctggttatc

SEQ ID NO: 257 - DNA, 60 bp, Artificial Sequence
  Nucleotide sequence encoding Myostatin Binding Peptide
cgtatgtccgctgctccgcgtccgctgacctaccgtgacatcatggaccagtactggcac

SEQ ID NO: 258 - DNA, 60 bp, Artificial Sequence
  Nucleotide sequence encoding Myostatin Binding Peptide
aacgacaaagctcacttcttcgaaatgttcatgttcgacgttcacaacttcgttgaatcc

SEQ ID NO: 259 - DNA, 60 bp, Artificial Sequence
  Nucleotide sequence encoding Myostatin Binding Peptide
cagacccaggctcagaaaatcgacggtctgtgggaactgctgcagtccatccgtaaccag

SEQ ID NO: 260 - DNA, 54 bp, Artificial Sequence
  Nucleotide sequence encoding Myostatin Binding Peptide
atgctgtccgaattcgaagaattcctgggtaacctggttcaccgtcaggaagct

SEQ ID NO: 261 - DNA, 60 bp, Artificial Sequence
  Nucleotide sequence encoding Myostatin Binding Peptide
tacaccccgaaaatgggttccgaatggacctccttctggcacaaccgtatccactacctg

SEQ ID NO: 262 - DNA, 60 bp, Artificial Sequence
  Nucleotide sequence encoding Myostatin Binding Peptide
ctgaacgacaccctgctgcgtgaactgaaaatggttctgaactccctgtccgacatgaaa

SEQ ID NO: 263 - DNA, 60 bp, Artificial Sequence
  Nucleotide sequence encoding Myostatin Binding Peptide
ttcgacgttgaacgtgacctgatgcgttggctggaaggtttcatgcagtccgctgctacc

SEQ ID NO: 264 - DNA, 60 bp, Artificial Sequence
  Nucleotide sequence encoding Myostatin Binding Peptide
caccacggttggaactacctgcgtaaaggttccgctccgcagtggttcgaagcttgggtt

SEQ ID NO: 265 - DNA, 60 bp, Artificial Sequence
  Nucleotide sequence encoding Myostatin Binding Peptide
gttgaatccctgcaccagctgcagatgtggctggaccagaaactggcttccggtccgcac

SEQ ID NO: 266 - DNA, 54 bp, Artificial Sequence
  Nucleotide sequence encoding Myostatin Binding Peptide
cgtgctaccctgctgaaagacttctggcagctggttgaaggttacggtgacaac

SEQ ID NO: 267 - DNA, 48 bp, Artificial Sequence
  Nucleotide sequence encoding Myostatin Binding Peptide
gaagaactgctgcgtgaattctaccgtttcgtttccgctttcgactac

SEQ ID NO: 268 - DNA, 60 bp, Artificial Sequence
  Nucleotide sequence encoding Myostatin Binding Peptide
ggtctgctggacgaattctcccacttcatcgctgaacagttctaccagatgccgggtggt

SEQ ID NO: 269 - DNA, 60 bp, Artificial Sequence
  Nucleotide sequence encoding Myostatin Binding Peptide
taccgtgaaatgtccatgctggaaggtctgctggacgttctggaacgtctgcagcactac

SEQ ID NO: 270 - DNA, 60 bp, Artificial Sequence
  Nucleotide sequence encoding Myostatin Binding Peptide
cacaactcctcccagatgctgctgtccgaactgatcatgctggttggttccatgatgcag

SEQ ID NO: 271 - DNA, 60 bp, Artificial Sequence
  Nucleotide sequence encoding Myostatin Binding Peptide
tggcgtgaacacttcctgaactccgactacatccgtgacaaactgatcgctatcgacggt

SEQ ID NO: 272 - DNA, 57 bp, Artificial Sequence
  Nucleotide sequence encoding Myostatin Binding Peptide
cagttcccgttctacgttttcgacgacctgccggctcagctggaatactggatcgct

SEQ ID NO: 273 - DNA, 60 bp, Artificial Sequence
  Nucleotide sequence encoding Myostatin Binding Peptide
gaattcttccactggctgcacaaccaccgttccgaagttaaccactggctggacatgaac

SEQ ID NO: 274 - DNA, 57 bp, Artificial Sequence
  Nucleotide sequence encoding Myostatin Binding Peptide
gaagctctttttcaaaatttttttcgtgatgttcttactctttctgaacgtgaatat

SEQ ID NO: 275 - DNA, 60 bp, Artificial Sequence
  Nucleotide sequence encoding Myostatin Binding Peptide
caatattgggaacaacaatggatgacttattttcgtgaaaatggtcttcatgttcaatat

SEQ ID NO: 276 - DNA, 60 bp, Artificial Sequence
  Nucleotide sequence encoding Myostatin Binding Peptide
aatcaacgtatgatgcttgaagatctttggcgtattatgactccaatgtttggtcgttct

SEQ ID NO: 277 - DNA, 60 bp, Artificial Sequence
  Nucleotide sequence encoding Myostatin Binding Peptide
tttcttgatgaacttaaagctgaactttctcgtcattatgctcttgatgatcttgatgaa

SEQ ID NO: 278 - DNA, 60 bp, Artificial Sequence
  Nucleotide sequence encoding Myostatin Binding Peptide
ggtaaacttattgaaggtcttcttaatgaacttatgcaacttgaaacttttatgccagat

SEQ ID NO: 279 - DNA, 45 bp, Artificial Sequence
  Nucleotide sequence encoding Myostatin Binding Peptide
attcttcttcttgatgaatataaaaaagattggaaatcttggttt

SEQ ID NO: 280 - DNA, 150 bp, Artificial Sequence
  Nucleotide sequence encoding Myostatin Binding Peptide
cagggccactgtactcgctggccgtggatgtgcccgccgtacggttctggttccgctaccggtggttctggttccactgcttcttctggttccggttctgctactggtcagggtcactgcactcgttggccatggatgtgtccaccgtat

SEQ ID NO: 281 - DNA, 129 bp, Artificial Sequence
  Nucleotide sequence encoding Myostatin Binding Peptide
tggtatccgtgttatgagggtcacttctggtgctacgatctgggttctggttccactgcttcttctggttccggttccgctactggttggtacccgtgctacgaaggtcacttttggtgttatgatctg

SEQ ID NO: 282 - DNA, 150 bp, Artificial Sequence
  Nucleotide sequence encoding Myostatin Binding Peptide
cacactccgtgtccgtggtttgctccgctgtgcgttgaatggggttctggttccgctactggtggttccggttccactgcttcttctggttccggttctgcaactggtcacaccccgtgcccgtggtttgcaccgctgtgtgtagagtgg

SEQ ID NO: 283 - DNA, 150 bp, Artificial Sequence
  Nucleotide sequence encoding Myostatin Binding Peptide
ccggattggtgtatcgacccggactggtggtgcaaattctggggttctggttccgctaccggtggttccggttccactgcttcttctggttccggttctgcaactggtccggactggtgcatcgacccggattggtggtgtaaattttgg

SEQ ID NO: 284 - DNA, 150 bp, Artificial Sequence; identical to SEQ ID NO: 283
  Nucleotide sequence encoding Myostatin Binding Peptide

SEQ ID NO: 285 - DNA, 129 bp, Artificial Sequence
  Nucleotide sequence encoding Myostatin Binding Peptide
accacttggtgcatctctccgatgtggttctgctctcagcagggttctggttccactgcttcttctggttccggttctgcaactggtactacttggtgtatctctccaatgtggttttgttctcagcaa

SEQ ID NO: 286 - DNA, 150 bp, Artificial Sequence
  Nucleotide sequence encoding Myostatin Binding Peptide
cactgggcatgtggctattggccgtggtcctgcaaatgggttggttctggttccgctaccggtggttccggttccactgcttcttctggttccggttctgcaactggtcactgggcttgcggttactggccgtggtcttgtaaatgggtt

SEQ ID NO: 287 - DNA, 150 bp, Artificial Sequence
  Nucleotide sequence encoding Myostatin Binding Peptide
aaaaaacactgtcagatctggacttggatgtgcgctccgaaaggttctggttccgctaccggtggttctggttccactgcttcttctggttccggttccgctactggtcagggtcactgcactcgttggccatggatgtgtccgccgtat

SEQ ID NO: 288 - DNA, 150 bp, Artificial Sequence
  Nucleotide sequence encoding Myostatin Binding Peptide
cagggtcactgcacccgttggccgtggatgtgcccgccgtacggttctggttccgctaccggtggttctggttccactgcttcttctggttccggttctgctactggtaaaaaacactgccagatctggacttggatgtgcgctccgaaa

SEQ ID NO: 289 - DNA, 150 bp, Artificial Sequence; identical to SEQ ID NO: 287
  Nucleotide sequence encoding Myostatin Binding Peptide

SEQ ID NO: 290 - DNA, 108 bp, Artificial Sequence
  Nucleotide sequence encoding Myostatin Binding Peptide
aaaaaacactgccagatctggacttggatgtgcgctccgaaaggtggtggtggtggtggcggtggccagggtcactgcacccgttggccgtggatgtgtccgccgtat

SEQ ID NO: 291 - DNA, 102 bp, Artificial Sequence
  Nucleotide sequence encoding Myostatin Binding Peptide
cagggtcactgcacccgttggccgtggatgtgcccgccgtacggtggtggtggtggtggcaaaaaacactgccagatctggacttggatgtgcgctccgaaa

SEQ ID NO: 292 - DNA, 37 bp, Artificial Sequence
  Nucleotide sequence encoding Myostatin Binding Peptide
gagagagagcatatgaatgagaacagtgagcaaaaag

SEQ ID NO: 293 - DNA, 34 bp, Artificial Sequence
  Nucleotide sequence encoding Myostatin Binding Peptide
agagagggatccattatgagcacccacagcggtc

SEQ ID NO: 294 - DNA, 25 bp, Artificial Sequence
  Nucleotide sequence encoding Myostatin Binding Peptide
cggcgcaactatcggtatcaagctg

SEQ ID NO: 295 - DNA, 26 bp, Artificial Sequence
  Nucleotide sequence encoding Myostatin Binding Peptide
catgtaccgtaacactgagtttcgtc

SEQ ID NO: 296 - protein, 227 aa, Artificial Sequence
  Myostatin Binding Peptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - DNA, 67 bp, Artificial Sequence
  Nucleotide sequence encoding Myostatin Binding Peptide
acaaacaaacatatgggtgcacagaaagcggccgcaaaaaaactcgagggtggaggcggtggggaca

SEQ ID NO: 298 - DNA, 20 bp, Artificial Sequence
  Nucleotide sequence encoding Myostatin Binding Peptide
ggtcattactggaccggatc

SEQ ID NO: 299 - DNA, 25 bp, Artificial Sequence
  Nucleotide sequence encoding Myostatin Binding Peptide
cgtacaggtttacgcaagaaaatgg

SEQ ID NO: 300 - DNA, 66 bp, Artificial Sequence
  Nucleotide sequence encoding Myostatin Binding Peptide
tttgttggatccattactcgagtttttttgcggccgctttctgtgcaccaccacctccacctttac

SEQ ID NO: 301 - DNA, 681 bp, Artificial Sequence
  Nucleotide sequence encoding Myostatin Binding Peptide
gacaaaactcacacatgtccaccttgcccagcacctgaactcctggggggaccgtcagttttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 302 - DNA, 15 bp, Artificial Sequence
  Nucleotide sequence encoding Myostatin Binding Peptide
ggtggaggtggtggt

SEQ ID NO: 303 - protein, 22 aa, Artificial Sequence
  Peptibody
MFCGGGGGKDKCKMWHWMCKPP

SEQ ID NO: 304 - DNA, 740 bp, Artificial Sequence
  Nucleotide sequence encoding Myostatin Binding Peptide
atggacaaaactcacacatgtccaccttgcccagcacctgaactcctggggggaccgtcagttttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggtggtggtaagacaaatgcaaaatgtggcactggatgtgcaaaccgccg

SEQ ID NO: 305 - protein, 20 aa, Artificial Sequence
  Myostatin Binding Peptide
VALHGQCTRWPWMCPPQREG

SEQ ID NO: 306 - protein, 20 aa, Artificial Sequence
  Myostatin Binding Peptide
YPEQGLCTRWPWMCPPQTLA

SEQ ID NO: 307 - protein, 20 aa, Artificial Sequence
  Myostatin Binding Peptide
GLNQGHCTRWPWMCPPQDSN

SEQ ID NO: 308 - protein, 20 aa, Artificial Sequence
  Myostatin Binding Peptide
MITQGQCTRWPWMCPPQPSG

SEQ ID NO: 309 - protein, 20 aa, Artificial Sequence
  Myostatin Binding Peptide
AGAQEHCTRWPWMCAPNDWI

SEQ ID NO: 310 - protein, 20 aa, Artificial Sequence
  Myostatin Binding Peptide
GVNQGQCTRWRWMCPPNGWE

SEQ ID NO: 311 - protein, 20 aa, Artificial Sequence
  Myostatin Binding Peptide
LADHGQCIRWPWMCPPEGWE

SEQ ID NO: 312 - protein, 20 aa, Artificial Sequence
  Myostatin Binding Peptide
ILEQAQCTRWPWMCPPQRGG

SEQ ID NO: 313 - protein, 20 aa, Artificial Sequence
  Myostatin Binding Peptide
TQTHAQCTRWPWMCPPQWEG

SEQ ID NO: 314 - protein, 20 aa, Artificial Sequence
  Myostatin Binding Peptide
VVTQGHCTLWPWMCPPQRWR

SEQ ID NO: 315 - protein, 20 aa, Artificial Sequence
  Myostatin Binding Peptide
IYPHDQCTRWPWMCPPQPYP

SEQ ID NO: 316 - protein, 20 aa, Artificial Sequence
  Myostatin Binding Peptide
SYWQGQCTRWPWMCPPQWRG

SEQ ID NO: 317 - protein, 20 aa, Artificial Sequence
  Myostatin Binding Peptide
MWQQGHCTRWPWMCPPQGWG

SEQ ID NO: 318 - protein, 20 aa, Artificial Sequence
  Myostatin Binding Peptide
EFTQWHCTRWPWMCPPQRSQ

SEQ ID NO: 319 - protein, 20 aa, Artificial Sequence
  Myostatin Binding Peptide
LDDQWQCTRWPWMCPPQGFS

SEQ ID NO: 320 - protein, 20 aa, Artificial Sequence
  Myostatin Binding Peptide
YQTQGLCTRWPWMCPPQSQR

SEQ ID NO: 321 - protein, 20 aa, Artificial Sequence
  Myostatin Binding Peptide
ESNQGQCTRWPWMCPPQGGW

SEQ ID NO: 322 - protein, 20 aa, Artificial Sequence
  Myostatin Binding Peptide
WTDRGPCTRWPWMCPPQANG

SEQ ID NO: 323 - protein, 20 aa, Artificial Sequence
  Myostatin Binding Peptide
VGTQGQCTRWPWMCPPYETG

SEQ ID NO: 324 - protein, 20 aa, Artificial Sequence
  Myostatin Binding Peptide
PYEQGKCTRWPWMCPPYEVE

SEQ ID NO: 325 - protein, 20 aa, Artificial Sequence
  Myostatin Binding Peptide
SEYQGLCTRWPWMCPPQGWK

SEQ ID NO: 326 - protein, 20 aa, Artificial Sequence
  Myostatin Binding Peptide
TFSQGHCTRWPWMCPPQGWG

SEQ ID NO: 327 - protein, 20 aa, Artificial Sequence
  Myostatin Binding Peptide
PGAHDHCTRWPWMCPPQSRY

SEQ ID NO: 328 - protein, 20 aa, Artificial Sequence
  Myostatin Binding Peptide
VAEEWHCRRWPWMCPPQDWR

SEQ ID NO: 329 - protein, 20 aa, Artificial Sequence
  Myostatin Binding Peptide
VGTQGHCTRWPWMCPPQPAG

SEQ ID NO: 330 - protein, 20 aa, Artificial Sequence
  Myostatin Binding Peptide
EEDQAHCRSWPWMCPPQGWV

SEQ ID NO: 331 - protein, 20 aa, Artificial Sequence
  Myostatin Binding Peptide
ADTQGHCTRWPWMCPPQHWF

SEQ ID NO: 332 - protein, 20 aa, Artificial Sequence
  Myostatin Binding Peptide
SGPQGHCTRWPWMCAPQGWF

SEQ ID NO: 333 - protein, 20 aa, Artificial Sequence
  Myostatin Binding Peptide
TLVQGHCTRWPWMCPPQRWV

SEQ ID NO: 334 - protein, 20 aa, Artificial Sequence
  Myostatin Binding Peptide
GMAHGKCTRWAWMCPPQSWK

SEQ ID NO: 335 - protein, 20 aa, Artificial Sequence
  Myostatin Binding Peptide
ELYHGQCTRWPWMCPPQSWA

SEQ ID NO: 336 - protein, 20 aa, Artificial Sequence
  Myostatin Binding Peptide
VADHGHCTRWPWMCPPQGWG

SEQ ID NO: 337 - protein, 20 aa, Artificial Sequence
  Myostatin Binding Peptide
PESQGHCTRWPWMCPPQGWG

SEQ ID NO: 338 - protein, 20 aa, Artificial Sequence
  Myostatin Binding Peptide
IPAHGHCTRWPWMCPPQRWR

SEQ ID NO: 339 - protein, 20 aa, Artificial Sequence
  Myostatin Binding Peptide
FTVHGHCTRWPWMCPPYGWV

SEQ ID NO: 340 - protein, 20 aa, Artificial Sequence
  Myostatin Binding Peptide
PDFPGHCTRWRWMCPPQGWE

SEQ ID NO: 341 - protein, 20 aa, Artificial Sequence
  Myostatin Binding Peptide
QLWQGPCTQWPWMCPPKGRY

SEQ ID NO: 342 - protein, 20 aa, Artificial Sequence
  Myostatin Binding Peptide
HANDGHCTRWQWMCPPQWGG

SEQ ID NO: 343 - protein, 20 aa, Artificial Sequence
  Myostatin Binding Peptide
ETDHGLCTRWPWMCPPYGAR

SEQ ID NO: 344 - protein, 20 aa, Artificial Sequence
  Myostatin Binding Peptide
GTWQGLCTRWPWMCPPQGWQ

SEQ ID NO: 345 - protein, 20 aa, Artificial Sequence
  Myostatin Binding Peptide
VATQGQCTRWPWMCPPQGWG

SEQ ID NO: 346 - protein, 20 aa, Artificial Sequence
  Myostatin Binding Peptide
VATQGQCTRWPWMCPPQRWG

SEQ ID NO: 347 - protein, 20 aa, Artificial Sequence
  Myostatin Binding Peptide
QREWYPCYGGHLWCYDLHKA

SEQ ID NO: 348 - protein, 20 aa, Artificial Sequence
  Myostatin Binding Peptide
ISAWYSCYAGHFWCWDLKQK

SEQ ID NO: 349 - protein, 20 aa, Artificial Sequence
  Myostatin Binding Peptide
WTGWYQCYGGHLWCYDLRRK

SEQ ID NO: 350 - protein, 20 aa, Artificial Sequence
  Myostatin Binding Peptide
KTFWYPCYDGHFWCYNLKSS

SEQ ID NO: 351 - protein, 20 aa, Artificial Sequence
  Myostatin Binding Peptide
ESRWYPCYEGHLWCFDLTET

SEQ ID NO: 352 - protein, 10 aa, Artificial Sequence
  Myostatin Binding Peptide
CXXWXWMCPP

SEQ ID NO: 353 - protein, 10 aa, Artificial Sequence
  Myostatin Binding Peptide
CXXWXWMCPP

SEQ ID NO: 354 - protein, 20 aa, Artificial Sequence
  Myostatin Binding Peptide
XXXXXXCXXWXWMCPPXXXX

SEQ ID NO: 355 - protein, 20 aa, Artificial Sequence
  Myostatin Binding Peptide
XXXXXXCXXWXWMCPPXXXX

SEQ ID NO: 356 - protein, 7 aa, Artificial Sequence
  Myostatin Binding Peptide
WYXXYXG

SEQ ID NO: 357 - protein, 22 aa, Artificial Sequence
  Myostatin Binding Peptide
RMEMLESLLELLKEIVPMSKAG

SEQ ID NO: 358 - protein, 22 aa, Artificial Sequence
  Myostatin Binding Peptide
RMEMLESLLELLKEIVPMSKAR

SEQ ID NO: 359 - protein, 22 aa, Artificial Sequence
  Myostatin Binding Peptide
RMEMLESLLELLKDIVPMSKPS

SEQ ID NO: 360 - protein, 22 aa, Artificial Sequence
  Myostatin Binding Peptide
GMEMLESLFELLQEIVPMSKAP

SEQ ID NO: 361 - protein, 22 aa, Artificial Sequence
  Myostatin Binding Peptide
RMEMLESLLELLKDIVPISNPP

SEQ ID NO: 362 - protein, 22 aa, Artificial Sequence
  Myostatin Binding Peptide
RIEMLESLLELLQEIVPISKAE

SEQ ID NO: 363 - protein, 22 aa, Artificial Sequence
  Myostatin Binding Peptide
RMEMLQSLLELLKDIVPMSNAR

SEQ ID NO: 364 - protein, 22 aa, Artificial Sequence
  Myostatin Binding Peptide
RMEMLESLLELLKEIVPTSNGT

SEQ ID NO: 365 - protein, 22 aa, Artificial Sequence
  Myostatin Binding Peptide
RMEMLESLFELLKEIVPMSKAG

SEQ ID NO: 366 - protein, 22 aa, Artificial Sequence
  Myostatin Binding Peptide
RMEMLGSLLELLKEIVPMSKAR

SEQ ID NO: 367 - protein, 22 aa, Artificial Sequence
  Myostatin Binding Peptide
QMELLDSLFELLKEIVPKSQPA

SEQ ID NO: 368 - protein, 22 aa, Artificial Sequence
  Myostatin Binding Peptide
RMEMLDSLLELLKEIVPMSNAR

SEQ ID NO: 369 - protein, 22 aa, Artificial Sequence
  Myostatin Binding Peptide
RMEMLESLLELLHEIVPMSQAG

SEQ ID NO: 370 - protein, 22 aa, Artificial Sequence
  Myostatin Binding Peptide
QMEMLESLLQLLKEIVPMSKAS

SEQ ID NO: 371 - protein, 22 aa, Artificial Sequence
  Myostatin Binding Peptide
RMEMLDSLLELLKDMVPMTTGA

SEQ ID NO: 372 - protein, 22 aa, Artificial Sequence
  Myostatin Binding Peptide
RIEMLESLLELLKDMVPMANAS

SEQ ID NO: 373 - protein, 22 aa, Artificial Sequence
  Myostatin Binding Peptide
RMEMLESLLQLLNEIVPMSRAR

SEQ ID NO: 374 - protein, 22 aa, Artificial Sequence
  Myostatin Binding Peptide
RMEMLESLFDLLKELVPMSKGV

SEQ ID NO: 375 - protein, 22 aa, Artificial Sequence
  Myostatin Binding Peptide
RIEMLESLLELLKDIVPIQKAR

SEQ ID NO: 376 - protein, 22 aa, Artificial Sequence
  Myostatin Binding Peptide
RMELLESLFELLKDMVPMSDSS

SEQ ID NO: 377 - protein, 22 aa, Artificial Sequence
  Myostatin Binding Peptide
RMEMLESLLEVLQEIVPRAKGA

SEQ ID NO: 378 - protein, 22 aa, Artificial Sequence
  Myostatin Binding Peptide
RMEMLDSLLQLLNEIVPMSHAR

SEQ ID NO: 379 - protein, 22 aa, Artificial Sequence
  Myostatin Binding Peptide
RMEMLESLLELLKDIVPMSNAG

SEQ ID NO: 380 - protein, 22 aa, Artificial Sequence
  Myostatin Binding Peptide
RMEMLQSLFELLKGMVPISKAG

SEQ ID NO: 381 - protein, 22 aa, Artificial Sequence
  Myostatin Binding Peptide
RMEMLESLLELLKEIVPNSTAA

SEQ ID NO: 382 - protein, 22 aa, Artificial Sequence
  Myostatin Binding Peptide
RMEMLQSLLELLKEIVPISKAG

SEQ ID NO: 383 - protein, 22 aa, Artificial Sequence
  Myostatin Binding Peptide
RIEMLDSLLELLNELVPMSKAR

SEQ ID NO: 384 - protein, 22 aa, Artificial Sequence
  Myostatin Binding Peptide
QVESLQQLLMWLDQKLASGPQG

SEQ ID NO: 385 - protein, 22 aa, Artificial Sequence
  Myostatin Binding Peptide
RMELLESLFELLKEMVPRSKAV

SEQ ID NO: 386 - protein, 22 aa, Artificial Sequence
  Myostatin Binding Peptide
QAVSLQHLLMWLDQKLASGPQH

SEQ ID NO: 387 - protein, 22 aa, Artificial Sequence
  Myostatin Binding Peptide
DEDSLQQLLMWLDQKLASGPQL

SEQ ID NO: 388 - protein, 22 aa, Artificial Sequence
  Myostatin Binding Peptide
PVASLQQLLIWLDQKLAQGPHA

SEQ ID NO: 389 - protein, 22 aa, Artificial Sequence
  Myostatin Binding Peptide
EVDELQQLLNWLDHKLASGPLQ

SEQ ID NO: 390 - protein, 22 aa, Artificial Sequence
  Myostatin Binding Peptide
DVESLEQLLMWLDHQLASGPHG

SEQ ID NO: 391 - protein, 22 aa, Artificial Sequence
  Myostatin Binding Peptide
QVDSLQQVLLWLEHKLALGPQV

SEQ ID NO: 392 - protein, 22 aa, Artificial Sequence
  Myostatin Binding Peptide
GDESLQHLLMWLEQKLALGPHG

SEQ ID NO: 393 - protein, 22 aa, Artificial Sequence
  Myostatin Binding Peptide
QIEMLESLLDLLRDMVPMSNAF

SEQ ID NO: 394 - protein, 22 aa, Artificial Sequence
  Myostatin Binding Peptide
EVDSLQQLLMWLDQKLASGPQA

SEQ ID NO: 395 - protein, 22 aa, Artificial Sequence
  Myostatin Binding Peptide
EDESLQQLLIYLDKMLSSGPQV

SEQ ID NO: 396 - protein, 22 aa, Artificial Sequence
  Myostatin Binding Peptide
AMDQLHQLLIWLDHKLASGPQA

SEQ ID NO: 397 - protein, 22 aa, Artificial Sequence
  Myostatin Binding Peptide
RIEMLESLLELLDEIALIPKAW

SEQ ID NO: 398 - protein, 22 aa, Artificial Sequence
  Myostatin Binding Peptide
EVVSLQHLLMWLEHKLASGPDG

SEQ ID NO: 399 - protein, 22 aa, Artificial Sequence
  Myostatin Binding Peptide
GGESLQQLLMWLDQQLASGPQR

SEQ ID NO: 400 - protein, 22 aa, Artificial Sequence
  Myostatin Binding Peptide
GVESLQQLLIFLDHMLVSGPHD

SEQ ID NO: 401 - protein, 22 aa, Artificial Sequence
  Myostatin Binding Peptide
NVESLEHLMMWLERLLASGPYA

SEQ ID NO: 402 - protein, 22 aa, Artificial Sequence
  Myostatin Binding Peptide
QVDSLQQLLIWLDHQLASGPKR

SEQ ID NO: 403 - protein, 22 aa, Artificial Sequence
  Myostatin Binding Peptide
EVESLQQLLMWLEHKLAQGPQG

SEQ ID NO: 404 - protein, 22 aa, Artificial Sequence
  Myostatin Binding Peptide
EVDSLQQLLMWLDQKLASGPHA

SEQ ID NO: 405 - protein, 22 aa, Artificial Sequence
  Myostatin Binding Peptide
EVDSLQQLLMWLDQQLASGPQK

SEQ ID NO: 406 - protein, 22 aa, Artificial Sequence
  Myostatin Binding Peptide
GVEQLPQLLMWLEQKLASGPQR

SEQ ID NO: 407 - protein, 22 aa, Artificial Sequence
  Myostatin Binding Peptide
GEDSLQQLLMWLDQQLAAGPQV

SEQ ID NO: 408 - protein, 22 aa, Artificial Sequence
  Myostatin Binding Peptide
ADDSLQQLLMWLDRKLASGPHV

SEQ ID NO: 409 - protein, 22 aa, Artificial Sequence
  Myostatin Binding Peptide
PVDSLQQLLIWLDQKLASGPQG

SEQ ID NO: 410 - protein, 22 aa, Artificial Sequence
  Myostatin Binding Peptide
DWRATLLKEFWQLVEGLGDNLV

SEQ ID NO: 411 - protein, 22 aa, Artificial Sequence
  Myostatin Binding Peptide
QSRATLLKEFWQLVEGLGDKQA

SEQ ID NO: 412 - protein, 22 aa, Artificial Sequence
  Myostatin Binding Peptide
DGRATLLTEFWQLVQGLGQKEA

SEQ ID NO: 413 - protein, 22 aa, Artificial Sequence
  Myostatin Binding Peptide
LARATLLKEFWQLVEGLGEKVV

SEQ ID NO: 414 - protein, 22 aa, Artificial Sequence
  Myostatin Binding Peptide
GSRDTLLKEFWQLVVGLGDMQT

SEQ ID NO: 415 - protein, 22 aa, Artificial Sequence
  Myostatin Binding Peptide
DARATLLKEFWQLVDAYGDRMV

SEQ ID NO: 416 - protein, 22 aa, Artificial Sequence
  Myostatin Binding Peptide
NDRAQLLRDFWQLVDGLGVKSW

SEQ ID NO: 417 - protein, 22 aa, Artificial Sequence
  Myostatin Binding Peptide
GVRETLLYELWYLLKGLGANQG

SEQ ID NO: 418 - protein, 22 aa, Artificial Sequence
  Myostatin Binding Peptide
QARATLLKEFCQLVGCQGDKLS

SEQ ID NO: 419 - protein, 22 aa, Artificial Sequence
  Myostatin Binding Peptide
QERATLLKEFWQLVAGLGQNMR

SEQ ID NO: 420 - protein, 22 aa, Artificial Sequence
  Myostatin Binding Peptide
SGRATLLKEFWQLVQGLGEYRW

SEQ ID NO: 421 - protein, 22 aa, Artificial Sequence
  Myostatin Binding Peptide
TMRATLLKEFWLFVDGQREMQW

SEQ ID NO: 422 - protein, 22 aa, Artificial Sequence
  Myostatin Binding Peptide
GERATLLNDFWQLVDGQGDNTG

SEQ ID NO: 423 - protein, 22 aa, Artificial Sequence
  Myostatin Binding Peptide
DERETLLKEFWQLVHGWGDNVA

SEQ ID NO: 424 - protein, 22 aa, Artificial Sequence
  Myostatin Binding Peptide
GGRATLLKELWQLLEGQGANLV

SEQ ID NO: 425 - protein, 22 aa, Artificial Sequence
  Myostatin Binding Peptide
TARATLLNELVQLVKGYGDKLV

SEQ ID NO: 426 - protein, 22 aa, Artificial Sequence
  Myostatin Binding Peptide
GMRATLLQEFWQLVGGQGDNWM

SEQ ID NO: 427 - protein, 22 aa, Artificial Sequence
  Myostatin Binding Peptide
STRATLLNDLWQLMKGWAEDRG

SEQ ID NO: 428 - protein, 22 aa, Artificial Sequence
  Myostatin Binding Peptide
SERATLLKELWQLVGGWGDNFG

SEQ ID NO: 429 - protein, 22 aa, Artificial Sequence
  Myostatin Binding Peptide
VGRATLLKEFWQLVEGLVGQSR

SEQ ID NO: 430 - protein, 22 aa, Artificial Sequence
  Myostatin Binding Peptide
EIRATLLKEFWQLVDEWREQPN

SEQ ID NO: 431 - protein, 22 aa, Artificial Sequence
  Myostatin Binding Peptide
QLRATLLKEFLQLVHGLGETDS

SEQ ID NO: 432 - protein, 22 aa, Artificial Sequence
  Myostatin Binding Peptide
TQRATLLKEFWQLIEGLGGKHV

SEQ ID NO: 433 - protein, 22 aa, Artificial Sequence
  Myostatin Binding Peptide
HYRATLLKEFWQLVDGLREQGV

SEQ ID NO: 434 - protein, 22 aa, Artificial Sequence
  Myostatin Binding Peptide
QSRVTLLREFWQLVESYRPIVN

SEQ ID NO: 435 - protein, 22 aa, Artificial Sequence
  Myostatin Binding Peptide
LSRATLLNEFWQFVDGQRDKRM

SEQ ID NO: 436 - protein, 22 aa, Artificial Sequence
  Myostatin Binding Peptide
WDRATLLNDFWHLMEELSQKPG

SEQ ID NO: 437 - protein, 22 aa, Artificial Sequence
  Myostatin Binding Peptide
QERATLLKEFWRMVEGLGKNRG

SEQ ID NO: 438 - protein, 22 aa, Artificial Sequence
  Myostatin Binding Peptide
NERATLLREFWQLVGGYGVNQR

SEQ ID NO: 439 - protein, 22 aa, Artificial Sequence
  Myostatin Binding Peptide
HQRDMSMLWELLDVLDGLRQYS

SEQ ID NO: 440 - protein, 22 aa, Artificial Sequence
  Myostatin Binding Peptide
TQRDMSMLDGLLEVLDQLRQQR

SEQ ID NO: 441 - protein, 22 aa, Artificial Sequence
  Myostatin Binding Peptide
TSRDMSLLWELLEELDRLGHQR

SEQ ID NO: 442 - protein, 22 aa, Artificial Sequence
  Myostatin Binding Peptide
MQHDMSMLYGLVELLESLGHQI

SEQ ID NO: 443 - protein, 22 aa, Artificial Sequence
  Myostatin Binding Peptide
WNRDMRMLESLFEVLDGLRQQV

SEQ ID NO: 444 - protein, 22 aa, Artificial Sequence
  Myostatin Binding Peptide
GYRDMSMLEGLLAVLDRLGPQL

SEQ ID NO: 445 - protein, 22 aa, Artificial Sequence
  Myostatin Binding Peptide
TQRDMSMLEGLLEVLDRLGQQR

SEQ ID NO: 446 - protein, 22 aa, Artificial Sequence
  Myostatin Binding Peptide
WYRDMSMLEGLLEVLDRLGQQR

SEQ ID NO: 447 - protein, 22 aa, Artificial Sequence
  Myostatin Binding Peptide
TQNSRQMLLSDFMMLVGSMIQG

SEQ ID NO: 448 - protein, 22 aa, Artificial Sequence
  Myostatin Binding Peptide
MQTSRHILLSEFMMLVGSIMHG

SEQ ID NO: 449 - protein, 22 aa, Artificial Sequence
  Myostatin Binding Peptide
HDNSRQMLLSDLLHLVGTMIQG

SEQ ID NO: 450 - protein, 22 aa, Artificial Sequence
  Myostatin Binding Peptide
MENSRQNLLRELIMLVGNMSHQ

SEQ ID NO: 451 - protein, 22 aa, Artificial Sequence
  Myostatin Binding Peptide
QDTSRHMLLREFMMLVGEMIQG

SEQ ID NO: 452 - protein, 22 aa, Artificial Sequence
  Myostatin Binding Peptide
DQNSRQMLLSDLMILVGSMIQG

SEQ ID NO: 453 - protein, 22 aa, Artificial Sequence
  Myostatin Binding Peptide
NVFFQWVQKHGRVVYQWLDINV

SEQ ID NO: 454 - protein, 22 aa, Artificial Sequence
  Myostatin Binding Peptide
FDFLQWLQNHRSEVEHWLVMDV

SEQ ID NO: 455 - protein, 11 aa, Artificial Sequence
  Myostatin Binding Peptide
XEMLXSLXXLL

SEQ ID NO: 456 - protein, 8 aa, Artificial Sequence
  Myostatin Binding Peptide
LXXLLXXL

SEQ ID NO: 457 - protein, 9 aa, Artificial Sequence
  Myostatin Binding Peptide
XXXXXXXXX

SEQ ID NO: 458 - DNA, 66 bp, Artificial Sequence
  Nucleotide sequence encoding Myostatin Binding Peptide
cgtatggaaatgcttgaatctcttcttgaacttcttaaagaaattgttccaatgtctaaagctggt

SEQ ID NO: 459 - DNA, 66 bp, Artificial Sequence
  Nucleotide sequence encoding Myostatin Binding Peptide
cgtatggaaatgcttgaatctcttcttgaacttcttaaagaaattgttccaatgtctaaagctcgt

SEQ ID NO: 460 - DNA, 66 bp, Artificial Sequence
  Nucleotide sequence encoding Myostatin Binding Peptide
cgtatggaaatgcttgaatctcttcttgaacttcttaaagatattgttccaatgtctaaaccatct

SEQ ID NO: 461 - DNA, 66 bp, Artificial Sequence
  Nucleotide sequence encoding Myostatin Binding Peptide
ggtatggaaatgcttgaatctctttttgaacttcttcaagaaattgttccaatgtctaaagctcca

SEQ ID NO: 462 - DNA, 66 bp, Artificial Sequence
  Nucleotide sequence encoding Myostatin Binding Peptide
cgtatggaaatgcttgaatctcttcttgaacttcttaaagatattgttccaatttctaatccacca

SEQ ID NO: 463 - DNA, 66 bp, Artificial Sequence
  Nucleotide sequence encoding Myostatin Binding Peptide
cgtattgaaatgcttgaatctcttcttgaacttcttcaagaaattgttccaatttctaaagctgaa

SEQ ID NO: 464 - DNA, 66 bp, Artificial Sequence
  Nucleotide sequence encoding Myostatin Binding Peptide
cgtatggaaatgcttcaatctcttcttgaacttcttaaagatattgttccaatgtctaatgctcgt

SEQ ID NO: 465 - DNA, 66 bp, Artificial Sequence
  Nucleotide sequence encoding Myostatin Binding Peptide
cgtatggaaatgcttgaatctcttcttgaacttcttaaagaaattgttccaacttctaatggtact

SEQ ID NO: 466 - DNA, 66 bp, Artificial Sequence
  Nucleotide sequence encoding Myostatin Binding Peptide
cgtatggaaatgcttgaatctctttttgaacttcttaaagaaattgttccaatgtctaaagctggt

SEQ ID NO: 467 - DNA, 66 bp, Artificial Sequence
  Nucleotide sequence encoding Myostatin Binding Peptide
cgtatggaaatgcttggttctcttcttgaacttcttaaagaaattgttccaatgtctaaagctcgt

SEQ ID NO: 468 - DNA, 66 bp, Artificial Sequence
  Nucleotide sequence encoding Myostatin Binding Peptide
caaatggaacttcttgattctctttttgaacttcttaaagaaattgttccaaaatctcaaccagct

SEQ ID NO: 469 - DNA, 66 bp, Artificial Sequence
  Nucleotide sequence encoding Myostatin Binding Peptide
cgtatggaaatgcttgattctcttcttgaacttcttaaagaaattgttccaatgtctaatgctcgt

SEQ ID NO: 470 - DNA, 66 bp, Artificial Sequence
  Nucleotide sequence encoding Myostatin Binding Peptide
cgtatggaaatgcttgaatctcttcttgaacttcttcatgaaattgttccaatgtctcaagctggt

SEQ ID NO: 471 - DNA, 66 bp, Artificial Sequence
  Nucleotide sequence encoding Myostatin Binding Peptide
caaatggaaatgcttgaatctcttcttcaacttcttaaagaaattgttccaatgtctaaagcttct

SEQ ID NO: 472 - DNA, 66 bp, Artificial Sequence
  Nucleotide sequence encoding Myostatin Binding Peptide
cgtatggaaatgcttgattctcttcttgaacttcttaaagatatggttccaatgactactggtgct

SEQ ID NO: 473 - DNA, 66 bp, Artificial Sequence
  Nucleotide sequence encoding Myostatin Binding Peptide
cgtattgaaatgcttgaatctcttcttgaacttcttaaagatatggttccaatggctaatgcttct

SEQ ID NO: 474 - DNA, 66 bp, Artificial Sequence
  Nucleotide sequence encoding Myostatin Binding Peptide
cgtatggaaatgcttgaatctcttcttcaacttcttaatgaaattgttccaatgtctcgtgctcgt

SEQ ID NO: 475 - DNA, 66 bp, Artificial Sequence
  Nucleotide sequence encoding Myostatin Binding Peptide
cgtatggaaatgcttgaatctctttttgatcttcttaaagaacttgttccaatgtctaaaggtgtt

SEQ ID NO: 476 - DNA, 66 bp, Artificial Sequence
  Nucleotide sequence encoding Myostatin Binding Peptide
cgtattgaaatgcttgaatctcttcttgaacttcttaaagatattgttccaattcaaaaagctcgt

SEQ ID NO: 477 - DNA, 66 bp, Artificial Sequence
  Nucleotide sequence encoding Myostatin Binding Peptide
cgtatggaacttcttgaatctctttttgaacttcttaaagatatggttccaatgtctgattcttct

SEQ ID NO: 478 - DNA, 66 bp, Artificial Sequence
  Nucleotide sequence encoding Myostatin Binding Peptide
cgtatggaaatgcttgaatctcttcttgaagttcttcaagaaattgttccacgtgctaaaggtgct

SEQ ID NO: 479 - DNA, 66 bp, Artificial Sequence
  Nucleotide sequence encoding Myostatin Binding Peptide
cgtatggaaatgcttgattctcttcttcaacttcttaatgaaattgttccaatgtctcatgctcgt

SEQ ID NO: 480 - DNA, 66 bp, Artificial Sequence
  Nucleotide sequence encoding Myostatin Binding Peptide
cgtatggaaatgcttgaatctcttcttgaacttcttaaagatattgttccaatgtctaatgctggt

SEQ ID NO: 481 - DNA, 66 bp, Artificial Sequence
  Nucleotide sequence encoding Myostatin Binding Peptide
cgtatggaaatgcttcaatctctttttgaacttcttaaaggtatggttccaatttctaaagctggt

SEQ ID NO: 482 - DNA, 66 bp, Artificial Sequence
  Nucleotide sequence encoding Myostatin Binding Peptide
cgtatggaaatgcttgaatctcttcttgaacttcttaaagaaattgttccaaattctactgctgct

SEQ ID NO: 483 - DNA, 66 bp, Artificial Sequence
  Nucleotide sequence encoding Myostatin Binding Peptide
cgtatggaaatgcttcaatctcttcttgaacttcttaaagaaattgttccaatttctaaagctggt

SEQ ID NO: 484 - DNA, 66 bp, Artificial Sequence
  Nucleotide sequence encoding Myostatin Binding Peptide
cgtattgaaatgcttgattctcttcttgaacttcttaatgaacttgttccaatgtctaaagctcgt

SEQ ID NO: 485 - DNA, 66 bp, Artificial Sequence
  Nucleotide sequence encoding Myostatin Binding Peptide
gattggcgtgctactcttcttaaagaattttggcaacttgttgaaggtcttggtgataatcttgtt

SEQ ID NO: 486 - DNA, 66 bp, Artificial Sequence
  Nucleotide sequence encoding Myostatin Binding Peptide
gatggtcgtgctactcttcttactgaattttggcaacttgttcaaggtcttggtcaaaaagaagct

SEQ ID NO: 487 - DNA, 66 bp, Artificial Sequence
  Nucleotide sequence encoding Myostatin Binding Peptide
cttgctcgtgctactcttcttaaagaattttggcaacttgttgaaggtcttggtgaaaaagttgtt

SEQ ID NO: 488 - DNA, 66 bp, Artificial Sequence
  Nucleotide sequence encoding Myostatin Binding Peptide
ggttctcgtgatactcttcttaaagaattttggcaacttgttgttggtcttggtgatatgcaaact

SEQ ID NO: 489 - DNA, 66 bp, Artificial Sequence
  Nucleotide sequence encoding Myostatin Binding Peptide
gatgctcgtgctactcttcttaaagaattttggcaacttgttgatgcttatggtgatcgtatggtt

SEQ ID NO: 490 - DNA, 66 bp, Artificial Sequence
  Nucleotide sequence encoding Myostatin Binding Peptide
aatgatcgtgctcaacttcttcgtgatttttggcaacttgttgatggtcttggtgttaaatcttgg

SEQ ID NO: 491 - DNA, 66 bp, Artificial Sequence
  Nucleotide sequence encoding Myostatin Binding Peptide
ggtgttcgtgaaactcttctttatgaactttggtatcttcttaaaggtcttggtgctaatcaaggt

SEQ ID NO: 492 - DNA, 66 bp, Artificial Sequence
  Nucleotide sequence encoding Myostatin Binding Peptide
caagctcgtgctactcttcttaaagaattttgtcaacttgttggttgtcaaggtgataaactttct

SEQ ID NO: 493 - DNA, 66 bp, Artificial Sequence
  Nucleotide sequence encoding Myostatin Binding Peptide
caagaacgtgctactcttcttaaagaattttggcaacttgttgctggtcttggtcaaaatatgcgt

SEQ ID NO: 494 - DNA, 66 bp, Artificial Sequence
  Nucleotide sequence encoding Myostatin Binding Peptide
tctggtcgtgctactcttcttaaagaattttggcaacttgttcaaggtcttggtgaatatcgttgg

SEQ ID NO: 495 - DNA, 66 bp, Artificial Sequence
  Nucleotide sequence encoding Myostatin Binding Peptide
actatgcgtgctactcttcttaaagaattttggctttttgttgatggtcaacgtgaaatgcaatgg

SEQ ID NO: 496 - DNA, 66 bp, Artificial Sequence
  Nucleotide sequence encoding Myostatin Binding Peptide
ggtgaacgtgctactcttcttaatgatttttggcaacttgttgatggtcaaggtgataatactggt

SEQ ID NO: 497 - DNA, 66 bp, Artificial Sequence
  Nucleotide sequence encoding Myostatin Binding Peptide
gatgaacgtgaaactcttcttaaagaattttggcaacttgttcatggttggggtgataatgttgct

SEQ ID NO: 498 - DNA, 66 bp, Artificial Sequence
  Nucleotide sequence encoding Myostatin Binding Peptide
ggtggtcgtgctactcttcttaaagaactttggcaacttcttgaaggtcaaggtgctaatcttgtt

SEQ ID NO: 499 - DNA, 66 bp, Artificial Sequence
  Nucleotide sequence encoding Myostatin Binding Peptide
actgctcgtgctactcttcttaatgaacttgttcaacttgttaaaggttatggtgataaacttgtt

SEQ ID NO: 500 - DNA, 66 bp, Artificial Sequence
  Nucleotide sequence encoding Myostatin Binding Peptide
ggtatgcgtgctactcttcttcaagaattttggcaacttgttggtggtcaaggtgataattggatg

SEQ ID NO: 501 - DNA, 66 bp, Artificial Sequence
  Nucleotide sequence encoding Myostatin Binding Peptide
tctactcgtgctactcttcttaatgatctttggcaacttatgaaaggttgggctgaagatcgtggt

SEQ ID NO: 502 - DNA, 66 bp, Artificial Sequence
  Nucleotide sequence encoding Myostatin Binding Peptide
tctgaacgtgctactcttcttaaagaactttggcaacttgttggtggttggggtgataattttggt

SEQ ID NO: 503 - DNA, 66 bp, Artificial Sequence
  Nucleotide sequence encoding Myostatin Binding Peptide
gttggtcgtgctactcttcttaaagaattttggcaacttgttgaaggtcttgttggtcaatctcgt

SEQ ID NO: 504 - protein, 61 aa, Artificial Sequence
  Peptibody
MGAQWYPCYEGHFWCYDLGSGSATGGSGSTASSGSGSATGWYPCYEGHFWCYDLLEGGGGG

SEQ ID NO: 505 - DNA, 129 bp, Artificial Sequence; identical to SEQ ID NO: 281
  Nucleotide sequence encoding Myostatin Binding Peptide

SEQ ID NO: 506 - protein, 59 aa, Artificial Sequence
  Peptibody
GGGGGAQWYPCYEGHFWCYDLGSGSATGGSGSTASSGSGSATGWYPCYEGHFWCYDLLE

SEQ ID NO: 507 - DNA, 129 bp, Artificial Sequence; identical to SEQ ID NO: 281
  Nucleotide sequence encoding Myostatin Binding Peptide

SEQ ID NO: 508 - protein, 61 aa, Artificial Sequence
  Peptibody
MGAQIFGCKWWDVQCYQFGSGSATGGSGSTASSGSGSATGIFGCKWWDVQCYQFLEGGGGG

SEQ ID NO: 509 - DNA, 129 bp, Artificial Sequence
  Nucleotide sequence encoding Myostatin Binding Peptide
atctttggctgtaaatggtgggacgttcagtgctaccagttcggttctggttccactgcttcttctggttccggttccgctactggtatcttcggttgcaagtggtgggatgtacagtgttatcagttt

SEQ ID NO: 510 - protein, 59 aa, Artificial Sequence
  Peptibody
GGGGGAQIFGCKWWDVQCYQFGSGSATGGSGSTASSGSGSATGIFGCKWWDVQCYQFLE

SEQ ID NO: 511 - DNA, 129 bp, Artificial Sequence; identical to SEQ ID NO: 509
  Nucleotide sequence encoding Myostatin Binding Peptide

SEQ ID NO: 512 - protein, 61 aa, Artificial Sequence
  Peptibody
MGAQIFGCKWWDVDCYQFGSGSATGGSGSTASSGSGSATGIFGCKWWDVDCYQFLEGGGGG

SEQ ID NO: 513 - DNA, 129 bp, Artificial Sequence
  Nucleotide sequence encoding Myostatin Binding Peptide
atctttggctgtaagtggtgggacgttgactgctaccagttcggttctggttccactgcttcttctggttccggttccgctactggtatcttcggttgcaaatggtgggacgttgattgttatcagttt

SEQ ID NO: 514 - protein, 59 aa, Artificial Sequence
  Peptibody
GGGGGAQIFGCKWWDVDCYQFGSGSATGGSGSTASSGSGSATGIFGCKWWDVDCYQFLE

SEQ ID NO: 515 - DNA, 129 bp, Artificial Sequence; identical to SEQ ID NO: 513
  Nucleotide sequence encoding Myostatin Binding Peptide

SEQ ID NO: 516 - DNA, 66 bp, Artificial Sequence
  Nucleotide sequence encoding Myostatin Binding Peptide
caggttgaatccctgcagcagctgctgatgtggctggaccagaaactggcttccggtccgcagggt

SEQ ID NO: 517 - DNA, 66 bp, Artificial Sequence
  Nucleotide sequence encoding Myostatin Binding Peptide
cgtatggaactgctggaatccctgttcgaactgctgaaagaaatggttccgcgttccaaagctgtt

SEQ ID NO: 518 - DNA, 66 bp, Artificial Sequence
  Nucleotide sequence encoding Myostatin Binding Peptide
caggctgtttccctgcagcacctgctgatgtggctggaccagaaactggcttccggtccgcagcac

SEQ ID NO: 519 - DNA, 66 bp, Artificial Sequence
  Nucleotide sequence encoding Myostatin Binding Peptide
gacgaagactccctgcagcagctgctgatgtggctggaccagaaactggcttccggtccgcagctg

SEQ ID NO: 520 - DNA, 66 bp, Artificial Sequence
  Nucleotide sequence encoding Myostatin Binding Peptide
ccggttgcttccctgcagcagctgctgatctggctggaccagaaactggctcagggtccgcacgct

SEQ ID NO: 521 - DNA, 66 bp, Artificial Sequence
  Nucleotide sequence encoding Myostatin Binding Peptide
gaagttgacgaactgcagcagctgctgaactggctggaccacaaactggcttccggtccgctgcag

SEQ ID NO: 522 - DNA, 66 bp, Artificial Sequence
  Nucleotide sequence encoding Myostatin Binding Peptide
gacgttgaatccctggaacagctgctgatgtggctggaccaccagctggcttccggtccgcacggt

SEQ ID NO: 523 - DNA, 66 bp, Artificial Sequence
  Nucleotide sequence encoding Myostatin Binding Peptide
caggttgactccctgcagcaggttctgctgtggctggaacacaaactggctctgggtccgcaggtt

SEQ ID NO: 524 - DNA, 66 bp, Artificial Sequence
  Nucleotide sequence encoding Myostatin Binding Peptide
ggtgacgaatccctgcagcacctgctgatgtggctggaacagaaactggctctgggtccgcacggt

SEQ ID NO: 525 - DNA, 66 bp, Artificial Sequence
  Nucleotide sequence encoding Myostatin Binding Peptide
cagatcgaaatgctggaatccctgctggacctgctgcgtgacatggttccgatgtccaacgctttc

SEQ ID NO: 526 - DNA, 66 bp, Artificial Sequence
  Nucleotide sequence encoding Myostatin Binding Peptide
gaagttgactccctgcagcagctgctgatgtggctggaccagaaactggcttccggtccgcaggct

SEQ ID NO: 527 - DNA, 66 bp, Artificial Sequence
  Nucleotide sequence encoding Myostatin Binding Peptide
gaagacgaatccctgcagcagctgctgatctacctggacaaaatgctgtcctccggtccgcaggtt

SEQ ID NO: 528 - DNA, 66 bp, Artificial Sequence
  Nucleotide sequence encoding Myostatin Binding Peptide
gctatggaccagctgcaccagctgctgatctggctggaccacaaactggcttccggtccgcaggct

SEQ ID NO: 529 - DNA, 66 bp, Artificial Sequence
  Nucleotide sequence encoding Myostatin Binding Peptide
cgtatcgaaatgctggaatccctgctggaactgctggacgaaatcgctctgatcccgaaagcttgg

SEQ ID NO: 530 - DNA, 66 bp, Artificial Sequence
  Nucleotide sequence encoding Myostatin Binding Peptide
gaagttgtttccctgcagcacctgctgatgtggctggaacacaaactggcttccggtccggacggt

SEQ ID NO: 531 - DNA, 66 bp, Artificial Sequence
  Nucleotide sequence encoding Myostatin Binding Peptide
ggtggtgaatccctgcagcagctgctgatgtggctggaccagcagctggcttccggtccgcagcgt

SEQ ID NO: 532 - DNA, 66 bp, Artificial Sequence
  Nucleotide sequence encoding Myostatin Binding Peptide
ggtgttgaatccctgcagcagctgctgatcttcctggaccacatgctggtttccggtccgcacgac

SEQ ID NO: 533 - DNA, 66 bp, Artificial Sequence
  Nucleotide sequence encoding Myostatin Binding Peptide
aacgttgaatccctggaacacctgatgatgtggctggaacgtctgctggcttccggtccgtacgct

SEQ ID NO: 534 - DNA, 66 bp, Artificial Sequence
  Nucleotide sequence encoding Myostatin Binding Peptide
caggttgactccctgcagcagctgctgatctggctggaccaccagctggcttccggtccgaaacgt

SEQ ID NO: 535 - DNA, 66 bp, Artificial Sequence
  Nucleotide sequence encoding Myostatin Binding Peptide
gaagttgaatccctgcagcagctgctgatgtggctggaacacaaactggctcagggtccgcagggt

SEQ ID NO: 536 - DNA, 66 bp, Artificial Sequence
  Nucleotide sequence encoding Myostatin Binding Peptide
gaagttgactccctgcagcagctgctgatgtggctggaccagaaactggcttccggtccgcacgct

SEQ ID NO: 537 - DNA, 66 bp, Artificial Sequence
  Nucleotide sequence encoding Myostatin Binding Peptide
gaagttgactccctgcagcagctgctgatgtggctggaccagcagctggcttccggtccgcagaaa

SEQ ID NO: 538 - DNA, 66 bp, Artificial Sequence
  Nucleotide sequence encoding Myostatin Binding Peptide
ggtgttgaacagctgccgcagctgctgatgtggctggaacagaaactggcttccggtccgcagcgt

SEQ ID NO: 539 - DNA, 66 bp, Artificial Sequence
  Nucleotide sequence encoding Myostatin Binding Peptide
ggtgaagactccctgcagcagctgctgatgtggctggaccagcagctggctgctggtccgcaggtt

SEQ ID NO: 540 - DNA, 66 bp, Artificial Sequence
  Nucleotide sequence encoding Myostatin Binding Peptide
gctgacgactccctgcagcagctgctgatgtggctggaccgtaaactggcttccggtccgcacgtt

SEQ ID NO: 541 - DNA, 66 bp, Artificial Sequence
  Nucleotide sequence encoding Myostatin Binding Peptide
ccggttgactccctgcagcagctgctgatctggctggaccagaaactggcttccggtccgcagggt

SEQ ID NO: 542 - DNA, 66 bp, Artificial Sequence
  Nucleotide sequence encoding Myostatin Binding Peptide
cagtcccgtgctaccctgctgaaagaattctggcagctggttgaaggtctgggtgacaaacaggct

SEQ ID NO: 543 - DNA, 66 bp, Artificial Sequence
  Nucleotide sequence encoding Myostatin Binding Peptide
gaaatccgtgctaccctgctgaaagaattctggcagctggttgacgaatggcgtgaacagccgaac

SEQ ID NO: 544 - DNA, 66 bp, Artificial Sequence
  Nucleotide sequence encoding Myostatin Binding Peptide
cagctgcgtgctaccctgctgaaagaattcctgcagctggttcacggtctgggtgaaaccgactcc

SEQ ID NO: 545 - DNA, 66 bp, Artificial Sequence
  Nucleotide sequence encoding Myostatin Binding Peptide
acccagcgtgctaccctgctgaaagaattctggcagctgatcgaaggtctgggtggtaaacacgtt

SEQ ID NO: 546 - DNA, 66 bp, Artificial Sequence
  Nucleotide sequence encoding Myostatin Binding Peptide
cactaccgtgctaccctgctgaaagaattctggcagctggttgacggtctgcgtgaacagggtgtt

SEQ ID NO: 547 - DNA, 66 bp, Artificial Sequence
  Nucleotide sequence encoding Myostatin Binding Peptide
cagtcccgtgttaccctgctgcgtgaattctggcagctggttgaatcctaccgtccgatcgttaac

SEQ ID NO: 548 - DNA, 66 bp, Artificial Sequence
  Nucleotide sequence encoding Myostatin Binding Peptide
ctgtcccgtgctaccctgctgaacgaattctggcagttcgttgacggtcagcgtgacaaacgtatg

SEQ ID NO: 549 - DNA, 66 bp, Artificial Sequence
  Nucleotide sequence encoding Myostatin Binding Peptide
tgggaccgtgctaccctgctgaacgacttctggcacctgatggaagaactgtcccagaaaccgggt

SEQ ID NO: 550 - DNA, 66 bp, Artificial Sequence
  Nucleotide sequence encoding Myostatin Binding Peptide
caggaacgtgctaccctgctgaaagaattctggcgtatggttgaaggtctgggtaaaaaccgtggt

SEQ ID NO: 551 - DNA, 66 bp, Artificial Sequence
  Nucleotide sequence encoding Myostatin Binding Peptide
aacgaacgtgctaccctgctgcgtgaattctggcagctggttggtggttacggtgttaaccagcgt

SEQ ID NO: 552 - DNA, 60 bp, Artificial Sequence
  Nucleotide sequence encoding Myostatin Binding Peptide
cagcgtgaatggtacccgtgctacggtggtcacctgtggtgctacgacctgcacaaagct

SEQ ID NO: 553 - DNA, 60 bp, Artificial Sequence
  Nucleotide sequence encoding Myostatin Binding Peptide
atctccgcttggtactcctgctacgctggtcacttctggtgctgggacctgaaacagaaa

SEQ ID NO: 554 - DNA, 60 bp, Artificial Sequence
  Nucleotide sequence encoding Myostatin Binding Peptide
tggaccggttggtaccagtgctacggtggtcacctgtggtgctacgacctgcgtcgtaaa

SEQ ID NO: 555 - DNA, 60 bp, Artificial Sequence
  Nucleotide sequence encoding Myostatin Binding Peptide
aaaaccttctggtacccgtgctacgacggtcacttctggtgctacaacctgaaatcctcc

SEQ ID NO: 556 - DNA, 60 bp, Artificial Sequence
  Nucleotide sequence encoding Myostatin Binding Peptide
gaatcccgttggtacccgtgctacgaaggtcacctgtggtgcttcgacctgaccgaaacc

SEQ ID NO: 557 - DNA, 66 bp, Artificial Sequence
  Nucleotide sequence encoding Myostatin Binding Peptide
aatgttttttttcaatgggttcaaaaacatggtcgtgttgtttatcaatggcttgatattaatgtt

SEQ ID NO: 558 - DNA, 66 bp, Artificial Sequence
  Nucleotide sequence encoding Myostatin Binding Peptide
tttgattttcttcaatggcttcaaaatcatcgttctgaagttgaacattggcttgttatggatgtt

SEQ ID NO: 559 - DNA, 66 bp, Artificial Sequence
  Nucleotide sequence encoding Myostatin Binding Peptide
catcaacgtgatatgtctatgctttgggaacttcttgatgttcttgatggtcttcgtcaatattct

SEQ ID NO: 560 - DNA, 66 bp, Artificial Sequence
  Nucleotide sequence encoding Myostatin Binding Peptide
actcaacgtgatatgtctatgcttgatggtcttcttgaagttcttgatcaacttcgtcaacaacgt

SEQ ID NO: 561 - DNA, 66 bp, Artificial Sequence
  Nucleotide sequence encoding Myostatin Binding Peptide
acctcccgtgacatgtccctgctgtgggaactgctggaagaactggaccgtctgggtcaccagcgt

SEQ ID NO: 562 - DNA, 66 bp, Artificial Sequence
  Nucleotide sequence encoding Myostatin Binding Peptide
atgcaacatgatatgtctatgctttatggtcttgttgaacttcttgaatctcttggtcatcaaatt

SEQ ID NO: 563 - DNA, 66 bp, Artificial Sequence
  Nucleotide sequence encoding Myostatin Binding Peptide
tggaatcgtgatatgcgtatgcttgaatctctttttgaagttcttgatggtcttcgtcaacaagtt

SEQ ID NO: 564 - DNA, 66 bp, Artificial Sequence
  Nucleotide sequence encoding Myostatin Binding Peptide
ggttatcgtgatatgtctatgcttgaaggtcttcttgctgttcttgatcgtcttggtccacaactt

SEQ ID NO: 565 - DNA, 66 bp, Artificial Sequence
  Nucleotide sequence encoding Myostatin Binding Peptide
actcaacgtgatatgtctatgcttgaaggtcttcttgaagttcttgatcgtcttggtcaacaacgt

SEQ ID NO: 566 - DNA, 66 bp, Artificial Sequence
  Nucleotide sequence encoding Myostatin Binding Peptide
tggtaccgtgacatgtccatgctggaaggtctgctggaagttctggaccgtctgggtcagcagcgt

SEQ ID NO: 567 - DNA, 66 bp, Artificial Sequence
  Nucleotide sequence encoding Myostatin Binding Peptide
actcaaaattctcgtcaaatgcttctttctgattttatgatgcttgttggttctatgattcaaggt

SEQ ID NO: 568 - DNA, 66 bp, Artificial Sequence
  Nucleotide sequence encoding Myostatin Binding Peptide
atgcaaacttctcgtcatattcttctttctgaatttatgatgcttgttggttctattatgcatggt

SEQ ID NO: 569 - DNA, 66 bp, Artificial Sequence
  Nucleotide sequence encoding Myostatin Binding Peptide
cacgacaactcccgtcagatgctgctgtccgacctgctgcacctggttggtaccatgatccagggt

SEQ ID NO: 570 - DNA, 66 bp, Artificial Sequence
  Nucleotide sequence encoding Myostatin Binding Peptide
atggaaaactcccgtcagaacctgctgcgtgaactgatcatgctggttggtaacatgtcccaccag

SEQ ID NO: 571 - DNA, 66 bp, Artificial Sequence
  Nucleotide sequence encoding Myostatin Binding Peptide
caggacacctcccgtcacatgctgctgcgtgaattcatgatgctggttggtgaaatgatccagggt

SEQ ID NO: 572 - DNA, 66 bp, Artificial Sequence
  Nucleotide sequence encoding Myostatin Binding Peptide
gaccagaactcccgtcagatgctgctgtccgacctgatgatcctggttggttccatgatccagggt

SEQ ID NO: 573 - DNA, 60 bp, Artificial Sequence
  Nucleotide sequence encoding Myostatin Binding Peptide
gttgctcttcatggtcaatgtactcgttggccatggatgtgtccaccacaacgtgaaggt

SEQ ID NO: 574 - DNA, 60 bp, Artificial Sequence
  Nucleotide sequence encoding Myostatin Binding Peptide
tatccagaacaaggtctttgtactcgttggccatggatgtgtccaccacaaactcttgct

SEQ ID NO: 575 - DNA, 60 bp, Artificial Sequence
  Nucleotide sequence encoding Myostatin Binding Peptide
ggtctgaaccagggtcactgcacccgttggccgtggatgtgcccgccgcaggactccaac

SEQ ID NO: 576 - DNA, 60 bp, Artificial Sequence
  Nucleotide sequence encoding Myostatin Binding Peptide
atgattactcaaggtcaatgtactcgttggccatggatgtgtccaccacaaccatctggt

SEQ ID NO: 577 - DNA, 60 bp, Artificial Sequence
  Nucleotide sequence encoding Myostatin Binding Peptide
gctggtgctcaggaacactgcacccgttggccgtggatgtgcgctccgaacgactggatc

SEQ ID NO: 578 - DNA, 60 bp, Artificial Sequence
  Nucleotide sequence encoding Myostatin Binding Peptide
ggtgttaaccagggtcagtgcacccgttggcgttggatgtgcccgccgaacggttgggaa

SEQ ID NO: 579 - DNA, 60 bp, Artificial Sequence
  Nucleotide sequence encoding Myostatin Binding Peptide
ctggctgaccacggtcagtgcatccgttggccgtggatgtgcccgccggaaggttgggaa

SEQ ID NO: 580 - DNA, 60 bp, Artificial Sequence
  Nucleotide sequence encoding Myostatin Binding Peptide
atcctggaacaggctcagtgcacccgttggccgtggatgtgcccgccgcagcgtggtggt

SEQ ID NO: 581 - DNA, 60 bp, Artificial Sequence
  Nucleotide sequence encoding Myostatin Binding Peptide
actcaaactcatgctcaatgtactcgttggccatggatgtgtccaccacaatgggaaggt

SEQ ID NO: 582 - DNA, 60 bp, Artificial Sequence
  Nucleotide sequence encoding Myostatin Binding Peptide
gttgttactcaaggtcattgtactctttggccatggatgtgtccaccacaacgttggcgt

SEQ ID NO: 583 - DNA, 60 bp, Artificial Sequence
  Nucleotide sequence encoding Myostatin Binding Peptide
atttatccacatgatcaatgtactcgttggccatggatgtgtccaccacaaccatatcca

SEQ ID NO: 584 - DNA, 60 bp, Artificial Sequence
  Nucleotide sequence encoding Myostatin Binding Peptide
tcttattggcaaggtcaatgtactcgttggccatggatgtgtccaccacaatggcgtggt

SEQ ID NO: 585 - DNA, 60 bp, Artificial Sequence
  Nucleotide sequence encoding Myostatin Binding Peptide
atgtggcaacaaggtcattgtactcgttggccatggatgtgtccaccacaaggttggggt

SEQ ID NO: 586 - DNA, 60 bp, Artificial Sequence
  Nucleotide sequence encoding Myostatin Binding Peptide
gaattcacccagtggcactgcacccgttggccgtggatgtgcccgccgcagcgttcccag

SEQ ID NO: 587 - DNA, 60 bp, Artificial Sequence
  Nucleotide sequence encoding Myostatin Binding Peptide
ctggacgaccagtggcagtgcacccgttggccgtggatgtgcccgccgcagggtttctcc

SEQ ID NO: 588 - DNA, 60 bp, Artificial Sequence
  Nucleotide sequence encoding Myostatin Binding Peptide
tatcaaactcaaggtctttgtactcgttggccatggatgtgtccaccacaatctcaacgt

SEQ ID NO: 589 - DNA, 60 bp, Artificial Sequence
  Nucleotide sequence encoding Myostatin Binding Peptide
gaatctaatcaaggtcaatgtactcgttggccatggatgtgtccaccacaaggtggttgg

SEQ ID NO: 590 - DNA, 60 bp, Artificial Sequence
  Nucleotide sequence encoding Myostatin Binding Peptide
tggaccgaccgtggtccgtgcacccgttggccgtggatgtgcccgccgcaggctaacggt

SEQ ID NO: 591 - DNA, 60 bp, Artificial Sequence
  Nucleotide sequence encoding Myostatin Binding Peptide
gttggtacccagggtcagtgcacccgttggccgtggatgtgcccgccgtacgaaaccggt

SEQ ID NO: 592 - DNA, 60 bp, Artificial Sequence
  Nucleotide sequence encoding Myostatin Binding Peptide
ccgtacgaacagggtaaatgcacccgttggccgtggatgtgcccgccgtacgaagttgaa

SEQ ID NO: 593 - DNA, 60 bp, Artificial Sequence
  Nucleotide sequence encoding Myostatin Binding Peptide
tccgaataccagggtctgtgcacccgttggccgtggatgtgcccgccgcagggttggaaa

SEQ ID NO: 594 - DNA, 60 bp, Artificial Sequence
  Nucleotide sequence encoding Myostatin Binding Peptide
accttctcccagggtcactgcacccgttggccgtggatgtgcccgccgcagggttggggt

SEQ ID NO: 595 - DNA, 60 bp, Artificial Sequence
  Nucleotide sequence encoding Myostatin Binding Peptide
ccgggtgctcacgaccactgcacccgttggccgtggatgtgcccgccgcagtcccgttac

SEQ ID NO: 596 - DNA, 60 bp, Artificial Sequence
  Nucleotide sequence encoding Myostatin Binding Peptide
gttgctgaagaatggcactgccgtcgttggccgtggatgtgcccgccgcaggactggcgt

SEQ ID NO: 597 - DNA, 60 bp, Artificial Sequence
  Nucleotide sequence encoding Myostatin Binding Peptide
gttggtacccagggtcactgcacccgttggccgtggatgtgcccgccgcagccggctggt

SEQ ID NO: 598 - DNA, 60 bp, Artificial Sequence
  Nucleotide sequence encoding Myostatin Binding Peptide
gaagaagaccaggctcactgccgttcctggccgtggatgtgcccgccgcagggttgggtt

SEQ ID NO: 599 - DNA, 60 bp, Artificial Sequence
  Nucleotide sequence encoding Myostatin Binding Peptide
gctgacacccagggtcactgcacccgttggccgtggatgtgcccgccgcagcactggttc

SEQ ID NO: 600 - DNA, 60 bp, Artificial Sequence
  Nucleotide sequence encoding Myostatin Binding Peptide
tccggtccgcagggtcactgcacccgttggccgtggatgtgcgctccgcagggttggttc

SEQ ID NO: 601 - DNA, 60 bp, Artificial Sequence
  Nucleotide sequence encoding Myostatin Binding Peptide
accctggttcagggtcactgcacccgttggccgtggatgtgcccgccgcagcgttgggtt

SEQ ID NO: 602 - DNA, 60 bp, Artificial Sequence
  Nucleotide sequence encoding Myostatin Binding Peptide
ggtatggctcacggtaaatgcacccgttgggcttggatgtgcccgccgcagtcctggaaa

SEQ ID NO: 603 - DNA, 60 bp, Artificial Sequence
  Nucleotide sequence encoding Myostatin Binding Peptide
gaactgtaccacggtcagtgcacccgttggccgtggatgtgcccgccgcagtcctgggct

SEQ ID NO: 604 - DNA, 60 bp, Artificial Sequence
  Nucleotide sequence encoding Myostatin Binding Peptide
gttgctgaccacggtcactgcacccgttggccgtggatgtgcccgccgcagggttggggt

SEQ ID NO: 605 - DNA, 60 bp, Artificial Sequence
  Nucleotide sequence encoding Myostatin Binding Peptide
ccggaatcccagggtcactgcacccgttggccgtggatgtgcccgccgcagggttggggt

SEQ ID NO: 606 - DNA, 60 bp, Artificial Sequence
  Nucleotide sequence encoding Myostatin Binding Peptide
atcccggctcacggtcactgcacccgttggccgtggatgtgcccgccgcagcgttggcgt

SEQ ID NO: 607 - DNA, 60 bp, Artificial Sequence
  Nucleotide sequence encoding Myostatin Binding Peptide
ttcaccgttcacggtcactgcacccgttggccgtggatgtgcccgccgtacggttgggtt

SEQ ID NO: 608 - DNA, 60 bp, Artificial Sequence
  Nucleotide sequence encoding Myostatin Binding Peptide
ccagattttccaggtcattgtactcgttggcgttggatgtgtccaccacaaggttgggaa

SEQ ID NO: 609 - DNA, 60 bp, Artificial Sequence
  Nucleotide sequence encoding Myostatin Binding Peptide
cagctgtggcagggtccgtgcacccagtggccgtggatgtgcccgccgaaaggtcgttac

SEQ ID NO: 610 - DNA, 60 bp, Artificial Sequence
  Nucleotide sequence encoding Myostatin Binding Peptide
cacgctaacgacggtcactgcacccgttggcagtggatgtgcccgccgcagtggggtggt

SEQ ID NO: 611 - DNA, 60 bp, Artificial Sequence
  Nucleotide sequence encoding Myostatin Binding Peptide
gaaaccgaccacggtctgtgcacccgttggccgtggatgtgcccgccgtacggtgctcgt

SEQ ID NO: 612 - DNA, 60 bp, Artificial Sequence
  Nucleotide sequence encoding Myostatin Binding Peptide
ggtacctggcagggtctgtgcacccgttggccgtggatgtgcccgccgcagggttggcag

SEQ ID NO: 613 - DNA, 60 bp, Artificial Sequence
  Nucleotide sequence encoding Myostatin Binding Peptide
gttgctacccagggtcagtgcacccgttggccgtggatgtgcccgccgcagggttggggt

SEQ ID NO: 614 - DNA, 60 bp, Artificial Sequence
  Nucleotide sequence encoding Myostatin Binding Peptide
gttgctacccagggtcagtgcacccgttggccgtggatgtgcccgccgcagcgttggggt

SEQ ID NO: 615 - protein, 73 aa, Artificial Sequence
  Peptibody
GGGGGAQLADHGQCIRWPWMCPPEGWELEGSGSATGGSGSTASSGSGSATGLADHGQCIRWPWMCPPEGWELE

SEQ ID NO: 616 - DNA, 198 bp, Artificial Sequence
  Nucleotide sequence encoding Myostatin Binding Peptide
cttgctgatcatggtcaatgtattcgttggccatggatgtgtccaccagaaggttgggaactcgagggttccggttccgctaccggcggctctggctccactgcttcttccggttccggttctgctactggtctggctgaccacggtcagtgcatccgttggccgtggatgtgcccgccggaaggttgggaactggaa

SEQ ID NO: 617 - protein, 69 aa, Artificial Sequence
  Peptibody
GGGGGAQLADHGQCIRWPWMCPPEGWEGSGSATGGSGGGASSGSGSATGLADHGQCIRWPWMCPPEGWE

SEQ ID NO: 618 - DNA, 186 bp, Artificial Sequence
  Nucleotide sequence encoding Myostatin Binding Peptide
cttgctgatcatggtcaatgtattcgttggccatggatgtgtccaccagaaggttgggaaggttccggttccgctaccggcggctctggcggtggcgcttcttccggttccggttctgctactggtctggctgaccacggtcagtgcatccgttggccgtggatgtgtccaccagaaggttgggaa

SEQ ID NO: 619 - protein, 73 aa, Artificial Sequence
  Peptibody
GGGGGAQSEYQGLCTRWPWMCPPQGWKLEGSGSATGGSGSTASSGSGSATGSEYQGLCTRWPWMCPPQGWKLE

SEQ ID NO: 620 - DNA, 198 bp, Artificial Sequence
  Nucleotide sequence encoding Myostatin Binding Peptide
tctgaatatcaaggtctttgtactcgttggccatggatgtgtccaccacaaggttggaaactcgagggttccggttccgctaccggcggctctggctccactgcttcttccggttccggttctgctactggttctgagtatcaaggcctctgtactcgctggccatggatgtgtccaccacaaggctggaagctggaa

SEQ ID NO: 621 - protein, 69 aa, Artificial Sequence
  Peptibody
GGGGGAQSEYQGLCTRWPWMCPPQGWKGSGSATGGSGGGASSGSGSATGSEYQGLCTRWPWMCPPQGWK

SEQ ID NO: 622 - DNA, 186 bp, Artificial Sequence
  Nucleotide sequence encoding Myostatin Binding Peptide
tctgaatatcaaggtctttgtactcgttggccatggatgtgtccaccacaaggttggaaaggttccggttccgctaccggcggctctggcggtggcgcttcttccggttccggttctgctactggttctgagtatcaaggcctctgtactcgctggccatggatgtgtccaccacaaggttggaaa

SEQ ID NO: 623 - protein, 73 aa, Artificial Sequence
  Peptibody
GGGGGAQTFSQGHCTRWPWMCPPQGWGLEGSGSATGGSGSTASSGSGSATGTFSQGHCTRWPWMCPPQGWGLE

SEQ ID NO: 624 - DNA, 198 bp, Artificial Sequence
  Nucleotide sequence encoding Myostatin Binding Peptide
actttttctcaaggtcattgtactcgttggccatggatgtgtccaccacaaggttggggtctcgagggttccggttccgctaccggcggctctggctccactgcttcttccggttccggttctgctactggtactttttctcaaggccattgtactcgctggccatggatgtgtccaccacaaggctggggcctggaa

SEQ ID NO: 625 - protein, 73 aa, Artificial Sequence
  Peptibody
GGGGGAQVADHGHCTRWPWMCPPQGWGLEGSGSATGGSGSTASSGSGSATGVADHGHCTRWPWMCPPQGWGLE

SEQ ID NO: 626 - DNA, 198 bp, Artificial Sequence
  Nucleotide sequence encoding Myostatin Binding Peptide
gttgctgatcatggtcattgtactcgttggccatggatgtgtccaccacaaggttggggtctcgagggttccggttccgcaaccggcggctctggctccactgcttcttccggttccggttctgctactggtgttgctgaccacggtcactgcacccgttggccgtggatgtgcccgccgcagggttggggtctggaa

SEQ ID NO: 627 - protein, 69 aa, Artificial Sequence
  Peptibody
GGGGGAQVADHGHCTRWPWMCPPQGWGGSGSATGGSGGGASSGSGSATGVADHGHCTRWPWVCPPQGWG

SEQ ID NO: 628 - DNA, 186 bp, Artificial Sequence
  Nucleotide sequence encoding Myostatin Binding Peptide
gttgctgatcatggtcattgtactcgttggccatggatgtgtccaccacaaggttggggtggttccggttccgctaccggcggctctggcggtggtgcttcttccggttccggttctgctactggtgttgctgaccacggtcactgcacccgttggccgtgggtgtgtccaccacaaggttggggt

SEQ ID NO: 629 - protein, 73 aa, Artificial Sequence
  Peptibody
GGGGGAQPESQGHCTRWPWMCPPQGWGLEGSGSATGGSGSTASSGSGSATGPESQGHCTRWPWMCPPQGWGLE

SEQ ID NO: 630 - DNA, 198 bp, Artificial Sequence
  Nucleotide sequence encoding Myostatin Binding Peptide
ccagaatctcaaggtcattgtactcgttggccatggatgtgtccaccacaaggttggggtctcgagggttccggttccgctaccggcggctctggctccactgcttcttccggttccggttctgctactggtccggaatcccagggtcactgcacccgttggccgtggatgtgcccgccgcagggttggggtctggaa

SEQ ID NO: 631 - protein, 69 aa, Artificial Sequence
  Peptibody
GGGGGAQPESQGHCTRWPWMCPPQGWGGSGSATGGSGGGASSGSGSATGPESQGHCTRWPWMCPPQGWG

SEQ ID NO: 632 - DNA, 186 bp, Artificial Sequence
  Nucleotide sequence encoding Myostatin Binding Peptide
ccagaatctcaaggtcattgtactcgttggccatggatgtgtccaccacaaggttggggtggttccggttccgctaccggcggctctggcggtggtgcttcttccggttccggttctgctactggtccggaatcccagggtcactgcacccgttggccgtggatgtgtccaccacaaggttggggt

SEQ ID NO: 633 - protein, 5 aa, Artificial Sequence
  Myostatin Binding Peptide
WMCPP

SEQ ID NO: 634 - DNA, 90 bp, Artificial Sequence
  Randomly generated nucleotide sequence
cacagtgcacagggtnnknnknnkcakggkcaktgkackcgktgkccktgkatktgkcckccktaknnknnknnkcattctctcgagatc

SEQ ID NO: 635 - protein, 5 aa, Artificial Sequence
  linker
GGGGG

(19) United States
(12) Patent Application Publication
(10) Pub. No.: US 2014/0294866 A1
(43) Pub. Date: Oct. 2, 2014

(54) Uses of Myostatin Antagonists

(71) Applicant: Amgen, Inc., Thousand Oaks, CA (US)

(72) Inventors: Huiquan Han, Thousand Oaks, CA (US); Alexander DePaoli, Santa Barbara, CA (US); John Zhao-Nian Lu, Culver City, CA (US); Jin Lin Wang, Moorpark, CA (US)

(21) Appl. No.: 14/309,590

(22) Filed: Jun. 19, 2014

Related U.S. Application Data
(63) Continuation of application No. 12/891,741, filed on Sep. 27, 2010, now abandoned.
Application No. 12/891,741 is a continuation of application No. 11/635,731, filed on Dec. 6, 2006, now abandoned.
(60) Provisional application No. 60/742,731, filed on Dec. 6, 2005.

(51) Int. Cl. C07K 14/435; C07K 16/22

(52) U.S. Cl. CPC C07K 14/435; C07K 16/22
USPC 424/179.1; 514/8.9

(57) ABSTRACT

The present invention provides methods for treating disorders arising from hypogonadism by administering myostatin antagonists to subjects suffering from such disorders.

### CROSS-REFERENCE TO RELATED APPLICATIONS

This application is a continuation of U.S. patent application Ser. No. 12/891,741, filed Sep. 27, 2010, (pending), which is a continuation of U.S. patent application Ser. No. 11/635,731, filed Dec. 6, 2006 (abandoned), which claims the benefit of U.S. provisional application No. 60/742,731 filed Dec. 6, 2005, the entire disclosures of which are relied upon and incorporated by reference.

### REFERENCE TO A SEQUENCE LISTING

The instant application contains a Sequence Listing which has been submitted via EFS-Web and is hereby incorporated by reference in its entirety. Said ASCII copy, created on Jun. 18, 2014, is named 27145US_sequencelisting.txt, and is 196,608 bytes in size.

### FIELD OF THE INVENTION

The invention relates to the transforming growth factor-β (TGF-β) family member myostatin, myostatin antagonists, and the uses of these antagonists for the treatment of a variety of diseases.

### BACKGROUND

Myostatin, also known as growth/differentiation factor 8 (GDF-8), is a transforming growth factor-β (TGF-β) family member known to be involved in regulation of skeletal muscle mass. Most members of the TGF-β-GDF family are expressed non-specifically in many tissue types and exert a variety of pleiotropic actions. However, myostatin is largely expressed in the cells of developing and adult skeletal muscle tissue and plays an essential role in negatively controlling skeletal muscle growth (McPherron et al. Nature (London) 387, 83-90 (1997)). Recent studies indicate that myostatin expression can also be measured in cardiac, adipose and pre-adipose tissues.

The myostatin protein has been highly conserved evolutionarily (McPherson et al. PNAS USA 94:12457-12461 (1997)). The biologically active C-terminal region of myostatin has 100 percent sequence identity between human, mouse, rat, cow, chicken, and turkey sequences. The function of myostatin also appears to be conserved across species as well. This is evident from the phenotypes of animals having a mutation in the myostatin gene. Two breeds of cattle, the Belgian Blue (Hanset R., Muscle Hypertrophy of Genetic Origin and its Use to Improve Beef Production, eds, King, J. W. G. & Menissier, F. (Nijhoff, The Hague, The Netherlands) pp. 437-449) and the Piedmontese (Masoero, G. & Poujardieu, B, Muscle Hypertrophy of Genetic Origin and its Use to Improve Beef Production., eds, King, J. W. G. & Menissier, F. (Nijhoff, The Hague, The Netherlands) pp. 450-459) are characterized by a “double muscling” phenotype and increase in muscle mass. These breeds were shown to contain mutations in the coding region of the myostatin gene (McPherron et al. PNAS (1997) supra). In addition, mice containing a targeted deletion of the gene encoding myostatin (Mstn) demonstrate a dramatic increase in muscle mass without a corresponding increase in fat. Individual muscles of Mstn^{-/-} mice weigh approximately 100 to 200 percent more than those of control animals as a result of muscle fiber hypertrophy and hyperplasia (Zimmers et al. Science 296, 1486 (2002)).

The use of myostatin antagonists for treating certain muscle-wasting and metabolic disorders have been described in U.S. application Ser. No. 10/742,379, publication number US 2004/0181033, which is herein incorporated by reference. It has now been discovered that myostatin antagonists can be used to treat additional disorders. The present invention provides methods of treatments for these additional disorders using myostatin antagonists.

### SUMMARY OF THE INVENTION

The present invention provides methods of treatments for various disease conditions. These treatments comprise administering one or more myostatin antagonists to subjects in need of such treatment. The myostatin antagonists can also be administered prophylactically to prevent the development of such condition, and can be administered to a subject either before or after a condition has developed, as needed.

In one embodiment, the invention provides a method of treating the effects of hypogonadism in a subject in need thereof comprising administering a therapeutically effective amount of at least one myostatin antagonist in admixture with a pharmaceutically acceptable carrier to the subject. In one embodiment, the hypogonadism results from androgen deprivation therapy. In another embodiment, the hypogonadism results from age-related decrease in gonadal functioning.

The present invention also provides a method of treating rheumatoid cachexia in a subject suffering from such a condition comprising administering a therapeutically effective amount of at least one myostatin antagonist in admixture with a pharmaceutically acceptable carrier to the subject.

The present invention also provides a method of treating cachexia due to burn injuries in a subject in need of such a treatment comprising administering a therapeutically effective amount of at least one myostatin antagonist in admixture with a pharmaceutically acceptable carrier to the subject.

The present invention also provides a method of treating cachexia due to treatment with chemical agents such as chemotherapeutic agents to a subject in need to such a treatment comprising administering a therapeutically effective amount of at least one myostatin antagonist in admixture with a pharmaceutically acceptable carrier to the subject.

The present invention also provides a method of treating cachexia due to diabetes to a subject in need of such a treatment comprising administering a therapeutically effective amount of at least one myostatin antagonist in admixture with a pharmaceutically acceptable carrier to the subject.

The present invention also provides a method of treating diabetic nephropathy in a subject suffering from such a condition comprising administering a therapeutically effective amount of at least one myostatin antagonist in admixture with a pharmaceutically acceptable carrier to the subject.

The present invention also provides an alternative method of treating diseases or conditions currently treated by growth hormone, insulin growth factor-1 (IGF-1), growth hormone secretagogues, and other agents related to the growth hormone-IGF-1 axis. Myostatin antagonists provide a method of treating such diseases without the potentially dangerous side-effects of growth hormone. In one embodiment, the present invention provides a method of treating the effects of Prader-Willi syndrome in a subject suffering from such a condition comprising administering a therapeutically effective amount of one or more myostatin antagonists in admixture with a pharmaceutically acceptable carrier to the subject.

The present invention also provides a method of reducing TNF-α in a subject suffering from an inflammatory disorder comprising administering a therapeutically effective amount of one or more myostatin antagonists to the subject.

For the methods of treatment listed above, myostatin antagonists include, but are not limited to the following antagonists: follistatin, myostatin prodomain, GDF-11 prodomain, prodomain fusion proteins, antagonistic antibodies or antibody fragments that bind myostatin, antagonistic antibodies or antibody fragments that bind to the activin type IIB receptor, soluble activin type IIB receptor, soluble activin type IIB receptor fusion proteins, soluble myostatin analogs, oligonucleotides, small molecules, peptidomimetics, and myostatin binding agents.

Myostatin binding agents are described extensively in the Detailed Description provided below. As used herein the term “myostatin binding agent” includes all binding agents described herein. For example, a myostatin antagonist useful for the treatments described herein is an exemplary binding agent comprises at least one peptide comprising the amino acid sequence WMCPP (SEQ ID NO: 633). In another embodiment, the myostatin binding agent comprises the amino acid sequence Ca₁a₂Wa₃WMCPP (SEQ ID NO: 352), wherein a₁, a₂ and a₃ are selected from a neutral hydrophobic, neutral polar, or basic amino acid. In another embodiment the myostatin binding agent comprises the sequence Cb₁b₂Wb₃WMCPP (SEQ ID NO: 353), wherein b₁ is selected from any one of the amino acids T, I, or R; b_{z} is selected from any one of R, S, Q; b₃ is selected from any one of P, R and Q, and wherein the peptide is between 10 and 50 amino acids in length, and physiologically acceptable salts thereof. In another embodiment, the myostatin binding agent comprises the formula:

(SEQ ID NO: 354)
c₁c₂c₃c₄c₅c₆Cc₇c₈Wc₉WMCPPc₁₀c₁₁c₁₂C₁₃,

wherein:

c₁ is absent or any amino acid;

c₂ is absent or a neutral hydrophobic, neutral polar, or acidic amino acid;

c₃ is absent or a neutral hydrophobic, neutral polar, or acidic amino acid;

c₄ is absent or any amino acid;

c₅ is absent or a neutral hydrophobic, neutral polar, or acidic amino acid;

c₆ is absent or a neutral hydrophobic, neutral polar, or basic amino acid;

c₇ is a neutral hydrophobic, neutral polar, or basic amino acid;

c₈ is a neutral hydrophobic, neutral polar, or basic amino acid;

c₉ is a neutral hydrophobic, neutral polar or basic amino acid; and

c₁₀ to c₁₃ is any amino acid; and wherein the peptide is between 20 and 50 amino acids in length, and physiologically acceptable salts thereof.

In another embodiment the myostatin binding agent comprises the formula:

(SEQ ID NO: 355)
d₁d₂d₃d₄d₅d₆Cd₇d₈Wd₉WMCPP d₁₀d₁₁d₁₂d₁₃,

wherein

d₁ is absent or any amino acid;

d₂ is absent or a neutral hydrophobic, neutral polar, or acidic amino acid;

d₃ is absent or a neutral hydrophobic, neutral polar, or acidic amino acid;

d₄ is absent or any amino acid;

d₅ is absent or a neutral hydrophobic, neutral polar, or acidic amino acid;

d₆ is absent or a neutral hydrophobic, neutral polar, or basic amino acid;

d₇ is selected from any one of the amino acids T, I, or R;

d₈ is selected from any one of R, S, Q;

d₉ is selected from any one of P, R and Q, and

d₁₀ to d₁₃ is selected from any amino acid,

and wherein the peptide is between 20 and 50 amino acids in length, and physiologically acceptable salts thereof.

Additional embodiments of binding agents useful as myostatin antagonists for treatment of the disorders described herein comprise at least one of the following peptides:

(1) a peptide capable of binding myostatin, wherein the peptide comprises the sequence WYe₁e₂Ye₃G, (SEQ ID NO: 356)

wherein e₁ is P, S or Y,

e₂ is C or Q, and

e₃ is G or H, wherein the peptide is between 7 and 50 amino acids in length, and physiologically acceptable salts thereof;

(2) a peptide capable of binding myostatin, wherein the peptide comprises the sequence f₁EMLf₂SLf₃f₄LL, (SEQ ID NO: 455),

wherein f₁ is M or I,

f₂ is any amino acid,

f₃ is L or F,

f₄ is E, Q or D;

and wherein the peptide is between 7 and 50 amino acids in length, and physiologically acceptable salts thereof;

(3) a peptide capable of binding myostatin wherein the peptide comprises the sequence Lg₁g₂LLg₃g₄L, (SEQ ID NO: 456), wherein

g₁ is Q, D or E,

g₂ is S, Q, D or E,

g₃ is any amino acid,

g₄ is L, W, F, or Y, and wherein the peptide is between 8 and 50 amino acids in length, and physiologically acceptable salts thereof;

(4) a peptide capable of binding myostatin, wherein the peptide comprises the sequence h₁h₂h₃h₄h₅h₆h₇h₈h₉ (SEQ ID NO: 457), wherein

h₁ is R or D,

h₂ is any amino acid,

h₃ is A, T S or Q,

h₄ is L or M,

h₅ is L or S,

h₆ is any amino acid,

h₇ is F or E,

h₈ is W, F or C,

h₉ is L, F, M or K, and wherein the peptide is between 9 and 50 amino acids in length, and physiologically acceptable salts thereof

In another embodiment, described more completely in the Detailed Description below, the binding agents useful as myostatin antagonists comprise at least one vehicle such as a polymer or an Fc domain, and may further comprise at least one linker sequence. In this embodiment, the binding agents of the present invention are constructed so that at least one myostatin binding peptide is attached to at least one vehicle. The peptide or peptides are attached directly or indirectly through a linker sequence, to the vehicle at the N-terminal, C-terminal or an amino acid side chain of the peptide, thereby providing peptibodies. In this embodiment, the binding agents of the present invention have the following generalized structure:

(X¹)ₐ—F¹—(X²)_{b}, or multimers thereof;

wherein F¹ is a vehicle; and X¹ and X² are each independently selected from

-(L¹)_{c}-P¹;

-(L¹)_{c}-P¹-(L²)_{d}-P²;

-(L¹)_{c}-P¹-(L²)_{d}-P²-(L³)ₑ-P³;

and -(L¹)_{c}-P¹-(L²)_{d}-P²-(L³)ₑ-P³-(L⁴)_{f}-P⁴;

wherein P¹, P², P³, and P⁴ are peptides capable of binding myostatin; and L¹, L², L³, and L⁴ are each linkers; and a, b, c, d, e, and f are each independently 0 or 1, provided that at least one of a and b is 1, and physiologically acceptable salts thereof. In embodiments of binding agents having this generalized structure, the peptides P¹, P², P³, and P⁴ can be independently selected from one or more of any of the peptide sequences provided herein, as described in the Detailed Description below. For example, in exemplary embodiments, P¹, P², P³, and P⁴ are independently selected from one or more peptides comprising any of the following sequences: SEQ ID NO: 633, SEQ ID NO: 352, SEQ ID NO: 353, SEQ ID NO: 354, SEQ ID NO: 355, SEQ ID NO: 356, SEQ ID NO: 455, SEQ ID NO: 456, and SEQ ID NO: 457. In another embodiment, P P¹, P², P³, and P⁴ are independently selected from one or more peptides comprising any of the following sequences SEQ ID NO: 305 through 351 and SEQ ID NO: 357 through 454. Additional embodiments of myostatin binding agents are provided in the Detailed Description of the Invention below.

The present invention also provides pharmaceutically acceptable compositions comprising one or more myostatin antagonists for treating hypogonadism, rheumatoid cachexia, cachexia due to burns, cachexia due to chemical agents, cachexia due to diabetes, diabetic nephropathy, Prader Willi syndrome, excessive TNF-α in a subject, and other disorders.

### BRIEF DESCRIPTION OF THE FIGURES

FIG. 1 shows myostatin activity as measured by expressed luciferase activity (y-axis) vs. concentration (x-axis) for the TN8-19 peptide QGHCTRWPWMCPPY (SEQ ID NO: 32) and the TN8-19 peptibody (pb) to determine the IC₅₀ for each using the C2C12 pMARE luciferase assay described in the Examples below. The peptibody has a lower IC₅₀ value compared with the peptide.

FIG. 2 is a graph showing the increase in total body weight for CD1 nu/nu mice treated with increasing dosages of the 1x mTN8-19-21 peptibody over a fourteen day period compared with mice treated with a huFc control, as described in Example 8.

FIG. 3A shows the increase in the mass of the gastrocnemius muscle mass at necropsy of the mice treated in FIG. 2 (Example 8). FIG. 3B shows the increase in lean mass as determined by NMR on day 0 compared with day 13 of the experiment described in Example 8.

FIG. 4 shows the increase in lean body mass as for CD1 nu/nu mice treated with biweekly injections of increasing dosages of 1x mTN8-19-32 peptibody as determined by NMR on day 0 and day 13 of the experiment described in Example 8.

FIG. 5A shows the increase in body weight for CD1 nu/nu mice treated with biweekly injections of 1x mTN8-19-7 compared with 2x mTN8-19-7 and the control animal for 35 days as described in Example 8. FIG. 5B shows the increase in lean carcass weight at necropsy for the 1x and 2x versions at 1 mg/kg and 3 mg/kg compared with the animals receiving the vehicle (huFc) (controls).

FIG. 6A shows the increase in lean muscle mass yrs. body weight for aged mdx mice treated with either affinity matured 1x mTN8-19-33 peptibody or huFc vehicle at 10 mg/kg subcutaneously every other day for three months. FIG. 6B shows the change in fat mass compared to body weight as determined by NMR for the same mice after 3 months of treatment.

FIG. 7 shows the change in body mass over time in grams for collagen-induced arthritis (CIA) animals treated with the peptibody 2x mTN8-19-21/muFc or muFc vehicle, as well as normal non-CIA animals.

FIG. 8 shows the relative body weight change over time in streptozotocin (STZ)-induced diabetic mice treated with the peptibody 2x mTN8-19-21/muFc or the muFc vehicle control.

FIG. 9 shows creatine clearance rate in streptozotocin (STZ)-induced diabetic mice and age-matched normal mice after treatment with peptibody 2x mTN8-19-21/muFc or the muFc vehicle.

FIG. 10A shows urine albumin excretion in streptozotocin (STZ)-induced diabetic mice and age-matched normal mice after treatment with peptibody 2x mTN8-19-21/muFc or the muFc vehicle. FIG. 10B shows the 24 hour urine volume in streptozotocin (STZ)-induced diabetic mice and age-matched normal mice after treatment with peptibody 2x mTN8-19-21/muFc or the muFc vehicle.

FIG. 11 shows body weight change over time for 4 groups of C57B1/6 mice; 2 groups pretreated for 1 week with peptibody 2x mTN8-19-21/muFc, then treated with 5-fluoruracil (5-Fu) or vehicle (PBS); and 2 groups pretreated for 2 weeks with 2x mTN8-19-21/muFc, and then treated with 5-fluorouracil or vehicle (PBS). The triangles along the bottom of the Figure show times of administration of 2 week pretreatment with 2x mTN8-19-21/muFc, times of administration of 1 week pretreatment with 2x mTN8-19-21/muFc, and times of administration of 5-Fu.

FIG. 12 shows the survival rate percentages the animals described in FIG. 11 above, showing normal mice not treated, animals treated with 5-Fu only, animals pretreated with 2x mTN8-19-21/muFc for 1 week and then treated with 5-Fu, and animals pretreated with 2x mTN8-19-21/muFc for 2 weeks and then treated with 5-Fu.

### DETAILED DESCRIPTION OF THE INVENTION

The present invention provides pharmaceutical compositions and methods of treating various disorders using myostatin antagonists including the myostatin binding agents. The invention provides a method of treating the effects of hypogonadism in a subject in need thereof comprising administering a therapeutically effective amount of at least one myostatin antagonist to the subject in admixture with a pharmaceutically acceptable carrier. In one embodiment the hypogonadism results from androgen deprivation therapy. In a second embodiment, the hypogonadism results from age-related decrease in gonadal functioning.

The present invention also provides a method of treating rheumatoid cachexia in a subject suffering from such a condition comprising administering a therapeutically effective amount of at least one myostatin antagonists to the subject in admixture with a pharmaceutically acceptable carrier. The present invention also provides a method of reducing TNF-α in a subject suffering from an inflammatory condition characterized by excessive TNF-α. The present invention also provides a method of treating cachexia due to burn injuries in a subject in need thereof comprising administering a therapeutically effective amount of at least one myostatin antagonist to the subject in admixture with a pharmaceutically acceptable carrier.

The present invention also provides a method of treating cachexia due to treatment with chemical agents such as chemotherapeutic agents to a subject in need to such a treatment comprising administering a therapeutically effective amount of at least one myostatin antagonist in admixture with a pharmaceutically acceptable carrier to the subject.

The present invention also provides a method of treating cachexia due to diabetes to a subject in need of such a treatment comprising administering a therapeutically effective amount of at least one myostatin antagonist in admixture with a pharmaceutically acceptable carrier to the subject. The present invention also provides a method of treating diabetic nephropathy in a subject suffering from such a condition comprising administering a therapeutically effective amount of at least one myostatin antagonist in admixture with a pharmaceutically acceptable carrier to the subject.

The present invention also provides an alternative method of treating diseases or conditions formerly treated by growth hormone, insulin growth factor-1 (IGF-1), growth hormone secretagogues, and other agents related to the growth hormone-IGF-1 axis. Myostatin antagonists provide a method of treating such diseases without the potentially dangerous side-effects of these agents. In one embodiment, the present invention provides a method of treating the effects of Prader-Willi syndrome in a subject suffering from such a condition comprising administering a therapeutically effective amount of at least one myostatin antagonists to the subject in admixture with a pharmaceutically acceptable carrier.

According to the present invention, myostatin antagonists include, but are not limited to, follistatin, myostatin prodomain, GDF-11 prodomain, other TGF-β prodomains, prodomain fusion proteins, antagonistic antibodies or antibody fragments that bind myostatin, antagonistic antibodies or antibody fragments that bind to the activin type IIB receptor, soluble activin type IIB receptor, soluble activin type IIB receptor fusion proteins, soluble myostatin analogs, oligonucleotides, small molecules, peptidomimetics, and myostatin binding agents. These antagonists are described more completely below.

In one embodiment, the myostatin antagonists are myostatin binding agents. Myostatin binding agents have been described in U.S. application Ser. No. 10/742,379, publication number US 2004/0181033, which is herein incorporated by reference herein, and are also described herein.

### Myostatin

Myostatin, a growth factor also known as GDF-8, is a member of the TGF-β family. Myostatin known to be a negative regulator of skeletal muscle tissue. Myostatin is synthesized as an inactive preproprotein which is activated by proteolyic cleavage (Zimmers et al., supra (2002)). The precurser protein is cleaved to produce an NH₂-terminal inactive prodomain and an approximately 109 amino acid COOH-terminal protein in the form of a homodimer of about 25 kDa, which is the mature, active form (Zimmers et al, supra (2002)). It is now believed that the mature dimer circulates in the blood as an inactive latent complex bound to the propeptide (Zimmers et al, supra (2002)).

As used herein the term “full-length myostatin” refers to the full-length human preproprotein sequence described in McPherson et al. PNAS USA 94, 12457 (1997), as well as related full-length polypeptides including allelic variants and interspecies homologs (McPherron et al. supra (1997)). As used herein, the term “prodomain” or “propeptide” refers to the inactive NH₂-terminal protein which is cleaved off to release the active COOH-terminal protein. As used herein the term “myostatin” or “mature myostatin” refers to the mature, biologically active COOH-terminal polypeptide, in monomer, dimer, multimeric form or other form. “Myostatin” or “mature myostatin” also refers to fragments of the biologically active mature myostatin, as well as related polypeptides including allelic variants, splice variants, and fusion peptides and polypeptides. The mature myostatin COOH-terminal protein has been reported to have 100% sequence identity among many species including human, mouse, chicken, porcine, turkey, and rat (Lee et al., PNAS 98, 9306 (2001)). Myostatin may or may not include additional terminal residues such as targeting sequences, or methionine and lysine residues and/or tag or fusion protein sequences, depending on how it is prepared.

### Myostatin Antagonists

As used herein the term “myostatin antagonist” is used interchangeably with “myostatin inhibitor”. A myostatin antagonist according to the present invention inhibits or blocks at least one activity of myostatin, or alternatively, blocks expression of myostatin or its receptor Inhibiting or blocking myostatin activity can be achieved, for example, by employing one or more inhibitory agents which interfere with the binding of myostatin to its receptor, and/or blocks signal transduction resulting from the binding of myostatin to its receptor. Antagonists include agents which bind to myostatin itself, or agents which bind to a myostatin receptor. For example, myostatin antagonists include but are not limited to follistatin, the myostatin prodomain, growth and differentiation factor 11 (GDF-11) prodomain, prodomain fusion proteins, antagonistic antibodies that bind to myostatin, antagonistic antibodies or antibody fragments that bind to the activin type IIB receptor, soluble activin type IIB receptor, soluble activin type IIB receptor fusion proteins, soluble myostatin analogs (soluble ligands), oligonucleotides, small molecules, peptidomimetics, and myostatin binding agents. These are described in more detail below.

Follistastin inhibits myostatin, as described, for example, in Amthor et al., Dev Biol 270, 19-30 (2004), and U.S. Pat. No. 6,004,937, which is herein incorporated by reference. Other inhibitors include, for example, TGF-β binding proteins including growth and differentiation factor-associated serum protein-1 (GASP) as described in Hill et al., Mol. Endo. 17 (6): 1144-1154 (2003). Myostatin antagonists include the propeptide region of myostatin and related GDF proteins including GDF-11, as described in PCT publication WO 02/09641, which is herein incorporated by reference. Myostatin antagonists further include modified and stabilized propeptides including Fc fusions of the prodomain as described, for example, in Bogdanovisch et al, FASEB J 19, 543-549 (2005). Additional myostatin antagonists include antibodies or antibody fragments which bind to and inhibit or neutralize myostatin, including the myostatin proprotein and/or mature protein, which in monomeric or dimeric form. Such antibodies are described, for example, in US patent application US 2004/0142383, and US patent application 2003/1038422, and PCT publication WO 2005/094446, PCT publication WO 2006/116269, all of which are incorporated by reference herein. Antagonistic myostatin antibodies further include antibodies which bind to the myostatin proprotein and prevent cleavage into the mature active form.

As used herein, the term “antibody” refers to refers to intact antibodies including polyclonal antibodies (see, for example Antibodies: A Laboratory Manual, Harlow and Lane (eds), Cold Spring Harbor Press, (1988)), and monoclonal antibodies (see, for example, U.S. Pat. Nos. RE 32,011, 4,902,614, 4,543,439, and 4,411,993, and Monoclonal Antibodies: A New Dimension in Biological Analysis, Plenum Press, Kennett, McKearn and Bechtol (eds.) (1980)). As used herein, the term “antibody” also refers to a fragment of an antibody such as F(ab), F(ab′), F(ab′)₂, Fv, Fc, and single chain antibodies, or combinations of these, which are produced by recombinant DNA techniques or by enzymatic or chemical cleavage of intact antibodies. The term “antibody” also refers to bispecific or bifunctional antibodies which are an artificial hybrid antibody having two different heavy/light chain pairs and two different binding sites. Bispecific antibodies can be produced by a variety of methods including fusion of hybridomas or linking of Fab′ fragments. (See Songsivilai et al, Clin. Exp. Immunol. 79:315-321 (1990), Kostelny et al., J. Immunol. 148:1547-1553 (1992)). As used herein the term “antibody” also refers to chimeric antibodies, that is, antibodies having a human constant antibody immunoglobulin domain is coupled to one or more non-human variable antibody immunoglobulin domain, or fragments thereof (see, for example, U.S. Pat. No. 5,595,898 and U.S. Pat. No. 5,693,493). The term “antibodies” also refers to “humanized” antibodies (see, for example, U.S. Pat. No. 4,816,567 and WO 94/10332), minibodies (WO 94/09817), single chain Fv-Fc fusions (Powers et al., J Immunol. Methods 251:123-135 (2001)), and antibodies produced by transgenic animals, in which a transgenic animal containing a proportion of the human antibody producing genes but deficient in the production of endogenous antibodies are capable of producing human antibodies (see, for example, Mendez et al., Nature Genetics 15:146-156 (1997), and U.S. Pat. No. 6,300,129). The term “antibodies” also includes multimeric antibodies, or a higher order complex of proteins such as heterdimeric antibodies. “Antibodies” also includes anti-idiotypic antibodies.

Myostatin antagonists further include soluble receptors which bind to myostatin and inhibit at least one activity. As used herein the term “soluble receptor” includes truncated versions or fragments of the myostatin receptor, modified or otherwise, capable of specifically binding to myostatin, and blocking or inhibiting myostatin signal transduction. These truncated versions of the myostatin receptor, for example, includes naturally occurring soluble domains, as well as variations due to proteolysis of the N- or C-termini. The soluble domain includes all or part of the extracellular domain of the receptor, alone or attached to additional peptides or modifications. Myostatin binds activin receptors including activin type IIB receptor (ActRIIB) and activin type IIA receptor (ActRIIA), as described in Lee et al, PNAS 98 (16), 9306-9311 (2001). Soluble receptor fusion proteins can also act as antagonists, for example soluble receptor Fc as described in US patent application publication 2004/0223966, and PCT publication WO 2006/012627, both of which are herein incorporated by reference.

Myostatin antagonists further include soluble ligands which compete with myostatin for binding to myostatin receptors. As used herein the term “soluble ligand antagonist” refers to soluble peptides, polypeptides or peptidomimetics capable of binding the myostatin activin type IIB receptor (or ActRIIA) and blocking myostatin-receptor signal transduction by competing with myostatin. Soluble ligand antagonists include variants of myostatin, also referred to as “myostatin analogs” that maintain substantial homology to, but not the activity of the ligand, including truncations such an N- or C-terminal truncations, substitutions, deletions, and other alterations in the amino acid sequence, such as substituting a non-amino acid peptidomimetic for an amino acid residue. Soluble ligand antagonists, for example, may be capable of binding the receptor, but not allowing signal transduction. For the purposes of the present invention a protein is “substantially similar” to another protein if they are at least 80%, preferably at least about 90%, more preferably at least about 95% identical to each other in amino acid sequence.

Myostatin antagonists further includes polynucleotide antagonists. These antagonists include antisense or sense oligonucleotides comprising a single-stranded polynucleotide sequence (either RNA or DNA) capable of binding to target mRNA (sense) or DNA (antisense) sequences. Antisense or sense oligonucleotides, according to the invention, comprise fragments of the targeted polynucleotide sequence encoding myostatin or its receptor, transcription factors, or other polynucleotides involved in the expression of myostatin or its receptor. Such a fragment generally comprises at least about 14 nucleotides, typically from about 14 to about 30 nucleotides. The ability to derive an antisense or a sense oligonucleotide, based upon a nucleic acid sequence encoding a given protein is described in, for example, Stein and Cohen, Cancer Res. 48:2659, 1988, and van der Krol et al. BioTechniques 6:958, 1988. Binding of antisense or sense oligonucleotides to target nucleic acid sequences results in the formation of duplexes that block or inhibit protein expression by one of several means, including enhanced degradation of the mRNA by RNAse H, inhibition of splicing, premature termination of transcription or translation, or by other means. The antisense oligonucleotides thus may be used to block expression of proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences. Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L)-lysine. Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid by any gene transfer method, including, for example, lipofection, CaPO₄-mediated DNA transfection, electroporation, or by using gene transfer vectors such as Epstein-Barr virus or adenovirus. Sense or antisense oligonucleotides also may be introduced into a cell containing the target nucleic acid by formation of a conjugate with a ligand-binding molecule, as described in WO 91/04753. Suitable ligand binding molecules include, but are not limited to, cell surface receptors, growth factors, other cytokines, or other ligands that bind to cell surface receptors. Preferably, conjugation of the ligand-binding molecule does not substantially interfere with the ability of the ligand-binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid by formation of an oligonucleotide-lipid complex, as described in WO 90/10448. The sense or antisense oligonucleotide-lipid complex is preferably dissociated within the cell by an endogenous lipase.

Additional methods for preventing expression of myostatin or myostatin receptors is RNA interference (RNAi) produced by the introduction of specific small interfering RNA (siRNA), as described, for example in Bosher et al., Nature Cell Biol 2, E31-E36 (2000).

The antagonistic nucleic acid molecules according to the present invention are capable of inhibiting or eliminating the functional activity of myostatin in vivo or in vitro. In one embodiment, the selective antagonist will inhibit the functional activity of myostatin by at least about 10%, in another embodiment by at least about 50%, in another embodiment by at least about 80%.

Myostatin antagonists further include small molecule antagonists which bind to either myostatin or its receptor. Small molecules are selected by screening for binding to myostatin or its receptor followed by specific and non-specific elutions similarly to the selection of binding agents described herein.

Myostatin binding agents are described below.

As used herein the term “capable of binding to myostatin” or “having a binding affinity for myostatin” refers to a myostatin antagonist such as a binding agent described herein which binds to myostatin as demonstrated by as the phage ELISA assay, the BIAcore® or KinExA™ assays described in the Examples below.

As used herein, the term “capable of modifying myostatin activity” refers to the action of an agent as either an agonist or an antagonist with respect to at least one biological activity of myostatin. As used herein, “agonist” or “mimetic” activity refers an agent having biological activity comparable to a protein that interacts with the protein of interest, as described, for example, in International application WO 01/83525, filed May 2, 2001, which is incorporated herein by reference.

As used herein, the term “inhibiting myostatin activity” or “antagonizing myostatin activity” refers to the ability of myostatin antagonist to reduce or block myostatin activity or signaling as demonstrated or in vitro assays such as, for example, the pMARE C2C12 cell-based myostatin activity assay or by in vivo animal testing as described below.

The present invention contemplates the use of combinations of myostatin antagonists for example, those described herein, in a pharmaceutical composition to treat the disorders discussed herein.

### Myostatin Binding Agents

The myostatin binding agents of the present invention comprise at least one myostatin binding peptide. In one embodiment, the binding agents of the present invention comprise at least one myostatin binding peptide covalently attached to at least one vehicle such as a polymer or an Fc domain. The attachment of the myostatin-binding peptides to at least one vehicle is intended to increase the effectiveness of the binding agent as a therapeutic by increasing the biological activity of the agent and/or decreasing degradation in vivo, increasing half-life in vivo, reducing toxicity or immunogenicity in vivo. The binding agents may further comprise a linker sequence connecting the peptide and the vehicle. The peptide or peptides are attached directly or indirectly through a linker sequence to the vehicle at the N-terminal, C-terminal or an amino acid sidechain of the peptide. In this embodiment, the binding agents of the present invention have the following structure:
(X¹)ₐ—F¹—(X²)_{b}, or multimers thereof;
wherein F¹ is a vehicle; and X¹ and X² are each independently selected from
-(L¹)_{c}-P¹;
-(L¹)_{c}-P¹-(L²)_{d}-P²;
-(L¹)_{c}-P¹-(L²)_{d}-P²-(L³)ₑ-P³;
and -(L¹)_{c}-P¹-(L²)_{d}-P²-(L³)ₑ-P³-(L⁴)_{f}-P⁴;
wherein P¹, P², P³, and P⁴ are peptides capable of binding myostatin; and
L¹, L², L³, and L⁴ are each linkers; and a, b, c, d, e, and f are each independently 0 or 1, provided that at least one of a and b is 1.

Any peptide containing a cysteinyl residue may be cross-linked with another Cys-containing peptide, either or both of which may be linked to a vehicle. Any peptide having more than one Cys residue may form an intrapeptide disulfide bond, as well.

In one embodiment, the vehicle is an Fc domain, defined below. This embodiment is referred to as a “peptibody”. As used herein, the term “peptibody” refers to a molecule comprising an antibody Fc domain attached to at least one peptide. The production of peptibodies is generally described in PCT publication WO 00/24782, published May 4, 2000, which is herein incorporated by reference. Exemplary peptibodies are provided as 1x and 2x configurations with one copy and two copies of the peptide (attached in tandem) respectively, as described in the Examples below.

### Peptides

As used herein the term “peptide” refers to molecules of about 5 to about 90 amino acids linked by peptide bonds. The peptides of the present invention are preferably between about 5 to about 50 amino acids in length, more preferably between about 10 and 30 amino acids in length, and most preferably between about 10 and 25 amino acids in length, and are capable of binding to the myostatin protein.

The peptides of the present invention may comprise part of a sequence of naturally occurring proteins, may be randomized sequences derived from naturally occurring proteins, or may be entirely randomized sequences. The peptides of the present invention may be generated by any methods known in the art including chemical synthesis, digestion of proteins, or recombinant technology. Phage display and RNA-peptide screening, and other affinity screening techniques are particularly useful for generating peptides capable of binding myostatin.

Phage display technology is described, for example, in Scott et al. Science 249: 386 (1990); Devlin et al., Science 249: 404 (1990); U.S. Pat. No. 5,223,409, issued Jun. 29, 1993; U.S. Pat. No. 5,733,731, issued Mar. 31, 1998; U.S. Pat. No. 5,498,530, issued Mar. 12, 1996; U.S. Pat. No. 5,432,018, issued Jul. 11, 1995; U.S. Pat. No. 5,338,665, issued Aug. 16, 1994; U.S. Pat. No. 5,922,545, issued Jul. 13, 1999; WO 96/40987, published Dec. 19, 1996; and WO 98/15833, published Apr. 16, 1998, each of which is incorporated herein by reference. Using phage libraries, random peptide sequences are displayed by fusion with coat proteins of filamentous phage. Typically, the displayed peptides are affinity-eluted either specifically or non-specifically against the target molecule. The retained phages may be enriched by successive rounds of affinity purification and repropagation. The best binding peptides are selected for further analysis, for example, by using phage ELISA, described below, and then sequenced. Optionally, mutagenesis libraries may be created and screened to further optimize the sequence of the best binders (Lowman, Ann Rev Biophys Biomol Struct 26:401-24 (1997)).

Other methods of generating the myostatin binding peptides include additional affinity selection techniques known in the art. A peptide library can be fused in the carboxyl terminus of the lac repressor and expressed in E. coli. Another E. coli-based method allows display on the cell's outer membrane by fusion with a peptidoglycan-associated lipoprotein (PAL). Hereinafter, these and related methods are collectively referred to as “E. coli display.” In another method, translation of random RNA is halted prior to ribosome release, resulting in a library of polypeptides with their associated RNA still attached. Hereinafter, this and related methods are collectively referred to as “ribosome display.” Other methods employ chemical linkage of peptides to RNA. See, for example, Roberts and Szostak, Proc Natl Acad Sci USA, 94: 12297-303 (1997). Hereinafter, this and related methods are collectively referred to as “RNA-peptide screening.” Yeast two-hybrid screening methods also may be used to identify peptides of the invention that bind to myostatin. In addition, chemically derived peptide libraries have been developed in which peptides are immobilized on stable, non-biological materials, such as polyethylene rods or solvent-permeable resins. Another chemically derived peptide library uses photolithography to scan peptides immobilized on glass slides. Hereinafter, these and related methods are collectively referred to as “chemical-peptide screening.” Chemical-peptide screening may be advantageous in that it allows use of D-amino acids and other analogues, as well as non-peptide elements. Both biological and chemical methods are reviewed in Wells and Lowman, Curr Opin Biotechnol 3: 355-62 (1992).

Additionally, selected peptides capable of binding myostatin can be further improved through the use of “rational design”. In this approach, stepwise changes are made to a peptide sequence and the effect of the substitution on the binding affinity or specificity of the peptide or some other property of the peptide is observed in an appropriate assay. One example of this technique is substituting a single residue at a time with alanine, referred to as an “alanine walk” or an “alanine scan”. When two residues are replaced, it is referred to as a “double alanine walk”. The resultant peptide containing amino acid substitutions are tested for enhanced activity or some additional advantageous property.

In addition, analysis of the structure of a protein-protein interaction may also be used to suggest peptides that mimic the interaction of a larger protein. In such an analysis, the crystal structure of a protein may suggest the identity and relative orientation of critical residues of the protein, from which a peptide may be designed. See, for example, Takasaki et al., Nature Biotech 15:1266 (1977). These methods may also be used to investigate the interaction between a targeted protein and peptides selected by phage display or other affinity selection processes, thereby suggesting further modifications of peptides to increase binding affinity and the ability of the peptide to inhibit the activity of the protein.

In one embodiment, the peptides of the present invention are generated as families of related peptides. Exemplary peptides are represented by SEQ ID NO: 1 through 132. These exemplary peptides were derived through an selection process in which the best binders generated by phage display technology were further analyzed by phage ELISA to obtain candidate peptides by an affinity selection technique such as phage display technology as described herein. However, the peptides of the present invention may be produced by any number of known methods including chemical synthesis as described below.

The peptides of the present invention can be further improved by the process of “affinity maturation”. This procedure is directed to increasing the affinity or the activity of the peptides and peptibodies of the present invention using phage display or other selection technologies. Based on a consensus sequence, directed secondary phage display libraries, for example, can be generated in which the “core” amino acids (determined from the consensus sequence) are held constant or are biased in frequency of occurrence. Alternatively, an individual peptide sequence can be used to generate a biased, directed phage display library. Panning of such libraries under more stringent conditions can yield peptides with enhanced binding to myostatin, selective binding to myostatin, or with some additional desired property. However, peptides having the affinity matured sequences may then be produced by any number of known methods including chemical synthesis or recombinantly. These peptides are used to generate binding agents such as peptibodies of various configurations which exhibit greater inhibitory activity in cell-based assays and in vivo assays.

Example 6 below describes affinity maturation of the “first round” peptides described above to produce affinity matured peptides. Exemplary affinity matured peptibodies are presented in Tables IV and V. The resultant 1x and 2x peptibodies made from these peptides were then further characterized for binding affinity, ability to neutralize myostatin activity, specificity to myostatin as opposed to certain other TGF-β family members such as activin, and for additional in vitro and in vivo activity, as described below. Affinity-matured peptides and peptibodies are referred to by the prefix “m” before their family name to distinguish them from first round peptides of the same family.

Exemplary first round peptides chosen for further affinity maturation according to the present invention included the following peptides: TN8-19 QGHCTRWPWMCPPY (SEQ ID NO: 33), and the linear peptides Linear-2 MEMLDSLFELLKDMVPISKA (SEQ ID NO: 104), Linear-15 HHGWNYLRKGSAPQWFEAWV (SEQ ID NO: 117), Linear-17, RATLLKDFWQLVEGYGDN (SEQ ID NO: 119), Linear-20 YREMSMLEGLLDVLERLQHY (SEQ ID NO: 122), Linear-21 HNSSQMLLSELIMLVGSMMQ (SEQ ID NO: 123), Linear-24 EFFHWLHNHRSEVNHWLDMN (SEQ ID NO: 126). The affinity matured families of each of these is presented below in Tables IV and V.

The peptides of the present invention also encompass variants and derivatives of the selected peptides which are capable of binding myostatin. As used herein the term “variant” refers to peptides having one or more amino acids inserted, deleted, or substituted into the original amino acid sequence, and which are still capable of binding to myostatin. Insertional and substitutional variants may contain natural amino acids as well as non-naturally occuring amino acids. As used herein the term “variant” includes fragments of the peptides which still retain the ability to bind to myostatin. As used herein, the term “derivative” refers to peptides which have been modified chemically in some manner distinct from insertion, deletion, and substitution variants. Variants and derivatives of the peptides and peptibodies of the present invention are described more fully below.

### Vehicles

As used herein the term “vehicle” refers to a molecule that may be attached to one or more peptides of the present invention. Preferably, vehicles confer at least one desired property on the binding agents of the present invention. Peptides alone are likely to be removed in vivo either by renal filtration, by cellular clearance mechanisms in the reticuloendothelial system, or by proteolytic degradation. Attachment to a vehicle improves the therapeutic value of a binding agent by reducing degradation of the binding agent and/or increasing half-life, reducing toxicity, reducing immunogenicity, and/or increasing the biological activity of the binding agent.

Exemplary vehicles include Fc domains; linear polymers such as polyethylene glycol (PEG), polylysine, dextran; a branched chain polymer (see for example U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid; a cholesterol group (such as a steroid); a carbohydrate or oligosaccharide; or any natural or synthetic protein, polypeptide or peptide that binds to a salvage receptor.

In one embodiment, the myostatin binding agents of the present invention have at least one peptide attached to at least one vehicle (F¹, F²) through the N-terminus, C-terminus or a side chain of one of the amino acid residues of the peptide(s). Multiple vehicles may also be used; such as an Fc domain at each terminus or an Fc domain at a terminus and a PEG group at the other terminus or a side chain.

An Fc domain is one preferred vehicle. As used herein, the term “Fc domain” encompasses native Fc and Fc variant molecules and sequences as defined below. As used herein the term “native Fc” refers to a non-antigen binding fragment of an antibody or the amino acid sequence of that fragment which is produced by recombinant DNA techniques or by enzymatic or chemical cleavage of intact antibodies. A preferred Fc is a fully human Fc and may originate from any of the immunoglobulins, such as IgG1 and IgG2. However, Fc molecules that are partially human, or originate from non-human species are also included herein. Native Fc molecules are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucl Acids Res 10: 4071-9). The term “native Fc” as used herein is used to refer to the monomeric, dimeric, and multimeric forms.

As used herein, the term “Fc variant” refers to a modified form of a native Fc sequence provided that binding to the salvage receptor is maintained, as described, for example, in WO 97/34631 and WO 96/32478, both of which are incorporated herein by reference. Fc variants may be constructed for example, by substituting or deleting residues, inserting residues or truncating portions containing the site. The inserted or substituted residues may also be altered amino acids, such as peptidomimetics or D-amino acids. Fc variants may be desirable for a number of reasons, several of which are described below. Exemplary Fc variants include molecules and sequences in which:

1. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., alanyl, seryl). Even when cysteine residues are removed, the single chain Fc domains can still form a dimeric Fc domain that is held together non-covalently.

2. A native Fc is modified to make it more compatible with a selected host cell. For example, one may remove the PA sequence near the N-terminus of a typical native Fc, which may be recognized by a digestive enzyme in E. coli such as proline iminopeptidase. One may also add an N-terminal methionyl residue, especially when the molecule is expressed recombinantly in a bacterial cell such as E. coli.

3. A portion of the N-terminus of a native Fc is removed to prevent N-terminal heterogeneity when expressed in a selected host cell. For this purpose, one may delete any of the first 20 amino acid residues at the N-terminus, particularly those at positions 1, 2, 3, 4 and 5.

4. One or more glycosylation sites are removed. Residues that are typically glycosylated (e.g., asparagine) may confer cytolytic response. Such residues may be deleted or substituted with unglycosylated residues (e.g., alanine).

5. Sites involved in interaction with complement, such as the Clq binding site, are removed. For example, one may delete or substitute the EKK sequence of human IgG1. Complement recruitment may not be advantageous for the molecules of this invention and so may be avoided with such an Fc variant.

6. Sites are removed that affect binding to Fc receptors other than a salvage receptor. A native Fc may have sites for interaction with certain white blood cells that are not required for the fusion molecules of the present invention and so may be removed.

7. The ADCC site is removed. ADCC sites are known in the art. See, for example, Molec Immunol 29 (5):633-9 (1992) with regard to ADCC sites in IgG1. These sites, as well, are not required for the fusion molecules of the present invention and so may be removed.

8. When the native Fc is derived from a non-human antibody, the native Fc may be humanized. Typically, to humanize a native Fc, one will substitute selected residues in the non-human native Fc with residues that are normally found in human native Fc. Techniques for antibody humanization are well known in the art.

The term “Fe domain” includes molecules in monomeric or multimeric form, whether digested from whole antibody or produced by other means. As used herein the term “multimer” as applied to Fc domains or molecules comprising Fc domains refers to molecules having two or more polypeptide chains associated covalently, noncovalently, or by both covalent and non-covalent interactions. IgG molecules typically form dimers; IgM, pentamers; IgD, dimers; and IgA, monomers, dimers, trimers, or tetramers. Multimers may be formed by exploiting the sequence and resulting activity of the native Ig source of the Fc or by derivatizing such a native Fc. The term “dimer” as applied to Fc domains or molecules comprising Fc domains refers to molecules having two polypeptide chains associated covalently or non-covalently.

Additionally, an alternative vehicle according to the present invention is a non-Fc domain protein, polypeptide, peptide, antibody, antibody fragment, or small molecule (e.g., a peptidomimetic compound) capable of binding to a salvage receptor. For example, one could use as a vehicle a polypeptide as described in U.S. Pat. No. 5,739,277, issued Apr. 14, 1998 to Presta et al. Peptides could also be selected by phage display for binding to the FcRn salvage receptor. Such salvage receptor-binding compounds are also included within the meaning of “vehicle” and are within the scope of this invention. Such vehicles should be selected for increased half-life (e.g., by avoiding sequences recognized by proteases) and decreased immunogenicity (e.g., by favoring non-immunogenic sequences, as discovered in antibody humanization).

In addition, polymer vehicles may also be used to construct the binding agents of the present invention. Various means for attaching chemical moieties useful as vehicles are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water soluble polymers to the N-terminus of proteins.

A preferred polymer vehicle is polyethylene glycol (PEG). The PEG group may be of any convenient molecular weight and may be linear or branched. The average molecular weight of the PEG will preferably range from about 2 kDa to about 100 kDa, more preferably from about 5 kDa to about 50 kDa, most preferably from about 5 kDa to about 10 kDa. The PEG groups will generally be attached to the compounds of the invention via acylation or reductive alkylation through a reactive group on the PEG moiety (e.g., an aldehyde, amino, thiol, or ester group) to a reactive group on the inventive compound (e.g., an aldehyde, amino, or ester group). A useful strategy for the PEGylation of synthetic peptides consists of combining, through forming a conjugate linkage in solution, a peptide and a PEG moiety, each bearing a special functionality that is mutually reactive toward the other. The peptides can be easily prepared with conventional solid phase synthesis as known in the art. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water soluble polymer which may be used for protein modification. Dextrans are polysaccharide polymers comprised of individual subunits of glucose predominantly linked by a1-6 linkages. The dextran itself is available in many molecular weight ranges, and is readily available in molecular weights from about 1 kDa to about 70 kDa. Dextran is a suitable water-soluble polymer for use in the present invention as a vehicle by itself or in combination with another vehicle (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference. Dextran of about 1 kDa to about 20 kDa is preferred when dextran is used as a vehicle in accordance with the present invention.

### Linkers

The binding agents of the present invention may optionally further comprise a “linker” group. Linkers serve primarily as a spacer between a peptide and a vehicles or between two peptides of the binding agents of the present invention. In one embodiment, the linker is made up of amino acids linked together by peptide bonds, preferably from 1 to 20 amino acids linked by peptide bonds, wherein the amino acids are selected from the 20 naturally occurring amino acids. One or more of these amino acids may be glycosylated, as is understood by those in the art. In one embodiment, the 1 to 20 amino acids are selected from glycine, alanine, proline, asparagine, glutamine, and lysine. Preferably, a linker is made up of a majority of amino acids that are sterically unhindered, such as glycine and alanine. Thus, exemplary linkers are polyglycines (particularly (Gly)₅, (Gly)₈), poly(Gly-Ala), and polyalanines. As used herein, the designation “g” refers to a glycine homopeptide linkers. As shown in Table II, “gn” refers to a 5x gly linker at the N terminus, while “gc” refers to 5x gly linker at the C terminus Combinations of Gly and Ala are also preferred. One exemplary linker sequence useful for constructing the binding agents of the present invention is the following: gsgsatggsgstassgsgsatg (SEQ ID NO: 305). This linker sequence is referred to as the “k” or lk sequence. The designations “kc”, as found in Table II, refers to the k linker at the C-terminus, while the designation “kn”, refers to the k linker at the N-terminus

The linkers of the present invention may also be non-peptide linkers. For example, alkyl linkers such as —NH—(CH₂)s-C(O)—, wherein s=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker, and has a molecular weight of 100 to 5000 kDa, preferably 100 to 500 kDa. The peptide linkers may be altered to form derivatives in the same manner as above.

### Exemplary Binding Agents

The binding agents described herein comprise at least one peptide capable of binding myostatin. In one embodiment, the myostatin binding peptide is between about 5 and about 50 amino acids in length, in another, between about 10 and 30 amino acids in length, and in another, between about 10 and 25 amino acids in length. In one embodiment the myostatin binding peptide comprises the amino acid sequence WMCPP (SEQ ID NO: 633). In other embodiment, the myostatin binding peptide comprises the amino acid sequence Ca₁a₂Wa₃WMCPP (SEQ ID NO: 352), wherein a₁, a₂ and a₃ are selected from a neutral hydrophobic, neutral polar, or basic amino acid. In another embodiment the myostatin binding peptide comprises the amino acid sequence Cb₁b₂Wb₃WMCPP (SEQ ID NO: 353), wherein b₁ is selected from any one of the amino acids T, I, or R; b₂ is selected from any one of R, S, Q; b₃ is selected from any one of P, R and Q, and wherein the peptide is between 10 and 50 amino acids in length, and physiologically acceptable salts thereof.

In another embodiment, the myostatin binding peptide comprises the formula:

(SEQ ID NO: 354)
c₁c₂c₃c₄c₅c₆Cc₇c₈Wc₉WMCPPc₁₀c₁₁c₁₂C₁₃,

wherein:

c₁ is absent or any amino acid;

c₂ is absent or a neutral hydrophobic, neutral polar, or acidic amino acid;

c₃ is absent or a neutral hydrophobic, neutral polar, or acidic amino acid;

c₄ is absent or any amino acid;

c₅ is absent or a neutral hydrophobic, neutral polar, or acidic amino acid;

c₆ is absent or a neutral hydrophobic, neutral polar, or basic amino acid;

c₇ is a neutral hydrophobic, neutral polar, or basic amino acid;

c₈ is a neutral hydrophobic, neutral polar, or basic amino acid;

c₉ is a neutral hydrophobic, neutral polar or basic amino acid; and

c₁₀ to c₁₃ is any amino acid; and wherein the peptide is between 20 and 50 amino acids in length, and physiologically acceptable salts thereof.

In a related embodiment the myostatin binding peptide comprises the formula:

(SEQ ID NO: 355)
d₁d₂d₃d₄d₅d₆Cd₇d₈Wd₉WMCPP d₁₀d₁₁d₁₂d₁₃,

wherein

d₁ is absent or any amino acid;

d₂ is absent or a neutral hydrophobic, neutral polar, or acidic amino acid;

d₃ is absent or a neutral hydrophobic, neutral polar, or acidic amino acid;

d₄ is absent or any amino acid;

d₅ is absent or a neutral hydrophobic, neutral polar, or acidic amino acid;

d₆ is absent or a neutral hydrophobic, neutral polar, or basic amino acid;

d₇ is selected from any one of the amino acids T, I, or R;

d₈ is selected from any one of R, S, Q;

d₉ is selected from any one of P, R and Q, and

d₁₀ to d₁₃ is selected from any amino acid,

and wherein the peptide is between 20 and 50 amino acids in length, and physiologically acceptable salts thereof

Additional embodiments of binding agents comprise at least one of the following peptides:

(1) a peptide capable of binding myostatin, wherein the peptide comprises the sequence WYe₁e₂Ye₃G, (SEQ ID NO: 356)

wherein e₁ is P, S or Y,

e₂ is C or Q, and

e₃ is G or H, wherein the peptide is between 7 and 50 amino acids in length, and physiologically acceptable salts thereof

(2) a peptide capable of binding myostatin, wherein the peptide comprises the sequence f₁EMLf₇SLf₃f₄LL, (SEQ ID NO: 455),

wherein f₁ is M or I,

f₂ is any amino acid,

f₃ is L or F,

f₄ is E, Q or D;

and wherein the peptide is between 7 and 50 amino acids in length, and physiologically acceptable salts thereof.

(3) a peptide capable of binding myostatin wherein the peptide comprises the sequence Lg₁g₂LLg₃g₄L, (SEQ ID NO: 456), wherein

g₁ is Q, D or E,

g₂ is S, Q, D or E,

g₃ is any amino acid,

g₄ is L, W, F, or Y, and wherein the peptide is between 8 and 50 amino acids in length, and physiologically acceptable salts thereof

(4) a peptide capable of binding myostatin, wherein the peptide comprises the sequence h₁h₂h₃h₄h₅h₆h₇h₈h₉ (SEQ ID NO: 457), wherein

h₁ is R or D,

h₂ is any amino acid,

h₃ is A, T S or Q,

h₄ is L or M,

h₅ is L or S,

h₆ is any amino acid,

h₇ is F or E,

h₈ is W, F or C,

h₉ is L, F, M or K, and wherein the peptide is between 9 and 50 amino acids in length, and physiologically acceptable salts thereof.

In one embodiment, the binding agents of the present invention have the following generalized structure:

(X¹)ₐ—F¹—(X²)_{b}, or multimers thereof;

wherein F¹ is a vehicle; and X¹ and X² are each independently selected from

-(L¹)_{c}-P¹;

-(L¹)_{c}-P¹-(L²)_{d}-P²;

-(L¹)_{c}-P¹-(L²)_{d}-P²-(L³)ₑ-P³;

and -(L¹)_{c}-P¹-(L²)_{d}-P²-(L³)ₑ-P³-(L⁴)_{f}-P⁴;

wherein P¹, P², P³, and P⁴ are peptides capable of binding myostatin; and
L¹, L², L³, and L⁴ are each linkers; and a, b, c, d, e, and f are each independently 0 or 1, provided that at least one of a and b is 1.

In one embodiment of the binding agents having this generalized structure, the peptides P¹, P², P³, and P⁴ can be selected from the peptides provided can be selected from one or more peptides comprising any of the following sequences: SEQ ID NO: 633, SEQ ID NO: 352, SEQ ID NO: 353, SEQ ID NO: 354, SEQ ID NO: 355, SEQ ID NO: 356, SEQ ID NO: 455, SEQ ID NO: 456, or SEQ ID NO: 457. In another embodiment, P P¹, P², P³, and P⁴ are independently selected from one or more peptides comprising any of the following sequences SEQ ID NO: 305 through 351 and SEQ ID NO: 357 through 454.

In a further embodiment, the vehicles of binding agents having the general formula above are Fc domains. The peptides are therefore fused to an Fc domain, either directly or indirectly, thereby providing peptibodies. The peptibodies of the present invention display a high binding affinity for myostatin and can inhibit the activity of myostatin as demonstrated by in vitro assays and in vivo testing in animals provided herein.

The present invention also provides nucleic acid molecules comprising polynucleotides encoding the peptides, peptibodies, and peptide and peptibody variants and derivatives of the present invention. Exemplary nucleotides sequences are given below.

### Variants and Derivatives of Peptides and Peptibodies

The binding agents described herein also encompass variants and derivatives of the peptides and peptibodies described herein. Since both the peptides and peptibodies of the present invention can be described in terms of their amino acid sequence, the terms “variants” and “derivatives” can be said to apply to a peptide alone, or a peptide as a component of a peptibody. As used herein, the term “peptide variants” refers to peptides or peptibodies having one or more amino acid residues inserted, deleted or substituted into the original amino acid sequence and which retain the ability to bind to myostatin and modify its activity. As used herein, fragments of the peptides or peptibodies are included within the definition of “variants”.

It is understood that any given peptide or peptibody may contain one or two or all three types of variants. Insertional and substitutional variants may contain natural amino acids, as well as non-naturally occuring amino acids or both.

Peptide and peptibody variants also include mature peptides and peptibodies wherein leader or signal sequences are removed, and the resulting proteins having additional amino terminal residues, which amino acids may be natural or non-natural. Peptibodies with an additional methionyl residue at amino acid position −1 (Met⁻¹-peptibody) are contemplated, as are peptibodies with additional methionine and lysine residues at positions −2 and −1 (Met⁻²-Lys⁻¹-). Variants having additional Met, Met-Lys, Lys residues (or one or more basic residues, in general) are particularly useful for enhanced recombinant protein production in bacterial host cells.

Peptide or peptibody variants of the present invention also includes peptides having additional amino acid residues that arise from use of specific expression systems. For example, use of commercially available vectors that express a desired polypeptide as part of glutathione-S-transferase (GST) fusion product provides the desired polypeptide having an additional glycine residue at amino acid position-1 after cleavage of the GST component from the desired polypeptide. Variants which result from expression in other vector systems are also contemplated, including those wherein histidine tags are incorporated into the amino acid sequence, generally at the carboxy and/or amino terminus of the sequence.

In one example, insertional variants are provided wherein one or more amino acid residues, either naturally occurring or non-naturally occuring amino acids, are added to a peptide amino acid sequence. Insertions may be located at either or both termini of the protein, or may be positioned within internal regions of the peptibody amino acid sequence.

Insertional variants with additional residues at either or both termini can include, for example, fusion proteins and proteins including amino acid tags or labels. Insertional variants include peptides in which one or more amino acid residues are added to the peptide amino acid sequence or fragment thereof.

Insertional variants also include fusion proteins wherein the amino and/or carboxy termini of the peptide or peptibody is fused to another polypeptide, a fragment thereof or amino acids which are not generally recognized to be part of any specific protein sequence. Examples of such fusion proteins are immunogenic polypeptides, proteins with long circulating half lives, such as immunoglobulin constant regions, marker proteins, proteins or polypeptides that facilitate purification of the desired peptide or peptibody, and polypeptide sequences that promote formation of multimeric proteins (such as leucine zipper motifs that are useful in dimer formation/stability).

This type of insertional variant generally has all or a substantial portion of the native molecule, linked at the N- or C-terminus, to all or a portion of a second polypeptide. For example, fusion proteins typically employ leader sequences from other species to permit the recombinant expression of a protein in a heterologous host. Another useful fusion protein includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

There are various commercially available fusion protein expression systems that may be used in the present invention. Particularly useful systems include but are not limited to the glutathione-S-transferase (GST) system (Pharmacia), the maltose binding protein system (NEB, Beverley, Mass.), the FLAG system (IBI, New Haven, Conn.), and the 6xHis system (Qiagen, Chatsworth, Calif.). These systems are capable of producing recombinant peptides and/or peptibodies bearing only a small number of additional amino acids, which are unlikely to significantly affect the activity of the peptide or peptibody. For example, both the FLAG system and the 6xHis system add only short sequences, both of which are known to be poorly antigenic and which do not adversely affect folding of a polypeptide to its native conformation. Another N-terminal fusion that is contemplated to be useful is the fusion of a Met-Lys dipeptide at the N-terminal region of the protein or peptides. Such a fusion may produce beneficial increases in protein expression or activity.

Other fusion systems produce polypeptide hybrids where it is desirable to excise the fusion partner from the desired peptide or peptibody. In one embodiment, the fusion partner is linked to the recombinant peptibody by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

The invention also provides fusion polypeptides which comprise all or part of a peptide or peptibody of the present invention, in combination with truncated tissue factor (tTF). tTF is a vascular targeting agent consisting of a truncated form of a human coagulation-inducing protein that acts as a tumor blood vessel clotting agent, as described U.S. Pat. Nos. 5,877,289; 6,004,555; 6,132,729; 6,132,730; 6,156,321; and European Patent No. EP 0988056. The fusion of tTF to the anti-myostatin peptibody or peptide, or fragments thereof facilitates the delivery of anti-myostatin antagonists to target cells, for example, skeletal muscle cells, cardiac muscle cells, fibroblasts, pre-adipocytes, and possibly adipocytes.

In another aspect, the invention provides deletion variants wherein one or more amino acid residues in a peptide or peptibody are removed. Deletions can be effected at one or both termini of the peptibody, or from removal of one or more residues within the peptibody amino acid sequence. Deletion variants necessarily include all fragments of a peptide or peptibody.

In still another aspect, the invention provides substitution variants of peptides and peptibodies of the invention. Substitution variants include those peptides and peptibodies wherein one or more amino acid residues are removed and replaced with one or more alternative amino acids, which amino acids may be naturally occurring or non-naturally occurring. Substitutional variants generate peptides or peptibodies that are “similar” to the original peptide or peptibody, in that the two molecules have a certain percentage of amino acids that are identical. Substitution variants include substitutions of 1, 2, 3, 4, 5, 6, 7, 8, 9, 10, 15, and 20 amino acids within a peptide or peptibody, wherein the number of substitutions may be up to ten percent of the amino acids of the peptide or peptibody. In one aspect, the substitutions are conservative in nature, however, the invention embraces substitutions that are also non-conservative and also includes unconventional amino acids.

Identity and similarity of related peptides and peptibodies can be readily calculated by known methods. Such methods include, but are not limited to, those described in Computational Molecular Biology, Lesk, A. M., ed., Oxford University Press, New York (1988); Biocomputing: Informatics and Genome Projects, Smith, D. W., ed., Academic Press, New York (1993); Computer Analysis of Sequence Data, Part 1, Griffin, A. M., and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., M. Stockton Press, New York (1991); and Carillo et al., SIAM J. Applied Math., 48:1073 (1988).

Preferred methods to determine the relatedness or percent identity of two peptides or polypeptides, or a polypeptide and a peptide, are designed to give the largest match between the sequences tested. Methods to determine identity are described in publicly available computer programs. Preferred computer program methods to determine identity between two sequences include, but are not limited to, the GCG program package, including GAP (Devereux et al., Nucl. Acid. Res., 12:387 (1984); Genetics Computer Group, University of Wisconsin, Madison, Wis., BLASTP, BLASTN, and FASTA (Altschul et al., J. Mol. BioL, 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra (1990)). The well-known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full-length sequences. Accordingly, in certain embodiments, the selected alignment method will result in an alignment that spans at least ten percent of the full length of the target polypeptide being compared, i.e., at least 40 contiguous amino acids where sequences of at least 400 amino acids are being compared, 30 contiguous amino acids where sequences of at least 300 to about 400 amino acids are being compared, at least 20 contiguous amino acids where sequences of 200 to about 300 amino acids are being compared, and at least 10 contiguous amino acids where sequences of about 100 to 200 amino acids are being compared. For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). In certain embodiments, a gap opening penalty (which is typically calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see Dayhoff et al., Atlas of Protein Sequence and Structure, 5(3)(1978) for the PAM 250 comparison matrix; Henikoff et al., Proc. Natl. Acad. Sci USA, 89:10915-10919 (1992) for the BLOSUM 62 comparison matrix) is also used by the algorithm.

In certain embodiments, for example, the parameters for a polypeptide sequence comparison can be made with the following: Algorithm: Needleman et al., J. Mol. Biol., 48:443-453 (1970); Comparison matrix: BLOSUM 62 from Henikoff et al., supra (1992); Gap Penalty: 12; Gap Length Penalty: 4; Threshold of Similarity: 0, along with no penalty for end gaps.

In certain embodiments, the parameters for polynucleotide molecule sequence (as opposed to an amino acid sequence) comparisons can be made with the following: Algorithm: Needleman et al., supra (1970); Comparison matrix: matches=+10, mismatch=0; Gap Penalty: 50: Gap Length Penalty: 3

Other exemplary algorithms, gap opening penalties, gap extension penalties, comparison matrices, thresholds of similarity, etc. may be used, including those set forth in the Program Manual, Wisconsin Package, Version 9, September, 1997. The particular choices to be made will be apparent to those of skill in the art and will depend on the specific comparison to be made, such as DNA-to-DNA, protein-to-protein, protein-to-DNA; and additionally, whether the comparison is between given pairs of sequences (in which case GAP or BestFit are generally preferred) or between one sequence and a large database of sequences (in which case FASTA or BLASTA are preferred).

Stereoisomers (e.g., D-amino acids) of the twenty conventional (naturally occuring) amino acids, non-naturally occuring amino acids such as α-, α-disubstituted amino acids, N-alkyl amino acids, lactic acid, and other unconventional amino acids may also be suitable components for peptides of the present invention. Examples of non-naturally occuring amino acids include, for example: aminoadipic acid, beta-alanine, beta-aminopropionic acid, aminobutyric acid, piperidinic acid, aminocaprioic acid, aminoheptanoic acid, aminoisobutyric acid, aminopimelic acid, diaminobutyric acid, desmosine, diaminopimelic acid, diaminopropionic acid, N-ethylglycine, N-ethylaspargine, hyroxylysine, all0-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline).

Naturally occurring residues may be divided into (overlapping) classes based on common side chain properties:

1) neutral hydrophobic: Met, Ala, Val, Leu, Ile, Pro, Trp, Met, Phe;
2) neutral polar: Cys, Ser, Thr, Asn, Gln, Tyr, Gly;
3) acidic: Asp, Glu;
4) basic: His, Lys, Arg;
5) residues that influence chain orientation: Gly, Pro; and
6) aromatic: Trp, Tyr, Phe.

Substitutions of amino acids may be conservative, which produces peptides having functional and chemical characteristics similar to those of the original peptide. Conservative amino acid substitutions involve exchanging a member of one of the above classes for another member of the same class. Conservative changes may encompass unconventional amino acid residues, which are typically incorporated by chemical peptide synthesis rather than by synthesis in biological systems. These include peptidomimetics and other reversed or inverted forms of amino acid moieties.

Non-conservative substitutions may involve the exchange of a member of one of these classes for a member from another class. These changes can result in substantial modification in the functional and/or chemical characteristics of the peptides. In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional peptibody or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One may also identify epitopes from primary amino acid sequences on the basis of hydrophilicity. These regions are also referred to as “epitopic core regions.”

TABLE A
Exemplary Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln, Glu, Asp | Gln |
| Asp | Glu, Gln, Asp | Glu |
| Cys | Ser, Ala | Ser |
| Gln | Asn, Glu, Asp | Asn |
| Glu | Asp, Gln, Asn | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

One skilled in the art will be able to produce variants of the peptides and peptibodies of the present invention by random substitution, for example, and testing the resulting peptide or peptibody for binding activity using the assays described herein.

Additionally, one skilled in the art can review structure-function studies or three-dimensional structural analysis in order to identify residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues. The variants can then be screened using activity assays as described herein.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given protein and that once a critical number of structures have been resolved, structural prediction will become dramatically more accurate.

Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, peptide or peptibody variants include glycosylation variants wherein one or more glycosylation sites such as a N-linked glycosylation site, has been added to the peptibody. An N-linked glycosylation site is characterized by the sequence: Asn-X-Ser or Asn-X-Thr, wherein the amino acid residue designated as X may be any amino acid residue except proline. The substitution or addition of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created.

The invention also provides “derivatives” of the peptides or peptibodies of the present invention. As used herein the term “derivative” refers to modifications other than, or in addition to, insertions, deletions, or substitutions of amino acid residues which retain the ability to bind to myostatin.

Preferably, the modifications made to the peptides of the present invention to produce derivatives are covalent in nature, and include for example, chemical bonding with polymers, lipids, other organic, and inorganic moieties. Derivatives of the invention may be prepared to increase circulating half-life of a peptibody, or may be designed to improve targeting capacity for the peptibody to desired cells, tissues, or organs.

The invention further embraces derivative binding agents covalently modified to include one or more water soluble polymer attachments, such as polyethylene glycol, polyoxyethylene glycol, or polypropylene glycol, as described U.S. Pat. Nos. 4,640,835; 4,496,689; 4,301,144; 4,670,417; 4,791,192; and 4,179,337. Still other useful polymers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are peptibodies covalently modified with polyethylene glycol (PEG) subunits. Water-soluble polymers may be bonded at specific positions, for example at the amino terminus of the peptibodies, or randomly attached to one or more side chains of the polypeptide. The use of PEG for improving the therapeutic capacity for binding agents, e.g. peptibodies, and for humanized antibodies in particular, is described in U.S. Pat. No. 6,133,426 to Gonzales et al., issued Oct. 17, 2000.

The invention also contemplates derivatizing the peptide and/or vehicle portion of the myostatin binding agents. Such derivatives may improve the solubility, absorption, biological half-life, and the like of the compounds. The moieties may alternatively eliminate or attenuate any undesirable side-effect of the compounds and the like. Exemplary derivatives include compounds in which:

1. The derivative or some portion thereof is cyclic. For example, the peptide portion may be modified to contain two or more Cys residues (e.g., in the linker), which could cyclize by disulfide bond formation.

2. The derivative is cross-linked or is rendered capable of cross-linking between molecules. For example, the peptide portion may be modified to contain one Cys residue and thereby be able to form an intermolecular disulfide bond with a like molecule. The derivative may also be cross-linked through its C-terminus

3. One or more peptidyl [—C(O)NR-] linkages (bonds) is replaced by a non-peptidyl linkage. Exemplary non-peptidyl linkages are —CH₂-carbamate [—CH₂—OC(O)NR—], phosphonate, —CH₂-sulfonamide [—CH₂—S(O)₂NR-], urea [—NHC(O)NH-], —CH₂-secondary amine, and alkylated peptide [—C(O)NR₆— wherein R₆ is lower alkyl].

4. The N-terminus is derivatized. Typically, the N-terminus may be acylated or modified to a substituted amine Exemplary N-terminal derivative groups include —NRR₁ (other than —NH₂), —NRC(O)R₁, —NRC(O)OR₁, —NRS(O)₂R₁, —NHC(O)NHR₁, succinimide, or benzyloxycarbonyl-NH— (CBZ—NH—), wherein R and R1 are each independently hydrogen or lower alkyl and wherein the phenyl ring may be substituted with 1 to 3 substituents selected from the group consisting of C₁-C₄ alkyl, C₁-C₄ alkoxy, chloro, and bromo.

5. The free C-terminus is derivatized. Typically, the C-terminus is esterified or amidated. For example, one may use methods described in the art to add (NH—CH₂—CH₂—NH₂)₂ to compounds of this invention at the C-terminus. Likewise, one may use methods described in the art to add —NH₂, (or “capping” with an —NH₂ group) to compounds of this invention at the C-terminus Exemplary C-terminal derivative groups include, for example, —C(O)R₂ wherein R₂ is lower alkoxy or —NR₃R₄ wherein R₃ and R₄ are independently hydrogen or C₁-C₈ alkyl (preferably C₁-C₄ alkyl).

6. A disulfide bond is replaced with another, preferably more stable, cross-linking moiety (e.g., an alkylene). See, e.g., Bhatnagar et al., J Med Chem 39: 3814-9 (1996), Alberts et al., Thirteenth Am Pep Symp, 357-9 (1993).

7. One or more individual amino acid residues is modified. Various derivatizing agents are known to react specifically with selected side chains or terminal residues, as described in detail below.

Lysinyl residues and amino terminal residues may be reacted with succinic or other carboxylic acid anhydrides, which reverse the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with any one or combination of several conventional reagents, including phenylglyoxal, 2,3-butanedione, 1,2-cyclohexanedione, and ninhydrin. Derivatization of arginyl residues requires that the reaction be performed in alkaline conditions because of the high pKa of the guanidine functional group. Furthermore, these reagents may react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl side chain groups (aspartyl or glutamyl) may be selectively modified by reaction with carbodiimides (R′—N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Cysteinyl residues can be replaced by amino acid residues or other moieties either to eliminate disulfide bonding or, conversely, to stabilize cross-linking. See, e.g., Bhatnagar et al., (supra).

Derivatization with bifunctional agents is useful for cross-linking the peptides or their functional derivatives to a water-insoluble support matrix or to other macromolecular vehicles. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains [see, for example, Creighton, Proteins: Structure and Molecule Properties (W. H. Freeman & Co., San Francisco), pp. 79-86 (1983)].

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Additional derivatives include non-peptide analogs that provide a stabilized structure or lessened biodegradation, are also contemplated. Peptide mimetic analogs can be prepared based on a selected inhibitory peptide by replacement of one or more residues by nonpeptide moieties. Preferably, the nonpeptide moieties permit the peptide to retain its natural confirmation, or stabilize a preferred, e.g., bioactive, confirmation which retains the ability to recognize and bind myostatin. In one aspect, the resulting analog/mimetic exhibits increased binding affinity for myostatin. One example of methods for preparation of nonpeptide mimetic analogs from peptides is described in Nachman et al., Regul Pept 57:359-370 (1995). If desired, the peptides of the invention can be modified, for instance, by glycosylation, amidation, carboxylation, or phosphorylation, or by the creation of acid addition salts, amides, esters, in particular C-terminal esters, and N-acyl derivatives of the peptides of the invention. The peptibodies also can be modified to create peptide derivatives by forming covalent or noncovalent complexes with other moieties. Covalently-bound complexes can be prepared by linking the chemical moieties to functional groups on the side chains of amino acids comprising the peptibodies, or at the N- or C-terminus

In particular, it is anticipated that the peptides can be conjugated to a reporter group, including, but not limited to a radiolabel, a fluorescent label, an enzyme (e.g., that catalyzes a colorimetric or fluorometric reaction), a substrate, a solid matrix, or a carrier (e.g., biotin or avidin). The invention accordingly provides a molecule comprising a peptibody molecule, wherein the molecule preferably further comprises a reporter group selected from the group consisting of a radiolabel, a fluorescent label, an enzyme, a substrate, a solid matrix, and a carrier. Such labels are well known to those of skill in the art, e.g., biotin labels are particularly contemplated. The use of such labels is well known to those of skill in the art and is described in, e.g., U.S. Pat. Nos. 3,817,837; 3,850,752; 3,996,345; and 4,277,437. Other labels that will be useful include but are not limited to radioactive labels, fluorescent labels and chemiluminescent labels. U.S. patents concerning use of such labels include, for example, U.S. Pat. Nos. 3,817,837; 3,850,752; 3,939,350; and 3,996,345. Any of the peptibodies of the present invention may comprise one, two, or more of any of these labels.

### Methods of Making Peptides and Peptibodies

The peptides of the present invention can be generated using a wide variety of techniques known in the art. For example, such peptides can be synthesized in solution or on a solid support in accordance with conventional techniques. Various automatic synthesizers are commercially available and can be used in accordance with known protocols. See, for example, Stewart and Young (supra); Tam et al., J Am Chem Soc, 105:6442, (1983); Merrifield, Science 232:341-347 (1986); Barany and Merrifield, The Peptides, Gross and Meienhofer, eds, Academic Press, New York, 1-284; Barany et al., Int J Pep Protein Res, 30:705-739 (1987); and U.S. Pat. No. 5,424,398, each incorporated herein by reference.

Solid phase peptide synthesis methods use a copoly(styrene-divinylbenzene) containing 0.1-1.0 mM amines/g polymer. These methods for peptide synthesis use butyloxycarbonyl (t-BOC) or 9-fluorenylmethyloxy-carbonyl(FMOC) protection of alpha-amino groups. Both methods involve stepwise syntheses whereby a single amino acid is added at each step starting from the C-terminus of the peptide (See, Coligan et al., Curr Prot Immunol, Wiley Interscience, 1991, Unit 9). On completion of chemical synthesis, the synthetic peptide can be deprotected to remove the t-BOC or FMOC amino acid blocking groups and cleaved from the polymer by treatment with acid at reduced temperature (e.g., liquid HF-10% anisole for about 0.25 to about 1 hours at 0° C.). After evaporation of the reagents, the peptides are extracted from the polymer with 1% acetic acid solution that is then lyophilized to yield the crude material. This can normally be purified by such techniques as gel filtration on Sephadex G-15 using 5% acetic acid as a solvent. Lyophilization of appropriate fractions of the column will yield the homogeneous peptides or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Phage display techniques can be particularly effective in identifying the peptides of the present invention as described above. Briefly, a phage library is prepared (using e.g. ml 13, fd, or lambda phage), displaying inserts from 4 to about 80 amino acid residues. The inserts may represent, for example, a completely degenerate or biased array. Phage-bearing inserts that bind to the desired antigen are selected and this process repeated through several cycles of reselection of phage that bind to the desired antigen. DNA sequencing is conducted to identify the sequences of the expressed peptides. The minimal linear portion of the sequence that binds to the desired antigen can be determined in this way. The procedure can be repeated using a biased library containing inserts containing part or all of the minimal linear portion plus one or more additional degenerate residues upstream or downstream thereof. These techniques may identify peptides of the invention with still greater binding affinity for myostatin than agents already identified herein.

Regardless of the manner in which the peptides are prepared, a nucleic acid molecule encoding each such peptide can be generated using standard recombinant DNA procedures. The nucleotide sequence of such molecules can be manipulated as appropriate without changing the amino acid sequence they encode to account for the degeneracy of the nucleic acid code as well as to account for codon preference in particular host cells.

The present invention also provides nucleic acid molecules comprising polynucleotide sequences encoding the peptides and peptibodies of the present invention. These nucleic acid molecules include vectors and constructs containing polynucleotides encoding the peptides and peptibodies of the present invention, as well as peptide and peptibody variants and derivatives. Exemplary nucleic acid molecules are provided in the Examples below.

Recombinant DNA techniques also provide a convenient method for preparing full length peptibodies and other large polypeptide binding agents of the present invention, or fragments thereof. A polynucleotide encoding the peptibody or fragment may be inserted into an expression vector, which can in turn be inserted into a host cell for production of the binding agents of the present invention. Preparation of exemplary peptibodies of the present invention are described in Example 2 below.

A variety of expression vector/host systems may be utilized to express the peptides and peptibodies of the invention. These systems include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. One preferred host cell line is E. coli strain 2596 (ATCC #202174), used for expression of peptibodies as described below in Example 2. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells.

The term “expression vector” refers to a plasmid, phage, virus or vector, for expressing a polypeptide from a polynucleotide sequence. An expression vector can comprise a transcriptional unit comprising an assembly of (1) a genetic element or elements having a regulatory role in gene expression, for example, promoters or enhancers, (2) a structural or sequence that encodes the binding agent which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionyl residue. This residue may or may not be subsequently cleaved from the expressed recombinant protein to provide a final peptide product.

For example, the peptides and peptibodies may be recombinantly expressed in yeast using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted peptide is purified from the yeast growth medium using the methods used to purify the peptide from bacterial and mammalian cell supernatants.

Alternatively, the cDNA encoding the peptide and peptibodies may be cloned into the baculovirus expression vector pVL1393 (PharMingen, San Diego, Calif.). This vector can be used according to the manufacturer's directions (PharMingen) to infect Spodoptera frugiperda cells in sF9 protein-free media and to produce recombinant protein. The recombinant protein can be purified and concentrated from the media using a heparin-Sepharose column (Pharmacia).

Alternatively, the peptide or peptibody may be expressed in an insect system. Insect systems for protein expression are well known to those of skill in the art. In one such system, Autographa californica nuclear polyhedrosis virus (AcNPV) can be used as a vector to express foreign genes in Spodoptera frugiperda cells or in Trichoplusia larvae. The peptide coding sequence can be cloned into a nonessential region of the virus, such as the polyhedrin gene, and placed under control of the polyhedrin promoter. Successful insertion of the peptide will render the polyhedrin gene inactive and produce recombinant virus lacking coat protein coat. The recombinant viruses can be used to infect S. frugiperda cells or Trichoplusia larvae in which the peptide is expressed (Smith et al., J Virol 46: 584 (1983); Engelhard et al., Proc Nat Acad Sci (USA) 91: 3224-7 (1994)).

In another example, the DNA sequence encoding the peptide can be amplified by PCR and cloned into an appropriate vector for example, pGEX-3X (Pharmacia). The pGEX vector is designed to produce a fusion protein comprising glutathione-S-transferase (GST), encoded by the vector, and a protein encoded by a DNA fragment inserted into the vector's cloning site. The primers for PCR can be generated to include for example, an appropriate cleavage site. Where the fusion moiety is used solely to facilitate expression or is otherwise not desirable as an attachment to the peptide of interest, the recombinant fusion protein may then be cleaved from the GST portion of the fusion protein. The pGEX-3X/specific binding agent peptide construct is transformed into E. coli XL-1 Blue cells (Stratagene, La Jolla Calif.), and individual transformants isolated and grown. Plasmid DNA from individual transformants can be purified and partially sequenced using an automated sequencer to confirm the presence of the desired specific binding agent encoding nucleic acid insert in the proper orientation.

The fusion protein, which may be produced as an insoluble inclusion body in the bacteria, can be purified as follows. Host cells are collected by centrifugation; washed in 0.15 M NaCl, 10 mM Tris, pH 8, 1 mM EDTA; and treated with 0.1 mg/ml lysozyme (Sigma, St. Louis, Mo.) for 15 minutes at room temperature. The lysate can be cleared by sonication, and cell debris can be pelleted by centrifugation for 10 minutes at 12,000×g. The fusion protein-containing pellet can be resuspended in 50 mM Tris, pH 8, and 10 mM EDTA, layered over 50% glycerol, and centrifuged for 30 min. at 6000×g. The pellet can be resuspended in standard phosphate buffered saline solution (PBS) free of Mg++ and Ca++. The fusion protein can be further purified by fractionating the resuspended pellet in a denaturing SDS-PAGE (Sambrook et al., supra). The gel can be soaked in 0.4 M KCl to visualize the protein, which can be excised and electroeluted in gel-running buffer lacking SDS. If the GST/fusion protein is produced in bacteria as a soluble protein, it can be purified using the GST Purification Module (Pharmacia).

The fusion protein may be subjected to digestion to cleave the GST from the peptide of the invention. The digestion reaction (20-40 mg fusion protein, 20-30 units human thrombin (4000 U/mg, Sigma) in 0.5 ml PBS can be incubated 16-48 hrs at room temperature and loaded on a denaturing SDS-PAGE gel to fractionate the reaction products. The gel can be soaked in 0.4 M KCl to visualize the protein bands. The identity of the protein band corresponding to the expected molecular weight of the peptide can be confirmed by amino acid sequence analysis using an automated sequencer (Applied Biosystems Model 473A, Foster City, Calif.). Alternatively, the identity can be confirmed by performing HPLC and/or mass spectometry of the peptides.

Alternatively, a DNA sequence encoding the peptide can be cloned into a plasmid containing a desired promoter and, optionally, a leader sequence (Better et al., Science 240:1041-43 (1988)). The sequence of this construct can be confirmed by automated sequencing. The plasmid can then be transformed into E. coli strain MC1061 using standard procedures employing CaCl2 incubation and heat shock treatment of the bacteria (Sambrook et al., supra). The transformed bacteria can be grown in LB medium supplemented with carbenicillin, and production of the expressed protein can be induced by growth in a suitable medium. If present, the leader sequence can effect secretion of the peptide and be cleaved during secretion.

Mammalian host systems for the expression of recombinant peptides and peptibodies are well known to those of skill in the art. Host cell strains can be chosen for a particular ability to process the expressed protein or produce certain post-translation modifications that will be useful in providing protein activity. Such modifications of the protein include, but are not limited to, acetylation, carboxylation, glycosylation, phosphorylation, lipidation and acylation. Different host cells such as CHO, HeLa, MDCK, 293, WI38, and the like have specific cellular machinery and characteristic mechanisms for such post-translational activities and can be chosen to ensure the correct modification and processing of the introduced, foreign protein.

It is preferable that transformed cells be used for long-term, high-yield protein production. Once such cells are transformed with vectors that contain selectable markers as well as the desired expression cassette, the cells can be allowed to grow for 1-2 days in an enriched media before they are switched to selective media. The selectable marker is designed to allow growth and recovery of cells that successfully express the introduced sequences. Resistant clumps of stably transformed cells can be proliferated using tissue culture techniques appropriate to the cell line employed.

A number of selection systems can be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr which confers resistance to methotrexate; gpt which confers resistance to mycophenolic acid; neo which confers resistance to the aminoglycoside G418 and confers resistance to chlorsulfuron; and hygro which confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, β-glucuronidase and its substrate, GUS, and luciferase and its substrate, luciferin.

### Purification and Refolding of Binding Agents

In some cases, the binding agents such as the peptides and/or peptibodies of this invention may need to be “refolded” and oxidized into a proper tertiary structure and disulfide linkages generated in order to be biologically active. Refolding can be accomplished using a number of procedures well known in the art. Such methods include, for example, exposing the solubilized polypeptide agent to a pH usually above 7 in the presence of a chaotropic agent. The selection of chaotrope is similar to the choices used for inclusion body solubilization, however a chaotrope is typically used at a lower concentration. Exemplary chaotropic agents are guanidine and urea. In most cases, the refolding/oxidation solution will also contain a reducing agent plus its oxidized form in a specific ratio to generate a particular redox potential which allows for disulfide shuffling to occur for the formation of cysteine bridges. Some commonly used redox couples include cysteine/cystamine, glutathione/dithiobisGSH, cupric chloride, dithiothreitol DTT/dithiane DTT, and 2-mercaptoethanol (bME)/dithio-bME. In many instances, a co-solvent may be used to increase the efficiency of the refolding. Commonly used cosolvents include glycerol, polyethylene glycol of various molecular weights, and arginine.

It may be desirable to purify the peptides and peptibodies of the present invention. Protein purification techniques are well known to those of skill in the art. These techniques involve, at one level, the crude fractionation of the proteinaceous and non-proteinaceous fractions. Having separated the peptide and/or peptibody from other proteins, the peptide or polypeptide of interest can be further purified using chromatographic and electrophoretic techniques to achieve partial or complete purification (or purification to homogeneity). Analytical methods particularly suited to the preparation of peptibodies and peptides or the present invention are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of a peptibody or peptide of the present invention. The term “purified peptibody or peptide” as used herein, is intended to refer to a composition, isolatable from other components, wherein the peptibody or peptide is purified to any degree relative to its naturally-obtainable state. A purified peptide or peptibody therefore also refers to a peptibody or peptide that is free from the environment in which it may naturally occur.

Generally, “purified” will refer to a peptide or peptibody composition that has been subjected to fractionation to remove various other components, and which composition substantially retains its expressed biological activity. Where the term “substantially purified” is used, this designation will refer to a peptide or peptibody composition in which the peptibody or peptide forms the major component of the composition, such as constituting about 50%, about 60%, about 70%, about 80%, about 90%, about 95% or more of the proteins in the composition.

Various methods for quantifying the degree of purification of the peptide or peptibody will be known to those of skill in the art in light of the present disclosure. These include, for example, determining the specific binding activity of an active fraction, or assessing the amount of peptide or peptibody within a fraction by SDS/PAGE analysis. A preferred method for assessing the purity of a peptide or peptibody fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the peptibody or peptide exhibits a detectable binding activity.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography steps such as affinity chromatography (e.g., Protein-A-Sepharose), ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified binding agent.

There is no general requirement that the binding agents of the present invention always be provided in their most purified state. Indeed, it is contemplated that less substantially purified binding agent products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low-pressure chromatography system. Methods exhibiting a lower degree of relative purification may have advantages in total recovery of the peptide or peptibody, or in maintaining binding activity of the peptide or peptibody.

It is known that the migration of a peptide or polypeptide can vary, sometimes significantly, with different conditions of SDS/PAGE (Capaldi et al., Biochem Biophys Res Comm, 76: 425 (1977)). It will therefore be appreciated that under differing electrophoresis conditions, the apparent molecular weights of purified or partially purified binding agent expression products may vary.

### Activity of Myostatin Binding Agents and Other Antagonists

The antagonists including the binding agents described herein were tested for their ability to bind myostatin and inhibit or block myostatin activity. Any number of assays or animal tests may be used to determine the ability of the agent to inhibit or block myostatin activity. Several assays used for characterizing the peptides and peptibodies of the present invention are described in the Examples below. One assay is the C2C12 pMARE-luc assay which makes use of a myostatin-responsive cell line (C2C12 myoblasts) transfected with a luciferase reporter vector containing myostatin/activin response elements (MARE). Exemplary peptibodies are assayed by pre-incubating a series of peptibody dilutions with myostatin, and then exposing the cells to the incubation mixture. The resulting luciferase activity is determined, and a titration curve is generated from the series of peptibody dilutions. The IC₅₀ (the concentration of peptibody to achieve 50% inhibition of myostatin activity as measured by luciferase activity) was then determined. A second assay described below is a BIAcore® assay to determine the kinetic parameters kₐ (association rate constant), k_{d} (dissociation rate constant), and K_{D} (dissociation equilibrium constant) for the myostatin binding agents and other antagonists such as antibodies capable of binding myostatin and its receptor. Lower dissociation equilibrium constants (K_{D}, expressed in nM) indicated a greater affinity of the peptibody for myostatin. Additional assays include blocking assays, to determine whether a binding agent such as a peptibody is neutralizing (prevents binding of myostatin to its receptor), or non-neutralizing (does not prevent binding of myostatin to its receptor); selectivity assays, which determine if the binding agents of the present invention bind selectively to myostatin and not to certain other TGF-β family members; and KinEx A™ assays or solution-based equilibrium assays, which also determine K_{D} and are considered to be more sensitive in some circumstances. These assays are described in Example 3.

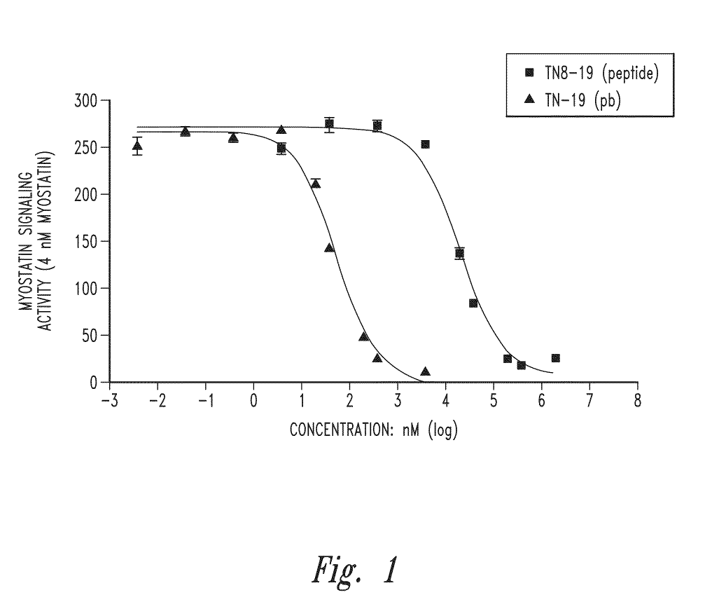

FIG. 1 shows the IC₅₀ of a peptide compared with the IC₅₀ of the peptibody form of the peptide. This demonstrates that the peptibody is significantly more effective at inhibiting myostatin activity than the peptide alone. In addition, affinity-matured peptibodies generally exhibit improved IC₅₀ and K_{D} values compared with the parent peptides and peptibodies. The IC₅₀ values for a number of exemplary affinity matured peptibodies are shown in Table VII, Example 7 below. Additionally, in some instances, making a 2x version of a peptibody, where two peptides are attached in tandem, increase the activity of the peptibody both in vitro and in vivo.

In vivo activities are demonstrated in the Examples below. The activities of the binding agents include but are not limited to increased lean muscle mass, increased muscle strength, and decreased fat mass with respect to total body weight in treated animal models. The in vivo activities described herein further include attenuation of wasting of lean muscle mass and strength in animal models including models of hypogonadism, rheumatoid cachexia, cancer cachexia, and inactivity.

### Uses of Myostatin Antagonists

The present invention provides methods and treatments for muscle related and other disorders by administering a therapeutic amount of a myostatin antagonist or antagonists to subjects in need of such a treatment. Myostatin antagonists can also be administered prophylactically to protect against future muscle wasting and related disorders in a subject in need of such as treatment. As used herein the term “subject” refers to any animal including mammals, and including human subjects in need of treatment for myostatin-related disorders. In one embodiment, the myostatin antagonists are the binding agents described herein.

These myostatin-related disorders include, but are not limited to, various forms of muscle wasting, as well as metabolic disorders such as diabetes and related disorders, and bone degenerative diseases such as osteoporosis. Myostatin antagonists also can be used to treat disorders resulting from hypogonadism, disorders resulting from inactivity, disorders which would otherwise be treated by growth hormones or growth hormone secretagogues, and various cachexias including tumor related cachexia, rheumatoid cachexia, and cachexia resulting from burns.

As shown in the examples below, myostatin antagonists such as the exemplary peptibodies described herein dramatically increases lean muscle mass, decreases fat mass, alters the ratio of muscle to fat, and increases muscle strength.

Muscle wasting disorders include muscular dystrophies and neuromuscular disorders. These disorders include but are not limited to Duchenne's muscular dystrophy, progressive muscular dystrophy, Becker's type muscular dystrophy, Dejerine-Landouzy muscular dystrophy, Erb's muscular dystrophy, Emery Dreifuss muscular dystrophy, limb girdle muscular dystrophy, rigid spine sydrome, muscle-eye-brain disease, amyotrophic lateral sclerosis, facioscapulohumeral muscular dytrophy, congenital muscular dystrophy, infantile neuroaxonal muscular dystrophy, myotonic dytrophy (Steinert's disease), nondytrophic myotonia, periodic paralyses spinal muscular atrophy, heredity motor and sensory neuropathy, Carcot-Marie-Tooth disease, chronic inflammatory neuropathy, distal myopathy, myotubular/centronuclear myopathy, nemaline myopathy, mini core disease, central core disease, desminopathy, inclusion body myositis, mitochondrial myopathy, congenital myasthenic syndrome, post-polio muscle dysfunction, and disorders described in Emery Lancet 359:687-695 (2002) and Khurana et al, Nat. Rev. Drug Disc 2:379-386 (2003). These disorders can be treated by adminstering a therapeutic amount of one or more myostatin antagonist to a subject in need thereof. This is demonstrated by administering an exemplary peptibody an aged mdx mouse model, as described in Example 11 below.

Myostatin antagonists are also useful for treating metabolic disorders including type 2 diabetes, noninsulin-dependent diabetes mellitus, hyperglycemia, and obesity. For example, myostatin may influence the development of diabetes in certain cases. It is known that, for example, skeletal muscle resistance to insulin-stimulated glucose uptake is the earliest known manifestation of non-insulin-dependent (type 2) diabetes mellitus (Corregan et al. Endocrinology 128:1682 (1991)). It has been shown that the lack of myostatin partially attenuates the obese and diabetes phenotypes of two mouse models, the agouti lethal yellow (Ar) (Yen et al. FASEB J. 8:479 (1994)), and obese (Lep^{ob/ob}). Fat accumulation and total body weight of the A^{y/a}, Mstn^{-/-} double mutant mouse was dramatically reduced compared with the A^{y/a} Mstn⁺¹⁺ mouse (McFerron et al., (2002) supra). In addition, blood glucose levels in the A^{y/a}, Mstn^{-/-} mice was dramatically lower than in A^{y/a} Mstn^{+/+} mice following exogenous glucose load, indicating that the lack of myostatin improved glucose metabolism. Similarly Lep^{ob/ob} Mstn^{-/-} mice showed decreased fat accumulation when compared with the Lep^{ob/ob} Mstn^{+/+} phenotype. It has been demonstrated in the Examples below that decreasing or blocking myostatin activity by administering an exemplary peptibody decreases the fat to muscle ratio in an aged animal model. Therefore, individuals suffering from the effects of diabetes, obesity, and hyperglycemic conditions can be treated with a therapeutically effective dose of one or more myostatin antagonist, such as the myostatin binding agents described herein.

Other complications from diabetes includes cachexia as well as diabetic nephropathy due to high blood glucose and other effects of diabetes. As can be seen in Example 15 below, administration of a myostatin antagonist exemplified by 2x mTN8-19-21 significantly attenuated the body weight loss and preserved skeletal muscle mass and lean body mass in STZ-induced diabetic mice. In addition to an increase in skeletal muscle and lean mass, the antagonist attenuated kidney hypertrophy, the increase in creatinine clearance rate and reduced 24 hour urine volume and urinary albumin excretion in STZ-induced diabetic mice. This shows improved kidney function in the early stage of development of diabetic nephropathy. Therefore myostatin antagonists are useful for treating cachexia caused by diabetes, and for treating diabetic nephropathy.

Additional muscle wasting disorders arise from chronic disease including congestive obstructive pulmonary disease (COPD) and cystic fibrosis (pulmonary cachexia), cardiac disease or failure (cardiac cachexia), cancer (cancer or tumor related cachexia), wasting due to AIDS, wasting due to renal failure, cachexia associated with dialysis, uremia, and rheumatoid arthritis (rheumatoid cachexia). For example, serum and intramuscular concentrations of myostatin-immunoreactive protein was found to be increased in men exhibiting AIDS-related muscle wasting and was inversely related to fat-free mass (Gonzalez-Cadavid et al., PNAS USA 95: 14938-14943 (1998)). As used herein the term “cachexia” refers to the condition of accelerated muscle wasting and loss of lean body mass resulting from a number of diseases such as those described above. Treatment of cachexia was demonstrated by treating a mouse model of tumor cachexia using an exemplary peptibody. Balb/c male mice (Charles River Labs, Wilmington, Mass.) bearing tumors generated by inoculation with murine colon-24 adenocarcinoma cell line (ATCC# CRL 2639) were treated with 2x mTN8-19-21 attached to murine Fc (2x mTN8-19-21/muFc) or a murine Fc vehicle. Animals treated with the peptibody showed attenuation of loss of body weight, lean body mass, and the preservation of skeletal muscle mass compared with the control animals treated with an Fc vehicle. This occured in both young (3 months) and older (12 months) mice. This demonstrated that cachexia such as cancer cachexia can be treated with a therapeutic dosage of one or more myostatin antagonists, such as the myostatin binding agents described herein.

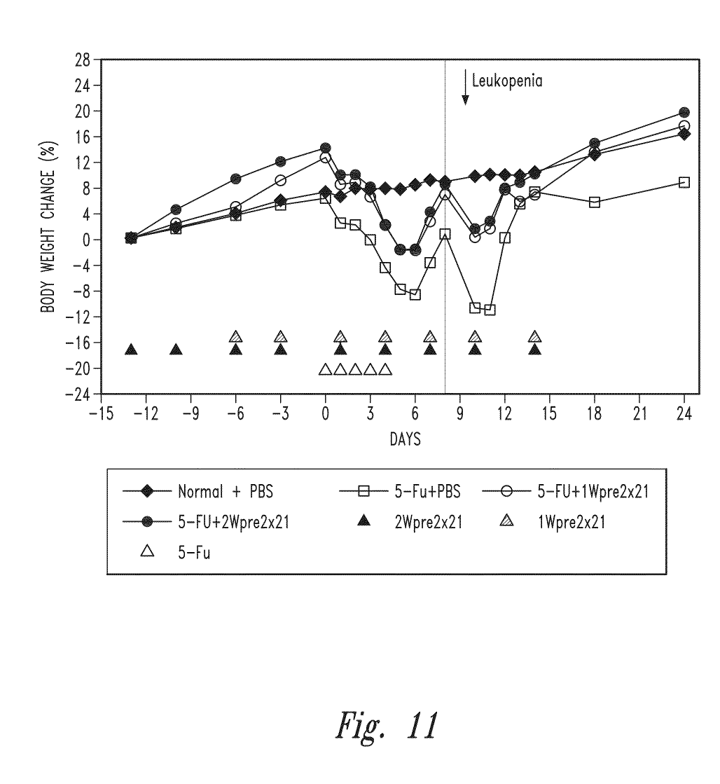

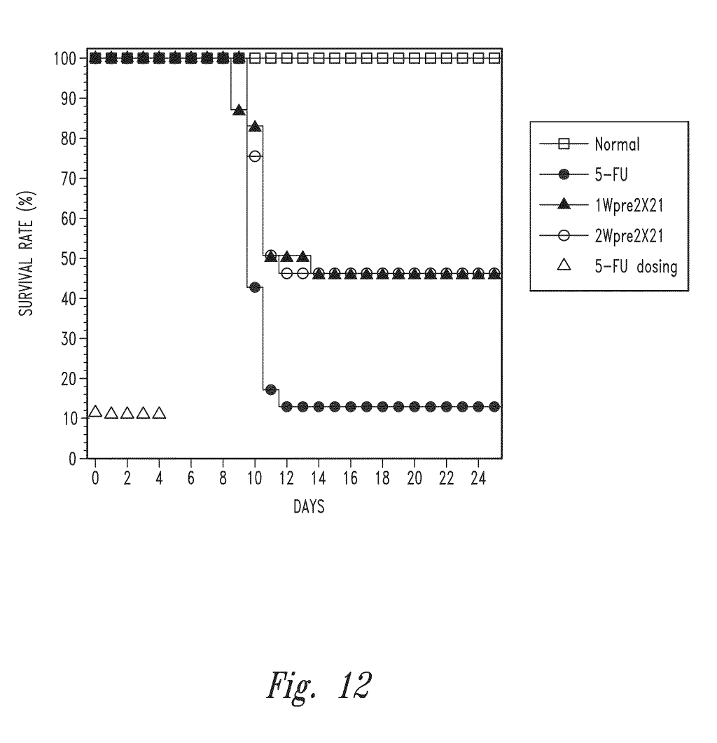

In addition, cachexia can be caused by chemotherapeutic agents themselves. Example 16 below shows the development of an chemotherapy cachexia animal model using 5-fluorouracil (5-Fu). Myostatin antagonists exemplified by 2x mTN8-19-21/muFc attenuated body weight loss in this model and increased survival in the animals treated with 5-Fu (see Example 16 and FIGS. 11 and 12). Chemotherapeutic agents refers to all chemical agents used to treat cancer.

### Treatment of Inflammation Related Cachexia

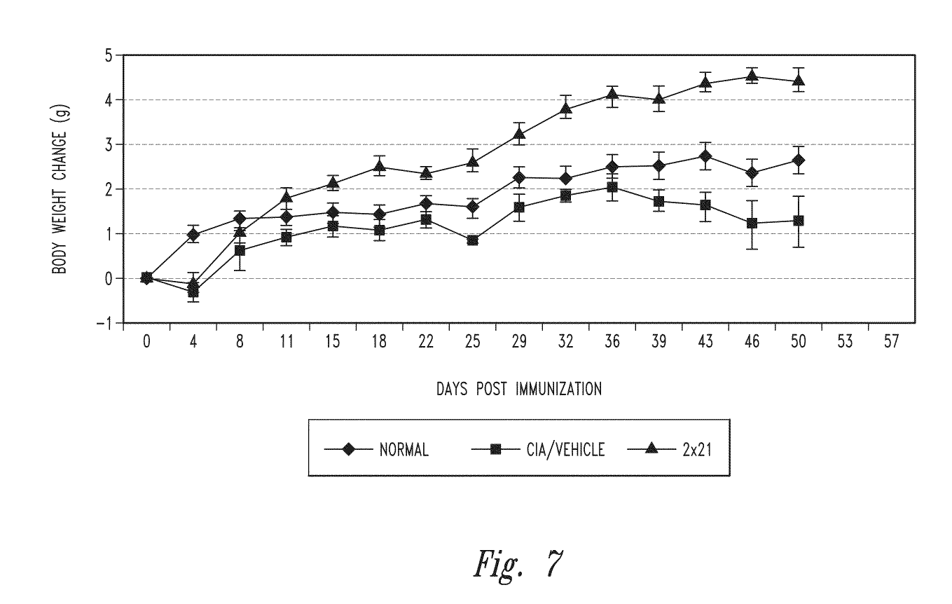

Myostatin antagonists including the binding agents described herein can be used to treat cachexia due inflammation or other immune responses including rheumatoid arthritis. Rheumatoid arthritis (RA) is a common systemic autoimmune disease that leads to joint inflammation, progressive cartilage/bone erosion, and rheumatoid cachexia. Rheumatoid cachexia is described as a loss of body cell mass, particularly muscle mass, that can occur in rheumatoid arthritis patients (Rall et al., Rheumatology 43, 1219-1223 (2004), Roubenoff et al, J Clin Invest 93, 2379-2386 (1994)). Collagen-induced arthritis (CIA) is a commonly used mouse model for RA. Example 12 describes the treatment of CIA mice with an exemplary peptibody which prevented the rapid body weight loss due to cachexia found in the control, as shown in FIG. 7. This example demonstrates that myostatin antagonists, including the peptibodies described herein, are useful for treating rheumatoid cachexia. Further, myostatin antagonists have also been demonstrated to decrease levels of TNF-α (tumor necrosis factor-α) in animals treated with LPS (E. coli lipopolysaccharide). This experiment is described in Example 14 below. This demonstrates that myostatin antagonists are also useful for treating the inflammatory component of the immune disorders such as RA.

In addition, injuries due to burns have been found to contribute to an increase in myostatin mRNA in animals (Land et al, FASEB 15 1807-1809 (2001). Myostatin antagonists including the binding agents described herein are useful for treatment of individuals from wasting resulting from burns injuries.

Additional conditions resulting in muscle wasting or atrophy may arise from inactivity due to disability such as confinement in a wheelchair or prolonged bedrest. Prolonged bedrest or inactivity may be due to stroke, heart disease, other chronic illness, spinal chord injury, coma, bone fracture or trauma, frailty due to old age or dementia, and recovery from surgeries such as hip or knee replacement. For example, plasma myostatin immunoreactive protein was found to increase after prolonged bedrest (Zachwiej a et al. J Gravit Physiol. 6(2):11(1999)). Prevention of loss of body weight, in particular lean body mass, has been demonstrated in a mouse model of disuse atrophy, a hindlimb suspension model. C57B1/6 male mice were tail suspended and received placebo or a peptibody 2x TN8-19-21 at 3 mg/kg every 3 days for 14 days. Treatment with the exemplary peptibody attenuated the loss of lean body mass and muscle strength in the suspended mice compared with suspended control mice receiving a placebo.

Other conditions resulting in muscle wasting is exposure to a microgravity environment (space flight). It was found, for example, that the muscles of rats exposed to a microgravity environment during a space shuttle flight expressed an increased amount of myostatin compared with the muscles of rats which were not exposed (Lalani et al., J. Endocrin 167 (3): 417-28 (2000)). Therefore, myostatin antagonists including the myostatin binding agents described herein can be used to prevent muscle loss and weakness due to space flight.

In addition, age related frailty/sarconpenia can be treated with myostatin antagonists including the myostatin binding agents described herein. These effects include age-related increases in fat to muscle ratios, and age-related muscular atrophy and weakness. As used herein the term “sarcopenia” refers to the loss of muscle mass that occurs with age. Average serum myostatin-immunoreactive protein increased with age in groups of young (19-35 yr old), middle-aged (36-75 yr old), and elderly (76-92 yr old) men and women, while the average muscle mass and fat-free mass declined with age in these groups (Yarasheski et al. J Nutr Aging 6(5):343-8 (2002)). It has also been shown that age-related increases in adipose tissue mass and decrease in muscle mass were proportional to myostatin levels, as determined by a comparison of fat and muscle mass in Mstn⁺¹⁺ when compared with Mstn adult knockout mice (McFerron et al. J. Clin. Invest 109, 595 (2002)). Mstn^{-/-} mice showed decreased fat accumulation with age compared with Mstn^{+/+} mice.

Reducing myostatin levels in the heart muscle may improve recovery of heart muscle after infarct, since myostatin levels are expressed at low levels in heart muscle and expression is upregulated in cardiomyocytes after infarct (Sharma et al., J Cell Physiol. 180 (1):1-9 (1999)).

In addition, increasing muscle mass by reducing myostatin levels may improve bone strength and reduce osteoporosis and other degenerative bone diseases. It has been found, for example, that myostatin-deficient mice showed increased mineral content and density of the mouse humerus and increased mineral content of both trabecular and cortical bone at the regions where the muscles attach, as well as increased muscle mass (Hamrick et al. Calcif Tissue Int 71(1):63-8 (2002)).

### Treatment Alternative to Growth Hormone

Myostatin antagonists including the binding agents of the present invention may be further used to as an alternative treatment for disorders currently treated by the growth hormone (GH), insulin growth factor-1, growth hormone secretagogues, or androgens. Treatment with GH or growth hormone secretagogues is the classic anabolic treatment for growth and muscle related disorders such as Prader-Willi disease described below. However, GH treatment will often have negative effects. Myostatin antagonists are useful as an alternative to this treatment, producing a more selective muscle response without the dangerous side-effects of GH related therapies. Myostatin antagonists are also useful for treating a GH resistant population, or aging individuals who have become resistant to GH.

Myostatin antagonists are useful, for example, for treating Prader-Willi syndrome, a genetic disorder usually involving chromosome 15. Prader-Willi is characterized by obesity, hypotonia, or poor muscle tone, and significant developmental delays in children afflicted with this disorder (Wattendorf et al, Amer Fam Physician 72 (5), 827-830 (2005)). This genetic disorder is currently treated with growth hormone, which can be dangerous to young children. (Riedl et al, Acta Paedriatr 94(7):97407 (2005), Miller J, J Clin Endocrinol Metab epub Nov. 29 (2005)). Myostatin antagonists including the binding agents described herein increase muscle mass and strength as well as decrease the ratio of fat to muscle, and are therefore useful for treating this condition.

### Treatment of Hypogonadism

Myostatin antagonists including the binding agents of the present invention can be used to treat the results of hypogonadism in subjects in need of such a treatment. As used herein, the term “hypogonadism” refers to inadequate or reduced gonad functioning in both males and females, resulting from deficiencies in the sexual organs or reduced secretion of gonadal hormones. As used herein hypogonadism includes the results of chemical or surgical castration (also referred to as orchiectomy or loss of one or both testes), and age-related hypgonadism. Androgen deprivation therapy through chemical or surgical castration is used to treat prostate cancer, other sex organ related cancers such as ovarian cancer, breast cancer, as well as endometriosis, and other disorders. Hypogonadism can result in decreased body weight, in particular by decreased lean body mass and increased fat mass over time, and decreased muscle strength. The treatment of orchietomized mice with a myostatin antagonist is described in Example 13 below. The orchiectomized animals treated with the myostatin peptibody antagonist show an attenuation or reversal of lean body mass loss when compared with the animals treated with the Fc vehicle. This demonstrates that myostatin antagonists are useful for treating the effects of hypogonadism, including patients subjected to androgen deprivation therapy. Myostatin antagonists can also prevent increases in fat mass in subjects suffering from hypogonadism.

The present invention also provides methods and compositions for increasing muscle mass in food animals by administering an effective dosage of myostatin antagonists such as the myostatin binding agents described herein to the animal. Since the mature C-terminal myostatin polypeptide is identical in all species tested, myostatin antagonists would be expected to be effective for increasing muscle mass and reducing fat in any agriculturally important species including cattle, chicken, turkeys, and pigs.

The myostatin antagonists of the present invention may be used alone or in combination with other therapeutic agents to enhance their therapeutic effects or decrease potential side effects. The binding agents are exemplary myostatin antagonists. The binding agents of the present invention possess one or more desirable but unexpected combination of properties to improve the therapeutic value of the agents. These properties include increased activity, increased solubility, reduced degradation, increased half-life, reduced toxicity, and reduced immunogenicity. Thus the binding agents of the present invention are useful for extended treatment regimes. In addition, the properties of hydrophilicity and hydrophobicity of the compounds of the invention are well balanced, thereby enhancing their utility for both in vitro and especially in vivo uses. Specifically, compounds of the invention have an appropriate degree of solubility in aqueous media that permits absorption and bioavailability in the body, while also having a degree of solubility in lipids that permits the compounds to traverse the cell membrane to a putative site of action, such as a particular muscle mass.

The binding agents of the present invention are useful for treating a “subject” or any animal, including humans, when administered in an effective dosages in a suitable composition.

In addition, the mystatin binding agents of the present invention are useful for detecting and quantitating myostatin in a number of assays. These assays are described in more detail below.

In general, the binding agents of the present invention are useful as capture agents to bind and immobilize myostatin in a variety of assays, similar to those described, for example, in Asai, ed., Methods in Cell Biology, 37, Antibodies in Cell Biology, Academic Press, Inc., New York (1993). The binding agent may be labeled in some manner or may react with a third molecule such as an anti-binding agent antibody which is labeled to enable myostatin to be detected and quantitated. For example, a binding agent or a third molecule can be modified with a detectable moiety, such as biotin, which can then be bound by a fourth molecule, such as enzyme-labeled streptavidin, or other proteins. (Akerstrom, J Immunol 135:2589 (1985); Chaubert, Mod Pathol 10:585 (1997)).

Throughout any particular assay, incubation and/or washing steps may be required after each combination of reagents. Incubation steps can vary from about 5 seconds to several hours, preferably from about 5 minutes to about 24 hours. However, the incubation time will depend upon the assay format, volume of solution, concentrations, and the like. Usually, the assays will be carried out at ambient temperature, although they can be conducted over a range of temperatures.

### Non-Competitive Binding Assays:

Binding assays can be of the non-competitive type in which the amount of captured myostatin is directly measured. For example, in one preferred “sandwich” assay, the binding agent can be bound directly to a solid substrate where it is immobilized. These immobilized agents then bind to myostatin present in the test sample. The immobilized myostatin is then bound with a labeling agent, such as a labeled antibody against myostatin, which can be detected. In another preferred “sandwich” assay, a second agent specific for the binding agent can be added which contains a detectable moiety, such as biotin, to which a third labeled molecule can specifically bind, such as streptavidin. (See, Harlow and Lane, Antibodies, A Laboratory Manual, Ch 14, Cold Spring Harbor Laboratory, NY (1988), which is incorporated herein by reference).

### Competitive Binding Assays:

Binding assays can be of the competitive type. The amount of myostatin present in the sample is measured indirectly by measuring the amount of myostatin displaced, or competed away, from a binding agent by the myostatin present in the sample. In one preferred competitive binding assay, a known amount of myostatin, usually labeled, is added to the sample and the sample is then contacted with the binding agent. The amount of labeled myostatin bound to the binding agent is inversely proportional to the concentration of myostatin present in the sample. (following the protocols found in, for example Harlow and Lane, Antibodies, A Laboratory Manual, Ch 14, pp. 579-583, supra).

In another preferred competitive binding assay, the binding agent is immobilized on a solid substrate. The amount of myostatin bound to the binding agent may be determined either by measuring the amount of myostatin present in a myostatin/binding agent complex, or alternatively by measuring the amount of remaining uncomplexed myostatin.

Other Binding Assays

The present invention also provides Western blot methods to detect or quantify the presence of myostatin in a sample. The technique generally comprises separating sample proteins by gel electrophoresis on the basis of molecular weight and transferring the proteins to a suitable solid support, such as nitrocellulose filter, a nylon filter, or derivatized nylon filter. The sample is incubated with the binding agents or fragments thereof that bind myostatin and the resulting complex is detected. These binding agents may be directly labeled or alternatively may be subsequently detected using labeled antibodies that specifically bind to the binding agent.

### Diagnostic Assays

The binding agents or fragments thereof of the present invention may be useful for the diagnosis of conditions or diseases characterized by increased amounts of myostatin. Diagnostic assays for high levels of myostatin include methods utilizing a binding agent and a label to detect myostatin in human body fluids, extracts of cells or specific tissue extracts. For example, serum levels of myostatin may be measured in an individual over time to determine the onset of muscle wasting associated with aging or inactivity, as described, for example, in Yarasheski et al., supra. Increased myostatin levels were shown to correlate with average decreased muscle mass and fat-free mass in groups of men and women of increasing ages (Yarasheski et al., supra). The binding agents of the present invention may be useful for monitoring increases or decreases in the levels of myostatin with a given individual over time, for example. The binding agents can be used in such assays with or without modification. In a preferred diagnostic assay, the binding agents will be labeled by attaching, e.g., a label or a reporter molecule. A wide variety of labels and reporter molecules are known, some of which have been already described herein. In particular, the present invention is useful for diagnosis of human disease.

A variety of protocols for measuring myostatin proteins using binding agents of myostatin are known in the art. Examples include enzyme-linked immunosorbent assay (ELISA), radioimmunoassay (RIA) and fluorescence activated cell sorting (FACS).

For diagnostic applications, in certain embodiments the binding agents of the present invention typically will be labeled with a detectable moiety. The detectable moiety can be any one that is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, βgalactosidase, or horseradish peroxidase (Bayer et al., Meth Enz, 184: 138 (1990)).

Pharmaceutical Compositions

The present invention also provides pharmaceutical compositions of one or more mysotatin antagonists described herein for treating the targeted disease conditions. Such compositions comprise a therapeutically or prophylactically effective amount of one or more myostatin antagonist in admixture with a pharmaceutically acceptable agent. The pharmaceutical compositions comprise antagonists that inhibit myostatin partially or completely in admixture with a pharmaceutically acceptable agent. Typically, the antagonists will be sufficiently purified for administration to an animal.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the binding agent.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present invention, binding agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The pharmaceutical compositions can be selected for parenteral delivery. Alternatively, the compositions may be selected for inhalation or for enteral delivery such as orally, aurally, opthalmically, rectally, or vaginally. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this invention may be in the form of a pyrogen-free, parenterally acceptable aqueous solution comprising the desired binding agent in a pharmaceutically acceptable vehicle. A particularly suitable vehicle for parenteral injection is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the ompounds and allow for the preparation of highly concentrated solutions. In another embodiment, a pharmaceutical composition may be formulated for inhalation. For example, a binding agent may be formulated as a dry powder for inhalation. Polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, binding agent molecules that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Another pharmaceutical composition may involve an effective quantity of binding agent in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules.

Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556 (1983), poly(2-hydroxyethyl-methacrylate) (Langer et al., J. Biomed. Mater. Res., 15:167-277, (1981); Langer et al., Chem. Tech., 12:98-105(1982)), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., PNAS (USA), 82:3688 (1985); EP 36,676; EP 88,046; EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 100 mg/kg.

For any compound, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient.

In other cases, a myostatin antagonist such as a peptibody can be delivered by implanting certain cells that have been genetically engineered, using methods such as those described herein, to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

Pharmaceutical compositions containing the myostatin antagonists of the present invention are administered to a subject in need thereof to treat any myostatin-related disorders. These include muscle-wasting disorders including but not limited to muscular dystrophy, muscle wasting in cancer, AIDS, muscle atrophy, rheumatoid arthritis, renal failure/uremia, chronic heart failure, prolonged bed-rest, spinal chord injury, stroke, and aging related sarcopenia. In addition these compositions are administed to treat obesity, diabetes, hyperglycemia, and increase bone density. The pharmaceutical compositions of the present invention are administered to a subject in need thereof to treat the effects of hypogonadism, rheumatoid cachexia, excessive TNF-α, cachexia due to burns injuries, diabetes, and chemical exposure such as chemotherapy, diabetic nephropathy, and treatment of disorders currently treated with GH or GH-related agents, such as Prader-Willi syndrome.

In addition, the pharmaceutical compositions can be administered in combination with existing treatments for the disorders listed above. These include, for example, denosomaub used for treating bone osteoporesis and frailty, in combination with myostatin antagonists.

The invention having been described, the following examples are offered by way of illustration, and not limitation.

### Example 1

### Identification of Myostatin Binding Peptides

Three filamentous phage libraries, TN8-IX (5×10⁹ independent transformants), TN12-I (1.4×10⁹ independent transformants), and linear (2.3×10⁹ independent transformants) (Dyax Corp.) were used to select for myostatin binding phage. Each library was incubated on myostatin-coated surfaces and subjected to different panning conditions: non-specific elution, and specific elution using recombinant human activin receptor IIB/Fc chimera (R&D Systems, Inc., Minneapolis, Minn.), or myostatin propeptide elution as described below. For all three libraries, the phages were eluted in a non-specific manner for the first round of selection, while the receptor and promyostatin was used in the second and third rounds of selection. The selection procedures were carried out as described below.

### Preparation of Myostatin

Myostatin protein was produced recombinantly in the E. coli K-12 strain 2596 (ATCC #202174) as follows. Polynucleotides encoding the human promyostatin molecule were cloned into the pAMG21 expression vector (ATCC No. 98113), which was derived from expression vector pCFM1656 (ATCC No. 69576) and the expression vector system described in U.S. Pat. No. 4,710,473, by following the procedure described in published International Patent Application WO 00/24782. The polynucleotides encoding promyostatin were obtained from a mammalian expression vector. The coding region was amplified using a standard PCR method and the following PCR primers to introduce the restriction site for NdeI and BamHI.

5′ primer:
(SEQ ID NO: 292)
5′-GAGAGAGAGCATATGAATGAGAACAGTGAGCAAAAAG-3′
3′primer:
(SEQ ID NO: 293)
5′-AGAGAGGGATCCATTATGAGCACCCACAGCGGTC-3′

The PCR product and vector were digested with both enzymes, mixed and ligated. The product of the ligation was transformed into E. coli strain #2596. Single colonies were checked microscopically for recombinant protein expression in the form of inclusion bodies. The plasmid was isolated and sequenced through the coding region of the recombinant gene to verify genetic fidelity.

Bacterial paste was generated from a 10 L fermentation using a batch method at 37° C. The culture was induced with HSL at a cell density of 9.6 OD₆₀₀ and harvested six hours later at a density of 104 OD₆₀₀. The paste was stored at −80° C. E. coli paste expressing promyostatin was lysed in a microfluidizer at 16,000 psi, centrifuged to isolate the insoluble inclusion body fraction. Inclusion bodies were resuspended in guanidine hydrochloride containing dithiothreitol and solubilized at room temperature. This was then diluted 30 fold in an aqueous buffer. The refolded promyostatin was then concentrated and buffer exchanged into 20 mM Tris pH 8.0, and applied to an anion exchange column. The anion exchange column was eluted with an increasing sodium chloride gradient. The fractions containing promyostatin were pooled. The promyostatin produced in E. coli is missing the first 23 amino acids and begins with a methionine before the residue 24 asparagine. To produce mature myostatin, the pooled promyostatin was enzymatically cleaved between the propeptide and mature myostatin C terminal. The resulting mixture was then applied to a C4-rpHPLC column using a increasing gradient of acetonitrile containing 0.1% trifluoroacetic acid. Fractions containing mature myostatin were pooled and dried in a speed-vac.

The recombinant mature myostatin produced from E. coli was tested in the myoblast C2C12 based assay described below and found to be fully active when compared with recombinant murine myostatin commercially produced in a mammalian cell system (R&D Systems, Inc., Minneapolis, Minn.). The E. coli-produced mature myostatin was used in the phage-display and screening assays described below.

### Preparation of Myostatin-Coated Tubes

Myostatin was immobilized on 5 ml Immuno™ Tubes (NUNC) at a concentration of 8 ug of myostatin protein in 1 ml of 0.1M sodium carbonate buffer (pH 9.6). The myostatin-coated Immuno™ Tube was incubated with orbital shaking for 1 hour at room temperature. Myostatin-coated Immuno™ Tube was then blocked by adding 5 ml of 2% milk-PBS and incubating at room temperature for 1 hour with rotation. The resulting myostatin-coated Immuno™ Tube was then washed three times with PBS before being subjected to the selection procedures. Additional Immuno™ Tubes were also prepared for negative selections (no myostatin). For each panning condition, five to ten Immuno™ Tubes were subjected to the above procedure except that the Immuno™ Tubes were coated with 1 ml of 2% BSA-PBS instead of myostatin protein.

### Negative Selection

For each panning condition, about 100 random library equivalents for TN8-IX and TN12-I libraries (5×10¹¹ pfu for TN8-IX, and 1.4×10¹¹ pfu for TN12-I) and about 10 random library equivalents for the linear library (2.3×10¹⁰ pfu) were aliquoted from the library stock and diluted to 1 ml with PBST (PBS with 0.05% Tween-20). The 1 ml of diluted library stock was added to an Immuno™ Tube prepared for the negative selection, and incubated for 10 minutes at room temperature with orbital shaking. The phage supernatant was drawn out and added to the second Immuno™ Tube for another negative selection step. In this way, five to ten negative selection steps were performed.

### Selection for Myostatin Binding

After the last negative selection step above, the phage supernatant was added to the prepared myostatin coated Immuno™ Tubes. The Immuno™ Tube was incubated with orbital shaking for one hour at room temperature, allowing specific phage to bind to myostatin. After the supernatant was discarded, the Immuno™ Tube was washed about 15 times with 2% milk-PBS, 10 times with PBST and twice with PBS for the three rounds of selection with all three libraries (TN8-IX, TN12-I, and Linear libraries) except that for the second round of selections with TN8-IX and TN12-I libraries, the Immuno™ Tube was washed about 14 times with 2% milk-PBS, twice with 2% BSA-PBS, 10 times with PBST and once with PBS.

### Non-Specific Elution

After the last washing step, the bound phages were eluted from the Immuno™ Tube by adding 1 ml of 100 mM triethylamine solution (Sigma, St. Louis, Mo.) with 10-minute incubation with orbital shaking. The pH of the phage containing solution was then neutralized with 0.5 ml of 1 M Tris-HCl (pH 7.5).

### Receptor (Human Activin Receptor) Elution of Bound Phage

For round 2 and 3, after the last washing step, the bound phages were eluted from the Immuno™ Tube by adding 1 ml of 1 μM of receptor protein (recombinant human activin receptor IIB/Fc chimera, R&D Systems, Inc., Minneapolis, Minn.) with a 1-hour incubation for each condition.

### Propeptide Elution of Bound Phage

For round 2 and 3, after the last washing step, the bound phages were eluted from the Immuno™ Tube by adding 1 ml of 1 μM propeptide protein (made as described above) with a 1-hour incubation for each condition.

### Phage Amplification

Fresh E. coli. (XL-1 Blue MRF′) culture was grown to OD₆₀₀=0.5 in LB media containing 12.5 ug/ml tetracycline. For each panning condition, 20 ml of this culture was chilled on ice and centrifuged. The bacteria pellet was resuspended in 1 ml of the min A salts solution.

Each mixture from different elution methods was added to a concentrated bacteria sample and incubated at 37° C. for 15 minutes. 2 ml of NZCYM media (2x NZCYM, 50 ug/ml Ampicillin) was added to each mixture and incubated at 37° C. for 15 minutes. The resulting 4 ml solution was plated on a large NZCYM agar plate containing 50 ug/ml ampicillin and incubated overnight at 37° C.

Each of the bacteria/phage mixture that was grown overnight on a large NZCYM agar plate was scraped off in 35 ml of LB media, and the agar plate was further rinsed with additional 35 ml of LB media. The resulting bacteria/phage mixture in LB media was centrifuged to pellet the bacteria away. 50 ul of the phage supernatant was transferred to a fresh tube, and 12.5 ml of PEG solution (20% PEG8000, 3.5M ammonium acetate) was added and incubated on ice for 2 hours to precipitate phages. The precipitated phages were centrifuged down and resuspended in 6 ml of the phage resuspension buffer (250 mM NaCl, 100 mM Tris pH8, 1 mM EDTA). This phage solution was further purified by centrifuging away the remaining bacteria and precipitating the phage for the second time by adding 1.5 ml of the PEG solution. After a centrifugation step, the phage pellet was resuspended in 400 ul of PBS. This solution was subjected to a final centrifugation to rid of remaining bacteria debris. The resulting phage preparation was titered by a standard plaque formation assay (Molecular Cloning, Maniatis et al., 3^{rd} Edition).

### Additional Rounds of Selection and Amplification

In the second round, the amplified phage (10¹¹ pfu) from the first round was used as the input phage to perform the selection and amplification steps. The amplified phage (10¹¹ pfu) from the second round in turn was used as the input phage to perform third round of selection and amplification. After the elution steps of the third round, a small fraction of the eluted phage was plated out as in the plaque formation assay above. Individual plaques were picked and placed into 96 well microtiter plates containing 100 ul of TE buffer in each well. These master plates were incubated at 4° C. overnight to allow phages to elute into the TE buffer.

### Clonal Analysis

Phage ELISA

The phage clones were subjected to phage ELISA and then sequenced. The sequences were ranked as discussed below.

Phage ELISA was performed as follows. An E. coli XL-1 Blue MRF′culture was grown until OD₆₀₀ reached 0.5. 30 ul of this culture was aliquoted into each well of a 96 well microtiter plate. 10 ul of eluted phage was added to each well and allowed to infect bacteria for 15 min at room temperature. About 120 ul of LB media containing 12.5 ug/ml of tetracycline and 50 ug/ml of ampicillin were added to each well. The microtiter plate was then incubated with shaking overnight at 37° C. Myostatin protein (2 ug/ml in 0.1M sodium carbonate buffer, pH 9.6) was allowed to coat onto a 96 well Maxisorp™ plates (NUNC) overnight at 4° C. As a control, a separate Maxisorp™ plate was coated with 2% BSA prepared in PBS.

On the following day, liquid in the protein coated Maxisorp™ plates was discarded, washed three times with PBS and each well was blocked with 300 ul of 2% milk solution at room temperature for 1 hour. The milk solution was discarded, and the wells were washed three times with the PBS solution. After the last washing step, about 50 ul of PBST-4% milk was added to each well of the protein-coated Maxisorp™ plates. About 50 ul of overnight cultures from each well in the 96 well microtiter plate was transferred to the corresponding wells of the myostatin coated plates as well as the control 2% BSA coated plates. The 100 ul mixture in the two kinds of plates were incubated for 1 hour at room temperature. The liquid was discarded from the Maxisorp™ plates, and the wells were washed about three times with PBST followed by two times with PBS. The HRP-conjugated anti-M13 antibody (Amersham Pharmacia Biotech) was diluted to about 1:7,500, and 100 ul of the diluted solution was added to each well of the Maxisorp™ plates for 1 hour incubation at room temperature. The liquid was again discarded and the wells were washed about three times with PBST followed by two time with PBS. 100 ul of LumiGlo™ Chemiluminescent substrate (KPL) was added to each well of the Maxisorp™ plates and incubated for about 5 minutes for reaction to occur. The chemiluminescent unit of the Maxisorp™ plates was read on a plate reader (Lab System).

Sequencing of the Phage Clones

For each phage clone, the sequencing template was prepared by a PCR method. The following oligonucleotide pair was used to amplify a 500 nucleotide fragment: primer #1: 5′-CGGCGCAACTATCGGTATCAAGCTG-3′ (SEQ ID NO:294) and primer #2: 5′-CATGTACCGTAACACTGAGTTTCGTC-3′(SEQ ID NO:295). The following mixture was prepared for each clone.

| Reagents | Volume (μL)/tube |
|---|---|
| distilled H₂O | 26.25 |
| 50% glycerol | 10 |
| 10X PCR Buffer (w/o MgCl₂) | 5 |
| 25 mM MgCl₂ | 4 |
| 10 mM dNTP mix | 1 |
| 100 μM primer 1 | 0.25 |
| 100 μM primer 2 | 0.25 |
| Taq polymerase | 0.25 |
| Phage in TE (section 4) | 3 |
| Final reaction volume | 50 |

A thermocycler (GeneAmp PCR System 9700, Applied Biosystem) was used to run the following program: [94° C. for 5 min; 94° C. for 30 sec, 55° C. for 30 sec, 72° C. for 45 sec.]×30 cycles; 72° C. for 7 min; cool to 4° C. The PCR product from each reaction was cleaned up using the QIAquick Multiwell PCR Purification kit (Qiagen), following the manufacturer's protocol. The PCR cleaned up product was checked by running 10 ul of each PCR reaction mixed with 1 ul of dye (10X BBXS agarose gel loading dye) on a 1% agarose gel. The remaining product was then sequenced using the ABI 377 Sequencer (Perkin Elmer) following the manufacturer recommended protocol.

Sequence Ranking and Analysis

The peptide sequences that were translated from the nucleotide sequences were correlated to ELISA data. The clones that showed high chemiluminescent units in the myostatin-coated wells and low chemiluminescent units in the 2% BSA-coated wells were identified. The sequences that occurred multiple times were identified. Candidate sequences chosen based on these criteria were subjected to further analysis as peptibodies. Approximately 1200 individual clones were analyzed. Of these approximately 132 peptides were chosen for generating the peptibodies of the present invention. These are shown in Table I below. The peptides having SEQ ID NO: 1 to 129 were used to generate peptibodies of the same name. The peptides having SEQ ID NO: 130 to 141 shown in Table I comprise two or more peptides from SEQ ID NO: 1 to 132 attached by a linker sequence. SEQ ID NO: 130 to 141 were also used to generate peptibodies of the same name.

Consensus sequences were determined for the TN-8 derived group of peptides. These are as follows:

(SEQ ID NO: 142)
KDXCXXWHWMCKPX
(SEQ ID NO: 143)
WXXCXXXGFWCXNX
(SEQ ID NO: 144)
IXGCXWWDXXCYXX
(SEQ ID NO: 145)
XXWCVSPXWFCXXX
(SEQ ID NO: 146)
XXXCPWFAXXCVDW

For all of the above consensus sequences, the underlined “core sequences” from each consensus sequence are the amino acid which always occur at that position. “X” refers to any naturally occurring or modified amino acid. The two cysteines contained with the core sequences were fixed amino acids in the TN8-IX library.

TABLE I
peptide sequence of peptibodies

| | SEQ ID | Peptide |
|---|---|---|
| PEPTIBODY NAME | NO | sequence |
| Myostatin-TN8-Con1 | 1 | KDKCKMWHWMCKPP |
| Myostatin-TN8-Con2 | 2 | KDLCAMWHWMCKPP |
| Myostatin-TN8-Con3 | 3 | KDLCKMWKWMCKPP |
| Myostatin-TN8-Con4 | 4 | KDLCKMWHWMCKPK |
| Myostatin-TN8-Con5 | 5 | WYPCYEFHFWCYDL |
| Myostatin-TN8-Con6 | 6 | WYPCYEGHFWCYDL |
| Myostatin-TN8-Con7 | 7 | IFGCKWWDVQCYQF |
| Myostatin-TN8-Con8 | 8 | IFGCKWWDVDCYQF |
| Myostatin-TN8-Con9 | 9 | ADWCVSPNWFCMVM |
| Myostatin-TN8-Con10 | 10 | HKFCPWWALFCWDF |
| Myostatin-TN8-1 | 11 | KDLCKMWHWMCKPP |
| Myostatin-TN8-2 | 12 | IDKCAIWGWMCPPL |
| Myostatin-TN8-3 | 13 | WYPCGEFGMWCLNV |
| Myostatin-TN8-4 | 14 | WFTCLWNCDNE |
| Myostatin-TN8-5 | 15 | HTPCPWFAPLCVEW |
| Myostatin-TN8-6 | 16 | KEWCWRWKWMCKPE |
| Myostatin-TN8-7 | 17 | FETCPSWAYFCLDI |
| Myostatin-TN8-8 | 18 | AYKCEANDWGCWWL |
| Myostatin-TN8-9 | 19 | NSWCEDQWHRCWWL |
| Myostatin-TN8-10 | 20 | WSACYAGHFWCYDL |
| Myostatin-TN8-11 | 21 | ANWCVSPNWFCMVM |
| Myostatin-TN8-12 | 22 | WTECYQQEFWCWNL |
| Myostatin-TN8-13 | 23 | ENTCERWKWMCPPK |
| Myostatin-TN8-14 | 24 | WLPCHQEGFWCMNF |
| Myostatin-TN8-15 | 25 | STMCSQWHWMCNPF |
| Myostatin-TN8-16 | 26 | IFGCHWWDVDCYQF |
| Myostatin-TN8-17 | 27 | IYGCKWWDIQCYDI |
| Myostatin-TN8-18 | 28 | PDWCIDPDWWCKFW |
| Myostatin-TN8-19 | 29 | QGHCTRWPWMCPPY |
| Myostatin-TN8-20 | 30 | WQECYREGFWCLQT |
| Myostatin-TN8-21 | 31 | WFDCYGPGFKCWSP |
| Myostatin-TN8-22 | 32 | GVRCPKGHLWCLYP |
| Myostatin-TN8-23 | 33 | HWACGYWPWSCKWV |
| Myostatin-TN8-24 | 34 | GPACHSPWWWCVFG |
| Myostatin-TN8-25 | 35 | TTWCISPMWFCSQQ |
| Myostatin-TN8-26 | 36 | HKFCPPWAIFCWDF |
| Myostatin-TN8-27 | 37 | PDWCVSPRWYCNMW |
| Myostatin-TN8-28 | 38 | VWKCHWFGMDCEPT |
| Myostatin-TN8-29 | 39 | KKHCQIWTWMCAPK |
| Myostatin-TN8-30 | 40 | WFQCGSTLFWCYNL |
| Myostatin-TN8-31 | 41 | WSPCYDHYFYCYTI |
| Myostatin-TN8-32 | 42 | SWMCGFFKEVCMWV |
| Myostatin-TN8-33 | 43 | EMLCMIHPVFCNPH |
| Myostatin-TN8-34 | 44 | LKTCNLWPWMCPPL |
| Myostatin-TN8-35 | 45 | VVGCKWYEAWCYNK |
| Myostatin-TN8-36 | 46 | PIHCTQWAWMCPPT |
| Myostatin-TN8-37 | 47 | DSNCPWYFLSCVIF |
| Myostatin-TN8-38 | 48 | HIWCNLAMMKCVEM |
| Myostatin-TN8-39 | 49 | NLQCIYFLGKCIYF |
| Myostatin-TN8-40 | 50 | AWRCMWFSDVCTPG |
| Myostatin-TN8-41 | 51 | WFRCFLDADWCTSV |
| Myostatin-TN8-42 | 52 | EKICQMWSWMCAPP |
| Myostatin-TN8-43 | 53 | WFYCHLNKSECTEP |
| Myostatin-TN8-44 | 54 | FWRCAIGIDKCKRV |
| Myostatin-TN8-45 | 55 | NLGCKWYEVWCFTY |
| Myostatin-TN8-46 | 56 | IDLCNMWDGMCYPP |
| Myostatin-TN8-47 | 57 | EMPCNIWGWMCPPV |
| Myostatin-TN12-1 | 58 | WFRCVLTGIVDWSECFGL |
| Myostatin-TN12-2 | 59 | GFSCTFGLDEFYVDCSPF |
| Myostatin-TN12-3 | 60 | LPWCHDQVNADWGFCMLW |
| Myostatin-TN12-4 | 61 | YPTCSEKFWIYGQTCVLW |
| Myostatin-TN12-5 | 62 | LGPCPIHHGPWPQYCVYW |
| Myostatin-TN12-6 | 63 | PFPCETHQISWLGHCLSF |
| Myostatin-TN12-7 | 64 | HWGCEDLMWSWHPLCRRP |
| Myostatin-TN12-8 | 65 | LPLCDADMMPTIGFCVAY |
| Myostatin-TN12-9 | 66 | SHWCETTFWMNYAKCVHA |
| Myostatin-TN12-10 | 67 | LPKCTHVPFDQGGFCLWY |
| Myostatin-TN12-11 | 68 | FSSCWSPVSRQDMFCVFY |
| Myostatin-TN12-13 | 69 | SHKCEYSGWLQPLCYRP |
| Myostatin-TN12-14 | 70 | PWWCQDNYVQHMLHCDSP |
| Myostatin-TN12-15 | 71 | WFRCMLMNSFDAFQCVSY |
| Myostatin-TN12-16 | 72 | PDACRDQPWYMFMGCMLG |
| Myostatin-TN12-17 | 73 | FLACFVEFELCFDS |
| Myostatin-TN12-18 | 74 | SAYCIITESDPYVLCVPL |
| Myostatin-TN12-19 | 75 | PSICESYSTMWLPMCQHN |
| Myostatin-TN12-20 | 76 | WLDCHDDSWAWTKMCRSH |
| Myostatin-TN12-21 | 77 | YLNCVMMNTSPFVECVFN |
| Myostatin-TN12-22 | 78 | YPWCDGFMIQQGITCMFY |
| Myostatin-TN12-23 | 79 | FDYCTWLNGFKDWKCWSR |
| Myostatin-TN12-24 | 80 | LPLCNLKEISHVQACVLF |
| Myostatin-TN12-25 | 81 | SPECAFARWLGIEQCQRD |
| Myostatin-TN12-26 | 82 | YPQCFNLHLLEWTECDWF |
| Myostatin-TN12-27 | 83 | RWRCEIYDSEFLPKCWFF |
| Myostatin-TN12-28 | 84 | LVGCDNVWHRCKLF |
| Myostatin-TN12-29 | 85 | AGWCHVWGEMFGMGCSAL |
| Myostatin-TN12-30 | 86 | HHECEWMARWMSLDCVGL |
| Myostatin-TN12-31 | 87 | FPMCGIAGMKDFDFCVWY |
| Myostatin-TN12-32 | 88 | RDDCTFWPEWLWKLCERP |
| Myostatin-TN12-33 | 89 | YNFCSYLFGVSKEACQLP |
| Myostatin-TN12-34 | 90 | AHWCEQGPWRYGNICMAY |
| Myostatin-TN12-35 | 91 | NLVCGKISAWGDEACARA |
| Myostatin-TN12-36 | 92 | HNVCTIMGPSMKWFCWND |
| Myostatin-TN12-37 | 93 | NDLCAMWGWRNTIWCQNS |
| Myostatin-TN12-38 | 94 | PPFCQNDNDMLQSLCKLL |
| Myostatin-TN12-39 | 95 | WYDCNVPNELLSGLCRLF |
| Myostatin-TN12-40 | 96 | YGDCDQNHWMWPFTCLSL |
| Myostatin-TN12-41 | 97 | GWMCHFDLHDWGATCQPD |
| Myostatin-TN12-42 | 98 | YFHCMFGGHEFEVHCESF |
| Myostatin-TN12-43 | 99 | AYWCWHGQCVRF |
| Myostatin-Linear-1 | 100 | SEHWTFTDWDGNEWWVRPF |
| Myostatin-Linear-2 | 101 | MEMLDSLFELLKDMVPISKA |
| Myostatin-Linear-3 | 102 | SPPEEALMEWLGWQYGKFT |
| Myostatin-Linear-4 | 103 | SPENLLNDLYILMTKQEWYG |
| Myostatin-Linear-5 | 104 | FHWEEGIPFHVVTPYSYDRM |
| Myostatin-Linear-6 | 105 | KRLLEQFMNDLAELVSGHS |
| Myostatin-Linear-7 | 106 | DTRDALFQEFYEFVRSRLVI |
| Myostatin-Linear-8 | 107 | RMSAAPRPLTYRDIMDQYWH |
| Myostatin-Linear-9 | 108 | NDKAHFFEMFMFDVHNFVES |
| Myostatin-Linear-10 | 109 | QTQAQKIDGLWELLQSIRNQ |
| Myostatin-Linear-11 | 110 | MLSEFEEFLGNLVHRQEA |
| Myostatin-Linear-12 | 111 | YTPKMGSEWTSFWHNRIHYL |
| Myostatin-Linear-13 | 112 | LNDTLLRELKMVLNSLSDMK |
| Myostatin-Linear-14 | 113 | FDVERDLMRWLEGFMQSAAT |
| Myostatin-Linear-15 | 114 | HHGWNYLRKGSAPQWFEAWV |
| Myostatin-Linear-16 | 115 | VESLHQLQMWLDQKLASGPH |
| Myostatin-Linear-17 | 116 | RATLLKDFWQLVEGYGDN |
| Myostatin-Linear-18 | 117 | EELLREFYRFVSAFDY |
| Myostatin-Linear-19 | 118 | GLLDEFSHFIAEQFYQMPGG |
| Myostatin-Linear-20 | 119 | YREMSMLEGLLDVLERLQHY |
| Myostatin-Linear-21 | 120 | HNSSQMLLSELIMLVGSMMQ |
| Myostatin-Linear-22 | 121 | WREHFLNSDYIRDKLIAIDG |
| Myostatin-Linear-23 | 122 | QFPFYVFDDLPAQLEYWIA |
| Myostatin-Linear-24 | 123 | EFFHWLHNHRSEVNHWLDMN |
| Myostatin-Linear-25 | 124 | EALFQNFFRDVLTLSEREY |
| Myostatin-Linear-26 | 125 | QYWEQQWMTYFRENGLHVQY |
| Myostatin-Linear-27 | 126 | NQRMMLEDLWRIMTPMFGRS |
| Myostatin-Linear-29 | 127 | FLDELKAELSRHYALDDLDE |
| Myostatin-Linear-30 | 128 | GKLIEGLLNELMQLETFMPD |
| Myostatin-Linear-31 | 129 | ILLLDEYKKDWKSWF |
| Myostatin-2xTN8-19 kc | 130 | QGHCTRWPWMCPPYGSGSAT |
| | | GGSGSTASSGSGSATGQGHC |
| | | TRWPWMCPPY |
| Myostatin-2xTN8-con6 | 131 | WYPCYEGHFWCYDLGSGSTA |
| | | SSGSGSATGWYPCYEGHFWC |
| | | YDL |
| Myostatin-2xTN8-5 kc | 132 | HTPCPWFAPLCVEWGSGSAT |
| | | GGSGSTASSGSGSATGHTPC |
| | | PWFAPLCVEW |
| Myostatin-2xTN8-18 kc | 133 | PDWCIDPDWWCKFWGSGSAT |
| | | GGSGSTASSGSGSATGPDWC |
| | | IDPDWWCKFW |
| Myostatin-2xTN8-11 kc | 134 | ANWCVSPNWFCMVMGSGSAT |
| | | GGSGSTASSGSGSATGANWC |
| | | VSPNWFCMVM |
| Myostatin-2xTN8-25 kc | 135 | PDWCIDPDWWCKFWGSGSAT |
| | | GGSGSTASSGSGSATGPDWC |
| | | IDPDWWCKFW |
| Myostatin-2xTN8-23 kc | 136 | HWACGYWPWSCKWVGSGSAT |
| | | GGSGSTASSGSGSATGHWAC |
| | | GYWPWSCKWV |
| Myostatin-TN8-29-19 kc | 137 | KKHCQIWTWMCAPKGSGSAT |
| | | GGSGSTASSGSGSATGQGHC |
| | | TRWPWMCPPY |
| Myostatin-TN8-19-29 kc | 138 | QGHCTRWPWMCPPYGSGSAT |
| | | GGSGSTASSGSGSATGKKHC |
| | | QIWTWMCAPK |
| Myostatin-TN8-29-19 kn | 139 | KKHCQIWTWMCAPKGSGSAT |
| | | GGSGSTASSGSGSATGQGHC |
| | | TRWPWMCPPY |
| Myostatin-TN8-29-19-8g | 140 | KKHCQIWTWMCAPKGGGGGG |
| | | GGQGHCTRWPWMCPPY |
| Myostatin-TN8-19-29-6gc | 141 | QGHCTRWPWMCPPYGGGGGG |
| | | KKHCQIWTWMCAPK |

### Example 2

### Generating Peptibodies

Construction of DNA Encoding Peptide-Fc Fusion Proteins

Peptides capable of binding myostatin were used alone or in combination with each other to construct fusion proteins in which a peptide was fused to the Fc domain of human IgG1. The amino acid sequence of the Fc portion of each peptibody is as follows (from amino terminus to carboxyl terminus):

(SEQ ID NO: 296)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK

The peptide was fused in the N configuration (peptide was attached to the N-terminus of the Fc region), the C configuration (peptide was attached to the C-terminus of the Fc region), or the N,C configuration (peptide attached both at the N and C terminus of the Fc region). Separate vectors were used to express N-terminal fusions and C-terminal fusions. Each peptibody was constructed by annealing pairs of oligonucleotides (“oligos”) to the selected phage nucleic acid to generate a double stranded nucleotide sequence encoding the peptide. These polynucleotide molecules were constructed as ApaL to XhoI fragments. The fragments were ligated into either the pAMG21-Fc N-terminal vector for the N-terminal orientation, or the pAMG21-Fc-C-terminal vector for the C-terminal orientation which had been previously digested with ApaLI and XhoI. The resulting ligation mixtures were transformed by electroporation into E. coli strain 2596 or 4167 cells (a hsdR- variant of strain 2596 cells) using standard procedures. Clones were screened for the ability to produce the recombinant protein product and to possess the gene fusion having a correct nucleotide sequence. A single such clone was selected for each of the modified peptides.

Many of constructs were created using an alternative vector designated pAMG21-2xBs-N(ZeoR) Fc. This vector is similar to the above-described vector except that the vector digestion was performed with BsmBI. Some constructs fused peptide sequences at both ends of the Fc. In those cases the vector was a composite of pAMG21-2xBs-N(ZeoR) Fc and pAMG21-2xBs-C-Fc.

Construction of pAMG21

Expression plasmid pAMG21 (ATCC No. 98113) is derived from expression vector pCFM1656 (ATCC No. 69576) and the expression vector system described in U.S. Pat. No. 4,710,473, by following the procedure described in published International Patent Application WO 00/24782, all of which are incorporated herein by reference.

Fc N-Terminal Vector

The Fc N-terminal vector was constructed using the pAMG21 Fc_GlyS_{—} Tpo vector as a template. A 5′ PCR primer (below) was designed to remove the Tpo peptide sequence in pAMG Tpo GlyS and replace it with a polylinker containing ApaLI and XhoI sites. Using this vector as a template, PCR was performed with Expand Long Polymerase, using the following 5′ primer and a universal 3′ primer:

5′primer:
(SEQ ID NO: 297)
5′ACAAACAAACATATGGGTGCACAGAAAGCGGCCGCAAAAAAACTCGAG
GGTGGAGGCGGTGGGGACA-3′
3′ primer:
(SEQ ID NO: 298)
5′GGTCATTACTGGACCGGATC-3′

The resulting PCR product was gel purified and digested with restriction enzymes NdeI and BsrGI. Both the plasmid and the polynucleotide encoding the peptide of interest together with its linker were gel purified using Qiagen (Chatsworth, Calif.) gel purification spin columns. The plasmid and insert were then ligated using standard ligation procedures, and the resulting ligation mixture was transformed into E. coli cells (strain 2596). Single clones were selected and DNA sequencing was performed. A correct clone was identified and this was used as a vector source for the modified peptides described herein.

Construction of Fc C-Terminal Vector

The Fc C-terminal vector was constructed using pAMG21 Fc_Gly5_Tpo vector as a template. A 3′ PCR primer was designed to remove the Tpo peptide sequence and to replace it with a polylinker containing ApaLI and XhoI sites. PCR was performed with Expand Long Polymerase using a universal 5′ primer and the 3′ primer.

5′ Primer:
(SEQ ID NO: 299)
5′CGTACAGGTTTACGCAAGAAAATGG-3′
3′ Primer:
(SEQ ID NO: 300)
5′TTTGTTGGATCCATTACTCGAGTTTTTTTGCGGCCGCTTTCTGTGCAC
CACCACCTCCACCTTTAC-3′

The resulting PCR product was gel purified and digested with restriction enzymes BsrGI and BamHI. Both the plasmid and the polynucleotide encoding each peptides of interest with its linker were gel purified via Qiagen gel purification spin columns. The plasmid and insert were then ligated using standard ligation procedures, and the resulting ligation mixture was transformed into E. coli (strain 2596) cells. Strain 2596 (ATCC #202174) is a strain of E. coli K-12 modified to contain the lux promoter and two lambda temperature sensitive repressors, the cI857s7 and the lac I Q repressor. Single clones were selected and DNA sequencing was performed. A correct clone was identified and used as a source of each peptibody described herein.

Expression in E. coli.

Cultures of each of the pAMG21-Fc fusion constructs in E. coli strain 2596 were grown at 37° C. in Terrific Broth medium (See Tartof and Hobbs, “Improved media for growing plasmid and cosmid clones”, Bethesda Research Labs Focus, Volume 9, page 12, 1987, cited in aforementioned Sambrook et al. reference). Induction of gene product expression from the luxPR promoter was achieved following the addition of the synthetic autoinducer, N-(3-oxohexanoyl)-DL-homoserine lactone, to the culture medium to a final concentration of 20 nanograms per milliliter (ng/ml). Cultures were incubated at 37° C. for an additional six hours. The bacterial cultures were then examined by microscopy for the presence of inclusion bodies and collected by centrifugation. Refractile inclusion bodies were observed in induced cultures, indicating that the Fc-fusions were most likely produced in the insoluble fraction in E. coli. Cell pellets were lysed directly by resuspension in Laemmli sample buffer containing 10% β-mercaptoethanol and then analyzed by SDS-PAGE. In most cases, an intense coomassie-stained band of the appropriate molecular weight was observed on an SDS-PAGE gel.

Folding and Purifying Peptibodies

Cells were broken in water ( 1/10 volume per volume) by high pressure homogenization (3 passes at 15,000 PSI) and inclusion bodies were harvested by centrifugation (4000 RPM in J-6B for 30 minutes). Inclusion bodies were solubilized in 6 M guanidine, 50 mM Tris, 8 mM DTT, pH 8.0 for 1 hour at a 1/10 ratio at ambient temperature. The solubilized mixture was diluted 25 times into 4 M urea, 20% glycerol, 50 mM Tris, 160 mM arginine, 3 mM cysteine, 1 mM cystamine, pH 8.5. The mixture was incubated overnight in the cold. The mixture was then dialyzed against 10 mM Tris pH 8.5, 50 mM NaCl, 1.5 M urea. After an overnight dialysis the pH of the dialysate was adjusted to pH 5 with acetic acid. The precipitate was removed by centrifugation and the supernatant was loaded onto a SP-Sepharose Fast Flow column equilibrated in 10 mM NaAc, 50 mM NaCl, pH 5, 4° C.). After loading the column was washed to baseline with 10 mM NaAc, 50 mM NaCl, pH 5.2. The column was developed with a 20 column volume gradient from 50 mM-500 mM NaCl in the acetate buffer. Alternatively, after the wash to baseline, the column was washed with 5 column volumes of 10 mM sodium phosphate pH 7.0 and the column developed with a 15 column volume gradient from 0-400 mM NaCl in phosphate buffer. Column fractions were analyzed by SDS-PAGE. Fractions containing dimeric peptibody were pooled. Fractions were also analyzed by gel filtration to determine if any aggregate was present.

A number of peptibodies were prepared from the peptides of Table I. The peptides were attached to the human IgG1 Fc molecule to form the peptibodies in Table II. Regarding the peptibodies in Table II, the C configuration indicates that the peptide named was attached at the C-termini of the Fc. The N configuration indicates that the peptide named was attached at the N-termini of the Fc. The N,C configuration indicates that one peptide was attached at the N-termini and one at the C-termini of each Fc molecule. The 2x designation indicates that the two peptides named were attached in tandem to each other and also attached at the N or the C termini, or both the N,C of the Fc, separated by the linker indicated. Two peptides attached in tandem separated by a linker, are indicated, for example, as Myostatin-TN8-29-19-8g, which indicates that TN8-29 peptide is attached via a (gly)8 linker to TN8-19 peptide. The peptide(s) were attached to the Fc via a (gly)5 linker sequence unless otherwise specified. In some instances the peptide(s) were attached via a k linker. The linker designated k or lk refers to the gsgsatggsgstassgsgsatg (SEQ ID NO: 301) linker sequence, with kc referring to the linker attached to the C-terminus of the Fc, and kn referring to the linker attached to the N-terminus of the Fc. In Table II below, column 4 refers to the linker sequence connecting the Fc to the first peptide and the fifth column refers to the configuration N or C or both.

Since the Fc molecule dimerizes in solution, a peptibody constructed so as to have one peptide will actually be a dimer with two copies of the peptide and two Fc molecules, and the 2X version having two peptides in tandem will actually be a dimer with four copies of the peptide and two Fc molecules.

Since the peptibodies given in Table II are expressed in E. coli, the first amino acid residue is Met (M). Therefore, the peptibodies in the N configuration are Met-peptide-linker-Fc, or Met-peptide-linker-peptide-linker-Fc, for example. Peptibodies in the C configuration are arranged as Met-Fc-linker-peptide or Met-Fc-linker-peptide-linker-peptide, for example. Peptibodies in the C,N configuration are a combination of both, for example, Met-peptide-linker-Fc-linker-peptide.

Nucleotide sequences encoding exemplary peptibodies are provided below in Table II. The polynucleotide sequences encoding an exemplary peptibody of the present invention includes a nucleotide sequence encoding the Fc polypeptide sequence such as the following:

(SEQ ID NO: 301)
5′GACAAAACTCACACATGTCCACCTTGCCCAGCACCTGAACTCCTGGGG
GGACCGTCAGTTTTCCTCTTCCCCCCAAAACCCAAGGACACCCTCATGAT
CTCCCGGACCCCTGAGGTCACATGCGTGGTGGTGGACGTGAGCCACGAAG
ACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGT
CAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCC
ATCCCGGGATGAGCTGACCAAGAACCAGGTCAGCCTGACCTGCCTGGTCA
AAGGCTTCTATCCCAGCGACATCGCCGTGGAGTGGGAGAGCAATGGGCAG
CCGGAGAACAACTACAAGACCACGCCTCCCGTGCTGGACTCCGACGGCTC
CTTCTTCCTCTACAGCAAGCTCACCGTGGACAAGAGCAGGTGGCAGCAGG
GGAACGTCTTCTCATGCTCCGTGATGCATGAGGCTCTGCACAACCACTAC
ACGCAGAAGAGCCTCTCCCTGTCTCCGGGTAAA-3′

In addition, the polynucleotides encoding the 5 glycine (5G) (GGGGG) (SEQ ID NO:635) linker such as the following are included:

(SEQ ID NO: 302)
5′-GGTGGAGGTGGTGGT-3′

The polynucleotide encoding the peptibody also includes the codon encoding the methionine ATG and a stop codon such as TAA.

Therefore, the structure of the first peptibody in Table II is TN8-Con1 with a C configuration and a (gly)5 linker is as follows: M-Fc-GGGGG-KDKCKMWHWMCKPP (SEQ ID NO: 303). Exemplary polynucleotides encoding this peptibody would be:

(SEQ ID NO: 304)
5′ATGGACAAAACTCACACATGTCCACCTTGCCCAGCACCTGAACTCCTG
GGGGGACCGTCAGTTTTCCTCTTCCCCCCAAAACCCAAGGACACCCTCAT
GATCTCCCGGACCCCTGAGGTCACATGCGTGGTGGTGGACGTGAGCCACG
AAGACCCTGAGGTCAAGTTCAACTGGTACGTGGACGGCGTGGAGGTGCAT
AATGCCAAGACAAAGCCGCGGGAGGAGCAGTACAACAGCACGTACCGTGT
GGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACC
ATCTCCAAAGCCAAAGGGCAGCCCCGAGAACCACAGGTGTACACCCTGCC
CCCATCCCGGGATGAGCTGACCAAGAACCAGGTCAGCCTGACCTGCCTGG
TCAAAGGCTTCTATCCCAGCGACATCGCCGTGGAGTGGGAGAGCAATGGG
CAGCCGGAGAACAACTACAAGACCACGCCTCCCGTGCTGGACTCCGACGG
CTCCTTCTTCCTCTACAGCAAGCTCACCGTGGACAAGAGCAGGTGGCAGC
AGGGGAACGTCTTCTCATGCTCCGTGATGCATGAGGCTCTGCACAACCAC
TACACGCAGAAGAGCCTCTCCCTGTCTCCGGGTAAAGGTGGAGGTGGTGG
TAAGACAAATGCAAAATGTGGCACTGGATGTGCAAACCGCCG-3′

TABLE II
peptibody peptide and nucleotide sequences

| Peptibody | | | | |
|---|---|---|---|---|
| Name | Peptide | Nucleotide Sequence (SEQ ID NO) | | |
| Myostatin-TN8- | KDKCKMWHWMCKPP | AAAGACAAATGCAAAATGTGGCACTGGAT | 5 gly | C |
| con1 | | GTGCAAACCGCCG (Seq. ID No: 147) | | |
| Myostatin-TN8- | KDLCAMWHWMCKPP | AAAGACCTGTGCGCTATGTGGCACTGGAT | 5 gly | C |
| con2 | | GTGCAAACCGCCG (Seq. ID No: 148) | | |
| Myostatin-TN8- | KDLCKMWKWMCKPP | AAAGACCTGTGCAAAATGTGGAAATGGAT | 5 gly | C |
| con3 | | GTGCAAACCGCCG (SEQ ID NO: 149) | | |
| Myostatin-TN8- | KDLCKMWHWMCKPK | AAAGACCTGTGCAAAATGTGGCACTGGAT | 5 gly | C |
| con4 | | GTGCAAACCGAAA (SEQ ID NO: 150) | | |
| Myostatin-TN8- | WYPCYEFHFWCYDL | TGGTACCCGTGCTACGAATTCCACTTCTGG | 5 gly | C |
| con5 | | TGCTACGACCTG (SEQ ID NO: 151) | | |
| Myostatin-TN8- | WYPCYEFHFWCYDL | TGGTACCCGTGCTACGAATTCCACTTCTGG | 5 gly | N |
| con5 | | TGCTACGACCTG (SEQ ID NO: 152) | | |
| Myostatin-TN8- | WYPCYEGHFWCYDL | TGGTACCCGTGCTACGAAGGTCACTTCTGG | 5 gly | C |
| con6 | | TGCTACGACCTG (SEQ ID NO: 153) | | |
| Myostatin-TN8- | WYPCYEGHFWCYDL | TGGTACCCGTGCTACGAAGGTCACTTCTGG | 5 gly | N |
| con6 | | TGCTACGACCTG (SEQ ID NO: 154) | | |
| Myostatin-TN8- | IFGCKWWDVQCYQF | ATCTTCGGTTGCAAATGGTGGGACGTTCAG | 5 gly | C |
| con7 | | TGCTACCAGTTC (SEQ ID NO: 155) | | |
| Myostatin-TN8- | IFGCKWWDVDCYQF | ATCTTCGGTTGCAAATGGTGGGACGTTGAC | 5 gly | C |
| con8 | | TGCTACCAGTTC (SEQ ID NO: 156) | | |
| Myostatin-TN8- | IFGCKWWDVDCYQF | ATCTTCGGTTGCAAATGGTGGGACGTTGAC | 5 gly | N |
| con8 | | TGCTACCAGTTC (SEQ ID NO: 157) | | |
| Myostatin-TN8- | ADWCVSPNWFCMVM | GCTGACTGGTGCGTTTCCCCGAACTGGTTC | 5 gly | C |
| con9 | | TGCATGGTTATG (SEQ ID NO: 158) | | |
| Myostatin-TN8- | HKFCPWWALFCWDF | CACAAATTCTGCCCGTGGTGGGCTCTGTTC | 5 gly | C |
| con10 | | TGCTGGGACTTC (SEQ ID NO: 159) | | |
| Myostatin-TN8-1 | KDLCKMWHWMCKPP | AAAGACCTGTGCAAAATGTGGCACTGGAT | 5 gly | C |
| | | GTGCAAACCGCCG (SEQ ID NO: 160 | | |
| Myostatin-TN8-2 | IDKCAIWGWMCPPL | ATCGACAAATGCGCTATCTGGGGTTGGAT | 5 gly | C |
| | | GTGCCCGCCGCTG (SEQ ID NO: 161) | | |
| Myostatin-TN8-3 | WYPCGEFGMWCLNV | TGGTACCCGTGCGGTGAATTCGGTATGTGG | 5 gly | C |
| | | TGCCTGAACGTT (SEQ ID NO: 162) | | |
| Myostatin-TN8-4 | WFTCLWNCDNE | TGGTTCACCTGCCTGTGGAACTGCGACAAC | 5 gly | C |
| | | GAA (SEQ ID NO: 163) | | |
| Myostatin-TN8-5 | HTPCPWFAPLCVEW | CACACCCCGTGCCCGTGGTTCGCTCCGCTG | 5 gly | C |
| | | TGCGTTGAATGG (SEQ ID NO: 164) | | |
| Myostatin-TN8-6 | KEWCWRWKWMCKPE | AAAGAATGGTGCTGGCGTTGGAAATGGAT | 5 gly | C |
| | | GTGCAAACCGGAA (SEQ ID NO: 165) | | |
| Myostatin-TN8-7 | FETCPSWAYFCLDI | TTCGAAACCTGCCCGTCCTGGGCTTACTTC | 5 gly | C |
| | | TGCCTGGACATC (SEQ ID NO: 166) | | |
| Myostatin-TN8-7 | FETCPSWAYFCLDI | TTCGAAACCTGCCCGTCCTGGGCTTACTTC | 5 gly | N |
| | | TGCCTGGACATC (SEQ ID NO: 167) | | |
| Myostatin-TN8-8 | AYKCEANDWGCWWL | GCTTACAAATGCGAAGCTAACGACTGGGG | 5 gly | C |
| | | TTGCTGGTGGCTG (SEQ ID NO: 168) | | |
| Myostatin-TN8-9 | NSWCEDQWHRCWWL | AACTCCTGGTGCGAAGACCAGTGGCACCG | 5 gly | C |
| | | TTGCTGGTGGCTG (SEQ ID NO: 169) | | |
| Myostatin-TN8-10 | WSACYAGHFWCYDL | TGGTCCGCTTGCTACGCTGGTCACTTCTGG | 5 gly | C |
| | | TGCTACGACCTG (SEQ ID NO: 170) | | |
| Myostatin-TN8-11 | ANWCVSPNWFCMVM | GCTAACTGGTGCGTTTCCCCGAACTGGTTC | 5 gly | C |
| | | TGCATGGTTATG (SEQ ID NO: 171) | | |
| Myostatin-TN8-12 | WTECYQQEFWCWNL | TGGACCGAATGCTACCAGCAGGAATTCTG | 5 gly | C |
| | | GTGCTGGAACCTG (SEQ ID NO: 172) | | |
| Myostatin-TN8-13 | ENTCERWKWMCPPK | GAAAACACCTGCGAACGTTGGAAATGGAT | 5 gly | C |
| | | GTGCCCGCCGAAA (SEQ ID NO: 173) | | |
| Myostatin-TN8-14 | WLPCHQEGFWCMNF | TGGCTGCCGTGCCACCAGGAAGGTTTCTG | 5 gly | C |
| | | GTGCATGAACTTC (SEQ ID NO: 174) | | |
| Myostatin-TN8-15 | STMCSQWHWMCNPF | TCCACCATGTGCTCCCAGTGGCACTGGATG | 5 gly | C |
| | | TGCAACCCGTTC (SEQ ID NO: 175) | | |
| Myostatin-TN8-16 | IFGCHWWDVDCYQF | ATCTTCGGTTGCCACTGGTGGGACGTTGAC | 5 gly | C |
| | | TGCTACCAGTTC (SEQ ID NO: 176) | | |
| Myostatin-TN8-17 | IYGCKWWDIQCYDI | ATCTACGGTTGCAAATGGTGGGACATCCA | 5 gly | C |
| | | GTGCTACGACATC (SEQ ID NO: 177) | | |
| Myostatin-TN8-18 | PDWCIDPDWWCKFW | CCGGACTGGTGCATCGATCCGGACTGGTG | 5 gly | C |
| | | GTGCAAATTCTGG (SEQ ID NO: 178) | | |
| Myostatin-TN8-19 | QGHCTRWPWMCPPY | CAGGGTCACTGCACCCGTTGGCCGTGGAT | 5 gly | C |
| | | GTGCCCGCCGTAC (SEQ ID NO: 179) | | |
| Myostatin-TN8-20 | WQECYREGFWCLQT | TGGCAGGAATGCTACCGTGAAGGTTTCTG | 5 gly | C |
| | | GTGCCTGCAGACC (SEQ ID NO: 180) | | |
| Myostatin-TN8-21 | WFDCYGPGFKCWSP | TGGTTCGACTGCTACGGTCCGGGTTTCAAA | 5 gly | C |
| | | TGCTGGTCCCCG (SEQ ID NO: 181) | | |
| Myostatin-TN8-22 | GVRCPKGHLWCLYP | GGTGTTCGTTGCCCGAAAGGTCACCTGTGG | 5 gly | C |
| | | TGCCTGTACCCG (SEQ ID NO: 182) | | |
| Myostatin-TN8-23 | HWACGYWPWSCKWV | CACTGGGCTTGCGGTTACTGGCCGTGGTCC | 5 gly | C |
| | | TGCAAATGGGTT (SEQ ID NO: 183) | | |
| Myostatin-TN8-24 | GPACHSPWWWCVFG | GGTCCGGCTTGCCACTCCCCGTGGTGGTGG | 5 gly | C |
| | | TGCGTTTTCGGT (SEQ ID NO: 184) | | |
| Myostatin-TN8-25 | TTWCISPMWFCSQQ | ACCACCTGGTGCATCTCCCCGATGTGGTTC | 5 gly | C |
| | | TGCTCCCAGCAG (SEQ ID NO: 185) | | |
| Myostatin-TN8-26 | HKFCPPWAIFCWDF | CACAAATTCTGCCCGCCGTGGGCTATCTTC | 5 gly | N |
| | | TGCTGGGACTTC (SEQ ID NO: 186) | | |
| Myostatin-TN8-27 | PDWCVSPRWYCNMW | CCGGACTGGTGCGTTTCCCCGCGTTGGTAC | 5 gly | N |
| | | TGCAACATGTGG (SEQ ID NO: 187) | | |
| Myostatin-TN8-28 | VWKCHWFGMDCEPT | GTTTGGAAATGCCACTGGTTCGGTATGGAC | 5 gly | N |
| | | TGCGAACCGACC (SEQ ID NO: 188) | | |
| Myostatin-TN8-29 | KKHCQIWTWMCAPK | AAAAAACACTGCCAGATCTGGACCTGGAT | 5 gly | N |
| | | GTGCGCTCCGAAA (SEQ ID NO: 189) | | |
| Myostatin-TN8-30 | WFQCGSTLFWCYNL | TGGTTCCAGTGCGGTTCCACCCTGTTCTGG | 5 gly | N |
| | | TGCTACAACCTG (SEQ ID NO: 190) | | |
| Myostatin-TN8-31 | WSPCYDHYFYCYTI | TGGTCCCCGTGCTACGACCACTACTTCTAC | 5 gly | N |
| | | TGCTACACCATC (SEQ ID NO: 191) | | |
| Myostatin-TN8-32 | SWMCGFFKEVCMWV | TCCTGGATGTGCGGTTTCTTCAAAGAAGTT | 5 gly | N |
| | | TGCATGTGGGTT (SEQ ID NO: 192) | | |
| Myostatin-TN8-33 | EMLCMIHPVFCNPH | GAAATGCTGTGCATGATCCACCCGGTTTTC | 5 gly | N |
| | | TGCAACCCGCAC (SEQ ID NO: 193) | | |
| Myostatin-TN8-34 | LKTCNLWPWMCPPL | CTGAAAACCTGCAACCTGTGGCCGTGGAT | 5 gly | N |
| | | GTGCCCGCCGCTG (SEQ ID NO: 194) | | |
| Myostatin-TN8-35 | VVGCKWYEAWCYNK | GTTGTTGGTTGCAAATGGTACGAAGCTTGG | 5 gly | N |
| | | TGCTACAACAAA (SEQ ID NO: 195) | | |
| Myostatin-TN8-36 | PIHCTQWAWMCPPT | CCGATCCACTGCACCCAGTGGGCTTGGAT | 5 gly | N |
| | | GTGCCCGCCGACC (SEQ ID NO: 196) | | |
| Myostatin-TN8-37 | DSNCPWYFLSCVIF | GACTCCAACTGCCCGTGGTACTTCCTGTCC | 5 gly | N |
| | | TGCGTTATCTTC (SEQ ID NO: 197) | | |
| Myostatin-TN8-38 | HIWCNLAMMKCVEM | CACATCTGGTGCAACCTGGCTATGATGAA | 5 gly | N |
| | | ATGCGTTGAAATG (SEQ ID NO: 198) | | |
| Myostatin-TN8-39 | NLQCIYFLGKCIYF | AACCTGCAGTGCATCTACTTCCTGGGTAAA | 5 gly | N |
| | | TGCATCTACTTC (SEQ ID NO: 199) | | |
| Myostatin-TN8-40 | AWRCMWFSDVCTPG | GCTTGGCGTTGCATGTGGTTCTCCGACGTT | 5 gly | N |
| | | TGCACCCCGGGT (SEQ ID NO: 200) | | |
| Myostatin-TN8-41 | WFRCFLDADWCTSV | TGGTTTCGTTGTTTTCTTGATGCTGATTGGT | 5 gly | N |
| | | GTACTTCTGTT (SEQ ID NO: 201) | | |
| Myostatin-TN8-42 | EKICQMWSWMCAPP | GAAAAAATTTGTCAAATGTGGTCTTGGAT | 5 gly | N |
| | | GTGTGCTCCACCA (SEQ ID NO: 202) | | |
| Myostatin-TN8-43 | WFYCHLNKSECTEP | TGGTTTTATTGTCATCTTAATAAATCTGAA | 5 gly | N |
| | | TGTACTGAACCA (SEQ ID NO: 203) | | |
| Myostatin-TN8-44 | FWRCAIGIDKCKRV | TTTTGGCGTTGTGCTATTGGTATTGATAAA | 5 gly | N |
| | | TGTAAACGTGTT (SEQ ID NO: 204) | | |
| Myostatin-TN8-45 | NLGCKWYEVWCFTY | AATCTTGGTTGTAAATGGTATGAAGTTTGG | 5 gly | N |
| | | TGTTTTACTTAT (SEQ ID NO: 205) | | |
| Myostatin-TN8-46 | IDLCNMWDGMCYPP | ATTGATCTTTGTAATATGTGGGATGGTATG | 5 gly | N |
| | | TGTTATCCACCA (SEQ ID NO: 206) | | |
| Myostatin-TN8-47 | EMPCNIWGWMCPPV | GAAATGCCATGTAATATTTGGGGTTGGAT | 5 gly | N |
| | | GTGTCCACCAGTT (SEQ ID NO: 207) | | |
| Myostatin-TN12-1 | WFRCVLTGIVDWSECF | TGGTTCCGTTGCGTTCTGACCGGTATCGTT | 5 gly | N |
| | GL | GACTGGTCCGAATGCTTCGGTCTG (SEQ ID | | |
| | | NO: 208) | | |
| Myostatin-TN12-2 | GFSCTFGLDEFYVDCSP | GGTTTCTCCTGCACCTTCGGTCTGGACGAA | 5 gly | N |
| | F | TTCTACGTTGACTGCTCCCCGTTC (SEQ ID | | |
| | | NO: 209) | | |
| Myostatin-TN12-3 | LPWCHDQVNADWGFC | CTGCCGTGGTGCCACGACCAGGTTAACGC | 5 gly | N |
| | MLW | TGACTGGGGTTTCTGCATGCTGTGG (SEQ | | |
| | | ID NO: 210) | | |
| Myostatin-TN12-4 | YPTCSEKFWIYGQTCV | TACCCGACCTGCTCCGAAAAATTCTGGATC | 5 gly | N |
| | LW | TACGGTCAGACCTGCGTTCTGTGG (SEQ ID | | |
| | | NO: 211) | | |
| Myostatin-TN12-5 | LGPCPIHHGPWPQYCV | CTGGGTCCGTGCCCGATCCACCACGGTCCG | 5 gly | N |
| | YW | TGGCCGCAGTACTGCGTTTACTGG (SEQ ID | | |
| | | NO: 212) | | |
| Myostatin-TN12-6 | PFPCETHQISWLGHCLS | CCGTTCCCGTGCGAAACCCACCAGATCTCC | 5 gly | N |
| | F | TGGCTGGGTCACTGCCTGTCCTTC (SEQ ID | | |
| | | NO: 213) | | |
| Myostatin-TN12-7 | HWGCEDLMWSWHPLC | CACTGGGGTTGCGAAGACCTGATGTGGTC | 5 gly | N |
| | RRP | CTGGCACCCGCTGTGCCGTCGTCCG (SEQ | | |
| | | ID NO: 214) | | |
| Myostatin-TN12-8 | LPLCDADMMPTIGFCV | CTGCCGCTGTGCGACGCTGACATGATGCC | 5 gly | N |
| | AY | GACCATCGGTTTCTGCGTTGCTTAC (SEQ | | |
| | | ID NO: 215) | | |
| Myostatin-TN12-9 | SHWCETTFWMNYAKC | TCCCACTGGTGCGAAACCACCTTCTGGATG | 5 gly | N |
| | VHA | AACTACGCTAAATGCGTTCACGCT (SEQ ID | | |
| | | NO: 216) | | |
| Myostatin-TN12- | LPKCTHVPFDQGGFCL | CTGCCGAAATGCACCCACGTTCCGTTCGAC | 5 gly | N |
| 10 | WY | CAGGGTGGTTTCTGCCTGTGGTAC (SEQ ID | | |
| | | NO: 217) | | |
| Myostatin-TN12- | FSSCWSPVSRQDMFCV | TTCTCCTCCTGCTGGTCCCCGGTTTCCCGTC | 5 gly | N |
| 11 | FY | AGGACATGTTCTGCGTTTTCTAC (SEQ ID | | |
| | | NO: 218) | | |
| Myostatin-TN12- | SHKCEYSGWLQPLCYR | TCCCACAAATGCGAATACTCCGGTTGGCTG | 5 gly | N |
| 13 | P | CAGCCGCTGTGCTACCGTCCG (SEQ ID NO: | | |
| | | 219) | | |
| Myostatin-TN12- | PWWCQDNYVQHMLH | CCGTGGTGGTGCCAGGACAACTACGTTCA | 5 gly | N |
| 14 | CDSP | GCACATGCTGCACTGCGACTCCCCG (SEQ | | |
| | | ID NO: 220) | | |
| Myostatin-TN12- | WFRCMLMNSFDAFQC | TGGTTCCGTTGCATGCTGATGAACTCCTTC | 5 gly | N |
| 15 | VSY | GACGCTTTCCAGTGCGTTTCCTAC (SEQ ID | | |
| | | NO: 221) | | |
| Myostatin-TN12- | PDACRDQPWYMFMGC | CCGGACGCTTGCCGTGACCAGCCGTGGTA | 5 gly | N |
| 16 | MLG | CATGTTCATGGGTTGCATGCTGGGT (SEQ | | |
| | | ID NO: 222) | | |
| Myostatin-TN12- | FLACFVEFELCFDS | TTCCTGGCTTGCTTCGTTGAATTCGAACTG | 5 gly | N |
| 17 | | TGCTTCGACTCC (SEQ ID NO: 223) | | |
| Myostatin-TN12- | SAYCIITESDPYVLCVP | TCCGCTTACTGCATCATCACCGAATCCGAC | 5 gly | N |
| 18 | L | CCGTACGTTCTGTGCGTTCCGCTG (SEQ ID | | |
| | | NO: 224) | | |
| Myostatin-TN12- | PSICESYSTMWLPMCQ | CCGTCCATCTGCGAATCCTACTCCACCATG | 5 gly | N |
| 19 | HN | TGGCTGCCGATGTGCCAGCACAAC (SEQ | | |
| | | ID NO: 225) | | |
| Myostatin-TN12- | WLDCHDDSWAWTKM | TGGCTGGACTGCCACGACGACTCCTGGGC | 5 gly | N |
| 20 | CRSH | TTGGACCAAAATGTGCCGTTCCCAC (SEQ | | |
| | | ID NO: 226) | | |
| Myostatin-TN12- | YLNCVMMNTSPFVEC | TACCTGAACTGCGTTATGATGAACACCTCC | 5 gly | N |
| 21 | VFN | CCGTTCGTTGAATGCGTTTTCAAC (SEQ ID | | |
| | | NO: 227) | | |
| Myostatin-TN12- | YPWCDGFMIQQGITCM | TACCCGTGGTGCGACGGTTTCATGATCCAG | 5 gly | N |
| 22 | FY | CAGGGTATCACCTGCATGTTCTAC (SEQ ID | | |
| | | NO: 228) | | |
| Myostatin-TN12- | FDYCTWLNGFKDWKC | TTCGACTACTGCACCTGGCTGAACGGTTTC | 5 gly | N |
| 23 | WSR | AAAGACTGGAAATGCTGGTCCCGT (SEQ | | |
| | | ID NO: 229) | | |
| Myostatin-TN12- | LPLCNLKEISHVQACVL | CTGCCGCTGTGCAACCTGAAAGAAATCTC | 5 gly | N |
| 24 | F | CCACGTTCAGGCTTGCGTTCTGTTC (SEQ | | |
| | | ID NO: 230) | | |
| Myostatin-TN12- | SPECAFARWLGIEQCQ | TCCCCGGAATGCGCTTTCGCTCGTTGGCTG | 5 gly | N |
| 25 | RD | GGTATCGAACAGTGCCAGCGTGAC (SEQ | | |
| | | ID NO: 231) | | |
| Myostatin-TN12- | YPQCFNLHLLEWTECD | TACCCGCAGTGCTTCAACCTGCACCTGCTG | 5 gly | N |
| 26 | WF | GAATGGACCGAATGCGACTGGTTC (SEQ | | |
| | | ID NO: 232) | | |
| Myostatin-TN12- | RWRCEIYDSEFLPKCW | CGTTGGCGTTGCGAAATCTACGACTCCGA | 5 gly | N |
| 27 | FF | ATTCCTGCCGAAATGCTGGTTCTTC (SEQ | | |
| | | ID NO: 233) | | |
| Myostatin-TN12- | LVGCDNVWHRCKLF | CTGGTTGGTTGCGACAACGTTTGGCACCGT | 5 gly | N |
| 28 | | TGCAAACTGTTC (SEQ ID NO: 234) | | |
| Myostatin-TN12- | AGWCHVWGEMFGMG | GCTGGTTGGTGCCACGTTTGGGGTGAAAT | 5 gly | N |
| 29 | CSAL | GTTCGGTATGGGTTGCTCCGCTCTG (SEQ | | |
| | | ID NO: 235) | | |
| Myostatin-TN12- | HHECEWMARWMSLD | CACCACGAATGCGAATGGATGGCTCGTTG | 5 gly | N |
| 30 | CVGL | GATGTCCCTGGACTGCGTTGGTCTG (SEQ | | |
| | | ID NO: 236) | | |
| Myostatin-TN12- | FPMCGIAGMKDFDFCV | TTCCCGATGTGCGGTATCGCTGGTATGAAA | 5 gly | N |
| 31 | WY | GACTTCGACTTCTGCGTTTGGTAC (SEQ ID | | |
| | | NO: 237) | | |
| Myostatin-TN12- | RDDCTFWPEWLWKLC | CGTGATGATTGTACTTTTTGGCCAGAATGG | 5 gly | N |
| 32 | ERP | CTTTGGAAACTTTGTGAACGTCCA (SEQ ID | | |
| | | NO: 238) | | |
| Myostatin-TN12- | YNFCSYLFGVSKEACQ | TATAATTTTTGTTCTTATCTTTTTGGTGTTT | 5 gly | N |
| 33 | LP | CTAAAGAAGCTTGTCAACTTCCA (SEQ ID | | |
| | | NO: 239) | | |
| Myostatin-TN12- | AHWCEQGPWRYGNIC | GCTCATTGGTGTGAACAAGGTCCATGGCG | 5 gly | N |
| 34 | MAY | TTATGGTAATATTTGTATGGCTTAT (SEQ | | C |
| | | ID NO: 240) | | |
| Myostatin-TN12- | NLVCGKISAWGDEACA | AATCTTGTTTGTGGTAAAATTTCTGCTTGG | 5 gly | N |
| 35 | RA | GGTGATGAAGCTTGTGCTCGTGCT (SEQ ID | | |
| | | NO: 241) | | |
| Myostatin-TN12- | HNVCTIMGPSMKWFC | CATAATGTTTGTACTATTATGGGTCCATCT | 5 gly | N |
| 36 | WND | ATGAAATGGTTTTGTTGGAATGAT | | C |
| | | (SEQ ID NO: 242) | | |
| Myostatin-TN12- | NDLCAMWGWRNTIWC | AATGATCTTTGTGCTATGTGGGGTTGGCGT | 5 gly | N |
| 37 | QNS | AATACTATTTGGTGTCAAAATTCT (SEQ ID | | C |
| | | NO: 243) | | |
| Myostatin-TN12- | PPFCQNDNDMLQSLCK | CCACCATTTTGTCAAAATGATAATGATATG | 5 gly | N |
| 38 | LL | CTTCAATCTCTTTGTAAACTTCTT (SEQ ID | | |
| | | NO: 244) | | |
| Myostatin-TN12- | WYDCNVPNELLSGLCR | TGGTATGATTGTAATGTTCCAAATGAACTT | 5 gly | N |
| 39 | LF | CTTTCTGGTCTTTGTCGTCTTTTT (SEQ ID | | |
| | | NO: 245) | | |
| Myostatin-TN12- | YGDCDQNHWMWPFTC | TATGGTGATTGTGATCAAAATCATTGGATG | 5 gly | N |
| 40 | LSL | TGGCCATTTACTTGTCTTTCTCTT (SEQ ID | | C |
| | | NO: 246) | | |
| Myostatin-TN12- | GWMCHFDLHDWGAT | GGTTGGATGTGTCATTTTGATCTTCATGAT | 5 gly | N |
| 41 | CQPD | TGGGGTGCTACTTGTCAACCAGAT (SEQ ID | | |
| | | NO: 247) | | |
| Myostatin-TN12- | YFHCMFGGHEFEVHCE | TATTTTCATTGTATGTTTGGTGGTCATGAA | 5 gly | N |
| 42 | SF | TTTGAAGTTCATTGTGAATCTTTT (SEQ ID | | C |
| | | NO: 248) | | |
| Myostatin-TN12- | AYWCWHGQCVRF | GCTTATTGGTGTTGGCATGGTCAATGTGTT | 5 gly | N |
| 43 | | CGTTTT (SEQ ID NO: 249) | | |
| Myostatin-Linear- | SEHWTFTDWDGNEW | TCCGAACACTGGACCTTCACCGACTGGGA | 5 gly | N |
| 1 | WVRPF | CGGTAACGAATGGTGGGTTCGTCCGTTC | | |
| | | (SEQ ID NO: 250) | | |
| Myostatin-Linear- | MEMLDSLFELLKDMVP | ATGGAAATGCTGGACTCCCTGTTCGAACTG | 5 gly | N |
| 2 | ISKA | CTGAAAGACATGGTTCCGATCTCCAAAGC | | |
| | | T (SEQ ID NO: 251) | | |
| Myostatin-Linear- | SPPEEALMEWLGWQY | TCCCCGCCGGAAGAAGCTCTGATGGAATG | 5 gly | N |
| 3 | GKFT | GCTGGGTTGGCAGTACGGTAAATTCACC | | |
| | | (SEQ ID NO: 252) | | |
| Myostatin-Linear- | SPENLLNDLYILMTKQ | TCCCCGGAAAACCTGCTGAACGACCTGTA | 5 gly | N |
| 4 | EWYG | CATCCTGATGACCAAACAGGAATGGTACG | | |
| | | GT (SEQ ID NO: 253) | | |
| Myostatin-Linear- | FHWEEGIPFHVVTPYS | TTCCACTGGGAAGAAGGTATCCCGTTCCAC | 5 gly | N |
| 5 | YDRM | GTTGTTACCCCGTACTCCTACGACCGTATG | | |
| | | (SEQ ID NO: 254) | | |
| Myostatin-Linear- | KRLLEQFMNDLAELVS | AAACGTCTGCTGGAACAGTTCATGAACGA | 5 gly | N |
| 6 | GHS | CCTGGCTGAACTGGTTTCCGGTCACTCC | | |
| | | (SEQ ID NO: 255) | | |
| Myostatin-Linear- | DTRDALFQEFYEFVRS | GACACCCGTGACGCTCTGTTCCAGGAATTC | 5 gly | N |
| 7 | RLVI | TACGAATTCGTTCGTTCCCGTCTGGTTATC | | |
| | | (SEQ ID NO: 256) | | |
| Myostatin-Linear- | RMSAAPRPLTYRDIMD | CGTATGTCCGCTGCTCCGCGTCCGCTGACC | 5 gly | N |
| 8 | QYWH | TACCGTGACATCATGGACCAGTACTGGCA | | |
| | | C (SEQ ID NO: 257) | | |
| Myostatin-Linear- | NDKAHFFEMFMFDVH | AACGACAAAGCTCACTTCTTCGAAATGTTC | 5 gly | N |
| 9 | NFVES | ATGTTCGACGTTCACAACTTCGTTGAATCC | | |
| | | (Seq Id No: 258) | | |
| Myostatin-Linear- | QTQAQKIDGLWELLQS | CAGACCCAGGCTCAGAAAATCGACGGTCT | 5 gly | N |
| 10 | IRNQ | GTGGGAACTGCTGCAGTCCATCCGTAACC | | |
| | | AG (SEQ ID NO: 259) | | |
| Myostatin-Linear- | MLSEFEEFLGNLVHRQ | ATGCTGTCCGAATTCGAAGAATTCCTGGGT | 5 gly | N |
| 11 | EA | AACCTGGTTCACCGTCAGGAAGCT (SEQ | | |
| | | ID NO: 260) | | |
| Myostatin-Linear- | YTPKMGSEWTSFWHN | TACACCCCGAAAATGGGTTCCGAATGGAC | 5 gly | N |
| 12 | RIHYL | CTCCTTCTGGCACAACCGTATCCACTACCT | | |
| | | G (SEQ ID NO: 261) | | |
| Myostatin-Linear- | LNDTLLRELKMVLNSL | CTGAACGACACCCTGCTGCGTGAACTGAA | 5 gly | N |
| 13 | SDMK | AATGGTTCTGAACTCCCTGTCCGACATGAA | | |
| | | A (SEQ ID NO: 262) | | |
| Myostatin-Linear- | FDVERDLMRWLEGFM | TTCGACGTTGAACGTGACCTGATGCGTTGG | 5 gly | N |
| 14 | QSAAT | CTGGAAGGTTTCATGCAGTCCGCTGCTACC | | |
| | | (SEQ ID NO: 263) | | |
| Myostatin-Linear- | HHGWNYLRKGSAPQW | CACCACGGTTGGAACTACCTGCGTAAAGG | 5 gly | N |
| 15 | FEAWV | TTCCGCTCCGCAGTGGTTCGAAGCTTGGGT | | |
| | | T (SEQ ID NO: 264) | | |
| Myostatin-Linear- | VESLHQLQMWLDQKL | GTTGAATCCCTGCACCAGCTGCAGATGTG | 5 gly | N |
| 16 | ASGPH | GCTGGACCAGAAACTGGCTTCCGGTCCGC | | |
| | | AC (SEQ ID NO: 265) | | |
| Myostatin-Linear- | RATLLKDFWQLVEGY | CGTGCTACCCTGCTGAAAGACTTCTGGCAG | 5 gly | N |
| 17 | GDN | CTGGTTGAAGGTTACGGTGACAAC (SEQ | | |
| | | ID NO: 266) | | |
| Myostatin-Linear- | EELLREFYRFVSAFDY | GAAGAACTGCTGCGTGAATTCTACCGTTTC | 5 gly | N |
| 18 | | GTTTCCGCTTTCGACTAC (SEQ ID NO: 267) | | |
| Myostatin-Linear- | GLLDEFSHFIAEQFYQ | GGTCTGCTGGACGAATTCTCCCACTTCATC | 5 gly | N |
| 19 | MPGG | GCTGAACAGTTCTACCAGATGCCGGGTGG | | |
| | | T (SEQ ID NO: 268) | | |
| Myostatin-Linear- | YREMSMLEGLLDVLER | TACCGTGAAATGTCCATGCTGGAAGGTCT | 5 gly | N |
| 20 | LQHY | GCTGGACGTTCTGGAACGTCTGCAGCACT | | |
| | | AC (SEQ ID NO: 269) | | |
| Myostatin-Linear- | HNSSQMLLSELIMLVG | CACAACTCCTCCCAGATGCTGCTGTCCGAA | 5 gly | N |
| 21 | SMMQ | CTGATCATGCTGGTTGGTTCCATGATGCAG | | |
| | | (SEQ ID NO: 270) | | |
| Myostatin-Linear- | WREHFLNSDYIRDKLI | TGGCGTGAACACTTCCTGAACTCCGACTAC | 5 gly | N |
| 22 | AIDG | ATCCGTGACAAACTGATCGCTATCGACGG | | |
| | | T (SEQ ID NO: 271) | | |
| Myostatin-Linear- | QFPFYVFDDLPAQLEY | CAGTTCCCGTTCTACGTTTTCGACGACCTG | 5 gly | N |
| 23 | WIA | CCGGCTCAGCTGGAATACTGGATCGCT | | |
| | | (SEQ ID NO: 272) | | |
| Myostatin-Linear- | EFFHWLHNHRSEVNH | GAATTCTTCCACTGGCTGCACAACCACCGT | 5 gly | N |
| 24 | WLDMN | TCCGAAGTTAACCACTGGCTGGACATGAA | | |
| | | C (SEQ ID NO: 273) | | |
| Myostatin-Linear- | EALFQNFFRDVLTLSER | GAAGCTCTTTTTCAAAATTTTTTTCGTGAT | 5 gly | N |
| 25 | EY | GTTCTTACTCTTTCTGAACGTGAATAT | | C |
| | | (SEQ ID NO: 274) | | |
| Myostatin-Linear- | QYWEQQWMTYFRENG | CAATATTGGGAACAACAATGGATGACTTA | 5 gly | N |
| 26 | LHVQY | TTTTCGTGAAAATGGTCTTCATGTTCAATA | | |
| | | T (SEQ ID NO: 275) | | |
| Myostatin-Linear- | NQRMMLEDLWRIMTP | AATCAACGTATGATGCTTGAAGATCTTTGG | 5 gly | N |
| 27 | MFGRS | CGTATTATGACTCCAATGTTTGGTCGTTCT | | C |
| | | (SEQ ID NO: 276) | | |
| Myostatin-Linear- | FLDELKAELSRHYALD | TTTCTTGATGAACTTAAAGCTGAACTTTCT | 5 gly | N |
| 29 | DLDE | CGTCATTATGCTCTTGATGATCTTGATGAA | | |
| | | (SEQ ID NO: 277) | | |
| Myostatin-Linear- | GKLIEGLLNELMQLETF | GGTAAACTTATTGAAGGTCTTCTTAATGAA | 5 gly | N |
| 30 | MPD | CTTATGCAACTTGAAACTTTTATGCCAGAT | | C |
| | | (SEQ ID NO: 278) | | |
| Myostatin-Linear- | ILLLDEYKKDWKSWF | ATTCTTCTTCTTGATGAATATAAAAAAGAT | 5 gly | N |
| 31 | | TGGAAATCTTGGTTT (SEQ ID NO: 279) | | |
| Myostatin- | QGHCTRWPWMCPPYG | CAGGGCCACTGTACTCGCTGGCCGTGGAT | 1k | N |
| 2XTN8-19 kc | SGSATGGSGSTASSGSG | GTGCCCGCCGTACGGTTCTGGTTCCGCTAC | | |
| | SATGQGHCTRWPWMC | CGGTGGTTCTGGTTCCACTGCTTCTTCTGG | | |
| | PPY | TTCCGGTTCTGCTACTGGTCAGGGTCACTG | | |
| | | CACTCGTTGGCCATGGATGTGTCCACCGTA | | |
| | | T (SEQ ID NO: 280) | | |
| Myostatin- | WYPCYEGHFWCYDLG | TGGTATCCGTGTTATGAGGGTCACTTCTGG | 5 gly | C |
| 2XTN8-CON6 | SGSTASSGSGSATGWY | TGCTACGATCTGGGTTCTGGTTCCACTGCT | | |
| | PCYEGHFWCYDL | TCTTCTGGTTCCGGTTCCGCTACTGGTTGG | | |
| | | TACCCGTGCTACGAAGGTCACTTTTGGTGT | | |
| | | TATGATCTG (SEQ ID NO: 281) | | |
| Myostatin- | HTPCPWFAPLCVEWGS | CACACTCCGTGTCCGTGGTTTGCTCCGCTG | 1k | C |
| 2XTN8-5 kc | GSATGGSGSTASSGSGS | TGCGTTGAATGGGGTTCTGGTTCCGCTACT | | |
| | ATGHTPCPWFAPLCVE | GGTGGTTCCGGTTCCACTGCTTCTTCTGGT | | |
| | W | TCCGGTTCTGCAACTGGTCACACCCCGTGC | | |
| | | CCGTGGTTTGCACCGCTGTGTGTAGAGTGG | | |
| | | (SEQ ID NO: 282) | | |
| Myostatin- | PDWCIDPDWWCKFWG | CCGGATTGGTGTATCGACCCGGACTGGTG | 1k | C |
| 2XTN8-18 kc | SGSATGGSGSTASSGSG | GTGCAAATTCTGGGGTTCTGGTTCCGCTAC | | |
| | SATGPDWCIDPDWWC | CGGTGGTTCCGGTTCCACTGCTTCTTCTGG | | |
| | KFW | TTCCGGTTCTGCAACTGGTCCGGACTGGTG | | |
| | | CATCGACCCGGATTGGTGGTGTAAATTTTG | | |
| | | G (SEQ ID NO: 283) | | |
| Myostatin- | ANWCVSPNWFCMVM | CCGGATTGGTGTATCGACCCGGACTGGTG | 1k | C |
| 2XTN8-11 kc | GSGSATGGSGSTASSGS | GTGCAAATTCTGGGGTTCTGGTTCCGCTAC | | |
| | GSATGANWCVSPNWF | CGGTGGTTCCGGTTCCACTGCTTCTTCTGG | | |
| | CMVM | TTCCGGTTCTGCAACTGGTCCGGACTGGTG | | |
| | | CATCGACCCGGATTGGTGGTGTAAATTTTG | | |
| | | G (SEQ ID NO; 284) | | |
| Myostatin- | PDWCIDPDWWCKFWG | ACCACTTGGTGCATCTCTCCGATGTGGTTC | 1k | C |
| 2XTN8-25 kc | SGSATGGSGSTASSGSG | TGCTCTCAGCAGGGTTCTGGTTCCACTGCT | | |
| | SATGPDWCIDPDWWC | TCTTCTGGTTCCGGTTCTGCAACTGGTACT | | |
| | KFW | ACTTGGTGTATCTCTCCAATGTGGTTTTGT | | |
| | | TCTCAGCAA (SEQ ID NO: 285) | | |
| Myostatin- | HWACGYWPWSCKWV | CACTGGGCATGTGGCTATTGGCCGTGGTCC | 1k | C |
| 2XTN8-23 kc | GSGSATGGSGSTASSGS | TGCAAATGGGTTGGTTCTGGTTCCGCTACC | | |
| | GSATGHWACGYWPWS | GGTGGTTCCGGTTCCACTGCTTCTTCTGGT | | |
| | CKWV | TCCGGTTCTGCAACTGGTCACTGGGCTTGC | | |
| | | GGTTACTGGCCGTGGTCTTGTAAATGGGTT | | |
| | | (SEQ ID NO: 286) | | |
| Myostatin-TN8- | KKHCQIWTWMCAPKG | AAAAAACACTGTCAGATCTGGACTTGGAT | 1k | C |
| 29-19 kc | SGSATGGSGSTASSGSG | GTGCGCTCCGAAAGGTTCTGGTTCCGCTAC | | |
| | SATGQGHCTRWPWMC | CGGTGGTTCTGGTTCCACTGCTTCTTCTGG | | |
| | PPY | TTCCGGTTCCGCTACTGGTCAGGGTCACTG | | |
| | | CACTCGTTGGCCATGGATGTGTCCGCCGTA | | |
| | | T (SEQ ID NO: 287) | | |
| Myostatin-TN8- | QGHCTRWPWMCPPYG | CAGGGTCACTGCACCCGTTGGCCGTGGAT | 1k | C |
| 19-29 kc | SGSATGGSGSTASSGSG | GTGCCCGCCGTACGGTTCTGGTTCCGCTAC | | |
| | SATGKKHCQIWTWMC | CGGTGGTTCTGGTTCCACTGCTTCTTCTGG | | |
| | APK | TTCCGGTTCTGCTACTGGTAAAAAACACTG | | |
| | | CCAGATCTGGACTTGGATGTGCGCTCCGA | | |
| | | AA (SEQ ID NO: 288) | | |
| Myostatin-TN8- | KKHCQIWTWMCAPKG | AAAAAACACTGTCAGATCTGGACTTGGAT | 1k | N |
| 29-19 kn | SGSATGGSGSTASSGSG | GTGCGCTCCGAAAGGTTCTGGTTCCGCTAC | | |
| | SATGQGHCTRWPWMC | CGGTGGTTCTGGTTCCACTGCTTCTTCTGG | | |
| | PPY | TTCCGGTTCCGCTACTGGTCAGGGTCACTG | | |
| | | CACTCGTTGGCCATGGATGTGTCCGCCGTA | | |
| | | T (SEQ ID NO: 289) | | |
| Myostatin-TN8- | KKHCQIWTWMCAPKG | AAAAAACACTGCCAGATCTGGACTTGGAT | 8 gly | C |
| 29-19-8g | GGGGGGGQGHCTRWP | GTGCGCTCCGAAAGGTGGTGGTGGTGGTG | | |
| | WMCPPY | GCGGTGGCCAGGGTCACTGCACCCGTTGG | | |
| | | CCGTGGATGTGTCCGCCGTAT (SEQ ID NO: | | |
| | | 290) | | |
| Myostatin-TN8- | QGHCTRWPWMCPPYG | CAGGGTCACTGCACCCGTTGGCCGTGGAT | 6 gly | C |
| 19-29-6gc | GGGGGKKHCQIWTWM | GTGCCCGCCGTACGGTGGTGGTGGTGGTG | | |
| | CAPK | GCAAAAAACACTGCCAGATCTGGACTTGG | | |
| | | ATGTGCGCTCCGAAA (SEQ ID NO: 291) | | |

### Example 3

### In Vitro Assays

C2C12 Cell Based Myostatin Activity Assay

This assay demonstrates the myostatin neutralizing capability of the inhibitor being tested by measuring the extent that binding of myostatin to its receptor is inhibited.

A myostatin-responsive reporter cell line was generated by transfection of C2C12 myoblast cells (ATCC No: CRL-1772) with a pMARE-luc construct. The pMARE-luc construct was made by cloning twelve repeats of the CAGA sequence, representing the myostatin/activin response elements (Dennler et al. EMBO 17: 3091-3100 (1998)) into a pLuc-MCS reporter vector (Stratagene cat #219087) upstream of the TATA box. The myoblast C2C12 cells naturally express myostatin/activin receptors on its cell surface. When myostatin binds the cell receptors, the Smad pathway is activated, and phosphorylated Smad binds to the response element (Macias-Silva et al. Cell 87:1215 (1996)), resulting in the expression of the lucerase gene. Luciferase activity is then measured using a commercial luciferase reporter assay kit (cat # E4550, Promega, Madison, Wis.) according to manufacturer's protocol. A stable line of C2C12 cells that had been transfected with pMARE-luc (C2C12/pMARE clone #44) was used to measure myostatin activity according to the following procedure.

Equal numbers of the reporter cells (C2C12/pMARE clone #44) were plated into 96 well cultures. A first round screening using two dilutions of peptibodies was performed with the myostatin concentration fixed at 4 nM. Recombinant mature myostatin was pre-incubated for 2 hours at room temperature with peptibodies at 40 nM and 400 nM respectively. The reporter cell culture was treated with the myostatin with or without peptibodies for six hours. Myostatin activity was measured by determining the luciferase activity in the treated cultures. This assay was used to initially identify peptibody hits that inhibited the myostatin signaling activity in the reporter assay. Subsequently, a nine point titration curve was generated with the myostatin concentration fixed at 4 nM. The myostatin was preincubated with each of the following nine concentrations of peptibodies: 0.04 mM, 0.4 nM, 4 nM, 20 nM, 40 nM, 200 nM, 400 nM, 2 uM and 4 uM for two hours before adding the mixture to the reporter cell culture. The IC₅₀ values were for a number of examplary peptibodies are provided in Tables III and for affinity matured peptibodies, in Table VIII.

BIAcore® Assay

An affinity analysis of each candidate myostatin peptibody was performed on a BIAcore®3000 (Biacore, Inc., Piscataway, N.J.), apparatus using sensor chip CM5, and 0.005 percent P20 surfactant (Biacore, Inc.) as running buffer. Recombinant mature myostatin protein was immobilized to a research grade CM5 sensor chip (Biacore, Inc.) via primary amine groups using the Amine Coupling Kit (Biacore, Inc.) according to the manufacturer's suggested protocol.

Direct binding assays were used to screen and rank the peptibodies in order of their ability to bind to immobilized myostatin. Binding assays were carried by injection of two concentrations (40 and 400 nM) of each candidate myostatin-binding peptibody to the immobilized myostatin surface at a flow rate of 50 μl/min for 3 minutes. After a dissociation time of 3 minutes, the surface was regenerated. Binding curves were compared qualitatively for binding signal intensity, as well as for dissociation rates. Peptibody binding kinetic parameters including kₐ (association rate constant), k_{d} (dissociation rate constant) and K_{D} (dissociation equilibrium constant) were determined using the BIA evaluation 3.1 computer program (Biacore, Inc.). The lower the dissociation equilibrium constants (expressed in nM), the greater the affinity of the peptibody for myostatin. Examples of peptibody K_{D} values are shown in Table III and Table VI for affinity-matured peptibodies below.

Blocking Assay on ActRIIB/Fc Surface

Blocking assays were carried out using immobilized ActRIIB/Fc (R&D Systems, Minneapolis, Minn.) and myostatin in the presence and absence of peptibodies with the BIAcore® assay system. Assays were used to classify peptibodies as non-neutralizing (those which did not prevent myostatin binding to ActRIIB/Fc) or neutralizing (those that prevented myostatin binding to ActRIIB/Fc). Baseline myostatin-ActRIIB/Fc binding was first determined in the absence of any peptibody.

For early screening studies, peptibodies were diluted to 4 nM, 40 nM, and 400 nM in sample buffer and incubated with 4 nM myostatin (also diluted in sample buffer). The peptibody: ligand mixtures were allowed to reach equilibrium at room temperature (at least 5 hours) and then were injected over the immobilized ActRIIB/Fc surface for 20 to 30 minutes at a flow rate of 10 uL/min. An increased binding response (over control binding with no peptibody) indicated that peptibody binding to myostatin was non-neutralizing. A decreased binding response (compared to the control) indicated that peptibody binding to myostatin blocked the binding of myostatin to ActRIIB/Fc. Selected peptibodies were further characterized using the blocking assay of a full concentration series in order to derive IC₅₀ values (for neutralizing peptibodies) or EC₅₀ (for non-neutralizing peptibodies). The peptibody samples were serially diluted from 200 nM to 0.05 mM in sample buffer and incubated with 4 mM myostatin at room temperature to reach equilibrium (minimum of five hours) before injected over the immobilized ActRIIB/Fc surface for 20 to 30 minutes at a flow rate of 10 uL/min. Following the sample injection, bound ligand was allowed to dissociate from the receptor for 3 minutes. Plotting the binding signal yrs. peptibody concentration, the IC₅₀ values for each peptibody in the presence of 4 nM myostatin were calculated. It was found, for example, that the peptibodies TN8-19, L2 and L17 inhibit myostatin activity in cell-based assay, but binding of TN-8-19 does not block myostatin/ActRIIB/Fc interactions, indicating that TN-8-19 binds to a different epitope than that observed for the other two peptibodies.

Epitope Binding for Peptibodies

A purified peptibody was immobilized on a BIAcore chip to capture myostatin before injection of a second peptibody, and the amount of secondary peptibody bound to the captured myostatin was determined Only peptibodies with distinct epitopes will bind to the captured myostatin, thus enabling the binding of peptibodies with similar or distinct epitope binding properties. For example, it was shown that peptibodies TN8-19 and L23 bind to different epitopes on myostatin.

Selectivity Assays

These assays were performed using BIAcore® technology, to determine the selectivity of binding of the peptibodies to other TGFβ family members. ActRIIB/Fc, TGFβRII/Fc and BMPR-1A/Fc (all obtained from R & D Systems, Minneapolis, Minn.) were covalently coupled to research grade sensor chips according to manufacturer's suggested protocol. Because BIAcore assays detects changes in the refractive index, the difference between the response detected with injection over the immobilized receptor surfaces compared with the response detected with injection over the control surface in the absence of any peptibody represents the actual binding of Activin A, TGFβ1, TGFδ3, and BMP4 to the receptors, respectively. With pre-incubation of peptibodies and TGFβ molecules, a change (increase or decrease) in binding response indicates peptibody binding to the TGFβ family of molecules. The peptibodies of the present invention all bind to myostatin but not to Activin A, TGFβ1, TGFβ3, or BMP4.

KinEx A™ Equilibrium Assays

Solution-based equilibrium-binding assays using KinExA™ technology (Sapidyne Instruments, Inc.) were used to determine the dissociation equilibrium (K_{D}) of myostatin binding to peptibody molecules. This solution-based assay is considered to be more sensitive than the BIAcore assay in some instances. Reacti-Gel™ 6X was pre-coated with about 50 ug/ml myostatin for over-night, and then blocked with BSA. 30 pM and 100 pM of peptibody samples were incubated with various concentrations (0.5 pM to 5 nM) of myostatin in sample buffer at room temperature for 8 hours before being run through the myostatin-coated beads. The amount of the bead-bound peptibody was quantified by fluorescent (Cy5) labeled goat anti-human-Fc antibody at 1 mg/ml in superblock. The binding signal is proportional to the concentration of free peptibody at equilibrium with a given myostatin concentration. K_{D} was obtained from the nonlinear regression of the competition curves using a dual-curve one-site homogeneous binding model provided in the KinEx ATM software (Sapidyne Instruments, Inc.).

### Example 4

### Comparison of Myostatin Inhibitors

The ability of three exemplary first-round peptibodies to bind to (K_{D}) and inhibit (IC₅₀) were compared with the K_{D} and IC₅₀ values obtained for the soluble receptor fusion protein actRIIB/Fc (R &D Systems, Inc., Minneapolis, Minn.). The IC₅₀ values were determined using the pMARE luc cell-based assay described in Example 3 and the K_{D} values were determined using the Biacore® assay described in Example 3.

TABLE III
K_{D} and IC₅₀ values

| Inhibitor | IC50 (nM) | KD (nM) |
|---|---|---|
| ActRIIB/Fc | ~83 | ~7 |
| 2xTN8-19-kc | ~9 | ~2 |
| TN8-19 | ~23 | ~2 |
| TN8-29 | ~26 | ~60 |
| TN12-34 | ~30 | — |
| Linear-20 | ~11 | — |

The peptibodies have an IC₅₀ that is improved over the receptor/Fc inhibitor and binding affinities which are comparable in two peptibodies with the receptor/Fc.

### Example 5

### Comparison of Ability of Peptide and Peptibody to Inhibit Myostatin

The following peptide sequence: QGHCTRWPWMCPPY (TN8-19) (SEQ ID NO: 33) was used to construct the corresponding peptibody TN8-19(pb) according to the procedure described in Example 2 above. Both the peptide alone and the peptibody were screened for myostatin inhibiting activity using the C2C12 based assay described in Example 3 above. It can be seen from FIG. 1 the IC₅₀ (effective concentration for fifty percent inhibition of myostatin) for the peptibody is significantly less than that of the peptide, and thus the ability of the peptide to inhibit myostatin activity has been substantially improved by placing it in the peptibody configuration.

### Example 6

### Generation of Affinity-Matured Peptides and Peptibodies

Several of the first round peptides used for peptibody generation were chosen for affinity maturation. The selected peptides included the following:

the cysteine constrained TN8-19,
(SEQ ID NO: 33)
QGHCTRWPWMCPPY,
and the linear peptides Linear-2
(SEQ ID NO: 104)
MEMLDSLFELLKDMVPISKA;
Linear-15
(SEQ. ID NO: 117)
HHGWNYLRKGSAPQWFEAWV;
Linear-17
(SEQ ID NO: 119)
RATLLKDFWQLVEGYGDN;
Linear-20
(SEQ ID NO: 122)
YREMSMLEGLLDVLERLQHY,
Linear-21
(SEQ ID NO: 123)
HNSSQMLLSELIMLVGSMMQ,
Linear-24
(SEQ ID NO: 126)
EFFHWLHNHRSEVNHWLDMN.

Based on a consensus sequence, directed secondary phage display libraries were generated in which the “core” amino acids (determined from the consensus sequence) were either held constant or biased in frequency of occurrence. Alternatively, an individual peptide sequence could be used to generate a biased, directed phage display library. Panning of such libraries under more stringent conditions can yield peptides with enhanced binding to myostatin, selective binding to myostatin, or with some additional desired property.

Production of Doped Oligos for Libraries

Oligonucleotides were synthesized in a DNA synthesizer which were 91% “doped” at the core sequences, that is, each solution was 91% of the represented base (A, G, C, or T), and 3% of each of the other 3 nucleotides. For the TN8-19 family, for example, a 91% doped oligo used for the construction of a secondary phage library was the following:

(SEQ ID NO: 634)
5′-CAC AGT GCA CAG GGT NNK NNK NNK caK ggK caK tgK
acK cgK tgK ccK tgK atK tgK ccK ccK taK NNK NNK
NNK CAT TCT CTC GAG ATC A-3′

wherein “N” indicates that each of the four nucleotides A, T, C, and G were equally represented, K indicates that G and T were equally represented, and the lower case letter represents a mixture of 91% of the indicated base and 3% of each of the other bases. The family of oligonucleotides prepared in this manner were PCR amplified as described above, ligated into a phagemid vectors, for example, a modified pCES1 plasmid (Dyax), or any available phagemid vector according to the protocol described above. The secondary phage libraries generated were all 91% doped and had between 1 and 6.5×10⁹ independent transformants. The libraries were panned as described above, but with the following conditions:

### Round 1 Panning:

Input phage number: 10¹²-10¹³ cfu of phagemid
Selection method: Nunc Immuno Tube selection
Negative selection: 2× with Nunc Immuno Tubes coated with 2% BSA at 10 min. each
Panning coating: Coat with 1 μg of Myostatin protein in 1 ml of 0.1M Sodium carbonate buffer (pH 9.6)
Binding time: 3 hours
Washing conditions: 6×2%-Milk-PBST; 6×PBST; 2×PBS
Elution condition: 100 mM TEA elution

### Round 2 Panning:

Input phage number: 10¹¹ cfu of phagemid
Selection method: Nunc Immuno Tube selection
Negative selection: 2× with Nunc Immuno Tubes coated with 2% BSA at 30 min. each
Panning coating: Coat with 1 μg of Myostatin protein in 1 ml of 0.1M Sodium carbonate buffer (pH 9.6)
Binding time: 1 hour
Washing conditions: 15×2%-Milk-PBST, 1×2%-Milk-PBST for 1 hr., 10×2%-BSA-PBST,
   1×2%-BSA-PBST for 1 hr., 10×PBST and 3×PBS
Elution condition: 100 mM TEA elution

### Round 3 Panning:

Input phage number: 10¹⁰ cfu of phagemid
Selection method: Nunc Immuno Tube selection
Negative selection: 6× with Nunc Immuno Tubes coated with 2% BSA at 10 min. each
Panning coating: Coat with 0.1 pg of Myostatin protein in 1 ml of 0.1M Sodium carbonate buffer (pH 9.6)
Binding time: 1 hour
Washing conditions: 15×2%-Milk-PBST, 1×2%-Milk-PBST for 1 hr., 10×2%-BSA-PBST,
   1×2%-BSA-PBST for 1 hr., 10×PBST and 3×PBS
Elution condition: 100 mM TEA elution

Panning of the secondary libraries yielded peptides with enhanced binding to myostatin. Individual selected clones were subjected phage ELISA, as described above, and sequenced.

The following affinity matured TN8-19 family of peptides are shown in Table IV below.

TABLE IV
affinity matured TN8-19 family of peptides

| Affinity-matured | SEQ ID | |
|---|---|---|
| peptibody | NO: | Peptide sequence |
| mTN8-19-1 | 305 | VALHGQCTRWPWMCPPQREG |
| mTN8-19-2 | 306 | YPEQGLCTRWPWMCPPQTLA |
| mTN8-19-3 | 307 | GLNQGHCTRWPWMCPPQDSN |
| mTN8-19-4 | 308 | MITQGQCTRWPWMCPPQPSG |
| mTN8-19-5 | 309 | AGAQEHCTRWPWMCAPNDWI |
| mTN8-19-6 | 310 | GVNQGQCTRWRWMCPPNGWE |
| mTN8-19-7 | 311 | LADHGQCIRWPWMCPPEGWE |
| mTN8-19-8 | 312 | ILEQAQCTRWPWMCPPQRGG |
| mTN8-19-9 | 313 | TQTHAQCTRWPWMCPPQWEG |
| mTN8-19-10 | 314 | VVTQGHCTLWPWMCPPQRWR |
| mTN8-19-11 | 315 | IYPHDQCTRWPWMCPPQPYP |
| mTN8-19-12 | 316 | SYWQGQCTRWPWMCPPQWRG |
| mTN8-19-13 | 317 | MWQQGHCTRWPWMCPPQGWG |
| mTN8-19-14 | 318 | EFTQWHCTRWPWMCPPQRSQ |
| mTN8-19-15 | 319 | LDDQWQCTRWPWMCPPQGFS |
| mTN8-19-16 | 320 | YQTQGLCTRWPWMCPPQSQR |
| mTN8-19-17 | 321 | ESNQGQCTRWPWMCPPQGGW |
| mTN8-19-18 | 322 | WTDRGPCTRWPWMCPPQANG |
| mTN8-19-19 | 323 | VGTQGQCTRWPWMCPPYETG |
| mTN8-19-20 | 324 | PYEQGKCTRWPWMCPPYEVE |
| mTN8-19-21 | 325 | SEYQGLCTRWPWMCPPQGWK |
| mTN8-19-22 | 326 | TFSQGHCTRWPWMCPPQGWG |
| mTN8-19-23 | 327 | PGAHDHCTRWPWMCPPQSRY |
| mTN8-19-24 | 328 | VAEEWHCRRWPWMCPPQDWR |
| mTN8-19-25 | 329 | VGTQGHCTRWPWMCPPQPAG |
| mTN8-19-26 | 330 | EEDQAHCRSWPWMCPPQGWV |
| mTN8-19-27 | 331 | ADTQGHCTRWPWMCPPQHWF |
| mTN8-19-28 | 332 | SGPQGHCTRWPWMCAPQGWF |
| mTN8-19-29 | 333 | TLVQGHCTRWPWMCPPQRWV |
| mTN8-19-30 | 334 | GMAHGKCTRWAWMCPPQSWK |
| mTN8-19-31 | 335 | ELYHGQCTRWPWMCPPQSWA |
| mTN8-19-32 | 336 | VADHGHCTRWPWMCPPQGWG |
| mTN8-19-33 | 337 | PESQGHCTRWPWMCPPQGWG |
| mTN8-19-34 | 338 | IPAHGHCTRWPWMCPPQRWR |
| mTN8-19-35 | 339 | FTVHGHCTRWPWMCPPYGWV |
| mTN8-19-36 | 340 | PDFPGHCTRWRWMCPPQGWE |
| mTN8-19-37 | 341 | QLWQGPCTQWPWMCPPKGRY |
| mTN8-19-38 | 342 | HANDGHCTRWQWMCPPQWGG |
| mTN8-19-39 | 343 | ETDHGLCTRWPWMCPPYGAR |
| mTN8-19-40 | 344 | GTWQGLCTRWPWMCPPQGWQ |
| mTN8-19 con1 | 345 | VATQGQCTRWPWMCPPQGWG |
| mTN8-19 con2 | 346 | VATQGQCTRWPWMCPPQRWG |
| mTN8 con6-1 | 347 | QREWYPCYGGHLWCYDLHKA |
| mTN8 con6-2 | 348 | ISAWYSCYAGHFWCWDLKQK |
| mTN8 con6-3 | 349 | WTGWYQCYGGHLWCYDLRRK |
| mTN8 con6-4 | 350 | KTFWYPCYDGHFWCYNLKSS |
| mTN8 con6-5 | 351 | ESRWYPCYEGHLWCFDLTET |

The consensus sequence derived from the affinity-matured TN-8-19-1 through Con2 (excluding the mTN8 con6 sequences) shown above is: Ca₁a₂Wa₃WMCPP (SEQ ID NO: 352). All of these peptide comprise the sequence WMCPP (SEQ ID NO: 633). The underlined amino acids represent the core amino acids present in all embodiments, and a₁, a₂ and a₃ are selected from a neutral hydrophobic, neutral polar, or basic amino acid. In one embodiment of this consensus sequence, Cb₁b₂Wb₃WMCPP (SEQ ID NO: 353), b₁ is selected from any one of the amino acids T, I, or R; b₂ is selected from any one of R, S, Q; and b₃ is selected from any one of P, R and Q. All of the peptides comprise the sequence WMCPP (SEQ ID NO: 633). A more detailed analysis of the affinity matured TN8 sequences comprising SEQ ID NO: 352 provides the following formula:

(SEQ ID NO: 354),
c₁c₂c₃c₄c₅c₆Cc₇c₈Wc₉WMCPPc₁₀c₁₁c₁₂c₁₃

wherein:

c₁ is absent or any amino acid;

c₂ is absent or a neutral hydrophobic, neutral polar, or acidic amino acid;

c₃ is absent or a neutral hydrophobic, neutral polar, or acidic amino acid;

c₄ is absent or any amino acid;

c₅ is absent or a neutral hydrophobic, neutral polar, or acidic amino acid;

c₆ is absent or a neutral hydrophobic, neutral polar, or basic amino acid;

c₇ is a neutral hydrophobic, neutral polar, or basic amino acid;

c₈ is a neutral hydrophobic, neutral polar, or basic amino acid;

c₉ is a neutral hydrophobic, neutral polar or basic amino acid; and wherein

c₁₀ to c₁₃ is any amino acid.

In one embodiment of the above formulation, b₇ is selected from any one of the amino acids T, I, or R; b₈ is selected from any one of R, S, Q; and b₉ is selected from any one of P, R and Q. This provides the following sequence:

(SEQ ID NO: 355).
d₁d₂d₃d₄d₅d₆Cd₇d₈Wd₉WMCPP d₁₀d₁₁d₁₂d₁₃

d₁ is absent or any amino acid;

d₂ is absent or a neutral hydrophobic, neutral polar, or acidic amino acid;

d₃ is absent or a neutral hydrophobic, neutral polar, or acidic amino acid;

d₄ is absent or any amino acid;

d₅ is absent or a neutral hydrophobic, neutral polar, or acidic amino acid;

d₆ is absent or a neutral hydrophobic, neutral polar, or basic amino acid;

d₇ is selected from any one of the amino acids T, I, or R;

d₈ is selected from any one of R, S, Q;

d₉ is selected from any one of P, R and Q

and d₁₀ through d₁₃ are selected from any amino acid.

The consensus sequence of the mTN8 con6 series is WYe₁e₂Ye₃G, (SEQ ID NO: 356) wherein e₁ is P, S or Y; e₂ is C or Q, and e₃ is G or H.

In addition to the TN-19 affinity matured family, additional affinity matured peptides were produced from the linear L-2, L-15, L-17, L-20, L-21, and L-24 first round peptides. These families are presented in Table V below.

TABLE V
additional affinity matured peptides

| Affinity | | |
|---|---|---|
| matured | | |
| peptibody | SEQ ID NO: | Peptide Sequence |
| L2 | 104 | MEMLDSLFELLKDMVPISKA |
| mL2-Con1 | 357 | RMEMLESLLELLKEIVPMSKAG |
| mL2-Con2 | 358 | RMEMLESLLELLKEIVPMSKAR |
| mL2-1 | 359 | RMEMLESLLELLKDIVPMSKPS |
| mL2-2 | 360 | GMEMLESLFELLQEIVPMSKAP |
| mL2-3 | 361 | RMEMLESLLELLKDIVPISNPP |
| mL2-4 | 362 | RIEMLESLLELLQEIVPISKAE |
| mL2-5 | 363 | RMEMLQSLLELLKDIVPMSNAR |
| mL2-6 | 364 | RMEMLESLLELLKEIVPTSNGT |
| mL2-7 | 365 | RMEMLESLFELLKEIVPMSKAG |
| mL2-8 | 366 | RMEMLGSLLELLKEIVPMSKAR |
| mL2-9 | 367 | QMELLDSLFELLKEIVPKSQPA |
| mL2-10 | 368 | RMEMLDSLLELLKEIVPMSNAR |
| mL2-11 | 369 | RMEMLESLLELLHEIVPMSQAG |
| mL2-12 | 370 | QMEMLESLLQLLKEIVPMSKAS |
| mL2-13 | 371 | RMEMLDSLLELLKDMVPMTTGA |
| mL2-14 | 372 | RIEMLESLLELLKDMVPMANAS |
| mL2-15 | 373 | RMEMLESLLQLLNEIVPMSRAR |
| mL2-16 | 374 | RMEMLESLFDLLKELVPMSKGV |
| mL2-17 | 375 | RIEMLESLLELLKDIVPIQKAR |
| mL2-18 | 376 | RMELLESLFELLKDMVPMSDSS |
| mL2-19 | 377 | RMEMLESLLEVLQEIVPRAKGA |
| mL2-20 | 378 | RMEMLDSLLQLLNEIVPMSHAR |
| mL2-21 | 379 | RMEMLESLLELLKDIVPMSNAG |
| mL2-22 | 380 | RMEMLQSLFELLKGMVPISKAG |
| mL2-23 | 381 | RMEMLESLLELLKEIVPNSTAA |
| mL2-24 | 382 | RMEMLQSLLELLKEIVPISKAG |
| mL2-25 | 383 | RIEMLDSLLELLNELVPMSKAR |
| L-15 | 117 | HHGWNYLRKGSAPQWFEAWV |
| mL15-con1 | 384 | QVESLQQLLMWLDQKLASGPQG |
| mL15-1 | 385 | RMELLESLFELLKEMVPRSKAV |
| mL15-2 | 386 | QAVSLQHLLMWLDQKLASGPQH |
| mL15-3 | 387 | DEDSLQQLLMWLDQKLASGPQL |
| mL15-4 | 388 | PVASLQQLLIWLDQKLAQGPHA |
| mL15-5 | 389 | EVDELQQLLNWLDHKLASGPLQ |
| mL15-6 | 390 | DVESLEQLLMWLDHQLASGPHG |
| mL15-7 | 391 | QVDSLQQVLLWLEHKLALGPQV |
| mL15-8 | 392 | GDESLQHLLMWLEQKLALGPHG |
| mL15-9 | 393 | QIEMLESLLDLLRDMVPMSNAF |
| mL15-10 | 394 | EVDSLQQLLMWLDQKLASGPQA |
| mL15-11 | 395 | EDESLQQLLIYLDKMLSSGPQV |
| mL15-12 | 396 | AMDQLHQLLIWLDHKLASGPQA |
| mL15-13 | 397 | RIEMLESLLELLDEIALIPKAW |
| mL15-14 | 398 | EVVSLQHLLMWLEHKLASGPDG |
| mL15-15 | 399 | GGESLQQLLMWLDQQLASGPQR |
| mL15-16 | 400 | GVESLQQLLIFLDHMLVSGPHD |
| mL15-17 | 401 | NVESLEHLMMWLERLLASGPYA |
| mL15-18 | 402 | QVDSLQQLLIWLDHQLASGPKR |
| mL15-19 | 403 | EVESLQQLLMWLEHKLAQGPQG |
| mL15-20 | 404 | EVDSLQQLLMWLDQKLASGPHA |
| mL15-21 | 405 | EVDSLQQLLMWLDQQLASGPQK |
| mL15-22 | 406 | GVEQLPQLLMWLEQKLASGPQR |
| mL15-23 | 407 | GEDSLQQLLMWLDQQLAAGPQV |
| mL15-24 | 408 | ADDSLQQLLMWLDRKLASGPHV |
| mL15-25 | 409 | PVDSLQQLLIWLDQKLASGPQG |
| L-17 | 119 | RATLLKDFWQLVEGYGDN |
| mL17-con1 | 410 | DWRATLLKEFWQLVEGLGDNLV |
| mL17-con2 | 411 | QSRATLLKEFWQLVEGLGDKQA |
| mL17-1 | 412 | DGRATLLTEFWQLVQGLGQKEA |
| mL17-2 | 413 | LARATLLKEFWQLVEGLGEKVV |
| mL17-3 | 414 | GSRDTLLKEFWQLVVGLGDMQT |
| mL17-4 | 415 | DARATLLKEFWQLVDAYGDRMV |
| mL17-5 | 416 | NDRAQLLRDFWQLVDGLGVKSW |
| mL17-6 | 417 | GVRETLLYELWYLLKGLGANQG |
| mL17-7 | 418 | QARATLLKEFCQLVGCQGDKLS |
| mL17-8 | 419 | QERATLLKEFWQLVAGLGQNMR |
| mL17-9 | 420 | SGRATLLKEFWQLVQGLGEYRW |
| mL17-10 | 421 | TMRATLLKEFWLFVDGQREMQW |
| mL17-11 | 422 | GERATLLNDFWQLVDGQGDNTG |
| mL17-12 | 423 | DERETLLKEFWQLVHGWGDNVA |
| mL17-13 | 424 | GGRATLLKELWQLLEGQGANLV |
| mL17-14 | 425 | TARATLLNELVQLVKGYGDKLV |
| mL17-15 | 426 | GMRATLLQEFWQLVGGQGDNWM |
| mL17-16 | 427 | STRATLLNDLWQLMKGWAEDRG |
| mL17-17 | 428 | SERATLLKELWQLVGGWGDNFG |
| mL17-18 | 429 | VGRATLLKEFWQLVEGLVGQSR |
| mL17-19 | 430 | EIRATLLKEFWQLVDEWREQPN |
| mL17-20 | 431 | QLRATLLKEFLQLVHGLGETDS |
| mL17-21 | 432 | TQRATLLKEFWQLIEGLGGKHV |
| mL17-22 | 433 | HYRATLLKEFWQLVDGLREQGV |
| mL17-23 | 434 | QSRVTLLREFWQLVESYRPIVN |
| mL17-24 | 435 | LSRATLLNEFWQFVDGQRDKRM |
| mL17-25 | 436 | WDRATLLNDFWHLMEELSQKPG |
| mL17-26 | 437 | QERATLLKEFWRMVEGLGKNRG |
| mL17-27 | 438 | NERATLLREFWQLVGGYGVNQR |
| L-20 | 122 | YREMSMLEGLLDVLERLQHY |
| mL20-1 | 439 | HQRDMSMLWELLDVLDGLRQYS |
| mL20-2 | 440 | TQRDMSMLDGLLEVLDQLRQQR |
| mL20-3 | 441 | TSRDMSLLWELLEELDRLGHQR |
| mL20-4 | 442 | MQHDMSMLYGLVELLESLGHQI |
| mL20-5 | 443 | WNRDMRMLESLFEVLDGLRQQV |
| mL20-6 | 444 | GYRDMSMLEGLLAVLDRLGPQL |
| mL20 con1 | 445 | TQRDMSMLEGLLEVLDRLGQQR |
| mL20 con2 | 446 | WYRDMSMLEGLLEVLDRLGQQR |
| L-21 | 123 | HNSSQMLLSELIMLVGSMMQ |
| mL21-1 | 447 | TQNSRQMLLSDFMMLVGSMIQG |
| mL21-2 | 448 | MQTSRHILLSEFMMLVGSIMHG |
| mL21-3 | 449 | HDNSRQMLLSDLLHLVGTMIQG |
| mL21-4 | 450 | MENSRQNLLREL IMLVGNMSHQ |
| mL21-5 | 451 | QDTSRHMLLREFMMLVGEMIQG |
| mL21 con1 | 452 | DQNSRQMLLSDLMILVGSMIQG |
| L-24 | 126 | EFFHWLHNHRSEVNHWLDMN |
| mL24-1 | 453 | NVFFQWVQKHGRVVYQWLDINV |
| mL24-2 | 454 | FDFLQWLQNHRSEVEHWLVMDV |

The affinity matured peptides provided in Tables IV and V are then assembed into peptibodies as described above and assayed using the in vivo assays.

The affinity matured L2 peptides comprise a consensus sequence of

f₁EMLf₂SLf₃f₄LL, (SEQ ID NO: 455), wherein f₁ is M or I; f₂ is any amino acid; f₃ is Lor F; and f₄ is E, Q or D.

The affinity matured L15 peptide family comprise the sequence Lg₁g₂LLg₃g₄L, (SEQ ID NO: 456), wherein g₁ is Q, D or E, g₂ is S, Q, D or E, g₃ is any amino acid, and g₄ is L, W, F, or Y. The affinity matured L17 family comprises the sequence: h₁h₂h₃h₄h₅h₆h₇h₈h₉ (SEQ ID NO: 457) wherein h₁ is R or D; h₂ is any amino acid; h₃ is A, T S or Q; h₄ is L or M; h₅ is L or S; h₆ is any amino acid; h₇ is F or E; h₈ is W, F or C; and h₉ is L, F, M or K. Consensus sequences may also be determined for the mL20, mL21 and mL24 families of peptides shown above.

Peptibodies were constructed from these affinity matured peptides as described above, using a linker attached to the Fc domain of human IgG1, having SEQ ID NO: 296, at the N-terminus (N configuration), at the C terminus (C configuration) of the Fc, or at both the N and C terminals (N,C configurations), as described in Example 2 above. The peptides named were attached to the C or N terminals via a 5 glycine (5G), 8 glycine or k linker sequence. In the 2X peptibody version the peptides were linked with linkers such as 5 gly, 8 gly or k. Affinity matured peptides and peptibodies are designated with a small “m” such as mTN8-19-22 for example. Peptibodies of the present invention further contain two splice sites where the peptides were spliced into the phagemid vectors. The position of these splice sites are AQ-peptide-LE. The peptibodies generally include these additional amino acids (although they are not included in the peptide sequences listed in the tables). In some peptibodies the LE amino acids were removed from the peptides sequences. These peptibodies are designated -LE.

Exemplary peptibodies, and exemplary polynucleotide sequences encoding them, are provided in Table VI below. This table includes examples of peptibody sequences (as opposed to peptide only), such as the 2x mTN8-19-7 (SEQ ID NO: 615) and the peptibody with the LE sequences deleted (SEQ ID NO: 617). By way of explanation, the linker sequences in the 2x versions refers to the linker between the tandem peptides. These peptibody sequences contain the Fc, linkers, AQ and LE sequences. The accompanying nucleotide sequence encodes the peptide sequence in addition to the AQ/LE linker sequences, if present, but does not encode the designated linker.

TABLE VI
Exemplary peptibodies, and exemplary polynucleotide sequences encoding them

| | | | | Term- |
|---|---|---|---|---|
| Peptibody Name | Peptide | Nucleotide Sequence (SEQ ID NO) | Linker | inus |
| mL2-Con1 | RMEMLESLLELL | CGTATGGAAATGCTTGAATCTCTTC | 5 gly | N |
| | KEIVPMSKAG | TTGAACTTCTTAAAGAAATTGTTCC | | |
| | | AATGTCTAAAGCTGGT (SEQ ID NO: | | |
| | | 458) | | |
| mL2-Con2 | RMEMLESLLELL | CGTATGGAAATGCTTGAATCTCTTC | 5 gly | N |
| | KEIVPMSKAR | TTGAACTTCTTAAAGAAATTGTTCC | | |
| | | AATGTCTAAAGCTCGT (SEQ ID NO: | | |
| | | 459) | | |
| mL2-1 | RMEMLESLLELL | CGTATGGAAATGCTTGAATCTCTTC | 5 gly | N |
| | KDIVPMSKPS | TTGAACTTCTTAAAGATATTGTTCC | | |
| | | AATGTCTAAACCATCT (SEQ ID NO: | | |
| | | 460) | | |
| mL2-2 | GMEMLESLFELL | GGTATGGAAATGCTTGAATCTCTTT | 5 gly | N |
| | QEIVPMSKAP | TTGAACTTCTTCAAGAAATTGTTCC | | |
| | | AATGTCTAAAGCTCCA (SEQ ID NO: | | |
| | | 461) | | |
| mL2-3 | RMEMLESLLELL | CGTATGGAAATGCTTGAATCTCTTC | 5 gly | N |
| | KDIVPISNPP | TTGAACTTCTTAAAGATATTGTTCC | | |
| | | AATTTCTAATCCACCA (SEQ ID NO: | | |
| | | 462) | | |
| mL2-4 | RIEMLESLLELLQ | CGTATTGAAATGCTTGAATCTCTTC | 5 gly | N |
| | EIVPISKAE | TTGAACTTCTTCAAGAAATTGTTCC | | |
| | | AATTTCTAAAGCTGAA (SEQ ID NO: | | |
| | | 463) | | |
| mL2-5 | RMEMLQSLLELL | CGTATGGAAATGCTTCAATCTCTTC | 5 gly | N |
| | KDIVPMSNAR | TTGAACTTCTTAAAGATATTGTTCC | | |
| | | AATGTCTAATGCTCGT (SEQ ID NO: | | |
| | | 464) | | |
| mL2-6 | RMEMLESLLELL | CGTATGGAAATGCTTGAATCTCTTC | 5 gly | N |
| | KEIVPTSNGT | TTGAACTTCTTAAAGAAATTGTTCC | | |
| | | AACTTCTAATGGTACT (SEQ ID NO: | | |
| | | 465) | | |
| mL2-7 | RMEMLESLFELL | CGTATGGAAATGCTTGAATCTCTTT | 5 gly | N |
| | KEIVPMSKAG | TTGAACTTCTTAAAGAAATTGTTCC | | |
| | | AATGTCTAAAGCTGGT (SEQ ID NO: | | |
| | | 466) | | |
| mL2-8 | RMEMLGSLLELL | CGTATGGAAATGCTTGGTTCTCTTC | 5 gly | N |
| | KEIVPMSKAR | TTGAACTTCTTAAAGAAATTGTTCC | | |
| | | AATGTCTAAAGCTCGT (SEQ ID NO: | | |
| | | 467) | | |
| mL2-9 | QMELLDSLFELL | CAAATGGAACTTCTTGATTCTCTTT | 5 gly | N |
| | KEIVPKSQPA | TTGAACTTCTTAAAGAAATTGTTCC | | |
| | | AAAATCTCAACCAGCT (SEQ ID NO: | | |
| | | 468) | | |
| mL2-10 | RMEMLDSLLELL | CGTATGGAAATGCTTGATTCTCTTC | 5 gly | N |
| | KEIVPMSNAR | TTGAACTTCTTAAAGAAATTGTTCC | | |
| | | AATGTCTAATGCTCGT (SEQ ID NO: | | |
| | | 469) | | |
| mL2-11 | RMEMLESLLELL | CGTATGGAAATGCTTGAATCTCTTC | 5 gly | N |
| | HEIVPMSQAG | TTGAACTTCTTCATGAAATTGTTCC | | |
| | | AATGTCTCAAGCTGGT (SEQ ID NO: | | |
| | | 470) | | |
| mL2-12 | QMEMLESLLQLL | CAAATGGAAATGCTTGAATCTCTTC | 5 gly | N |
| | KEIVPMSKAS | TTCAACTTCTTAAAGAAATTGTTCC | | |
| | | AATGTCTAAAGCTTCT (SEQ ID NO: | | |
| | | 471) | | |
| mL2-13 | RMEMLDSLLELL | CGTATGGAAATGCTTGATTCTCTTC | 5 gly | N |
| | KDMVPMTTGA | TTGAACTTCTTAAAGATATGGTTCC | | |
| | | AATGACTACTGGTGCT (SEQ ID NO: | | |
| | | 472) | | |
| mL2-14 | RIEMLESLLELLK | CGTATTGAAATGCTTGAATCTCTTC | 5 gly | N |
| | DMVPMANAS | TTGAACTTCTTAAAGATATGGTTCC | | |
| | | AATGGCTAATGCTTCT (SEQ ID NO: | | |
| | | 473) | | |
| mL2-15 | RMEMLESLLQLL | CGTATGGAAATGCTTGAATCTCTTC | 5 gly | N |
| | NEIVPMSRAR | TTCAACTTCTTAATGAAATTGTTCC | | |
| | | AATGTCTCGTGCTCGT (SEQ ID NO: | | |
| | | 474) | | |
| mL2-16 | RMEMLESLFDLL | CGTATGGAAATGCTTGAATCTCTTT | 5 gly | N |
| | KELVPMSKGV | TTGATCTTCTTAAAGAACTTGTTCC | | |
| | | AATGTCTAAAGGTGTT (SEQ ID NO: | | |
| | | 475) | | |
| mL2-17 | RIEMLESLLELLK | CGTATTGAAATGCTTGAATCTCTTC | 5 gly | N |
| | DIVPIQKAR | TTGAACTTCTTAAAGATATTGTTCC | | |
| | | AATTCAAAAAGCTCGT (SEQ ID | | |
| | | NO: 476) | | |
| mL2-18 | RMELLESLFELLK | CGTATGGAACTTCTTGAATCTCTTT | 5 gly | N |
| | DMVPMSDSS | TTGAACTTCTTAAAGATATGGTTCC | | |
| | | AATGTCTGATTCTTCT (SEQ ID NO: | | |
| | | 477) | | |
| mL2-19 | RMEMLESLLEVL | CGTATGGAAATGCTTGAATCTCTTC | 5 gly | N |
| | QEIVPRAKGA | TTGAAGTTCTTCAAGAAATTGTTCC | | |
| | | ACGTGCTAAAGGTGCT (SEQ ID | | |
| | | NO: 478) | | |
| mL2-20 | RMEMLDSLLQLL | CGTATGGAAATGCTTGATTCTCTTC | 5 gly | N |
| | NEIVPMSHAR | TTCAACTTCTTAATGAAATTGTTCC | | |
| | | AATGTCTCATGCTCGT (SEQ ID NO: | | |
| | | 479) | | |
| mL2-21 | RMEMLESLLELL | CGTATGGAAATGCTTGAATCTCTTC | 5 gly | N |
| | KDIVPMSNAG | TTGAACTTCTTAAAGATATTGTTCC | | |
| | | AATGTCTAATGCTGGT (SEQ ID NO: | | |
| | | 480) | | |
| mL2-22 | RMEMLQSLFELL | CGTATGGAAATGCTTCAATCTCTTT | 5 gly | N |
| | KGMVPISKAG | TTGAACTTCTTAAAGGTATGGTTCC | | |
| | | AATTTCTAAAGCTGGT (SEQ ID | | |
| | | NO: 481) | | |
| mL2-23 | RMEMLESLLELL | CGTATGGAAATGCTTGAATCTCTTC | 5 gly | N |
| | KEIVPNSTAA | TTGAACTTCTTAAAGAAATTGTTCC | | |
| | | AAATTCTACTGCTGCT (SEQ ID NO: | | |
| | | 482) | | |
| mL2-24 | RMEMLQSLLELL | CGTATGGAAATGCTTCAATCTCTTC | 5 gly | N |
| | KEIVPISKAG | TTGAACTTCTTAAAGAAATTGTTCC | | |
| | | AATTTCTAAAGCTGGT (SEQ ID NO: | | |
| | | 483) | | |
| mL2-25 | RIEMLDSLLELLN | CGTATTGAAATGCTTGATTCTCTTC | 5 gly | N |
| | ELVPMSKAR | TTGAACTTCTTAATGAACTTGTTCC | | |
| | | AATGTCTAAAGCTCGT (SEQ ID NO: | | |
| | | 484) | | |
| mL17-Con1 | DWRATLLKEFW | GATTGGCGTGCTACTCTTCTTAAAG | 5 gly | N |
| | QLVEGLGDNLV | AATTTTGGCAACTTGTTGAAGGTCT | | |
| | | TGGTGATAATCTTGTT (SEQ ID NO: | | |
| | | 485) | | |
| mL17-1 | DGRATLLTEFWQ | GATGGTCGTGCTACTCTTCTTACTG | 5 gly | N |
| | LVQGLGQKEA | AATTTTGGCAACTTGTTCAAGGTCT | | |
| | | TGGTCAAAAAGAAGCT (SEQ ID | | |
| | | NO: 486) | | |
| mL17-2 | LARATLLKEFWQ | CTTGCTCGTGCTACTCTTCTTAAAG | 5 gly | N |
| | LVEGLGEKVV | AATTTTGGCAACTTGTTGAAGGTCT | | |
| | | TGGTGAAAAAGTTGTT (SEQ ID NO: | | |
| | | 487) | | |
| mL17-3 | GSRDTLLKEFWQ | GGTTCTCGTGATACTCTTCTTAAAG | 5 gly | N |
| | LVVGLGDMQT | AATTTTGGCAACTTGTTGTTGGTCT | | |
| | | TGGTGATATGCAAACT (SEQ ID NO: | | |
| | | 488) | | |
| mL17-4 | DARATLLKEFWQ | GATGCTCGTGCTACTCTTCTTAAAG | 5 gly | N |
| | LVDAYGDRMV | AATTTTGGCAACTTGTTGATGCTTA | | |
| | | TGGTGATCGTATGGTT (SEQ ID NO: | | |
| | | 489) | | |
| mL17-5 | NDRAQLLRDFWQ | AATGATCGTGCTCAACTTCTTCGTG | 5 gly | N |
| | LVDGLGVKSW | ATTTTTGGCAACTTGTTGATGGTCT | | |
| | | TGGTGTTAAATCTTGG (SEQ ID NO: | | |
| | | 490) | | |
| mL17-6 | GVRETLLYELWY | GGTGTTCGTGAAACTCTTCTTTATG | 5 gly | N |
| | LLKGLGANQG | AACTTTGGTATCTTCTTAAAGGTCT | | |
| | | TGGTGCTAATCAAGGT (SEQ ID NO: | | |
| | | 491) | | |
| mL17-7 | QARATLLKEFCQ | CAAGCTCGTGCTACTCTTCTTAAAG | 5 gly | N |
| | LVGCQGDKLS | AATTTTGTCAACTTGTTGGTTGTCA | | |
| | | AGGTGATAAACTTTCT (SEQ ID NO: | | |
| | | 492) | | |
| mL17-8 | QERATLLKEFWQ | CAAGAACGTGCTACTCTTCTTAAA | 5 gly | N |
| | LVAGLGQNMR | GAATTTTGGCAACTTGTTGCTGGTC | | |
| | | TTGGTCAAAATATGCGT (SEQ ID | | |
| | | NO: 493) | | |
| mL17-9 | SGRATLLKEFWQ | TCTGGTCGTGCTACTCTTCTTAAAG | 5 gly | N |
| | LVQGLGEYRW | AATTTTGGCAACTTGTTCAAGGTCT | | |
| | | TGGTGAATATCGTTGG (SEQ ID NO: | | |
| | | 494) | | |
| mL17-10 | TMRATLLKEFWL | ACTATGCGTGCTACTCTTCTTAAAG | 5 gly | N |
| | FVDGQREMQW | AATTTTGGCTTTTTGTTGATGGTCA | | |
| | | ACGTGAAATGCAATGG (SEQ ID NO: | | |
| | | 495) | | |
| mL17-11 | GERATLLNDFWQ | GGTGAACGTGCTACTCTTCTTAATG | 5 gly | N |
| | LVDGQGDNTG | ATTTTTGGCAACTTGTTGATGGTCA | | |
| | | AGGTGATAATACTGGT (SEQ ID | | |
| | | NO: 496) | | |
| mL17-12 | DERETLLKEFWQ | GATGAACGTGAAACTCTTCTTAAA | 5 gly | N |
| | LVHGWGDNVA | GAATTTTGGCAACTTGTTCATGGTT | | |
| | | GGGGTGATAATGTTGCT (SEQ ID | | |
| | | NO: 497) | | |
| mL17-13 | GGRATLLKELWQ | GGTGGTCGTGCTACTCTTCTTAAAG | 5 gly | N |
| | LLEGQGANLV | AACTTTGGCAACTTCTTGAAGGTCA | | |
| | | AGGTGCTAATCTTGTT (SEQ ID NO: | | |
| | | 498) | | |
| mL17-14 | TARATLLNELVQ | ACTGCTCGTGCTACTCTTCTTAATG | 5 gly | N |
| | LVKGYGDKLV | AACTTGTTCAACTTGTTAAAGGTTA | | |
| | | TGGTGATAAACTTGTT (SEQ ID NO: | | |
| | | 499) | | |
| mL17-15 | GMRATLLQEFWQ | GGTATGCGTGCTACTCTTCTTCAAG | 5 gly | N |
| | LVGGQGDNWM | AATTTTGGCAACTTGTTGGTGGTCA | | |
| | | AGGTGATAATTGGATG (SEQ ID | | |
| | | NO: 500) | | |
| mL17-16 | STRATLLNDLWQ | TCTACTCGTGCTACTCTTCTTAATG | 5 gly | N |
| | LMKGWAEDRG | ATCTTTGGCAACTTATGAAAGGTTG | | |
| | | GGCTGAAGATCGTGGT (SEQ ID | | |
| | | NO: 501) | | |
| mL17-17 | SERATLLKELWQ | TCTGAACGTGCTACTCTTCTTAAAG | 5 gly | N |
| | LVGGWGDNFG | AACTTTGGCAACTTGTTGGTGGTTG | | |
| | | GGGTGATAATTTTGGT (SEQ ID NO: | | |
| | | 502) | | |
| mL17-18 | VGRATLLKEFWQ | GTTGGTCGTGCTACTCTTCTTAAAG | 5 gly | N |
| | LVEGLVGQSR | AATTTTGGCAACTTGTTGAAGGTCT | | |
| | | TGTTGGTCAATCTCGT (SEQ ID NO: | | |
| | | 503) | | |
| 2x mTN8-Con6- | M-GAQ- | TGGTATCCGTGTTATGAGGGTCACT | 1K | N |
| (N)-1K | WYPCYEGHFWC | TCTGGTGCTACGATCTGGGTTCTGG | | |
| | YDL- | TTCCACTGCTTCTTCTGGTTCCGGT | | |
| | GSGSATGGSGST | TCCGCTACTGGTTGGTACCCGTGCT | | |
| | ASSGSGSATG- | ACGAAGGTCACTTTTGGTGTTATGA | | |
| | WYPCYEGHFWC | TCTG (SEQ ID NO: 505) | | |
| | YDL-LE-5G-FC | | | |
| | (SEQ ID NO: 504) | | | |
| 2x mTN8-Con6- | FC-5G-AQ- | TGGTATCCGTGTTATGAGGGTCACT | 1K | C |
| (C)-1K | WYPCYEGHFWC | TCTGGTGCTACGATCTGGGTTCTGG | | |
| | YDL- | TTCCACTGCTTCTTCTGGTTCCGGT | | |
| | GSGSATGGSGST | TCCGCTACTGGTTGGTACCCGTGCT | | |
| | ASSGSGSATG- | ACGAAGGTCACTTTTGGTGTTATGA | | |
| | WYPCYEGHFWC | TCTG (SEQ ID NO: 507) | | |
| | YDL-LE (SEQ ID | | | |
| | NO: 506) | | | |
| 2x mTN8-Con7- | M-GAQ- | ATCTTTGGCTGTAAATGGTGGGAC | 1K | N |
| (N)-1K | IFGCKWWDVQC | GTTCAGTGCTACCAGTTCGGTTCTG | | |
| | YQF- | GTTCCACTGCTTCTTCTGGTTCCGG | | |
| | GSGSATGGSGST | TTCCGCTACTGGTATCTTCGGTTGC | | |
| | ASSGSGSATG- | AAGTGGTGGGATGTACAGTGTTAT | | |
| | IFGCKWWDVQC | CAGTTT (SEQ ID NO: 509) | | |
| | YQF-LE-5G-FC | | | |
| | (SEQ ID NO: 508) | | | |
| 2x mTN8-Con7- | FC-5G-AQ- | ATCTTTGGCTGTAAATGGTGGGAC | 1K | C |
| (C)-1K | IFGCKWWDVQC | GTTCAGTGCTACCAGTTCGGTTCTG | | |
| | YQF- | GTTCCACTGCTTCTTCTGGTTCCGG | | |
| | GSGSATGGSGST | TTCCGCTACTGGTATCTTCGGTTGC | | |
| | ASSGSGSATG- | AAGTGGTGGGATGTACAGTGTTAT | | |
| | IFGCKWWDVQC | CAGTTT (SEQ ID NO: 511) | | |
| | YQF-LE (SEQ ID | | | |
| | NO: 510) | | | |
| 2x mTN8-Con8- | M-GAQ- | ATCTTTGGCTGTAAGTGGTGGGAC | 1K | N |
| (N)-1K | IFGCKWWDVDC | GTTGACTGCTACCAGTTCGGTTCTG | | |
| | YQF- | GTTCCACTGCTTCTTCTGGTTCCGG | | |
| | GSGSATGGSGST | TTCCGCTACTGGTATCTTCGGTTGC | | |
| | ASSGSGSATG- | AAATGGTGGGACGTTGATTGTTAT | | |
| | IFGCKWWDVDC | CAGTTT (SEQ ID NO: 513) | | |
| | YQF-LE-5G-FC | | | |
| | (SEQ ID NO: 512) | | | |
| 2x mTN8-Con8- | FC-5G-AQ- | ATCTTTGGCTGTAAGTGGTGGGAC | 1K | C |
| (C)-1K | IFGCKWWDVDC | GTTGACTGCTACCAGTTCGGTTCTG | | |
| | YQF- | GTTCCACTGCTTCTTCTGGTTCCGG | | |
| | GSGSATGGSGST | TTCCGCTACTGGTATCTTCGGTTGC | | |
| | ASSGSGSATG- | AAATGGTGGGACGTTGATTGTTAT | | |
| | IFGCKWWDVDC | CAGTTT (SEQ ID NO: 515) | | |
| | YQF-LE (SEQ ID | | | |
| | NO: 514) | | | |
| ML15-Con1 | QVESLQQLLMWL | CAGGTTGAATCCCTGCAGCAGCTG | 5 gly | C |
| | DQKLASGPQG | CTGATGTGGCTGGACCAGAAACTG | | |
| | | GCTTCCGGTCCGCAGGGT (SEQ ID | | |
| | | NO: 516) | | |
| ML15-1 | RMELLESLFELLK | CGTATGGAACTGCTGGAATCCCTG | 5 gly | C |
| | EMVPRSKAV | TTCGAACTGCTGAAAGAAATGGTT | | |
| | | CCGCGTTCCAAAGCTGTT (SEQ ID | | |
| | | NO: 517) | | |
| mL15-2 | QAVSLQHLLMW | CAGGCTGTTTCCCTGCAGCACCTGC | 5 gly | C |
| | LDQKLASGPQH | TGATGTGGCTGGACCAGAAACTGG | | |
| | | CTTCCGGTCCGCAGCAC (SEQ ID | | |
| | | NO: 518) | | |
| mL15-3 | DEDSLQQLLMWL | GACGAAGACTCCCTGCAGCAGCTG | 5 gly | C |
| | DQKLASGPQL | CTGATGTGGCTGGACCAGAAACTG | | |
| | | GCTTCCGGTCCGCAGCTG (SEQ ID | | |
| | | NO: 519) | | |
| mL15-4 | PVASLQQLLIWL | CCGGTTGCTTCCCTGCAGCAGCTGC | 5 gly | C |
| | DQKLAQGPHA | TGATCTGGCTGGACCAGAAACTGG | | |
| | | CTCAGGGTCCGCACGCT (SEQ ID | | |
| | | NO: 520) | | |
| mL15-5 | EVDELQQLLNWL | GAAGTTGACGAACTGCAGCAGCTG | 5 gly | C |
| | DHKLASGPLQ | CTGAACTGGCTGGACCACAAACTG | | |
| | | GCTTCCGGTCCGCTGCAG (SEQ ID | | |
| | | NO: 521) | | |
| mL15-6 | DVESLEQLLMWL | GACGTTGAATCCCTGGAACAGCTG | 5 gly | C |
| | DHQLASGPHG | CTGATGTGGCTGGACCACCAGCTG | | |
| | | GCTTCCGGTCCGCACGGT (SEQ ID | | |
| | | NO: 522) | | |
| mL15-7 | QVDSLQQVLLWL | CAGGTTGACTCCCTGCAGCAGGTT | 5 gly | C |
| | EHKLALGPQV | CTGCTGTGGCTGGAACACAAACTG | | |
| | | GCTCTGGGTCCGCAGGTT (SEQ ID | | |
| | | NO: 523) | | |
| mL15-8 | GDESLQHLLMWL | GGTGACGAATCCCTGCAGCACCTG | 5 gly | C |
| | EQKLALGPHG | CTGATGTGGCTGGAACAGAAACTG | | |
| | | GCTCTGGGTCCGCACGGT (SEQ ID | | |
| | | NO: 524) | | |
| mL15-9 | QIEMLESLLDLLR | CAGATCGAAATGCTGGAATCCCTG | 5 gly | C |
| | DMVPMSNAF | CTGGACCTGCTGCGTGACATGGTTC | | |
| | | CGATGTCCAACGCTTTC (SEQ ID | | |
| | | NO: 525) | | |
| mL15-10 | EVDSLQQLLMWL | GAAGTTGACTCCCTGCAGCAGCTG | 5 gly | C |
| | DQKLASGPQA | CTGATGTGGCTGGACCAGAAACTG | | |
| | | GCTTCCGGTCCGCAGGCT (SEQ ID | | |
| | | NO: 526) | | |
| mL15-11 | EDESLQQLLIYLD | GAAGACGAATCCCTGCAGCAGCTG | 5 gly | C |
| | KMLSSGPQV | CTGATCTACCTGGACAAAATGCTG | | |
| | | TCCTCCGGTCCGCAGGTT (SEQ ID | | |
| | | NO: 527) | | |
| mL15-12 | AMDQLHQLLIWL | GCTATGGACCAGCTGCACCAGCTG | 5 gly | C |
| | DHKLASGPQA | CTGATCTGGCTGGACCACAAACTG | | |
| | | GCTTCCGGTCCGCAGGCT (SEQ ID | | |
| | | NO: 528) | | |
| mL15-13 | RIEMLESLLELLD | CGTATCGAAATGCTGGAATCCCTG | 5 gly | C |
| | EIALIPKAW | CTGGAACTGCTGGACGAAATCGCT | | |
| | | CTGATCCCGAAAGCTTGG (SEQ ID | | |
| | | NO: 529) | | |
| mL15-14 | EVVSLQHLLMWL | GAAGTTGTTTCCCTGCAGCACCTGC | 5 gly | C |
| | EHKLASGPDG | TGATGTGGCTGGAACACAAACTGG | | |
| | | CTTCCGGTCCGGACGGT (SEQ ID | | |
| | | NO: 530) | | |
| mL15-15 | GGESLQQLLMWL | GGTGGTGAATCCCTGCAGCAGCTG | 5 gly | C |
| | DQQLASGPQR | CTGATGTGGCTGGACCAGCAGCTG | | |
| | | GCTTCCGGTCCGCAGCGT (SEQ ID | | |
| | | NO: 531) | | |
| mL15-16 | GVESLQQLLIFLD | GGTGTTGAATCCCTGCAGCAGCTG | 5 gly | C |
| | HMLVSGPHD | CTGATCTTCCTGGACCACATGCTGG | | |
| | | TTTCCGGTCCGCACGAC (SEQ ID | | |
| | | NO: 532) | | |
| mL15-17 | NVESLEHLMMW | AACGTTGAATCCCTGGAACACCTG | 5 gly | C |
| | LERLLASGPYA | ATGATGTGGCTGGAACGTCTGCTG | | |
| | | GCTTCCGGTCCGTACGCT (SEQ ID | | |
| | | NO: 533) | | |
| mL15-18 | QVDSLQQLLIWL | CAGGTTGACTCCCTGCAGCAGCTG | 5 gly | C |
| | DHQLASGPKR | CTGATCTGGCTGGACCACCAGCTG | | |
| | | GCTTCCGGTCCGAAACGT (SEQ ID | | |
| | | NO: 534) | | |
| mL15-19 | EVESLQQLLMWL | GAAGTTGAATCCCTGCAGCAGCTG | 5 gly | C |
| | EHKLAQGPQG | CTGATGTGGCTGGAACACAAACTG | | |
| | | GCTCAGGGTCCGCAGGGT (SEQ ID | | |
| | | NO: 535) | | |
| mL15-20 | EVDSLQQLLMWL | GAAGTTGACTCCCTGCAGCAGCTG | 5 gly | C |
| | DQKLASGPHA | CTGATGTGGCTGGACCAGAAACTG | | |
| | | GCTTCCGGTCCGCACGCT (SEQ ID | | |
| | | NO: 536) | | |
| mL15-21 | EVDSLQQLLMWL | GAAGTTGACTCCCTGCAGCAGCTG | 5 gly | C |
| | DQQLASGPQK | CTGATGTGGCTGGACCAGCAGCTG | | |
| | | GCTTCCGGTCCGCAGAAA (SEQ ID | | |
| | | NO: 537) | | |
| mL15-22 | GVEQLPQLLMWL | GGTGTTGAACAGCTGCCGCAGCTG | 5 gly | C |
| | EQKLASGPQR | CTGATGTGGCTGGAACAGAAACTG | | |
| | | GCTTCCGGTCCGCAGCGT (SEQ ID | | |
| | | NO: 538) | | |
| mL15-23 | GEDSLQQLLMWL | GGTGAAGACTCCCTGCAGCAGCTG | 5 gly | C |
| | DQQLAAGPQV | CTGATGTGGCTGGACCAGCAGCTG | | |
| | | GCTGCTGGTCCGCAGGTT (SEQ ID | | |
| | | NO: 539) | | |
| mL15-24 | ADDSLQQLLMW | GCTGACGACTCCCTGCAGCAGCTG | 5 gly | C |
| | LDRKLASGPHV | CTGATGTGGCTGGACCGTAAACTG | | |
| | | GCTTCCGGTCCGCACGTT (SEQ ID | | |
| | | NO: 540) | | |
| mL15-25 | PVDSLQQLLIWL | CCGGTTGACTCCCTGCAGCAGCTG | 5 gly | C |
| | DQKLASGPQGCT | GATCTGGCTGGACCAGAAACTG | | |
| | | GCTTCCGGTCCGCAGGGT (SEQ ID | | |
| | | NO: 541) | | |
| mL17-Con2 | QSRATLLKEFWQ | CAGTCCCGTGCTACCCTGCTGAAA | 5 gly | C |
| | LVEGLGDKQA | GAATTCTGGCAGCTGGTTGAAGGT | | |
| | | CTGGGTGACAAACAGGCT (SEQ ID | | |
| | | NO: 542) | | |
| mL17-19 | EIRATLLKEFWQL | GAAATCCGTGCTACCCTGCTGAAA | 5 gly | C |
| | VDEWREQPN | GAATTCTGGCAGCTGGTTGACGAA | | |
| | | TGGCGTGAACAGCCGAAC (SEQ ID | | |
| | | NO: 543) | | |
| mL17-20 | QLRATLLKEFLQL | CAGCTGCGTGCTACCCTGCTGAAA | 5 gly | C |
| | VHGLGETDS | GAATTCCTGCAGCTGGTTCACGGTC | | |
| | | TGGGTGAAACCGACTCC (SEQ ID | | |
| | | NO: 544) | | |
| mL17-21 | TQRATLLKEFWQ | ACCCAGCGTGCTACCCTGCTGAAA | 5 gly | C |
| | LIEGLGGKHV | GAATTCTGGCAGCTGATCGAAGGT | | |
| | | CTGGGTGGTAAACACGTT (SEQ ID | | |
| | | NO: 545) | | |
| mL17-22 | HYRATLLKEFWQ | CACTACCGTGCTACCCTGCTGAAA | 5 gly | C |
| | LVDGLREQGV | GAATTCTGGCAGCTGGTTGACGGT | | |
| | | CTGCGTGAACAGGGTGTT (SEQ ID | | |
| | | NO: 546) | | |
| mL17-23 | QSRVTLLREFWQ | CAGTCCCGTGTTACCCTGCTGCGTG | 5 gly | C |
| | LVESYRPIVN | AATTCTGGCAGCTGGTTGAATCCTA | | |
| | | CCGTCCGATCGTTAAC (SEQ ID NO: | | |
| | | 547) | | |
| mL17-24 | LSRATLLNEFWQ | CTGTCCCGTGCTACCCTGCTGAACG | 5 gly | C |
| | FVDGQRDKRM | AATTCTGGCAGTTCGTTGACGGTCA | | |
| | | GCGTGACAAACGTATG (SEQ ID | | |
| | | NO: 548) | | |
| mL17-25 | WDRATLLNDFW | TGGGACCGTGCTACCCTGCTGAAC | 5 gly | C |
| | HLMEELSQKPG | GACTTCTGGCACCTGATGGAAGAA | | |
| | | CTGTCCCAGAAACCGGGT (SEQ ID | | |
| | | NO: 549) | | |
| mL17-26 | QERATLLKEFWR | CAGGAACGTGCTACCCTGCTGAAA | 5 gly | C |
| | MVEGLGKNRG | GAATTCTGGCGTATGGTTGAAGGT | | |
| | | CTGGGTAAAAACCGTGGT (SEQ ID | | |
| | | NO: 550) | | |
| mL17-27 | NERATLLREFWQ | AACGAACGTGCTACCCTGCTGCGT | 5 gly | C |
| | LVGGYGVNQR | GAATTCTGGCAGCTGGTTGGTGGTT | | |
| | | ACGGTGTTAACCAGCGT (SEQ ID | | |
| | | NO: 551) | | |
| mTN8Con6-1 | QREWYPCYGGHL | CAGCGTGAATGGTACCCGTGCTAC | 5 gly | C |
| | WCYDLHKA | GGTGGTCACCTGTGGTGCTACGAC | | |
| | | CTGCACAAAGCT (SEQ ID NO: 552) | | |
| mTN8Con6-2 | ISAWYSCYAGHF | ATCTCCGCTTGGTACTCCTGCTACG | 5 gly | C |
| | WCWDLKQK | CTGGTCACTTCTGGTGCTGGGACCT | | |
| | | GAAACAGAAA (SEQ ID NO: 553) | | |
| mTN8Con6-3 | WTGWYQCYGGH | TGGACCGGTTGGTACCAGTGCTAC | 5 gly | C |
| | LWCYDLRRK | GGTGGTCACCTGTGGTGCTACGAC | | |
| | | CTGCGTCGTAAA (SEQ ID NO: 554) | | |
| mTN8Con6-4 | KTFWYPCYDGHF | AAAACCTTCTGGTACCCGTGCTAC | 5 gly | C |
| | WCYNLKSS | GACGGTCACTTCTGGTGCTACAAC | | |
| | | CTGAAATCCTCC (SEQ ID NO: 545) | | |
| mTN8Con6-5 | ESRWYPCYEGHL | GAATCCCGTTGGTACCCGTGCTAC | 5 gly | C |
| | WCFDLTET | GAAGGTCACCTGTGGTGCTTCGAC | | |
| | | CTGACCGAAACC (SEQ ID NO: 546) | | |
| mL24-1 | NVFFQWVQKHG | AATGTTTTTTTTCAATGGGTTCAAA | 5 gly | C |
| | RVVYQWLDINV | AACATGGTCGTGTTGTTTATCAATG | | |
| | | GCTTGATATTAATGTT (SEQ ID NO: | | |
| | | 557) | | |
| mL24-2 | FDFLQWLQNHRS | TTTGATTTTCTTCAATGGCTTCAAA | 5 gly | C |
| | EVEHWLVMDV | ATCATCGTTCTGAAGTTGAACATTG | | |
| | | GCTTGTTATGGATGTT (SEQ ID NO: | | |
| | | 558) | | |
| mL20-1 | HQRDMSMLWEL | CATCAACGTGATATGTCTATGCTTT | 5 gly | C |
| | LDVLDGLRQYS | GGGAACTTCTTGATGTTCTTGATGG | | |
| | | TCTTCGTCAATATTCT (SEQ ID NO: | | |
| | | 559) | | |
| mL20-2 | TQRDMSMLDGLL | ACTCAACGTGATATGTCTATGCTTG | 5 gly | C |
| | EVLDQLRQQR | ATGGTCTTCTTGAAGTTCTTGATCA | | |
| | | ACTTCGTCAACAACGT (SEQ ID | | |
| | | NO: 560) | | |
| mL20-3 | TSRDMSLLWELL | ACCTCCCGTGACATGTCCCTGCTGT | 5 gly | C |
| | EELDRLGHQR | GGGAACTGCTGGAAGAACTGGACC | | |
| | | GTCTGGGTCACCAGCGT (SEQ ID | | |
| | | NO: 561) | | |
| mL20-4 | MQHDMSMLYGL | ATGCAACATGATATGTCTATGCTTT | 5 gly | C |
| | VELLESLGHQI | ATGGTCTTGTTGAACTTCTTGAATC | | |
| | | TCTTGGTCATCAAATT (SEQ ID NO: | | |
| | | 562) | | |
| mL20-5 | WNRDMRMLESL | TGGAATCGTGATATGCGTATGCTTG | 5 gly | C |
| | FEVLDGLRQQV | AATCTCTTTTTGAAGTTCTTGATGG | | |
| | | TCTTCGTCAACAAGTT (SEQ ID NO: | | |
| | | 563) | | |
| mL20-6 | GYRDMSMLEGLL | GGTTATCGTGATATGTCTATGCTTG | 5 gly | C |
| | AVLDRLGPQL | AAGGTCTTCTTGCTGTTCTTGATCG | | |
| | | TCTTGGTCCACAACTT (SEQ ID NO: | | |
| | | 564) | | |
| mL20 Con1 | TQRDMSMLEGLL | ACTCAACGTGATATGTCTATGCTTG | 5 gly | C |
| | EVLDRLGQQR | AAGGTCTTCTTGAAGTTCTTGATCG | | |
| | | TCTTGGTCAACAACGT (SEQ ID NO: | | |
| | | 565) | | |
| mL20 Con2 | WYRDMSMLEGL | TGGTACCGTGACATGTCCATGCTG | 5 gly | C |
| | LEVLDRLGQQR | GAAGGTCTGCTGGAAGTTCTGGAC | | |
| | | CGTCTGGGTCAGCAGCGT (SEQ ID | | |
| | | NO: 566) | | |
| mL21-1 | TQNSRQMLLSDF | ACTCAAAATTCTCGTCAAATGCTTC | 5 gly | C |
| | MMLVGSMIQG | TTTCTGATTTTATGATGCTTGTTGG | | |
| | | TTCTATGATTCAAGGT (SEQ ID NO: | | |
| | | 567) | | |
| mL21-2 | MQTSRHILLSEFM | ATGCAAACTTCTCGTCATATTCTTC | 5 gly | C |
| | MLVGSIMHG | TTTCTGAATTTATGATGCTTGTTGG | | |
| | | TTCTATTATGCATGGT (SEQ ID NO: | | |
| | | 568) | | |
| mL21-3 | HDNSRQMLLSDL | CACGACAACTCCCGTCAGATGCTG | 5 gly | C |
| | LHLVGTMIQG | CTGTCCGACCTGCTGCACCTGGTTG | | |
| | | GTACCATGATCCAGGGT (SEQ ID | | |
| | | NO: 569) | | |
| mL21-4 | MENSRQNLLRELI | ATGGAAAACTCCCGTCAGAACCTG | 5 gly | C |
| | MLVGNMSHQ | CTGCGTGAACTGATCATGCTGGTTG | | |
| | | GTAACATGTCCCACCAG (SEQ ID | | |
| | | NO: 570) | | |
| mL21-5 | QDTSRHMLLREF | CAGGACACCTCCCGTCACATGCTG | 5 gly | C |
| | MMLVGEMIQG | CTGCGTGAATTCATGATGCTGGTTG | | |
| | | GTGAAATGATCCAGGGT (SEQ ID | | |
| | | NO: 571) | | |
| mL21 Con1 | DQNSRQMLLSDL | GACCAGAACTCCCGTCAGATGCTG | 5 gly | C |
| | MILVGSMIQG | CTGTCCGACCTGATGATCCTGGTTG | | |
| | | GTTCCATGATCCAGGGT (SEQ ID | | |
| | | NO: 572) | | |
| mTN8-19-1 | VALHGQCTRWP | GTTGCTCTTCATGGTCAATGTACTC | 5 gly | C |
| | WMCPPQREG | GTTGGCCATGGATGTGTCCACCAC | | |
| | | AACGTGAAGGT (SEQ ID NO: 573) | | |
| mTN8-19-2 | YPEQGLCTRWPW | TATCCAGAACAAGGTCTTTGTACTC | 5 gly | C |
| | MCPPQTLA | GTTGGCCATGGATGTGTCCACCAC | | |
| | | AAACTCTTGCT (SEQ ID N: 574) | | |
| mTN8-19-3 | GLNQGHCTRWP | GGTCTGAACCAGGGTCACTGCACC | 5 gly | C |
| | WMCPPQDSN | CGTTGGCCGTGGATGTGCCCGCCG | | |
| | | CAGGACTCCAAC (SEQ ID NO: 575) | | |
| mTN8-19-4 | MITQGQCTRWPW | ATGATTACTCAAGGTCAATGTACTC | 5 gly | C |
| | MCPPQPSG | GTTGGCCATGGATGTGTCCACCAC | | |
| | | AACCATCTGGT (SEQ ID NO: 576) | | |
| mTN8-19-5 | AGAQEHCTRWP | GCTGGTGCTCAGGAACACTGCACC | 5 gly | C |
| | WMCAPNDWI | CGTTGGCCGTGGATGTGCGCTCCG | | |
| | | AACGACTGGATC (SEQ ID NO: 577) | | |
| mTN8-19-6 | GVNQGQCTRWR | GGTGTTAACCAGGGTCAGTGCACC | 5 gly | C |
| | WMCPPNGWE | CGTTGGCGTTGGATGTGCCCGCCG | | |
| | | AACGGTTGGGAA (SEQ ID NO: | | |
| | | 578) | | |
| mTN8-19-7 | LADHGQCIRWPW | | 5 gly | C |
| | MCPPEGWE | CTGGCTGACCACGGTCAGTGCATC | | |
| | | CGTTGGCCGTGGATGTGCCCGCCG | | |
| | | GAAGGTTGGGAA (SEQ ID NO: 579) | | |
| mTN8-19-8 | ILEQAQCTRWPW | ATCCTGGAACAGGCTCAGTGCACC | 5 gly | C |
| | MCPPQRGG | CGTTGGCCGTGGATGTGCCCGCCG | | |
| | | CAGCGTGGTGGT (SEQ ID NO: 580) | | |
| mTN8-19-9 | TQTHAQCTRWP | ACTCAAACTCATGCTCAATGTACTC | 5 gly | C |
| | WMCPPQWEG | GTTGGCCATGGATGTGTCCACCAC | | |
| | | AATGGGAAGGT (SEQ ID NO: 581) | | |
| mTN8-19-10 | VVTQGHCTLWP | GTTGTTACTCAAGGTCATTGTACTC | 5 gly | C |
| | WMCPPQRWR | TTTGGCCATGGATGTGTCCACCACA | | |
| | | ACGTTGGCGT (SEQ ID NO: 582) | | |
| mTN8-19-11 | IYPHDQCTRWPW | ATTTATCCACATGATCAATGTACTC | 5 gly | C |
| | MCPPQPYP | GTTGGCCATGGATGTGTCCACCAC | | |
| | | AACCATATCCA (SEQ ID NO: 583) | | |
| mTN8-19-12 | SYWQGQCTRWP | TCTTATTGGCAAGGTCAATGTACTC | 5 gly | C |
| | WMCPPQWRG | GTTGGCCATGGATGTGTCCACCAC | | |
| | | AATGGCGTGGT (SEQ ID NO: 584) | | |
| mTN8-19-13 | MWQQGHCTRWP | ATGTGGCAACAAGGTCATTGTACT | 5 gly | C |
| | WMCPPQGWG | CGTTGGCCATGGATGTGTCCACCA | | |
| | | CAAGGTTGGGGT (SEQ ID NO: 585) | | |
| mTN8-19-14 | EFTQWHCTRWP | GAATTCACCCAGTGGCACTGCACC | 5 gly | C |
| | WMCPPQRSQ | CGTTGGCCGTGGATGTGCCCGCCG | | |
| | | CAGCGTTCCCAG (SEQ ID NO: 586) | | |
| mTN8-19-15 | LDDQWQCTRWP | CTGGACGACCAGTGGCAGTGCACC | 5 gly | C |
| | WMCPPQGFS | CGTTGGCCGTGGATGTGCCCGCCG | | |
| | | CAGGGTTTCTCC (SEQ ID NO: 587) | | |
| mTN8-19-16 | YQTQGLCTRWP | TATCAAACTCAAGGTCTTTGTACTC | 5 gly | C |
| | WMCPPQSQR | GTTGGCCATGGATGTGTCCACCAC | | |
| | | AATCTCAACGT (SEQ ID NO: 588) | | |
| mTN8-19-17 | ESNQGQCTRWP | GAATCTAATCAAGGTCAATGTACT | 5 gly | C |
| | WMCPPQGGW | CGTTGGCCATGGATGTGTCCACCA | | |
| | | CAAGGTGGTTGG (SEQ ID NO: 589) | | |
| mTN8-19-18 | WTDRGPCTRWP | TGGACCGACCGTGGTCCGTGCACC | 5 gly | C |
| | WMCPPQANG | CGTTGGCCGTGGATGTGCCCGCCG | | |
| | | CAGGCTAACGGT (SEQ ID NO: 590) | | |
| mTN8-19-19 | VGTQGQCTRWP | GTTGGTACCCAGGGTCAGTGCACC | 5 gly | C |
| | WMCPPYETG | CGTTGGCCGTGGATGTGCCCGCCG | | |
| | | TACGAAACCGGT (SEQ ID NO: 591) | | |
| mTN8-19-20 | PYEQGKCTRWP | CCGTACGAACAGGGTAAATGCACC | 5 gly | C |
| | WMCPPYEVE | CGTTGGCCGTGGATGTGCCCGCCG | | |
| | | TACGAAGTTGAA (SEQ ID NO: 592) | | |
| mTN8-19-21 | SEYQGLCTRWPW | TCCGAATACCAGGGTCTGTGCACC | 5 gly | C |
| | MCPPQGWK | CGTTGGCCGTGGATGTGCCCGCCG | | |
| | | CAGGGTTGGAAA (SEQ ID NO: 593) | | |
| mTN8-19-22 | TFSQGHCTRWPW | ACCTTCTCCCAGGGTCACTGCACCC | 5 gly | C |
| | MCPPQGWG | GTTGGCCGTGGATGTGCCCGCCGC | | |
| | | AGGGTTGGGGT (SEQ ID NO: 594) | | |
| mTN8-19-23 | PGAHDHCTRWP | CCGGGTGCTCACGACCACTGCACC | 5 gly | C |
| | WMCPPQSRY | CGTTGGCCGTGGATGTGCCCGCCG | | |
| | | CAGTCCCGTTAC (SEQ ID NO: 595) | | |
| mTN8-19-24 | VAEEWHCRRWP | GTTGCTGAAGAATGGCACTGCCGT | 5 gly | C |
| | WMCPPQDWR | CGTTGGCCGTGGATGTGCCCGCCG | | |
| | | CAGGACTGGCGT (SEQ ID NO: 596) | | |
| mTN8-19-25 | VGTQGHCTRWP | GTTGGTACCCAGGGTCACTGCACC | 5 gly | C |
| | WMCPPQPAG | CGTTGGCCGTGGATGTGCCCGCCG | | |
| | | CAGCCGGCTGGT (SEQ ID NO: 597) | | |
| mTN8-19-26 | EEDQAHCRSWP | GAAGAAGACCAGGCTCACTGCCGT | 5 gly | C |
| | WMCPPQGWV | TCCTGGCCGTGGATGTGCCCGCCG | | |
| | | CAGGGTTGGGTT (SEQ ID NO: 598) | | |
| mTN8-19-27 | ADTQGHCTRWP | GCTGACACCCAGGGTCACTGCACC | 5 gly | C |
| | WMCPPQHWF | CGTTGGCCGTGGATGTGCCCGCCG | | |
| | | CAGCACTGGTTC (SEQ ID NO: 599) | | |
| mTN8-19-28 | SGPQGHCTRWPW | TCCGGTCCGCAGGGTCACTGCACC | 5 gly | C |
| | MCAPQGWF | CGTTGGCCGTGGATGTGCGCTCCG | | |
| | | CAGGGTTGGTTC (SEQ ID NO: 600) | | |
| mTN8-19-29 | TLVQGHCTRWP | ACCCTGGTTCAGGGTCACTGCACC | 5 gly | C |
| | WMCPPQRWV | CGTTGGCCGTGGATGTGCCCGCCG | | |
| | | CAGCGTTGGGTT (SEQ ID NO: 601) | | |
| mTN8-19-30 | GMAHGKCTRWA | GGTATGGCTCACGGTAAATGCACC | 5 gly | C |
| | WMCPPQSWK | CGTTGGGCTTGGATGTGCCCGCCG | | |
| | | CAGTCCTGGAAA (SEQ ID NO: 602) | | |
| mTN8-19-31 | ELYHGQCTRWP | GAACTGTACCACGGTCAGTGCACC | 5 gly | C |
| | WMCPPQSWA | CGTTGGCCGTGGATGTGCCCGCCG | | |
| | | CAGTCCTGGGCT (SEQ ID NO: 603) | | |
| mTN8-19-32 | VADHGHCTRWP | GTTGCTGACCACGGTCACTGCACC | 5 gly | C |
| | WMCPPQGWG | CGTTGGCCGTGGATGTGCCCGCCG | | |
| | | CAGGGTTGGGGT (SEQ ID NO: 604 | | |
| mTN8-19-33 | PESQGHCTRWPW | CCGGAATCCCAGGGTCACTGCACC | 5 gly | C |
| | MCPPQGWG | CGTTGGCCGTGGATGTGCCCGCCG | | |
| | | CAGGGTTGGGGT (SEQ ID NO: 605) | | |
| mTN8-19-34 | IPAHGHCTRWPW | | 5 gly | C |
| | MCPPQRWR | ATCCCGGCTCACGGTCACTGCACC | | |
| | | CGTTGGCCGTGGATGTGCCCGCCG | | |
| | | CAGCGTTGGCGT (SEQ ID NO: 606) | | |
| mTN8-19-35 | FTVHGHCTRWP | TTCACCGTTCACGGTCACTGCACCC | 5 gly | C |
| | WMCPPYGWV | GTTGGCCGTGGATGTGCCCGCCGT | | |
| | | ACGGTTGGGTT (SEQ ID NO: 607) | | |
| mTN8-19-36 | PDFPGHCTRWRW | CCAGATTTTCCAGGTCATTGTACTC | 5 gly | C |
| | MCPPQGWE | GTTGGCGTTGGATGTGTCCACCAC | | |
| | | AAGGTTGGGAA (SEQ ID NO: 608) | | |
| mTN8-19-37 | QLWQGPCTQWP | CAGCTGTGGCAGGGTCCGTGCACC | 5 gly | C |
| | WMCPPKGRY | CAGTGGCCGTGGATGTGCCCGCCG | | |
| | | AAAGGTCGTTAC (SEQ ID NO: 609) | | |
| mTN8-19-38 | HANDGHCTRWQ | CACGCTAACGACGGTCACTGCACC | 5 gly | C |
| | WMCPPQWGG | CGTTGGCAGTGGATGTGCCCGCCG | | |
| | | CAGTGGGGTGGT (SEQ ID NO: 610) | | |
| mTN8-19-39 | ETDHGLCTRWPW | GAAACCGACCACGGTCTGTGCACC | 5 gly | C |
| | MCPPYGAR | CGTTGGCCGTGGATGTGCCCGCCG | | |
| | | TACGGTGCTCGT (SEQ ID NO: 611) | | |
| mTN8-19-40 | GTWQGLCTRWP | GGTACCTGGCAGGGTCTGTGCACC | 5 gly | C |
| | WMCPPQGWQ | CGTTGGCCGTGGATGTGCCCGCCG | | |
| | | CAGGGTTGGCAG (SEQ ID NO: 612) | | |
| mTN8-19 Con1 | VATQGQCTRWP | GTTGCTACCCAGGGTCAGTGCACC | 5 gly | C |
| | WMCPPQGWG | CGTTGGCCGTGGATGTGCCCGCCG | | |
| | | CAGGGTTGGGGT (SEQ ID NO: 613) | | |
| mTN8-19 Con2 | VATQGQCTRWP | GTTGCTACCCAGGGTCAGTGCACC | 5 gly | C |
| | WMCPPQRWG | CGTTGGCCGTGGATGTGCCCGCCG | | |
| | | CAGCGTTGGGGT (SEQ ID NO: 614) | | |
| 2X mTN8-19-7 | FC-5G-AQ | CTTGCTGATCATGGTCAATGTATTC | 1K | C |
| | LADHGQCIRWPW | GTTGGCCATGGATGTGTCCACCAG | | |
| | MCPPEGWELEGS | AAGGTTGGGAACTCGAGGGTTCCG | | |
| | GSATGGSGSTASS | GTTCCGCTACCGGCGGCTCTGGCTC | | |
| | GSGSATGLADHG | CACTGCTTCTTCCGGTTCCGGTTCT | | |
| | QCIRWPWMCPPE | GCTACTGGTCTGGCTGACCACGGT | | |
| | GWE-LE (SEQ ID | CAGTGCATCCGTTGGCCGTGGATG | | |
| | NO: 615) | TGCCCGCCGGAAGGTTGGGAACTG | | |
| | | GAA (SEQ ID NO: 616) | | |
| 2X mTN8-19-7 | FC-5G-AQ- | CTTGCTGATCATGGTCAATGTATTC | 1K | C |
| ST-GG de12x | LADHGQCIRWPW | GTTGGCCATGGATGTGTCCACCAG | | |
| LE | MCPPEGWEGSGS | AAGGTTGGGAAGGTTCCGGTTCCG | | |
| | ATGGSGGGASSG | CTACCGGCGGCTCTGGCGGTGGCG | | |
| | SGSATGLADHGQ | CTTCTTCCGGTTCCGGTTCTGCTAC | | |
| | CIRWPWMCPPEG | TGGTCTGGCTGACCACGGTCAGTG | | |
| | WE (SEQ ID NO: | CATCCGTTGGCCGTGGATGTGTCCA | | |
| | 617) | CCAGAAGGTTGGGAA (SEQ ID NO: | | |
| | | 618) | | |
| 2X mTN8-19-21 | FC-5G-AQ- | TCTGAATATCAAGGTCTTTGTACTC | 1K | C |
| | SEYQGLCTRWPW | GTTGGCCATGGATGTGTCCACCAC | | |
| | MCPPQGWKLEGS | AAGGTTGGAAACTCGAGGGTTCCG | | |
| | GSATGGSGSTASS | GTTCCGCTACCGGCGGCTCTGGCTC | | |
| | GSGSATGSEYQG | CACTGCTTCTTCCGGTTCCGGTTCT | | |
| | LCTRWPWMCPPQ | GCTACTGGTTCTGAGTATCAAGGC | | |
| | GWK-LE (SEQ | CTCTGTACTCGCTGGCCATGGATGT | | |
| | ID NO: 619) | GTCCACCACAAGGCTGGAAGCTGG | | |
| | | AA (SEQ ID NO: 620) | | |
| 2X mTN8-19-21 | FC-5G-AQ- | TCTGAATATCAAGGTCTTTGTACTC | 1K | C |
| ST-GG de12x | SEYQGLCTRWPW | GTTGGCCATGGATGTGTCCACCAC | | |
| LE | MCPPQGWKGSGS | AAGGTTGGAAAGGTTCCGGTTCCG | | |
| | ATGGSGGGASSG | CTACCGGCGGCTCTGGCGGTGGCG | | |
| | SGSATGSEYQGL | CTTCTTCCGGTTCCGGTTCTGCTAC | | |
| | CTRWPWMCPPQ | TGGTTCTGAGTATCAAGGCCTCTGT | | |
| | GWK (SEQ ID NO: | ACTCGCTGGCCATGGATGTGTCCA | | |
| | 621) | CCACAAGGTTGGAAA (SEQ ID NO: | | |
| | | 622) | | |
| 2X mTN8-19-22 | FC-5G-AQ- | ACTTTTTCTCAAGGTCATTGTACTC | 1K | C |
| | TFSQGHCTRWPW | GTTGGCCATGGATGTGTCCACCAC | | |
| | MCPPQGWGLEGS | AAGGTTGGGGTCTCGAGGGTTCCG | | |
| | GSATGGSGSTASS | GTTCCGCTACCGGCGGCTCTGGCTC | | |
| | GSGSATGTFSQG | CACTGCTTCTTCCGGTTCCGGTTCT | | |
| | HCTRWPWMCPP | GCTACTGGTACTTTTTCTCAAGGCC | | |
| | QGWG-LE (SEQ | ATTGTACTCGCTGGCCATGGATGTG | | |
| | ID NO: 623) | TCCACCACAAGGCTGGGGCCTGGA | | |
| | | A (SEQ ID NO: 624) | | |
| 2X mTN8-19-32 | FC-5G-AQ- | GTTGCTGATCATGGTCATTGTACTC | 1K | C |
| | VADHGHCTRWP | GTTGGCCATGGATGTGTCCACCAC | | |
| | WMCPPQGWGLE | AAGGTTGGGGTCTCGAGGGTTCCG | | |
| | GSGSATGGSGST | GTTCCGCAACCGGCGGCTCTGGCT | | |
| | ASSGSGSATGVA | CCACTGCTTCTTCCGGTTCCGGTTC | | |
| | DHGHCTRWPWM | TGCTACTGGTGTTGCTGACCACGGT | | |
| | CPPQGWG-LE | CACTGCACCCGTTGGCCGTGGATG | | |
| | (SEQ ID NO: 625) | TGCCCGCCGCAGGGTTGGGGTCTG | | |
| | | GAA (SEQ ID NO: 626) | | |
| 2X mTN8-19-32 | FC-5G-AQ- | GTTGCTGATCATGGTCATTGTACTC | 1K | C |
| ST-GG de12x | VADHGHCTRWP | GTTGGCCATGGATGTGTCCACCAC | | |
| LE | WMCPPQGWGGS | AAGGTTGGGGTGGTTCCGGTTCCG | | |
| | GSATGGSGGGAS | CTACCGGCGGCTCTGGCGGTGGTG | | |
| | SGSGSATGVADH | CTTCTTCCGGTTCCGGTTCTGCTAC | | |
| | GHCTRWPWVCPP | TGGTGTTGCTGACCACGGTCACTGC | | |
| | QGWG (SEQ ID | ACCCGTTGGCCGTGGGTGTGTCCA | | |
| | NO: 627) | CCACAAGGTTGGGGT (SEQ ID NO: | | |
| | | 628) | | |
| 2X mTN8-19-33 | FC-5G-AQ- | CCAGAATCTCAAGGTCATTGTACTC | 1K | C |
| | PESQGHCTRWPW | GTTGGCCATGGATGTGTCCACCAC | | |
| | MCPPQGWGLEGS | AAGGTTGGGGTCTCGAGGGTTCCG | | |
| | GSATGGSGSTASS | GTTCCGCTACCGGCGGCTCTGGCTC | | |
| | GSGSATGPESQG | CACTGCTTCTTCCGGTTCCGGTTCT | | |
| | HCTRWPWMCPP | GCTACTGGTCCGGAATCCCAGGGT | | |
| | QGWGLE (SEQ | CACTGCACCCGTTGGCCGTGGATG | | |
| | ID NO: 629) | TGCCCGCCGCAGGGTTGGGGTCTG | | |
| | | GAA (SEQ ID NO: 630) | | |
| 2X mTN8-19-33 | FC-5G-AQ- | CCAGAATCTCAAGGTCATTGTACTC | 1K | C |
| ST-GG de12x | PESQGHCTRWPW | GTTGGCCATGGATGTGTCCACCAC | | |
| LE | MCPPQGWGGSGS | AAGGTTGGGGTGGTTCCGGTTCCG | | |
| | ATGGSGGGASSG | CTACCGGCGGCTCTGGCGGTGGTG | | |
| | SGSATGPESQGH | CTTCTTCCGGTTCCGGTTCTGCTAC | | |
| | CTRWPWMCP | TGGTCCGGAATCCCAGGGTCACTG | | |
| | PQGWG (SEQ ID | CACCCGTTGGCCGTGGATGTGTCC | | |
| | NO: 631) | ACCACAAGGTTGGGGT (SEQ ID | | |
| | | NO: 632) | | |

### Example 7

### In Vitro Screening of Affinity Matured Peptibodies

The following exemplary peptibodies were screened according to the protocols set forth above to obtain the following K_{D} and IC₅₀ values. Table VII shows the range of K_{D} values for selected affinity matured peptibodies compared with the parent peptibodies, as determined by KinExA™ solution based assays or BIAcore® assays. These values demonstrate increased binding affinity of the affinity matured peptibodies for myostatin compared with the parent peptibodies. Table VIII shows IC₅₀ values for a number of affinity matured peptibodies. A range of values is given in this table.

TABLE VII
K_{D} values for selected affinity matured peptibodies

| peptibodies | K_{D} |
|---|---|
| TN8-19 (parent) | >1 nM |
| 2xmTN8-19 (parent) | >1 nM |
| 1x mTN8-19-7 | 10 pM |
| 2x mTN8-19-7 | 12 pM |
| 1x mTN8-19-21 | 6 pM |
| 2x mTN8-19-21 | 6 pM |
| 1x mTN8-19-32 | 9 pM |
| 1x mTN8-19-33 | 21 pM |
| 2x mTN8-19-33 | 3 pM |
| 1x mTN8-19-22 | 4 pM |
| 1x mTN8-19-con1 | 20 pM |

TABLE VIII
IC₅₀ values for selected affinity matured peptibodies

| Affinity Matured Peptibody | IC₅₀ (nM) |
|---|---|
| mTN8-19 Con1 | 1.0-4.4 |
| mTN8-19-2 | 7.508-34.39 |
| mTN8-19-4 | 16.74 |
| mTN8-19-5 | 7.743-3.495 |
| mTN8-19-6 | 17.26 |
| mTN8-19-7 | 1.778 |
| mTN8-19-9 | 22.96-18.77 |
| mTN8-19-10 | 5.252-7.4 |
| mTN8-19-11 | 28.66 |
| mTN8-19-12 | 980.4 |
| mTN8-19-13 | 20.04 |
| mTN8-19-14 | 4.065-6.556 |
| mTN8-19-16 | 4.654 |
| mTN8-19-21 | 2.767-3.602 |
| mTN8-19-22 | 1.927-3.258 |
| mTN8-19-23 | 6.584 |
| mTN8-19-24 | 1.673-2.927 |
| mTN8-19-27 | 4.837-4.925 |
| mTN8-19-28 | 4.387 |
| mTN8-19-29 | 6.358 |
| mTN8-19-32 | 1.842-3.348 |
| mTN8-19-33 | 2.146-2.745 |
| mTN8-19-34 | 5.028-5.069 |
| mTN8Con6-3 | 86.81 |
| mTN8Con6-5 | 2385 |
| mTN8-19-7(-LE) | 1.75-2.677 |
| mTN8-19-21(-LE) | 2.49 |
| mTN8-19-33(-LE) | 1.808 |
| 2xmTN8-19-7 | 0.8572-2.649 |
| 2xmTN8-19-9 | 1.316-1.228 |
| 2xmTN8-19-14 | 1.18-1.322 |
| 2xmTN8-19-16 | 0.9903-1.451 |
| 2xmTN8-19-21 | 0.828-1.434 |
| 2xmTN8-19-22 | 0.9937-1.22 |
| 2xmTN8-19-27 | 1.601-3.931 |
| 2xmTN8-19-7(-LE) | 1.077-1.219 |
| 2xmTN8-19-21(-LE) | 0.8827-1.254 |
| 2xmTN8-19-33(-LE) | 1.12-1.033 |
| mL2-7 | 90.24 |
| mL2-9 | 105.5 |
| mL15-7 | 32.75 |
| mL15-9 | 354.2 |
| mL20-2 | 122.6 |
| mL20-3 | 157.9 |
| mL20-4 | 160 |

### Example 8

### In Vivo Anabolic Activity of Exemplary Peptibodies

The CD1 nu/nu mouse model (Charles River Laboratories, Massachusettes) was used to determine the in vivo efficacy of the peptibodies of the present invention which included the human Fc region (huFc). This model responded to the inhibitors of the present invention with a rapid anabolic response which was associated with increased dry muscle mass and an increase in myofibrillar proteins but was not associated with accumulation in body water content.

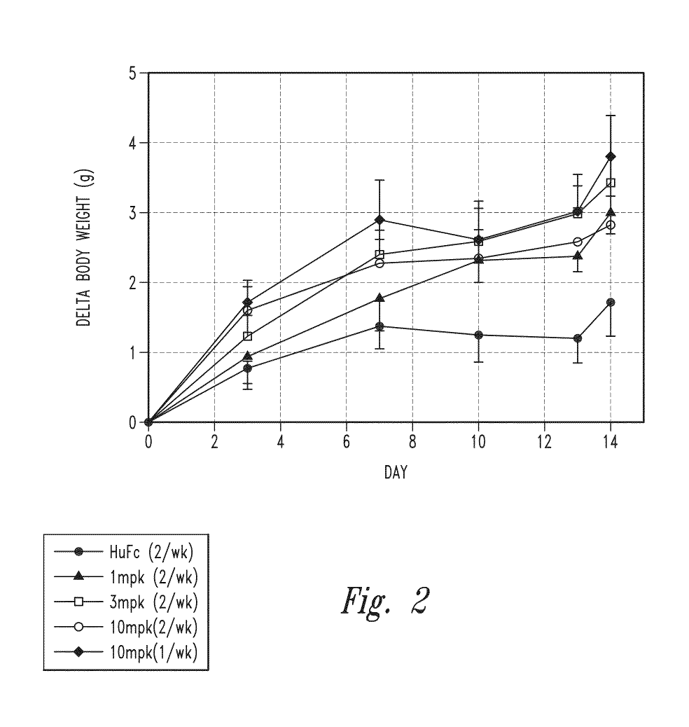

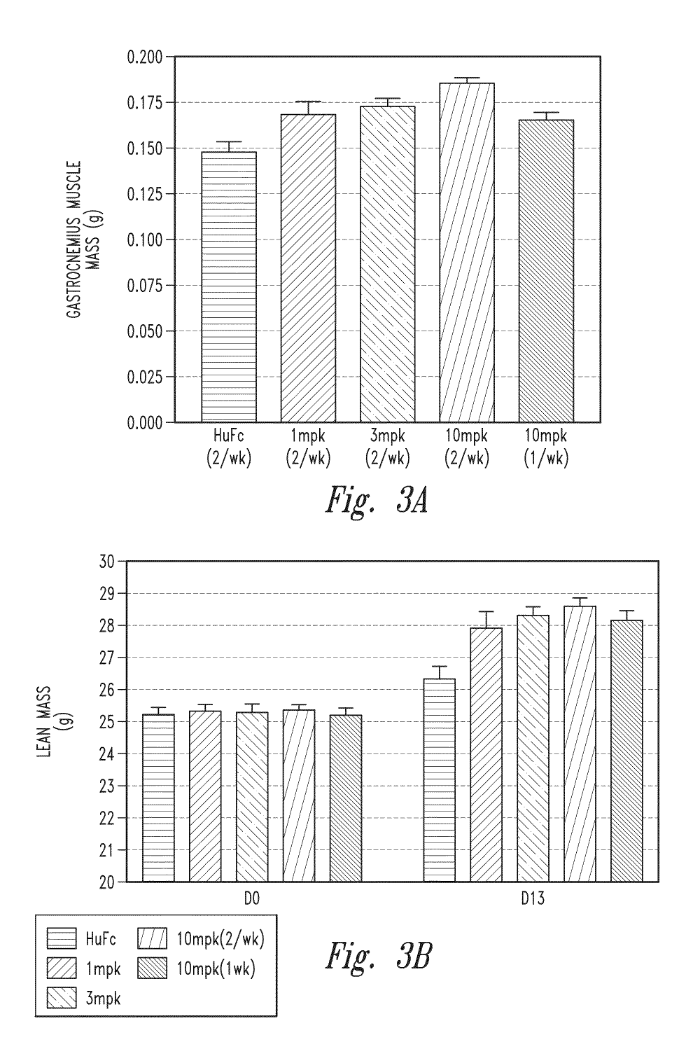

In one example, the efficacy of 1x peptibody mTN8-19-21 in vivo was demonstrated by the following experiment. A group of 10 8 week old CD1 nu/nu mice were treated twice weekly or once weekly with dosages of 1 mg/kg, 3 mg/kg and 10 mg/kg (subcutaneous injection). The control group of 10 8 week old CD1 nu/nu mice received a twice weekly (subcutaneous) injection of huFc (vehicle) at 10 mg/kg. The animals were weighed every other day and lean body mass determined by NMR on day 0 and day 13. The animals are then sacrified at day 14 and the size of the gastrocnemius muscle determined. The results are shown in FIGS. 2 and 3. FIG. 2 shows the increase in total body weight of the mice over 14 days for the various dosages of peptibody compared with the control. As can be seen from FIG. 2 all of the dosages have show an increase in body weight compared with the control, with all of the dosages showing statistically significant increases over the control by day 14. FIG. 3 shows the change in lean body mass on day 0 and day 13 as determined by nuclear magnetic resonance (NMR) imaging (EchoMRI 2003, Echo Medical Systems, Houston, Tx), as well as the change in weight of the gastrocnemius muscle dissected from the animals at day 14.

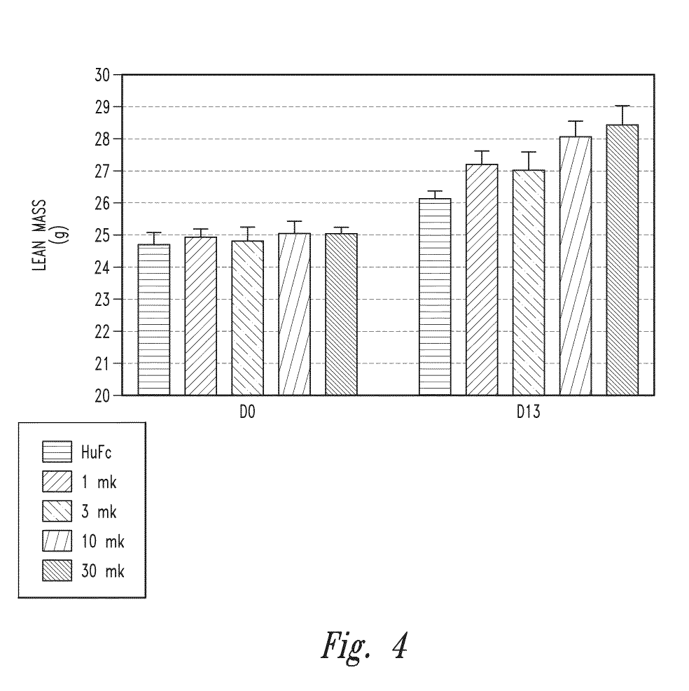

In another example, the 1x mTN8-19-32 peptibody was administered to CD1 nu/nu mice in a biweekly injection of 1 mg/kg, 3 mg/kg, 10 mg/kg, and 30 mg/kg compared with the huFc control (vehicle). The peptibody-treated animals show an increase in total body weight (not shown) as well as lean body mass on day 13 compared with day 0 as determined by NMR measurement. The increase in lean body mass is shown in FIG. 4.

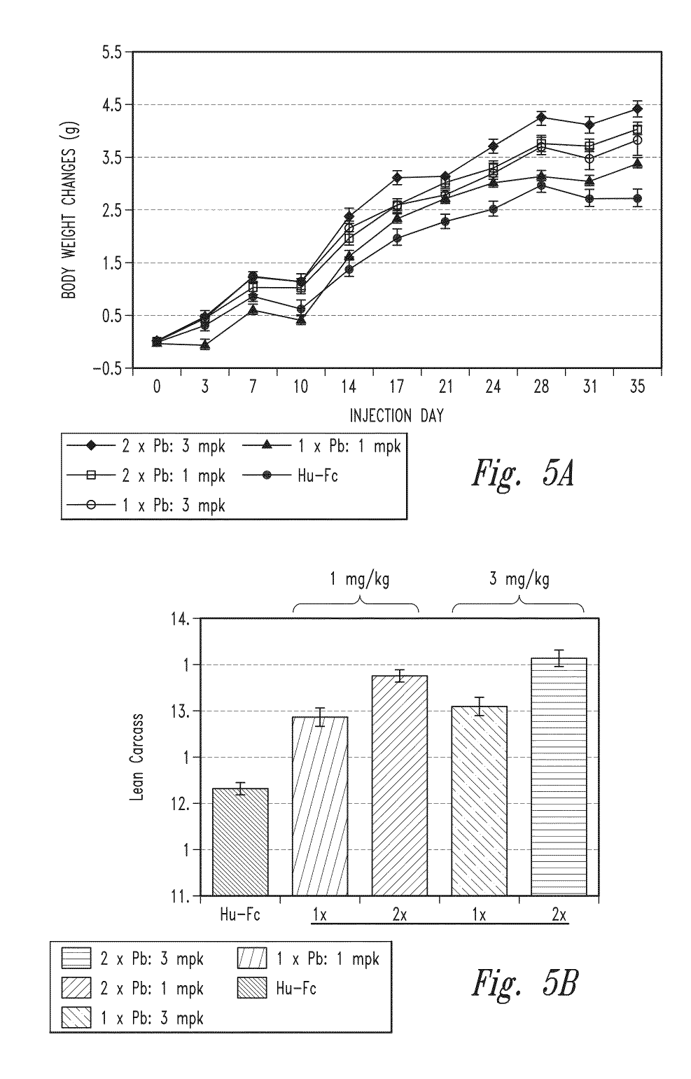

In another example, a 1x affinity-matured peptibody was compared with a 2x affinity-matured peptibody for in vivo anabolic efficacy. CD1 nu/nu male mice (10 animals per group) were treated with twice weekly injections of 1 mg/kg and 3 mg/kg of 1x mTN8-19-7 and 2x mTN8-19-7 for 35 days, while the control group (10 animals) received twice weekly injections of huFc (3 mg/kg). As shown in FIG. 5, treatment with the 2x peptibody resulted in a greater body weight gain and leans carcass weight at necropsy compared with the 1x peptibody or control.

### Example 9

### Increase in Muscular Strength

Normal age-matched male 4 month old male C57B1/6 mice were treated for 30 days with 2 injections per week subcutaneous injections 5 mg/kg per week of 2x mTN8-19-33, 2x mTN8-19-7, and huFc vehicle control group (10 animals/group). The animals were allowed to recover without any further injections. Gripping strength was measured on day 18 of the recovery period. Griping strength was measured using a Columbia Instruments meter, model 1027 dsm (Columbus, Ohio). Peptibody treatment resulted in significant increase in gripping strength, with 2x mTN8-19-33 pretreated animals showing a 14% increase in gripping strength compared with the control-treated mice, while 2x mTN8-19-7 showed a 15% increase in gripping strength compared with the control treated mice.

### Example 10

### Pharmacokinetics

In vivo phamacokinetics experiments were performed using representative peptibodies without the LE sequences. 10 mg/kg and 5 mg/kg dosages were administered to CD1 nu/nu mice and the following parameters determined: Cmax (ug/mL), area under the curve (AUC) (ug-hr/mL), and half-life (hr). It was found that the 2x versions of the affinity matured peptibodies have a significantly longer half-life than the 1x versions. For example 1x affinity matured mTN8-19-22 has a half-life in the animals of about 50.2 hours, whereas 2x mTN8-19-22 has a half-life of about 85.2 hours. Affinity matured 1x mTN8-7 has a half-life of about 65 hours, whereas 2x mTN8-19-7 has a half-life of about 106 hours.

### Example 11

### Treatment of Mdx Mice

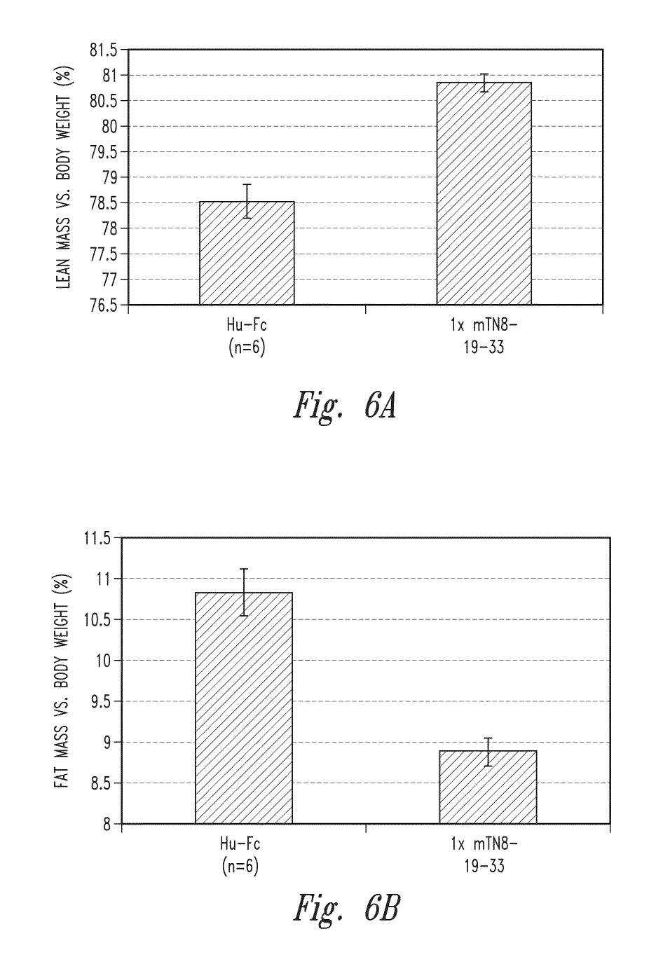

The peptibodies of the present invention have been shown to increase lean muscle mass in an animal and are useful for the treatment of a variety of disorders which involve muscle wasting. Muscular dystrophy is one of those disorders. The mouse model for Duchenne's muscular dystrophy is the Duchenne mdx mouse (Jackson Laboratories, Bar Harbor, Me.). Aged (10 month old) mdx mice were injected with either the peptibody 1x mTN8-19-33 (n=8/group) or with the vehicle huFc protein (N=6/group) for a three month period of time. The dosing schedule was every other day, 10 mg/kg, by subcutaneous injection. The peptibody treatment had a positive effect on increasing and maintaining body mass for the aged mdx mice. Significant increases in body weight were observed in the peptibody-treated group compared to the hu-Fc-treated control group, as shown in FIG. 6A. In addition, NMR analysis revealed that the lean body mass to fat mass ratio was also significantly increased in the aged mdx mice as a result of the peptibody treatment compared with the control group, and that the fat percentage of body weight decreased in the peptibody treated mice compared with the control group, as shown in FIG. 6B.

### Example 12

### Treatment of CIA Arthritis Mouse Model

The collagen-induced arthritis mouse model is widely used as a model for rheumatoid arthritis. 8 week old DBA/1J mice (Jackson Labs, Bar Harbor, Me.) were immunized on day 1 and day 21 of the experiment with 100 ug bovine collagen II (Chrondex, Redmond, Wash.) at the base of the tail to induce arthritis. Arthritic conditions of the mice were scored by joint and paw redness and/or swelling, and animals were selected on this basis. Three groups of animals were established: normal animals not receiving collagen (normal, 12 animals), animals receiving collagen plus a murine Fc vehicle (CIA/vehicle, 6 animals), and animals receiving collagen plus the peptibody 2x mTN8-19-21 attached to a murine Fc (2x mTN8-19-21/muFc, also referred to as 2x-21) (CIA/peptibody, 8 animals). The murine Fc used in these experiments and in the examples below is an Fc from a murine IgG. The CIA/vehicle animals and the CIA/peptibody animals, in addition to receiving collagen on day 1 and day 21, were injected subcutaneously (s.c.) with 5 mg/kg myostatin peptibody 2x mTN8-19-21/muFc or murine Fc vehicle alone twice a week begining on day 8 and continuing to day 50. The animals were weighed every four days. The results are shown in FIG. 7. FIG. 7 shows an increase in body weight for CIA/peptibody (2x21) animals compared with CIA/vehicle animals who lost weight, indicating that myostatin antagonists including the peptibodies described herein can counteract the rheumatoid cachexia displayed in the control animals.

### Example 13

### Treatment of Orchietomized Mice

The following example describes the treatment of orchietomized C57B1/6 mice with an exemplary peptibody. Two groups of age and weight matched six month old surgically orchiectomized C57B1/6 mice (Charles River Laboratories, Wilmington, Mass.) were treated with either murine Fc, or with peptibody 2x mTN8-19-21/muFc (11 animals per group). The two groups of mice were injected IP with 3 mg/kg peptibody or murine Fc IP 2x per week. Treatment began 3 weeks after surgery and continued for 10 weeks. Nuclear magnetic resonance (NMR) imaging was performed on each live animal to assess lean mass at the beginning of the study, at 7 weeks and at 10 weeks. As can be seen in the table below, orchietomized mice treated with the murine Fc are beginning to lose lean mass by week 10. Comparison of the orchiectomized group receiving the peptibody vs. the Fc vehicle indicated that the peptibody improved the gain of lean body weight in the orchietomized animals compared with animals treated with murine Fc. This result is shown in the Table below.

| | | lean | lean | | lean | Δ mass |
|---|---|---|---|---|---|---|
| | | mass (g) | mass (g) | Δ mass | mass (g) | week |
| group | | day 0 | week 7 | week 7 | week 10 | 10 |
| orchiectomized | mean wt. | 23.8809 | 24.5691 | 0.6882 | 24.5009 | 0.6200 |
| MuFc | | | | | | |
| orchiectomized | mean wt. | 23.7840 | 25.9473 | 1.7462 | 25.9473 | 2.2318 |
| 2x mTN8-19- | | | | | | |
| 21/muFc | | | | | | |

In addition, treatment of orchiectomized mice with the anti-myostatin peptibody did not result in an increase in testosterone levels. These results show that myostatin antagonists such as the peptibodies described herein can be used to treat androgen deprived states.

### Example 14

### Reduction of TNF-α Levels

Female BALB/c mice, 8-10 weeks, (Charles River Laboratories, Wilmington, Mass.) were pretreated with PBS control or 10 mg/kg of peptibody 2x TN8-19-21/muFc one day before the LPS challenge. There were 5 animals in each group. On day 1, LPS (lipopolysaccharide from E. coli 055, B5 (Sigma) was administered intravenously at 0.5 mg/kg (10 ug/mouse). Serum samples were collected 30 minutes after the LPS administration. mTNF-α (tumor necrosis factor α) levels were measured. The results showed that animals pretreated with the peptibody had reduced levels of mTNF-α in their blood. PBS treated animals averaged approximately 380 pg/ml of mTNF-α in their blood. Peptibody treated animals averaged only approximately 120 pg/ml mTNF-α in their blood. This demonstrates that myostatin antagonists can reduce at least one cytokine responsible for inflammation, contributing to the antagonist's effectiveness in treating rheumatoid arthritis and other immune disorders.

### Example 15

### STZ-Induced Model of Diabetes

The purpose of the following experiments was to determine the effects of myostatin antagonists in the streptozotocin-induced (STZ) induced diabetic animal model. In addition, the experiments were designed to determine if a myostatin antagonist will delay or prevent the progression or development of diabetic nephropathy. The peptibody used was 2x mTN8-19-21 attached to a murine Fc (2x mTN8-19-21/muFc or 2x-21). The control vehicle was murine Fc alone.

Streptozotocin-Induced Diabetes:

A diabetic animal model was created by multiple low dose streptozotocin injection. Eight week old C57B1/6 mice were purchased from Charles River Laboratory. All animals were hosted in individual cages for one week. The animal body weights were measured and then randomly divided into 2 groups (n=20/group). 20 mice were injected with low dose streptozotocin (STZ, Sigma Co.) at 40 mg/kg (dissolved in 0.1 ml of citrate buffer solution) for 5 consecutive days. Another group of 20 mice was injected with vehicle (0.1 ml citrate buffer solution) for 5 consecutive days. The blood glucose levels were measured using glucose oxidase method (Glucometer Elite, Bayer Corp., Elkhart, Ind.). The induction of diabetes was defined by measurement of the blood glucose levels. The blood glucose levels over 11 mmol/L or 200 mg/dl were considered as hyperglycemia. Then the diabetic and age-matched normal mice were maintained for another 4 months. The body weight, food intake and blood glucose levels were measured monthly. Four months after STZ injection, 16 out of 20 mice developed diabetes, and these were used in later studies. The diabetic mice were divided into two treatment groups according their body weight. The age-matched normal mice were also divided into two treatment groups.

Experimental Design:

Starting on day 0, both diabetic groups were subcutaneously injection with vehicle (mu-Fc) or 2x mTN8-19-21 at 5 mg/kg, 3 times per week for 6 weeks. The body weight and food intake were measured 3 times per week. The non-diabetic mice, which had not been injected with STZ were treated with vehicle (muFc) and at the same dose and same schedule for 6 weeks. The blood glucose levels were measured using glucose oxidase method at day 0, day 15, day 30, and at the end of the study. The design of the study is presented in the Table below.

| | | | | | | | Study |
|---|---|---|---|---|---|---|---|
| Group | Animal | Animal | | | Dose | Dosing | Dur- |
| No | group | No. | N | Treatment | (mg/kg) | Schedule | ation |
| 1 | STZ- | 1-8 | 8 | 2x mTN8- | 5 | 3x/week | 6 week |
| | diabetes | | | 19-21/muFc | | | |
| 2 | STZ- | 9-18 | 8 | Vehicle | 5 | 3x/week | 6 week |
| | diabetes | | | (muFc) | | | |
| 3 | Normal | 19-24 | 8 | 2x mTN8- | 5 | 3x/week | 6 week |
| | | | | 19-21/muFc | | | |
| 4 | Normal | 25-32 | 8 | Vehicle | 5 | 3x/week | 6 week |
| | | | | (muFc) | | | |

To assess changes in lean and fat masses in the diabetic and age matched normal mice treated with 2x mTN8-19-21/muFc, the body composition was measured using Bruker Minispec NMR (Echo Medical Systems, Houston, Tex.) at the beginning (day 0), 2 weeks (day 15), 4 weeks (day 30) and at the end of the study (day 45).

At the end of the study (day 45), the mice were detained in individual metabolic cages for 24 hours for urine collection. The 24-h urine volume was measured gravimetrically, and urinary albumin concentration was determined with an enzyme-linked immunosorbent assay using a murine microalbumin-uria assay kit (Alpha Diagnostic, San Antonio, Tex.).

Renal function was evaluated by calculating creatinine clearance rate. The plasma and urinary creatinine levels were measured by an enzymatic method (CRE, Mizuho medy, Saga, Japan) using the autoanalyzer Hitachi 717 Clinical Chemistry Auto Analyzer (Boehringer Mannheim, Indianapolis, Ind.). The blood urea nitrogen levels were measured by using the autoanalyzer.

All animals were terminated upon completion of the study (day 46). Mice were euthanized in CO₂ chamber and cardiac blood samples were collected and whole body tissue dissection was performed. Serum samples and stored at −80° C. for biochemistry analysis. Serum levels of blood glucose, blood urine nitrogen (BUN), creatinine levels were measured. Immediately following euthanization, the gastrocnemius muscle, and lean carcass mass were removed and weighted. Half middle portion of right side kidney was fixed with isopentane N₂ solution, and embedded in paraffin. The slices were stained with H&E and PSA (periodic acid-Schiff) for analysis glomerular structures.

The results were expressed as mean±standard error of the mean (SEM). Non-pair T-test was performed to determine statistical differences between groups. Statistical significant was considered when p value less than 0.05.

Results: Body Weight and Blood Glucose Changes in STZ Induced Diabetic Mice

Multiple low dose STZ injection on body weight and blood glucose of C57B1/6 mice resulted in STZ treated mice having significantly higher blood glucose levels than that the age matched normal mice group, the average of 20 animals beginning at normal levels of an average of about 120 mg/dl average blood sugar for 20 animals, increasing to an average of about 250-280 mg/dl at week 2 after STZ injection, and up to between 350 mg/dl 8 to 18 weeks after injection. Statistically significant differences were found on body weight changes between STZ treated and control group throughout the 4 month period before starting the anti-myostatin peptibody treatment. The control group steadily gained body weight, averaging a weight gain of up to 40% over 20 weeks (average of 25 g increasing up to 34 or 35 grams after 20 weeks), whereas the STZ group gained little weight over the 20 week period, increasing only about 12 to 14% over 20 weeks (25 g to about 28 or 29 g after 20 weeks).

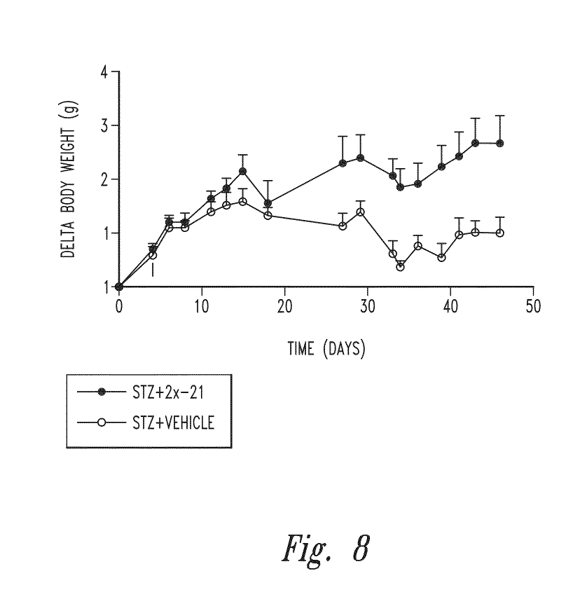

The six week treatment with 2x mTN8-19-21/muFc and vehicle in STZ diabetic and age matched normal mice treatment for 6 weeks resulted in significantly increased body weight gain in 2x-21 treated STZ diabetic mice compared to that of the vehicle treated diabetic group. Total body weight increased up to about 1.5 grams in addition for the STZ-treated mice receiving 2x-21 compared with the mice receiving the vehicle. The delta body weight are presented as the net changes in body weight after the 6 weeks treatment with 2x mTN8-19-21/muFc or vehicle compared to their respective day 0 baseline value. This is shown in FIG. 8. The 6 weeks treatment with 2x-21 significantly attenuated the body weight loss in diabetic animals.

Body Composition Changes in STZ Diabetic and Age Matched Normal Mice Treated with 2x-21

The lean body mass are presented as the net changes in lean body mass after the 6 week treatment with 2x-21 or vehicle compared to their day 0 baseline values. These values are presented in the Table below. Treatment with 2x-21 significantly increase (p<0.05) the net gain of lean body mass in both the STZ diabetic mice and age matched normal mice (6.16±0.81 g and 8.56±0.75 g) as compared to vehicle-treated control mice (0.94±1.94 g and 1.60±1.28 g). The % change of fat mass represent the net change after 6 week treatment with 2x-21 or vehicle compared to their baseline day 0 values in each group (see second Table below). The % of fat mass gain in STZ diabetic mice did not differ significantly between 2x-21 (−15.60±7.01) and vehicle treated group (−21.59±6.84). 2x-21 treatment decreased net fat mass gain in age matched normal mice (−1.53±3.42 vs. 7.13±3.38) but did not reach statistically significant amounts.

TABLE
Effect of 2X-21 on body lean mass in STZ-induced diabetic mice and age-
matched normal mice (NMR measurement)

| | | Body fat mass | | | |
|---|---|---|---|---|---|
| | Treatment | Baseline (g) | | | |
| | Sc. Injection | (g) | % Change | % Change | % Change |
| Animal | 5 mg/kg, 3/wk | D0 | D15 | D30 | D45 |
| STZ-diabetic | Mu-Fc | 20.33 ± 0.33 | (2.85 ± 1.79) | (2.50 ± 1.42) | (0.94 ± 1.93) |
| mice | | | | | |
| | 2x-21 | 20.16 ± 0.26 | (3.75 ± 1.34) | (6.50 ± 0.89)* | (6.16 ± 0.81)* |
| Normal | Mu-Fc | 22.38 ± 0.57 | (1.82 ± 1.18) | (3.87 ± 1.21) | (1.60 ± 1.28) |
| C57BL/6 | | | | | |
| Mice | | | | | |
| | 2x-21 | 21.82 ± 0.42 | (3.15 ± 0.74) | (7.60 ± 1.05)* | (8.56 ± 0.75)* |

TABLE
Effect of 2X-21 on body fat mass in STZ-induced diabetic mice and age-matched
normal mice (NMR measurement)

| | | Body fat mass | | | |
|---|---|---|---|---|---|
| | Treatment | Baseline | | | |
| | Sc. Injection | (g) | % Change | % Change | % Change |
| Animal | 5 mg/kg, 3/wk | D0 | D15 | D30 | D45 |
| STZ-diabetic | Mu-Fc | 3.13 ± 0.36 | (−12.73 ± 7.66) | (−16.61 ± 6.16) | (−21.59 ± 6.84) |
| mice | | | | | |
| | 2x-21 | 2.95 ± 0.22 | (−15.43 ± 4.14) | (−14.66 ± 6.83) | (−15.60 ± 7.01) |
| Normal | Mu-Fc | 8.43 ± 0.54 | (−4.76 ± 1.10) | (1.91 ± 2.74) | (7.13 ± 3.38) |
| C57BL/6 | | | | | |
| Mice | | | | | |
| | 2x-21 | 8.90 ± 0.56 | (−7.08 ± 0.52) | (−6.14 ± 2.75) | (−1.53 ± 3.42) |

Blood glucose changes in STZ diabetic and age matched normal mice treated with 2x -21

The Table below shows the effect of 2x mTN8-19-21/muFc on blood glucose changes in STZ diabetic and age matched normal mice. The blood glucose levels did not differ significantly between the 2x-21 treated and the vehicle treated groups in either STZ diabetic mice or in the age matched normal mice.

TABLE
Effect of 2X-21 on blood glucose level in STZ-induced diabetic mice and
age-matched normal mice

| | Treatment | | | |
|---|---|---|---|---|
| | Sc. | Blood Glucose | | |
| | Injection | Baseline | | |
| | 5 mg/ | (mg/dl) | | % Change |
| Animal | kg, 3/wk | D0 | D15 | D30 |
| STZ- | Mu-Fc | 430.50 ± 19.15 | (5.53 ± 7.81) | (9.44 ± 7.51) |
| diabetic | | | | |
| mice | | | | |
| | 2x-21 | 425.63 ± 20.99 | (6.68 ± 2.26) | (−3.70 ± 10.35) |
| Normal | Mu-Fc | 123.50 ± 3.26 | (9.56 ± 1.49) | (7.46 ± 5.80) |
| C57BL/6 | | | | |
| Mice | | | | |
| | 2x-21 | 122.88 ± 3.75 | (3.84 ± 2.83) | (6.20 ± 2.52) |

Kidney Weight/Body Weight:

The hyperglycemia in STZ diabetic mice appears to be associated with kidney hypertrophy. The kidney weight over body weight ratio of STZ diabetic mice was higher than that in age matched normal mice (0.98±0.04 vs. 0.67±0.02). 2x-21 treatment for 6 weeks significantly reduced the kidney/body weight ratio from 0.98±0.04 to the value of 0.84±0.04 (p<0.05) in vehicle treated diabetic mice.

Creatinine Clearance Rate

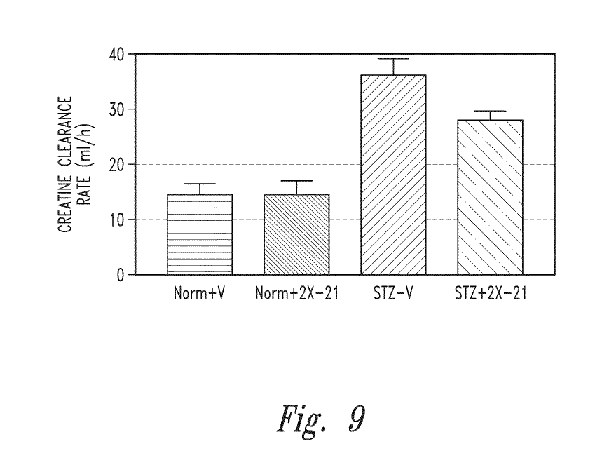

There was a trend for diabetic mice to increase creatinine clearance rate compared to non-diabetic normal control mice (FIG. 9). The average creatinine clearance rate of diabetic mice was more than two fold higher than the age matched normal mice. Treatment with 2x-21 decreased creatinine clearance rate in diabetic mice compared to vehicle treated diabetic mice as shown in FIG. 9, indicating kidney function.

24-Hour Urine Volume and Urinary Albumin Excretion:

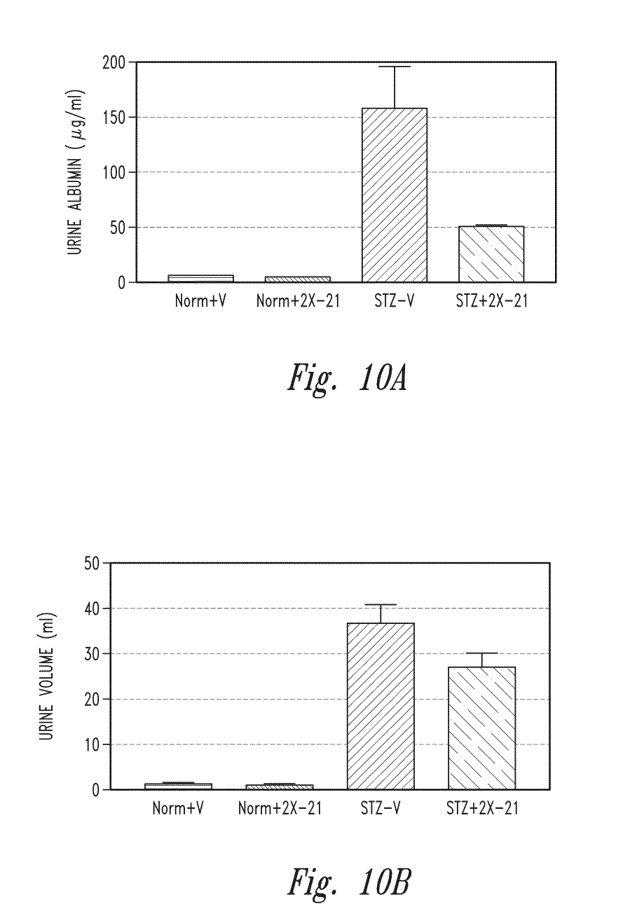

Urinary albumin excretion and 24-hour urine volume are very important biomarkers in determination of renal injury during the early stage of diabetic nephropathy. The results demonstrated that both urine albumin excretion (FIG. 10A) and 24 hour urine volume were increased in STZ diabetic mice as compared to age matched normal mice. 2x-21 treatment decreased urine albumin levels in diabetic mice and also reduced the 24 hour urine volume (FIG. 10B). This demonstrated a normalization of kidney function.

Administration of myostatin peptibody 2x mTNF8-19-21/muFc significantly attenuated the body weight loss and preserved skeletal muscle mass and lean body mass in STZ-induced diabetic mice. In addition to an increase in skeletal muscle and lean mass, 2x mTN8-19-21/muFc attenuated kidney hypertrophy, the increase in creatinine clearance rate and reduced 24 hour urine volume and urinary albumin excretion in STZ-induced diabetic mice. This shows improved kidney function in the early stage of development of diabetic nephropathy.

### Example 16

### Effects of Myostatin Antagonist in a Murine Model of 5-Fluorouracil Chemotherapy-Induced Cachexia

The compound 5-fluorouracil (5-Fu) is commonly used as a therapeutic agent in patients with colorectal, breast, stomach or pancreatic cancer. A side effect of 5-Fu therapy is body weight loss and muscle atrophy. The potential therapeutic benefit of anti-myostatin antagonist therapy in treating 5-Fu-induced cachexia was investigated. The peptibody used was 2x mTN8-19-21/muFc (also referred to as 2x-21) or 2x mTN8-19-21 attached to a murine Fc. The control vehicle was murine Fc alone.

In this study, normal male C57B1/6 mice were divided into 4 groups (n=24) and subjected to intraperitoneally (IP) administered 5-Fu (45 to 50 mg/kg) or vehicle phosphate-buffered solution (PBS) for 5 consecutive days (day 0 to day 4). Two groups were pretreated with 2x21, at 10 mg/kg twice weekly, starting at 2 weeks (day −13) or 1 week (day −6) before 5-Fu treatment began (on day 0), and continued after 5-Fu treatment to the end of the study on day 24. Body weight, lean body mass, and food intake were monitored twice per week or more frequently before and after 5-Fu therapy. Serum was collected at 0, 2, 24, 96, 168, 336 hours after last dosing for terminal study.

On day 0 and prior to 5-FU therapy, average body weight changes of the groups pretreated with 2x21 for 1 or 2 weeks were 12.6% and 13.9%, respectively, compared with 6.4% for the 5-Fu control group (both p<0.0001). This was paralleled with 14.7% and 16.2% increase in lean body mass in the groups pretreated for 1 or 2 weeks with peptibody compared with 7.4% increase in the 5-Fu only group (p=0.001 and p<0.0001). On day 6 post 5-Fu dosing, the body weight changes of the 1 or 2 weeks 2x21 pretreated groups were −1.9% and −1.4% compared with −8.6% of 5-Fu only group (both p values were <0.0001); lean body mass changed to −1.3% and −0.9% compared to −8.8% of 5-FU only group (both p values<0.0001). On day 8 during recovery, body weight changes of the 1 or 2 weeks 2x21 pretreated groups significantly increased to 6.8% and 8.5%, respectively, compared with the 0.6% increase in the 5-Fu only group (p=0.0006 and p<0.0001). Similarly, lean body mass changed to 4.9% and 6.0% in the 1 or 2 weeks. 2x21 pretreated groups compared to −3.3% for the 5-Fu only group (p=0.001 and p<0.0001 respectively). The results are summarized in FIG. 11.

From day 8 to day 24, almost all mice developed severe neutropenia and some mice died due to severe side effects. The survival rates for groups pretreated for 1 or 2 weeks with 2x21 prior to 5-Fu administration were 46%, compared to 13% survival rate for 5-Fu only group (p=0.001 and p=0.009, respectively). The survival results are summarized in FIG. 12.

Statistical analysis using ANOVA repeat measurement methods indicated that groups pretreated for 1 or 2 weeks with 2x21 peptibody prior to 5-Fu treatment, had significantly higher body weight and lean body mass throughout the course of the study, from day −13 to day 8, compared with the group treated with 5-Fu only (p values for both less than 0.0001).

Results from this study demonstrated that pretreatment with anti-myostatin peptibody, 2x21, at 10 mg/kg twice weekly, for 1 or 2 weeks was effective in significantly ameliorating 5-Fu induced body weight loss and muscle atrophy in C57B1/6 mice. In addition, pretreatment with the peptibody increased the survival rate and duration in response to the 5-Fu chemotherapy. Therefore, myostatin antagonists such as the myostatin binding agents of the present invention can be used prior to and during treatment with chemotherapeutics or other chemical agents to prevent or ameliorate chemical cachexia.

The present invention is not to be limited in scope by the specific embodiments described herein, which are intended as single illustrations of individual aspects of the invention, and functionally equivalent methods and components are invention. Indeed, various modifications of the invention, in addition to those shown and described herein will become apparent to those skilled in the art from the foregoing description and accompanying drawings. Such modifications are intended to fall within the scope of the appended claims.

1. A method of increasing lean muscle mass in a subject suffering from the effects of hypogonadism resulting from androgen deprivation therapy comprising administering a therapeutically effective amount of a myostatin antagonist in admixture with a pharmaceutically acceptable carrier to the subject, wherein the myostatin antagonist is a myostatin binding agent, wherein the binding agent comprises at least one peptide capable of binding myostatin, wherein the peptide comprises the sequence
(SEQ ID NO: 353),
Cb₁b₂Wb₃WMCPP
wherein b₁ is selected from any one of the amino acids T, I, or R; b₂ is selected from any one of R, S, Q; b₃ is selected from any one of P, R and Q,
and physiologically acceptable salts thereof.

2. The method of claim 1, wherein the peptide is between 20 and 50 amino acids in length.

3. The method of claim 1, wherein the peptide is selected from the group consisting of SEQ ID NO: 305-308, 310-313, 315-331, 333, and 335-346.

4. The method of claim 1, wherein the peptide is SEQ ID NO:325.

5. The method of claim 1, wherein the myostatin binding agent has the structure:
(X¹)ₐ—F¹—(X²)_{b}, or multimers thereof;
wherein F¹ is a vehicle; and X¹ and X² are each independently selected from
-(L¹)_{c}-P¹;
-(L¹)_{c}-P¹-(L²)_{d}-P²;
-(L¹)_{c}-P¹-(L²)_{d}-P²-(L³)ₑ-P³;
and -(L¹)_{c}-P¹-(L²)_{d}-P²-(L³)ₑ-P³-(L⁴)_{f}-P⁴;
wherein P¹, P², P³, and P⁴ are peptides capable of binding myostatin, and each comprise Cb₁b₂Wb₃WMCPP (SEQ ID NO: 353), wherein b₁ is selected from any one of the amino acids T, I, or R; b₂ is selected from any one of R, S, Q; b₃ is selected from any one of P, R and Q,
wherein L¹, L², L³, and L⁴ are each linkers;
and subscripts a, b, c, d, e, and f are each independently 0 or 1, provided that at least one of a and b is 1, and physiologically acceptable salts thereof.

6. The method of claim 5, wherein the peptide is between 20 and 50 amino acids in length.

7. The method of claim 5, wherein the peptide is selected from the group consisting of SEQ ID NO: 305-308, 310-313, 315-331, 333, and 335-346.

8. The method of claim 5, wherein the peptide is SEQ ID NO:325.

9. The method of claim 5, wherein the vehicle F¹ is an Fc domain.

10. The method of claim 5, wherein the vehicle F¹ is an Fc domain of human IgG1.

11. The method of claim 5, wherein a is 0 and b is 1.

12. The method of claim 5, wherein a is 1 and b is 0.

13. The method of claim 5, wherein the binding agent has the structure F¹-(L¹)_{c}-P¹ or multimers thereof.

14. The method of claim 5, wherein the linkers comprise (Gly)₅ (SEQ ID NO:635).

15. The method of claim 5, wherein the binding agent further comprises AQ between the linkers and the peptides.

16. The method of claim 5, wherein the binding agent further comprises AQ and LE between the linkers and the peptides.

17. A method of increasing lean muscle mass in a subject suffering from the effects of hypogonadism resulting from androgen depriviation therapy comprising administering a therapeutically effective amount of a myostatin binding agent in admixture with a pharmaceutically acceptable carrier to the subject, wherein the myostatin binding agent is a dimer of the structure F¹-(L¹)_{c}-P¹, wherein F¹ is a human IgG Fc domain, wherein P¹ is a peptide capable of binding myostatin, and comprising the sequence SEYQGLCTRWPWMCPPQGWK (SEQ ID NO: 325), wherein L¹ comprises (Gly)₅ (SEQ ID NO:635), and c is 1, and physiologically acceptable salts thereof.

18. The method of claim 17, wherein the binding agent further comprises AQ.
